(19) United States
(12) Patent Application Publication
(10) Pub. No.: US 2015/0238601 A1
(43) Pub. Date: Aug. 27, 2015

(54) TREATMENT OF OCULAR DISORDERS

(71) Applicant: KADMON CORPORATION, LLC, New York, NY (US)

(72) Inventors: Michael A. Boxer, East Hampton, NY (US); John L. Ryan, Philadelphia, PA (US); James R. Tonra, Skillman, NJ (US)

(21) Appl. No.: 14/431,948

(22) PCT Filed: Oct. 7, 2013
(86) PCT No.: PCT/US2013/063755

§ 371 Date: Mar. 27, 2015

Related U.S. Application Data
(60) Provisional application No. 61/710,467, filed on Oct. 5, 2012.

(51) Int. Cl. A61K 39/395; A61K 31/541; A61K 31/5377; A61K 31/506

(52) U.S. Cl. CPC A61K 39/3955; A61K 31/506; A61K 31/541; A61K 31/5377

(57) ABSTRACT

The invention provides methods of treatment of ocular disorders, including ocular disease with an angiogenic component. In certain embodiments, the treatment comprises administration of a ROCK2 inhibitor and an angiogenesis inhibitor. In certain embodiments, the ROCK2 inhibitor is ROCK2 selective. In certain embodiments, the angiogenesis inhibitor is a VEGF antagonist, for example, and VEGFR2 antibody.

### FIELD OF THE INVENTION

The invention relates to treatment of ocular disorders, including ocular disorders with an angiogenic component. In certain embodiments, the treatment comprises administration of a ROCK2 inhibitor and an angiogenesis inhibitor. In certain embodiments, the angiogenesis inhibitor is a VEGF antagonist. In certain embodiments, the angiogenesis inhibitor is a VEGFR2 antibody. The invention further provides novel ROCK inhibitors and novel VEGFR2 antibodies.

### CROSS-REFERENCE TO RELATED APPLICATIONS

This application claims priority to U.S. Application No. 61/710,467, filed Oct. 5, 2012, which is incorporated herein by reference in its entirety.

### BACKGROUND OF THE INVENTION

Rho-associated protein kinase (ROCK) is a key intracellular regulator of cytoskeletal dynamics and cell motility. Rho-kinase regulates a number of downstream targets of RhoA through phosphorylation, including, for example, myosin light chain, the myosin light chain phosphatase binding subunit and LIM-kinase 2. These substrates regulate actin filament organization and contractility. In smooth muscle cells Rho-kinase mediates calcium sensitization and smooth muscle contraction. Inhibition of Rho-kinase blocks 5-HT and phenylephrine agonist induced muscle contraction. When introduced into non-smooth muscle cells, Rho kinase induces stress fiber formation and is required for the cellular transformation mediated by RhoA. Rho kinase participates in a variety of cellular processes, including but not limited to cell adhesion, cell motility and migration, growth control, cell contraction, and cytokinesis. Rho kinase is also involved in Na/H exchange transport system activation, stress fiber formation, adducin activation, and physiological processes such as vasoconstriction, bronchial smooth muscle constriction, vascular smooth muscle and endothelial cell proliferation, platelet aggregation, and others.

Inhibition of Rho-kinase activity in animal models has demonstrated a number of benefits of Rho-kinase inhibition for the treatment of human diseases. These include models of cardiovascular diseases such as hypertension, atherosclerosis, restenosis, cardiac hypertrophy, ocular hypertension, cerebral ischemia, cerebral vasospasm, penile erectile dysfunction, central nervous system disorders such as neuronal degeneration and spinal cord injury, and in neoplasias. Inhibition of Rho-kinase activity has been shown to inhibit tumor cell growth and metastasis, angiogenesis, arterial thrombotic disorders such as platelet aggregation and leukocyte aggregation, asthma, regulation of intraoccular pressure, and bone resorption. The inhibition of Rho-kinase activity in patients has benefits for controlling cerebral vasospasms and ischemia following subarachnoid hemorrhage, reduction of intraocular pressure, increase in ocular aqueous outflow by relaxation of trabecular meshwork tissue, improving blood flow to the optic nerve, and protection of healthy ganglion cells.

In mammals, Rho-kinase consists of two isoforms, ROCK1 (ROCKβ; p160-ROCK) and ROCK2 (ROCKα). ROCK1 and ROCK2 are differentially expressed and regulated in specific tissues. For example, ROCK1 is ubiquitously expressed at relatively high levels, whereas ROCK2 is preferentially expressed in cardiac and brain and skeletal muscle. The isoforms are also expressed in some tissues and in a developmental stage specific manner. ROCK1 is a substrate for cleavage by caspase-3 during apoptosis, whereas ROCK2 is not. Smooth muscle specific basic calponin is phosphorylated only by ROCK2.

Angiogenesis is a highly complex process of developing new blood vessels that involves the proliferation and migration of, and tissue infiltration by capillary endothelial cells from pre-existing blood vessels, cell assembly into tubular structures, joining of newly forming tubular assemblies to closed-circuit vascular systems, and maturation of newly formed capillary vessels.

Angiogenesis is important in normal physiological processes including embryonic development, follicular growth, and wound healing. Undue angiogenesis also leads to neovascularization in neoplastic diseases, and in non-neoplastic diseases such as age-related macular degeneration, diabetic retinopathy, and neovascular glaucoma. Anti-angiogenic therapy that targets vascular endothelial growth factor (VEGF) with ranibizumab (Lucentis) has been shown to be effective in delaying progression of AMD. However, neovascularization is complex and multiple angiogenic mechanisms are likely to contribute. There remains a need to develop agents and therapies for treating diseases associated with neovascularization.

Glaucoma is associated with higher-than-normal pressure inside the eye (ocular hypertension), and if uncontrolled, leads first to loss of peripheral vision loss and eventually can lead to blindness. The two major types of glaucoma are chronic or primary open-angle glaucoma (POAG) and acute angle-closure glaucoma, both of which are characterized by high intraocular pressure (IOP). Normal tension glaucoma causes visual field loss due to optic nerve damage, but is characterized by normal IOP. The cause is thought to be poor blood flow to the optic nerve. Pigmentary glaucoma is characterized by reduced outflow from the eye caused by pigment that has broken loose from the iris and clogged the drainage angle. Secondary glaucoma develops from an eye injury, infection, inflammation, a tumor or enlargement of the lens due to a cataract. Lastly, congenital glaucoma results from a defect, typically narrow angles, that results in defective drainage of the eye.

### SUMMARY OF THE INVENTION

The invention relates to treatment of disorders which have an angiogenic component. In certain embodiments, the disorder is an ocular disorder. According to the invention, such disorders are treated with a ROCK2 inhibitor, which can be a non-selective “pan-ROCK” inhibitor or a selective ROCK2 inhibitor. In certain such embodiments, an angiogenesis inhibitor is also administered. The invention also relates to treatment of ocular disorders without an angiogenic component such as certain glaucomas, with a ROCK inhibitor. The invention provides agents for use in the therapies.

Angiogenesis occurs in ocular diseases such as macular degeneration (AMD), choroidal neovascularization (CNV), diabetic macular edema (DME), iris neovascularization, uveitis, neovascular glaucoma, or retinitis of prematurity (ROP). Angiogensis also occurs in diseases such as cancer, rheumatoid arthritis, psoriasis, and more than 70) other conditions. In these conditions, new blood vessels feed diseased tissues, destroy normal tissues, and in the case of cancer, the new vessels allow tumor cells to escape into the circulation and lodge in other organs (tumor metastases) Excessive angiogenesis occurs when diseased cells produce abnormal amounts of angiogenic growth factors, overwhelming the effects of natural angiogenesis inhibitors.

Thus, the invention provides a method of treating an ocular disorder having an angiogenic component in a subject, which comprises administering to the subject an effective amount of a rho kinase inhibitor that inhibits ROCK2 and an angiogenesis inhibitor. The invention also provides a method of treating a certain non-ocular disorders having an angiogenic component, which comprises administering to the subject an effective amount of a rho kinase inhibitor that inhibits ROCK2 and an angiogenesis inhibitor. In an embodiment of the invention, the ROCK2 inhibitor is ROCK2-selective. ROCK2 inhibitors for use according to the invention include, without limitation, compounds set forth by Formulae I-XXV described herein. Additional ROCK2 inhibitors are set forth by Formulae XXXI-XXXVI, including the ROCK2-selective compound SLx-2119 is employed.

Angiogenesis inhibitors are also provided, and include, without limitation, VEGF antagonists such as the VEGFR2 antibodies described herein. In some embodiments, the VEGF antagonist is VEGF-specific, including, but not limited to Avastin and Lucentis.

In certain of the therapies of the invention, the method of treatment further comprises administration of a TGF-β antagonist.

Certain compounds of the invention have the formula I:

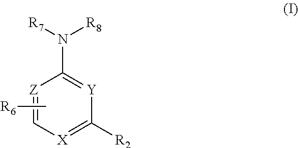

or a pharmaceutically acceptable salt thereof, wherein:
X is selected from N or C—R₁;
Y is selected from N or C—R₅;
Z is selected from N or C—R₃;
R₁ is selected from the group consisting of H, lower alkyl, CN, halo, hydroxy, lower alkoxy, amino, perfluoro lower alkyl, and (lower alkyl)-O-(lower alkyl);
R₂ is a group having the formula -A-R₁₀;
A is selected from the group consisting of a covalent bond, aryl, heteroaryl, cycloalkyl, and heterocyclyl;

R₁₀ is selected from the group consisting of H, CN, halo, hydroxy, lower alkoxy, amino, perfluoro lower alkyl, C₁-C₁₀ alkyl, C₂-C₁₀ alkenyl, and -(M)ₓ-(CH₂)_{y}—R₁₁;
M is selected from the group consisting of N—R₂₀, CR₂₁R₂₂, and C═O;
x is 0 or 1;
R₂₀ is selected from H and C₁₋₅ alkyl;
R₂₁ and R₂₂ are independently selected from the group consisting of H, halogen, and lower alkyl, or alternatively R₂₁ and R₂₂ may be taken together with the atom to which they are attached to form a C₃₋₆ cycloalkyl;
y is 0, 1, 2, 3, 4, 5, or 6;
R₁₁ is selected from the group consisting of H, C₁₋₆ alkyl, optionally substituted C₃₋₆ cycloalkyl, optionally substituted aryl, optionally substituted heteroaryl, optionally substituted heterocyclyl, wherein the optional substituents are selected from the group consisting of lower alkyl, C₁₋₆ cycloalkyl, oxo, CN, halo, hydroxy, lower alkoxy, amino, perfluoro lower alkyl, and (lower alkyl)-O-(lower alkyl);
alternatively R₁₁ is selected from the group consisting of —NR₁₃R₁₄, —C(═O)NR₁₃R₁₄, and —C(═O)R₁₂, and —CO₂R₁₂;
R¹² is selected from the group consisting of C₁-C₁₀ alkyl, aryl, heteroaryl, —(C₁-C₆ alkyl)-O—(C₁-C₆ alkyl), aralkyl, C₃-C₇ cycloalkyl, a three to twelve membered heterocyclic ring containing up to 3 heteroatoms, each of which may be optionally substituted by from 1 to 3 substituents independently selected from halo, oxo, C₁-C₆ alkyl, C₃-C₆ cycloalkyl, C₁-C₆ alkoxy, (C₁-C₆ alkyl)-O—(C₁-C₆ alkyl), hydroxy, cyano and C₁-C₃ perfluoro alkyl;
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), aryl, aralkyl, heteroaryl, C₃-C₆ cycloalkyl, a three to twelve membered heterocyclic ring containing up to 3 heteroatoms, each of which may be optionally substituted by from 1 to 3 substituents independently selected from halo, oxo, C₁-C₆ alkyl, C₂-C₆ alkenyl, C₃-C₇ cycloalkyl, C₁-C₆ alkoxy, (C₁-C₆ alkyl)-O—(C₁-C₆ alkyl), hydroxy, amino, cyano and C₁-C₃ perfluoro alkyl;
or R¹³ and R¹⁴ may be taken together form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, (C₁-C₆ alkyl)-O—(C₁-C₆ alkyl), oxo, hydroxy, cyano and C₁-C₃ perfluoro alkyl;
R₃ is selected from the group consisting of H, lower alkyl, CN, halo, hydroxy, lower alkoxy, amino, perfluoro lower alkyl, and (lower alkyl)-O-(lower alkyl);
R₅ is selected from the group consisting of H, lower alkyl, CN, halo, hydroxy, lower alkoxy, amino, perfluoro lower alkyl, and (lower alkyl)-O-(lower alkyl);
R₆ is selected from the group consisting of H, lower alkyl, CN, halo, hydroxy, lower alkoxy, amino, perfluoro lower alkyl, and (lower alkyl)-O-(lower alkyl);
R₇ is selected from the group consisting of H and lower alkyl; and
R₈ is a nitrogen-containing heterocyclic ring system ring which may comprise 0-2 additional ring heteroatoms selected from N, O and S, and may be unsubstituted or may be substituted with 1 to 3 substituents selected from halo, CN, oxo, hydroxy, amino, lower alkyl, perfluoro lower alkyl, and lower alkoxy.

Other compounds of the invention are set forth below.

The present invention includes pharmaceutical compositions comprising the compounds of the invention and a pharmaceutically acceptable carrier and/or diluents.

The invention also provides angiogenesis inhibitors useful in treating disorders with an angiogenic component, including novel anti-VEGFR2 antibodies, nucleic acids encoding such antibodies and compositions comprising such antibodies. In one embodiment the invention provides an isolated heavy chain variable region comprising a CDR-1H, CDR-2H, and CDR-3H sequence, wherein:

(i) the CDR-1H sequence is GFTFSWYX₁MX₂ (SEQ ID NO:185), wherein X₁ is V or I, X₂ is G or L,

(ii) the CDR-2H sequence is SIX₁X₂SGGX₃TX₄YADSVKG (SEQ ID NO:186), wherein X₁ is Y or G, X₂ is P or S, X₃ is A or F, X₄ is N or D, and

(iii) the CDR-3H sequence is GNYFDY (SEQ ID NO:3) or GLAAPRS (SEQ ID NO:11).

In one embodiment, the invention provides an isolated light chain variable region comprising a CDR-1L, CDR-2L, and CDR-3L, wherein

(i) the CDR-1L sequence is X₁GX₂X₃LX₄X₅X₆X₇X₈S (SEQ ID NO:187), wherein X₁ is S, Q, or T, X₂ is D, E, or Q, X₃ is K, S, N, I, or A, X₄ is G or R, X₅ is D, S, H, E, or N, X₆ is E, Y, Q, R, or N, X₇ is Y, F, or S, and X₈ is A or S, or SGSX₁SNX₂X₃X₄X₅X₆X₇X₈ (SEQ ID NO: 188), wherein X₁ is S, or T, X₂ is I or L, X₃ is E or G, X₄ is T, S, or N, X₅ is N or Y, X₆ is T. P, A, or Y, X₇ is V or L, and X₈ is N, I, or Y, or X₁GX₂SX₃DX₄GX₅YDYVS (SEQ ID NO: 189), wherein X₁ is A or T, X₂ is S or T, X₃ is H, S, or N, X₄ is I or V, and X₅ is S or A,

(ii) the CDR-2L sequence is X₁X₂X₃X₄X₅PS (SEQ ID NO:190), wherein X₁ is Q, D, T, Y, S, or A, X₂ is D, N, S, T, V, or V, X₃ is D, N, S, T, or Y, X₄ is Q, K, N, or L, and X₅ is R or L, and

(iii) wherein the CDR-3L sequence is QX₁WX₂X₃X₄X₅X₆X₇X₈ (SEQ ID NO:191), wherein X₁ is A or T, X₂ is D or G, X₃ is R or no amino acid, X₄ is S, F, on N, X₅ is S, T, on N, X₆ is S, T, or P, X₇ is A, V, L, I, or Y, and X₈ is V or L, or AX₁WDDX₂LX₃X₄X₅X₆ (SEQ ID NO:192), wherein X₁ is A, S, or T, X₂ is N or S, X₃ is N, I, or G, X₄ is G or S, X₅ is P, W, or V, and X₆ is V or L, or MYSTITX₁LL (SEQ ID NO:193), wherein X₁ is A or T.

In one embodiment, the invention provides an isolated light chain variable region comprising a CDR-1L, CDR-2L, and CDR-3L, wherein

(i) the CDR-1L sequence is RASX₁X₂X₃X₄X₅X₆X₇YX₅X₉ (SEQ ID NO:194), wherein X₁ is Q, E, or H, X₂ is S, R, or N, X₃ is V, I, or L, X₄ is S, R, G or N, X₅ is S or N, X₆ is S, N, W, or D, X₇ is G or no amino acid, X₈ is L or F, and X₉ is A, G, M, or S,

(ii) the CDR-2L sequence is GASX₁RAT (SEQ ID NO:195), wherein X₁ is S, T, I, or N, and

(iii) the CDR-3L sequence is QQX₁X₂X₃X₄X₅X₆X₇X₈ (SEQ ID NO: 196), wherein X₁ is F or Y, X₂ is D, G, or Y, X₃ is S, T, or N, X₄ is S, L, or W, X₅ is P or no amino acid, X₆ is P or T, X₇ is L, I, V, P, W, or Y, and X₈ is T or S.

In an embodiment of the invention, an antibody is provided which comprises a heavy chain variable domain comprising one, two, three, four, five, or six of the light chain variable domain and heavy chain variable domain CDR sequences set forth above. Examples of such antibodies are provided herein.

The invention also provides a method of treating glaucoma comprising administering an effective amount of a rho kinase inhibitor. Varieties of glaucoma to be treated include, without limitation, the two major types, chronic or primary open-angle glaucoma (POAG) and acute angle-closure glaucoma, as well as normal tension glaucoma, pigmentary glaucoma, secondary glaucoma that develops from an eye injury, infection, inflammation, a tumor or enlargement of the lens due to a cataract, and congenital glaucoma.

The invention also relates to non-ocular disorders having an angiogenic component. Thus, the invention provides a method of treating a disorder having an angiogenic component in a subject, which comprises administering to the subject an effective amount of a rho kinase inhibitor and an angiogenesis inhibitor. Non-limiting examples include atherosclerosis, rheumatoid arthritis (RA), hemangiomas, angiofibromas, psoriasis, corneal graft rejection, insulin-dependent diabetes mellitus, multiple sclerosis, myasthenia gravis, Chron's disease, autoimmune nephritis, primary biliary cirrhosis, acute pancreatitis, allograph rejection, allergic inflammation, contact dermatitis, delayed type hypersensitivity, inflammatory bowel disease, septic shock, osteoporosis, osteoarthritis, neuronal inflammation, Osler-Weber syndrome, restenosis, or fungal, parasitic or viral infection.

### BRIEF DESCRIPTION OF THE FIGURES

FIG. 1 shows compounds of the invention.

FIG. 2 shows compounds of the invention

FIG. 3 shows compounds of the invention.

FIG. 4 shows compounds of the invention.

FIG. 5 shows dose response curves for inhibition of ROCK1 vs. ROCK2. Compounds correspond to Examples herein, as follows: K100-5, Ex. 12; KD-25, SLx-2119; 3272, Ex. 28; K100-04, Ex. 14; K100-16, Ex. 43; K100-21; Ex. 38; K100-23 Ex. 52; K100-24, Ex. 111; K100-25, Ex. 56; K100-26, Ex. 13; 3266, Ex. 26.

FIG. 6 compares ROCK1 and ROCK2 inhibition among the compounds of Examples 43, 48, and 118.

FIG. 7 shows ROCK2 selective inhibitor, KD025 (SLx 2119), inhibits IL-17/IL-21 secretion (A) and proliferation (B) in human CD4⁺ T cells in vitro.

FIG. 8 shows ROCK2 siRNA, but not ROCK1 siRNA, inhibits IL-17 and IL-21 secretion.

FIG. 9 shows KD025 (SLx 2119) inhibits STAT3 phosphorylation. (A) Pre-treatment of T cells with KD025 followed by stimulation with anti-CD3/CD28 antibodies. (B) Cell culture under Th17-skewing conditions for 5 days followed by treatment with KD025 for 3 hours.

FIG. 10 shows ROCK2 selective inhibitor, KD025, inhibits IL-17, IL-21 and IFN-γ production ex vivo in CD3/CD28 stimulated CD4⁺ T cells from RA patients. Panel (A) shows reduced secretion of IL-17, IL-21 and IFN-γ in response to TCR stimulation. Panel (B) shows reduced frequency of both IL-17 and IFN-γ-producing cells demonstrated by intracellular staining.

FIGS. 11A-C show human heavy chain, lambda light chain, and kappa light chain variable region sequences, respectively, of anti-VEGFR2 antibodies identified by phage display.

FIG. 12 shows binding of antibodies of the invention to hVEGFR2 (top) and a construct containing domains 2 and 3 of hVEGFR2 (middle). The bottom panel shows ligand (VEGF₁₆₅) blocking.

FIG. 13 shows Mabs 101 and 102 of the invention inhibit VEGFA-stimulated phosphorylation of VEGFR2, AKT, and MAPK in porcine aortic endothelial (PAE) cells overexpressing KDR (human VEGFR2).

FIG. 14 shows binding to hVEGFR2 and VEGF₁₆₅ ligand blocking by Mabs 104, 105, 106, and 108. Similar results were obtained for Mabs 103, 107, 109, and 110 in a separate experiment. These Mabs contain the heavy chain variable domain of Mab 101, recombined with different light chain variable domains.

FIG. 15 shows Mabs 105 and 106 of the invention inhibit VEGFA-stimulated phosphorylation of VEGFR2, AKT, and MAPK in porcine aortic endothelial (PAE) cells overexpressing KDR (human VEGFR2).

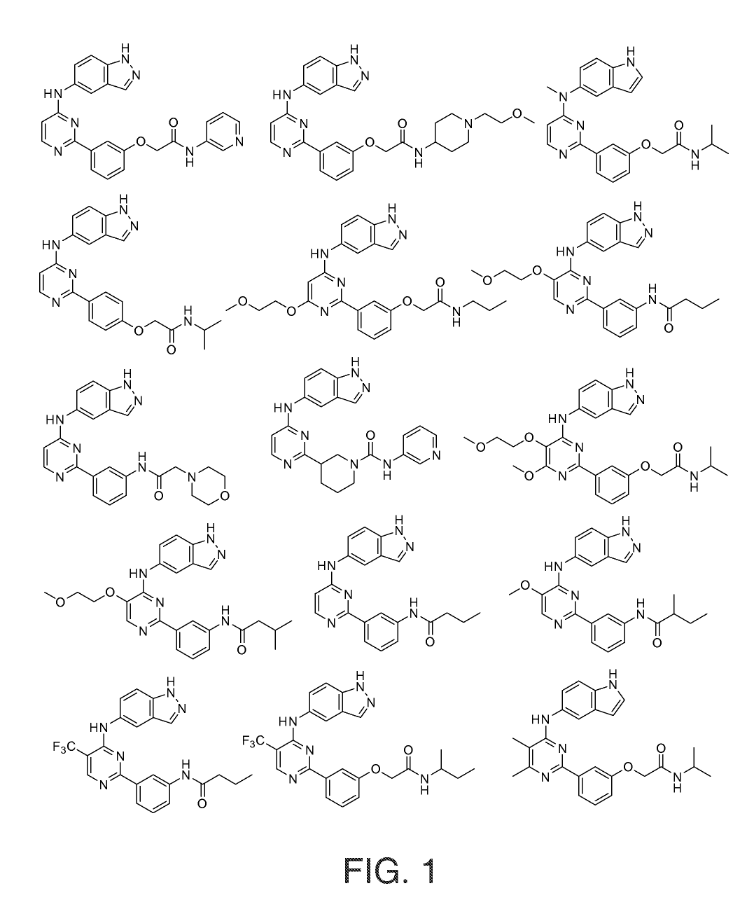

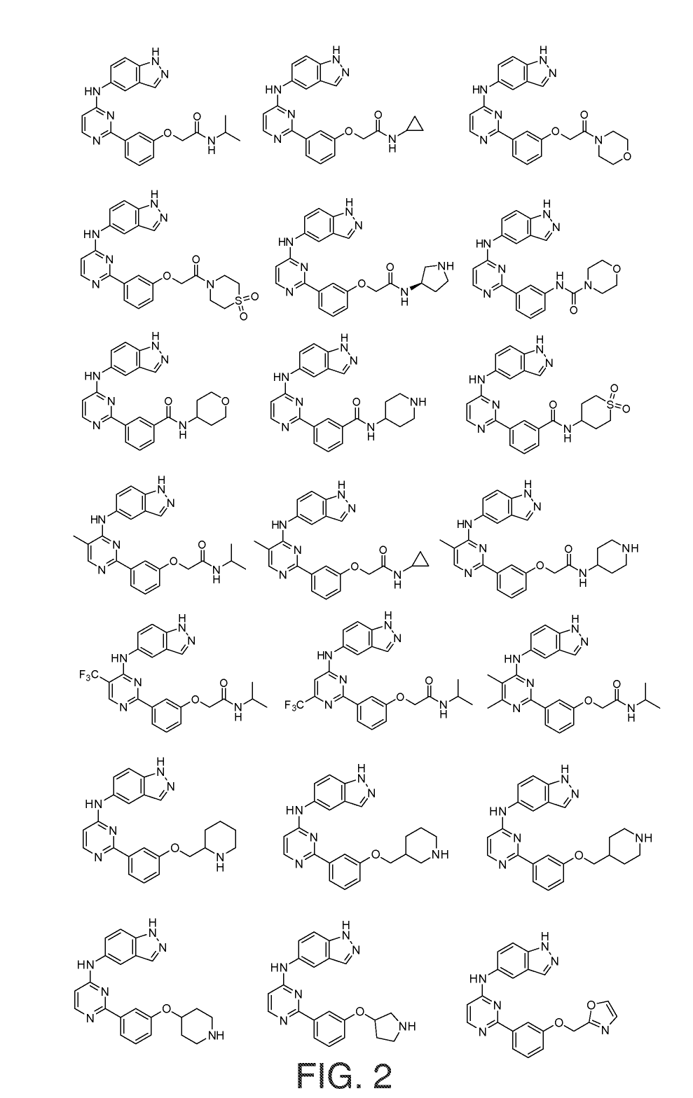

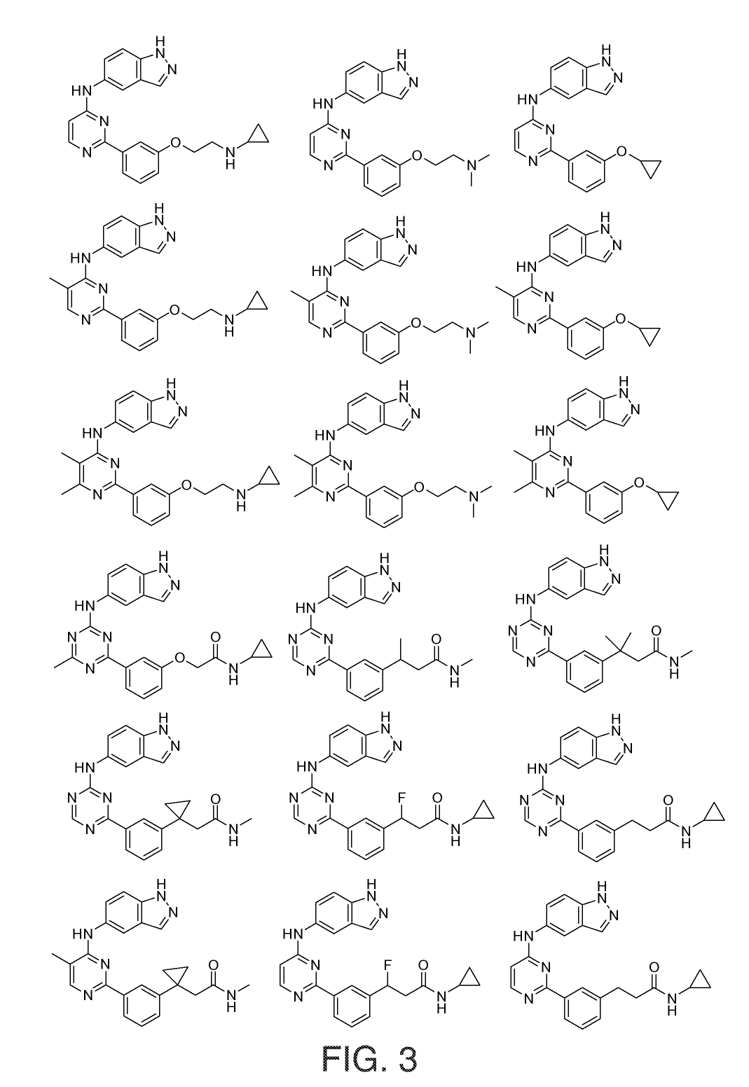

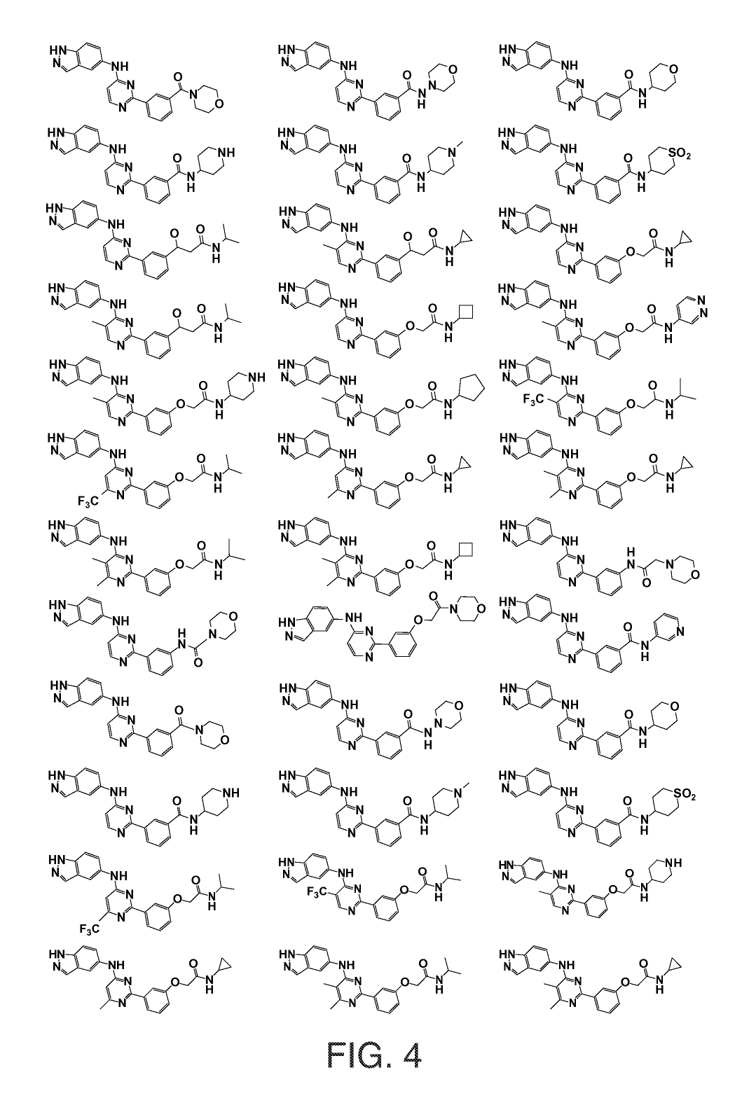

### DETAILED DESCRIPTION

The invention relates to treatment of disorders which have an angiogenic component. In certain embodiments, the disorder is an ocular disorder. According to the invention, such disorders are treated with a ROCK2 inhibitor, which can be a non-selective “pan-ROCK” inhibitor or a selective ROCK2 inhibitor. In certain such embodiments, an angiogenesis inhibitor is also administered. The invention also relates to treatment of ocular disorders without an angiogenic component such as certain glaucomas, with a ROCK inhibitor.

In one aspect, the present invention provides compounds of Formula I that are inhibitors of Rho-kinase. Rho kinase (ROCK), a serine/threonine kinase, serves as a target protein for small GTP-binding protein Rho, and is an important mediator of numerous cellular functions, including focal adhesions, motility, smooth muscle contraction, and cytokinesis. In smooth muscle, ROCK plays an important role in Ca²⁺ sensitization and the control of vascular tone. It modulates the level of phosphorylation of the myosin II light chain of myosin II, mainly through inhibition of myosin phosphatase, and contributes to agonist-induced Ca²⁺ sensitization in smooth muscle contraction. In certain embodiments, the compound of Formula I selectively inhibits ROCK2.

Compounds useful according to the present invention include those having the formula I:

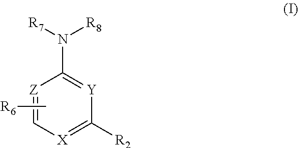

wherein:
X is selected from N or C—R₁;
Y is selected from N or C—R₅;
Z is selected from N or C—R₃;
R₁ is selected from the group consisting of H, lower alkyl, CN, halo, hydroxy, lower alkoxy, amino, perfluoro lower alkyl, and (lower alkyl)-O-(lower alkyl);
R₂ is a group having the formula -A-R₁₀;
A is selected from the group consisting of a covalent bond, aryl, heteroaryl, cycloalkyl, and heterocyclyl;
R₁₀ is selected from the group consisting of H, CN, halo, hydroxy, lower alkoxy, amino, perfluoro lower alkyl, C₁-C₁₀ alkyl, C₂-C₁₀ alkenyl, and -(M)ₓ-(CH₂)_{y}—R₁₁
M is selected from the group consisting of N—R₂₀, CR₂₁R₂₂, and C═O;
x is 0 or 1;
R₂₀ is selected from H and C₁₋₅ alkyl;
R₂₁ and R₂₂ are independently selected from the group consisting of H, halogen, and lower alkyl, or alternatively R₂₁ and R₂₂ may be taken together with the atom to which they are attached to form a C₃₋₆ cycloalkyl;
y is 0, 1, 2, 3, 4, 5, or 6;
R₁₁ is selected from the group consisting of H, C₁₋₆ alkyl, optionally substituted C₃₋₆ cycloalkyl, optionally substituted aryl, optionally substituted heteroaryl, optionally substituted heterocyclyl, wherein the optional substituents are selected from the group consisting of lower alkyl, C₁₋₆ cycloalkyl, oxo, CN, halo, hydroxy, lower alkoxy, amino, perfluoro lower alkyl, and (lower alkyl)-O-(lower alkyl);
alternatively R₁₁ is selected from the group consisting of —NR₁₃R₁₄, —C(═O)NR₁₃R₁₄, and —C(═O)R₁₂, and —CO₂R₁₂;
R¹² is selected from the group consisting of C₁-C₁₀ alkyl, aryl, heteroaryl, —(C₁-C₆ alkyl)-O—(C₁-C₆ alkyl), aralkyl, C₃-C₇ cycloalkyl, a three to twelve membered heterocyclic ring containing up to 3 heteroatoms, each of which may be optionally substituted by from 1 to 3 substituents independently selected from halo, oxo, C₁-C₆ alkyl, C₃-C₆ cycloalkyl, C₁-C₆ alkoxy, (C₁-C₆ alkyl)-O—(C₁-C₆ alkyl), hydroxy, cyano and C₁-C₃ perfluoro alkyl;
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), aryl, aralkyl, heteroaryl, C₃-C₆ cycloalkyl, a three to twelve membered heterocyclic ring containing up to 3 heteroatoms, each of which may be optionally substituted by from 1 to 3 substituents independently selected from halo, oxo, C₁-C₆ alkyl, C₂-C₆ alkenyl, C₃-C₇ cycloalkyl, C₁-C₆ alkoxy, (C₁-C₆ alkyl)-O—(C₁-C₆ alkyl), hydroxy, amino, cyano and C₁-C₃ perfluoro alkyl;
or R¹³ and R¹⁴ may be taken together form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, (C₁-C₆ alkyl)-O—(C₁-C₆ alkyl), oxo, hydroxy, cyano and C₁-C₃ perfluoroalkyl;
R₃ is selected from the group consisting of H, lower alkyl, CN, halo, hydroxy, lower alkoxy, amino, perfluoro lower alkyl, and (lower alkyl)-O-(lower alkyl);
R₅ is selected from the group consisting of H, lower alkyl, CN, halo, hydroxy, lower alkoxy, amino, perfluoro lower alkyl, and (lower alkyl)-O-(lower alkyl);
R₆ is selected from the group consisting of H, lower alkyl, CN, halo, hydroxy, lower alkoxy, amino, perfluoro lower alkyl, and (lower alkyl)-O-(lower alkyl);
R₇ is selected from the group consisting of H and lower alkyl; and
R₈ is a nitrogen-containing heterocyclic ring system ring which may comprise 0-2 additional ring heteroatoms selected from N, O and S, and may be unsubstituted or may be substituted with 1 to 3 substituents selected from halo, CN, oxo, hydroxy, amino, lower alkyl, perfluoro lower alkyl, and lower alkoxy.

In certain embodiments of the invention, the ring system of R₈ is saturated, contains one or more double bonds, or is aromatic. The ring system than comprises R₈ is preferably a monocyclic or a bicyclic ring system having 4 to 10 ring atoms. In certain aspects of the invention, R₈ is selected from:

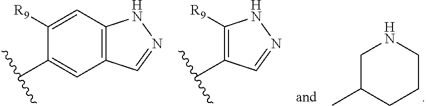

wherein R₉ is selected from H, halogen and lower alkyl.

In certain embodiments, R₂ is a substituted aryl group and is preferably a substituted phenyl group.

In certain aspects of the invention, the compounds useful according to the present invention include those having the formula II, III or IV:

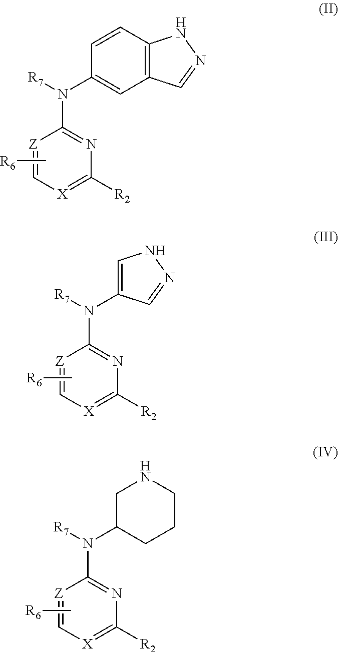

wherein R₂, R₆, R₇, X and Z are as defined above for formula I.

In other aspects of the invention, the compounds useful according to the present invention include those having the formula V or VI:

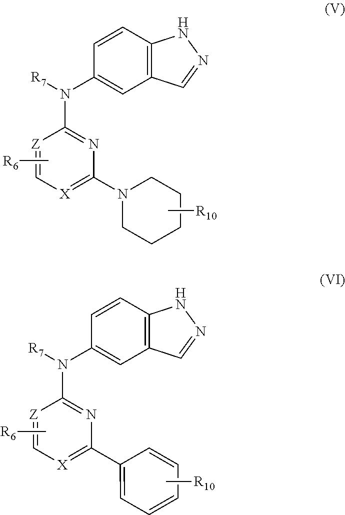

wherein R₆, R₇, X, Z and R₁₀ are as defined above for formula I.

In other aspects of the invention, the compounds useful according to the present invention include those having the formula VII:

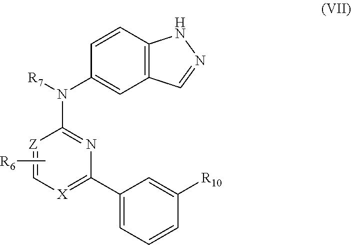

wherein R₆, R₇, X, Z, and R₁₀ are as defined above for formula I.

In other aspects of the invention, the compounds useful according to the present invention include those having the formula IX:

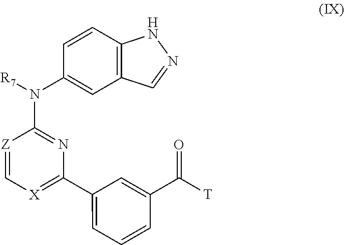

wherein R₆, R₇, X and Z are as defined above for formula I, and T is —(CH₂)_{y}—R₁₁ wherein y and R₁₁ are as defined above for formula I.

In other aspects of the invention, the compounds useful according to the present invention include those having the formula X:

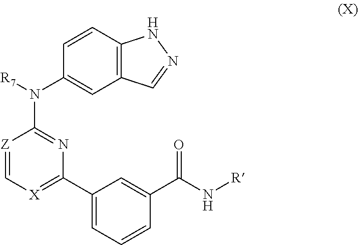

wherein R₆, R₇, X and Z are as defined above for formula I, and R′ is R₁₃ as defined above for formula I.

In other aspects of the invention, the compounds useful according to the present invention include those having the formula XI:

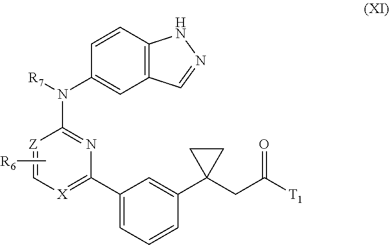

wherein R₆, R₇, X and Z are as defined above for formula I, and T₁ is R₁₂ as defined above for formula I.

In other aspects of the invention, the compounds useful according to the present invention include those having the formula XII:

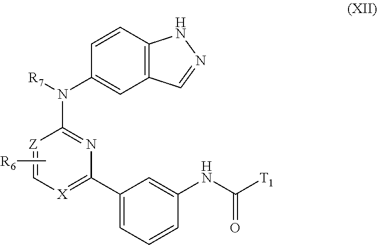

wherein R₆, R₇, X and Z are as defined above for formula I, and T₁ is R₁₂ as defined above for formula I.

In other aspects of the invention, the compounds useful according to the present invention include those having the formula XIII:

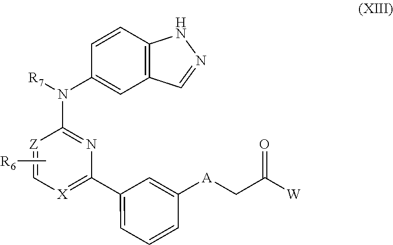

wherein R₆, R₇, X and Z are as defined above for formula I, A is M as defined above for formula 1 and W is R₁₂ as defined above for formula I.

In other aspects of the invention, the compounds useful according to the present invention include those having the formula XIV:

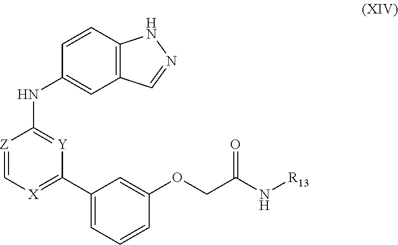

wherein X, Z and R¹³ are as defined above for formula I.

In certain aspects of the invention, for each of the compounds depicted above, the moiety

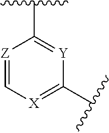

may be selected from a heteroaromatic group such that Y is N. In other aspects of the invention, both Y and X are N, and in still other aspects X, Y and Z are each N. In preferred aspects of the invention, this heteroaromatic group is selected from any one of the following groups:

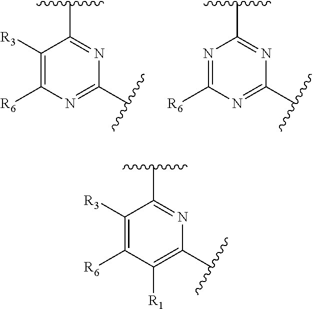

In other aspects of the invention, the compounds useful according to the present invention include those having the formula XVI:

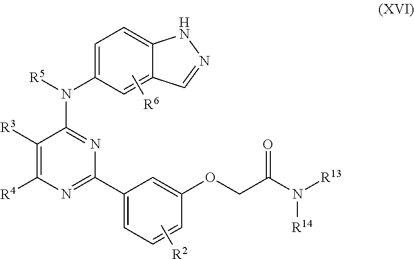

or pharmaceutically acceptable salt or stereoisomer thereof, wherein:
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), heteroaryl, C₃-C₇ cycloalkyl, a three to twelve membered heterocyclic or aromatic ring containing up to 3 heteroatoms, each of which may be optionally substituted by from 1 to 3 substituents independently selected from halo, oxo, C₁-C₆ alkyl, C₂-C₆ alkenyl, C₃-C₇ cycloalkyl, C₁-C₆ alkoxy, CN and C₁-C₃ perfluoro alkyl;
or R¹³ and R¹⁴ may be taken together form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, —OH, —NH₂, CN and C₁-C₃ perfluoro alkyl;
   R² is selected from H and halo;
   each R³ and R⁴ is independently selected from the group consisting of H, C₁-C₈ alkyl, —CN, halo, —OH, —O—(C₁-C₆ alkyl), —O—(C₁-C₆ alkyl)-O—(C₁-C₆ alkyl), —NR³¹R³², C₁-C₃ perfluoro alkyl, —O—(CH₂)ₐNR³¹R³², —NR³¹—(CH₂)ₐNR³³R³⁴, —NR³¹—(CH₂)ₐOR³³, aryl, C₃-C₇ cycloalkyl, a three to twelve membered heterocyclic ring having up to 3 heteroatoms which is optionally substituted from 1 to 3 substituents independently selected from halo, C₁-C₆ alkyl, and —(C₁-C₆ alkyl)-O—(C₁-C₆ alkyl);
R³¹ and R³² are independently selected from the group consisting of H, C₁-C₈ alkyl, C₂-C₈ alkenyl, C₂-C₈ alkynyl, and —(C₁-C₆ alkyl)-O—(C₁-C₆ alkyl);
   or R³¹ and R³² may be taken together to form a three to twelve membered heterocyclic ring having up to 3 heteroatoms which is optionally substituted from 1 to 3 substituents independently selected from halo and C₁-C₆ alkyl;
R³³ and R³⁴ are independently selected from the group consisting of H and C₁-C₈ alkyl;
a is selected from 0 to 6;
   R⁵ is selected from H and C₁-C₆ alkyl;
   R⁶ is selected from the group consisting of H, halo, and C₁-C₆ alkyl.

In an embodiment of the invention R¹³ is selected from the group consisting of C₁-C₈ alkyl, C₃-C₇ cycloalkyl and a three to twelve-membered heterocyclic ring. In an another embodiment of the invention R¹³ is selected from the group consisting of isopropyl, cycloalkyl, N-morpholino and 3-pyridine. In an embodiment of the invention R¹⁴ is H. In an embodiment of the invention R² is H. In another embodiment of the invention R² is F. In an embodiment of the invention R³ is selected from the group consisting of H, C₁-C₈ alkyl and C₁-C₃ perfluoro alkyl. In an another embodiment of the invention R³ is selected from the group consisting of H, CH₃ and CF₃. In an embodiment of the invention R⁴ is selected from the group consisting of H, C₁-C₈ alkyl, C₁-C₃ perfluoro alkyl and a three to twelve-membered heterocyclic ring. In an another embodiment of the invention R⁴ is selected from the group consisting of H, CH₃, CF₃, piperazinyl and N-morpholino.

In aspects of the invention, the compounds useful according to the present invention include those having the formula XVII:

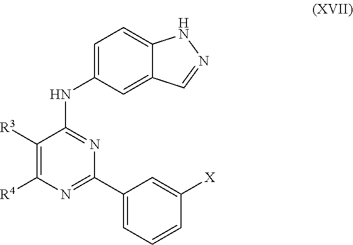

or pharmaceutically acceptable salt or stereoisomer thereof, wherein:
X is selected from the group consisting of —NH—C(═O)—CHR¹³R¹⁴; —NH—C(═O)—(CH₂)_{b}—NR¹³R¹⁴; —C(═O)NR¹³R¹⁴;
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), aryl, heteroaryl, C₃-C₇ cycloalkyl, a three to twelve membered heterocyclic ring containing up to 3 heteroatoms, each of which may be optionally substituted by from 1 to 3 substituents independently selected from halo, oxo, C₁-C₆ alkyl, C₂-C₆ alkenyl, C₃-C₇ cycloalkyl, C₁-C₆ alkoxy, CN and C₁-C₃ perfluoro alkyl;
   each R³ and R⁴ is independently selected from the group consisting of H, C₁-C₈ alkyl, —CN, halo, —OH, —O—(C₁-C₆ alkyl), —O—(C₁-C₆ alkyl)-O—(C₁-C₆ alkyl), —NR³¹R³², C₁-C₃ perfluoro alkyl, —O—(CH₂)ₐNR³¹R³², aryl, C₃-C₇ cycloalkyl, a three to twelve membered heterocyclic ring having up to 3 heteroatoms which is optionally substituted from 1 to 3 substituents independently selected from halo and C₁-C₆ alkyl;
R³¹ and R³² are independently selected from the group consisting of H, C₁-C₈ alkyl, C₂-C₈ alkenyl, C₂-C₈ alkynyl, and —(C₁-C₆ alkyl)-O—(C₁-C₆ alkyl);
a is selected from 0 to 6;
b is selected from 0 to 1.

In an embodiment of the invention R¹³ is a three to twelve-membered heterocyclic ring. In an another embodiment of the invention R¹³ is selected from the group consisting of isopropyl, cycloalkyl, N-morpholino, 3-pyridinyl, tetrahydropyranyl, piperdinyl, and tetrahydrothiopyranyl dioxide. In an another embodiment of the invention R¹³ is selected from the group consisting of:

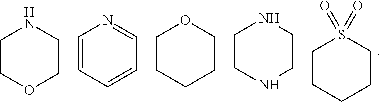

In an embodiment of the invention R¹⁴ is H. In an embodiment of the invention R³ and R⁴ are each H.

In another aspect of the invention, the compounds useful according to the present invention include those having the formula XVIII:

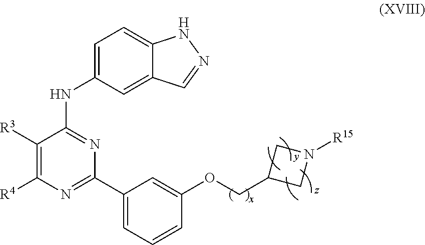

or pharmaceutically acceptable salt or stereoisomer thereof, wherein:
each R³ and R⁴ is independently selected from the group consisting of H, C₁-C₈ alkyl, —CN, halo, —OH, —O—(C₁-C₆ alkyl), —O—(C₁-C₆ alkyl)-O—(C₁-C₆ alkyl), —NR³¹R³², CF₃, —O—(CH₂)ₐNR³¹R³², aryl, C₃-C₇ cycloalkyl, a three to twelve membered heterocyclic ring having up to 3 heteroatoms which is optionally substituted from 1 to 3 substituents independently selected from halo and C₁-C₆ alkyl;
R³¹ and R³² are independently selected from the group consisting of H, C₁-C₈ alkyl, C₂-C₈ alkenyl, C₂-C₈ alkynyl, and —(C₁-C₆ alkyl)-O—(C₁-C₆ alkyl);
a is selected from 0 to 6;

R¹⁵ is selected from the group consisting of H, C₁-C₈ alkyl, —CN, halo, —OH, —O—(C₁-C₆ alkyl), —O—(C₁-C₆ alkyl)-O—(C₁-C₆ alkyl), —C(═O)—O—C(R)₃³¹, CF₃, C₃-C₇ cycloalkyl, a three to twelve membered heterocyclic or aromatic ring having up to 3 heteroatoms which is optionally substituted from 1 to 3 substituents independently selected from halo and C₁-C₆ alkyl;
x is selected from 1 to 3;
y is selected from 0 to 3;
z is selected from 0 to 3;
wherein y or z are independently selected and one of which is at least 1.

In aspects of the invention, the compounds useful according to the present invention include those having the formula XIX:

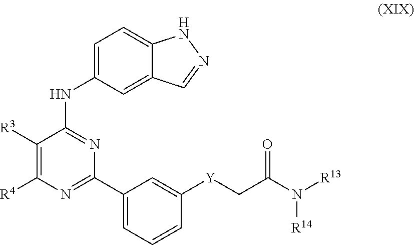

or pharmaceutically acceptable salt or stereoisomer thereof, wherein:
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), heteroaryl, C₃-C₇ cycloalkyl, a three to twelve membered heterocyclic or aromatic ring containing up to 3 heteroatoms, each of which may be optionally substituted by from 1 to 3 substituents independently selected from halo, oxo, C₁-C₆ alkyl, C₂-C₆ alkenyl, C₃-C₇ cycloalkyl, C₁-C₆ alkoxy, CN and C₁-C₃ perfluoro alkyl;
or R¹³ and R¹⁴ may be taken together form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, —OH, —NH₂, CN and C₁-C₃ perfluoro alkyl;
   Y is selected from the group consisting of S, CH₂, and —CR³¹R³²—
   R² is selected from H and halo;
   each R³ and R⁴ is independently selected from the group consisting of H, C₁-C₈ alkyl, —CN, halo, —OH, —O—(C₁-C₆ alkyl), —O—(C₁-C₆ alkyl)-O—(C₁-C₆ alkyl), —NR³¹R³², C₁-C₃ perfluoro alkyl, —O—(CH₂)ₐNR³¹R³², —NR³¹—(CH₂)ₐNR³³R³⁴, —NR³¹—(CH₂)ₐOR³³, aryl, C₃-C₇ cycloalkyl, a three to twelve membered heterocyclic ring having up to 3 heteroatoms which is optionally substituted from 1 to 3 substituents independently selected from halo, C₁-C₆ alkyl, and —(C₁-C₆ alkyl)-O—(C₁-C₆ alkyl);
R³¹ and R³² are independently selected from the group consisting of H, halo, C₁-C₈ alkyl, C₂-C₈ alkenyl, C₂-C₈ alkynyl, and —(C₁-C₆ alkyl)-O—(C₁-C₆ alkyl);
   or R³¹ and R³² may be taken together to form a three to twelve membered cycloalkyl or heterocyclic ring having up to 3 heteroatoms which is optionally substituted from 1 to 3 substituents independently selected from halo and C₁-C₆ alkyl;
R³³ and R³⁴ are independently selected from the group consisting of H and C₁-C₈ alkyl;
a is selected from 0 to 6.

In an embodiment of the invention Y forms a three-membered cycloalkane. In an another embodiment of the invention Y is fluoro;

In another aspect of the invention, the compounds useful according to the present invention include those having the formula XX:

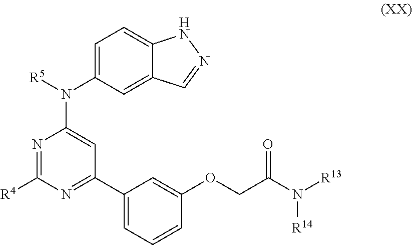

or pharmaceutically acceptable salt or stereoisomer thereof, wherein:
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), heteroaryl, C₃-C₇ cycloalkyl, a three to twelve membered heterocyclic or aromatic ring containing up to 3 heteroatoms, each of which may be optionally substituted by from 1 to 3 substituents independently selected from halo, oxo, C₁-C₆ alkyl, C₂-C₆ alkenyl, C₃-C₇ cycloalkyl, C₁-C₆ alkoxy, CN and C₁-C₃ perfluoro alkyl;
or R¹³ and R¹⁴ may be taken together form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, —OH, —NH₂, CN and C₁-C₃ perfluoro alkyl;
   R⁴ is selected from the group consisting of H, C₁-C₈ alkyl, —CN, halo, —OH, —O—(C₁-C₆ alkyl), —O—(C₁-C₆ alkyl)-O—(C₁-C₆ alkyl), —NR³¹R³², CF₃, —O—(CH₂)ₐNR³¹R³², aryl, C₃-C₇ cycloalkyl, a three to twelve membered heterocyclic ring having up to 3 heteroatoms which is optionally substituted from 1 to 3 substituents independently selected from halo and C₁-C₆ alkyl;
R³¹ and R³² are independently selected from the group consisting of H, C₁-C₈ alkyl, C₂-C₈ alkenyl, C₂-C₈ alkynyl, and —(C₁-C₆ alkyl)-O—(C₁-C₆ alkyl);
   or R³¹ and R³² may be taken together to form a three to twelve membered heterocyclic ring having up to 3 heteroatoms which is optionally substituted from 1 to 3 substituents independently selected from halo and C₁-C₆ alkyl;
   R⁵ is selected from H and C₁-C₆ alkyl.

In another aspect of the invention, the compounds useful according to the present invention include those having the formula XXI:

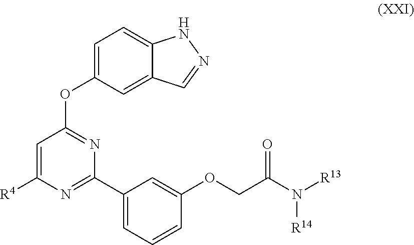

or pharmaceutically acceptable salt or stereoisomer thereof, wherein:
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), heteroaryl, C₃-C₇ cycloalkyl, a three to twelve membered heterocyclic or aromatic ring containing up to 3 heteroatoms, each of which may be optionally substituted by from 1 to 3 substituents independently selected from halo, oxo, C₁-C₆ alkyl, C₂-C₆ alkenyl, C₃-C₇ cycloalkyl, C₁-C₆ alkoxy, CN and C₁-C₃ perfluoro alkyl;
or R¹³ and R¹⁴ may be taken together form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, —OH, —NH₂, CN and C₁-C₃ perfluoro alkyl;
   R⁴ is selected from the group consisting of H, C₁-C₈ alkyl, —CN, halo, —OH, —O—(C₁-C₆ alkyl), —O—(C₁-C₆ alkyl)-O—(C₁-C₆ alkyl), —NR³¹R³², C₁-C₃ perfluoro alkyl, —O—(CH₂)ₐNR³¹R³², aryl, C₃-C₇ cycloalkyl, a three to twelve membered heterocyclic ring having up to 3 heteroatoms which is optionally substituted from 1 to 3 substituents independently selected from halo and C₁-C₆ alkyl;
R³¹ and R³² are independently selected from the group consisting of H, C₁-C₈ alkyl, C₂-C₈ alkenyl, C₂-C₈ alkynyl, and —(C₁-C₆ alkyl)-O—(C₁-C₆ alkyl);
   or R³¹ and R³² may be taken together to form a three to twelve membered heterocyclic ring having up to 3 heteroatoms which is optionally substituted from 1 to 3 substituents independently selected from halo and C₁-C₆ alkyl;
a is selected from 0 to 6.

In another aspect of the invention, the compounds useful according to the present invention include those having the formula XXII:

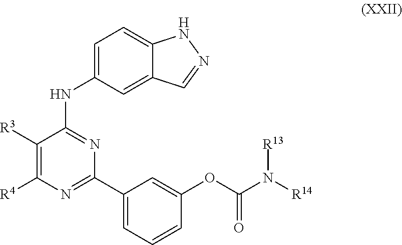

or pharmaceutically acceptable salt or stereoisomer thereof, wherein:
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), heteroaryl, C₃-C₇ cycloalkyl, a three to twelve membered heterocyclic or aromatic ring containing up to 3 heteroatoms, each of which may be optionally substituted by from 1 to 3 substituents independently selected from halo, oxo, C₁-C₆ alkyl, C₂-C₆ alkenyl, C₃-C₇ cycloalkyl, C₁-C₆ alkoxy, CN and C₁-C₃ perfluoro alkyl;
or R¹³ and R¹⁴ may be taken together form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, —OH, —NH₂, CN and C₁-C₃ perfluoro alkyl;

### R³ is H;

R⁴ is selected from the group consisting of H, C₁-C₈ alkyl, —CN, halo, —OH, —O—(C₁-C₆ alkyl), —O—(C₁-C₆ alkyl)-O—(C₁-C₆ alkyl), —NR³¹R³², C₁-C₃ perfluoro alkyl, —O—(CH₂)ₐNR³¹R³², aryl, C₃-C₇ cycloalkyl, a three to twelve membered heterocyclic ring having up to 3 heteroatoms which is optionally substituted from 1 to 3 substituents independently selected from halo and C₁-C₆ alkyl;
R³¹ and R³² are independently selected from the group consisting of H, C₁-C₈ alkyl, C₂-C₈ alkenyl, C₂-C₈ alkynyl, and —(C₁-C₆ alkyl)-O—(C₁-C₆ alkyl);
   or R³¹ and R³² may be taken together to form a three to twelve membered heterocyclic ring having up to 3 heteroatoms which is optionally substituted from 1 to 3 substituents independently selected from halo and C₁-C₆ alkyl;
a is selected from 0 to 6.

In another aspect of the invention, the compounds useful according to the present invention include those having the formula XXIII:

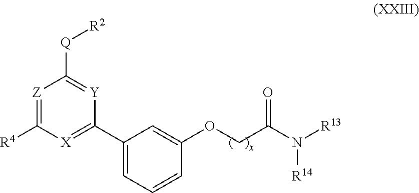

or pharmaceutically acceptable salt or stereoisomer thereof, wherein:
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), heteroaryl, C₃-C₇ cycloalkyl, a three to twelve membered heterocyclic or aromatic ring containing up to 3 heteroatoms, each of which may be optionally substituted by from 1 to 3 substituents independently selected from halo, oxo, C₁-C₆ alkyl, C₂-C₆ alkenyl, C₃-C₇ cycloalkyl, C₁-C₆ alkoxy, CN and C₁-C₃ perfluoro alkyl;
or R¹³ and R¹⁴ may be taken together form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, —OH, —NH₂, CN and C₁-C₃ perfluoro alkyl;
   x is selected from 0 to 1;
   R² is selected from the group consisting of cyclohexylpyridine, 1H-pyrazole, and pyridine;

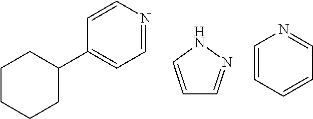

X is selected from N or CR³;
Y is selected from N or CR³;
Z is selected from N or CR⁴;
   wherein at least one of X, Y, and Z is N;
   R⁴ is selected from the group consisting of H, C₁-C₈ alkyl, —CN, halo, —OH, —O—(C₁-C₆ alkyl), —O—(C₁-C₆ alkyl)-O—(C₁-C₆ alkyl), —NR³¹R³², CF₃, —O—(CH₂)ₐNR³¹R³², —NR³¹—(CH₂)ₐNR³³R³⁴, —NR³¹—(CH₂)ₐOR³³, aryl, C₃-C₇ cycloalkyl, a three to twelve membered heterocyclic ring having up to 3 heteroatoms which is optionally substituted from 1 to 3 substituents independently selected from halo, C₁-C₆ alkyl, and —(C₁-C₆ alkyl)-O—(C₁-C₆ alkyl);
R³¹ and R³² are independently selected from the group consisting of H, C₁-C₈ alkyl, C₂-C₈ alkenyl, C₂-C₈ alkynyl, and —(C₁-C₆ alkyl)-O—(C₁-C₆ alkyl);
   or R³¹ and R³² may be taken together to form a three to twelve membered heterocyclic ring having up to 3 heteroatoms which is optionally substituted from 1 to 3 substituents independently selected from halo and C₁-C₆ alkyl;
a is selected from 0 to 6;
   Q is selected from the group NR⁵ and 0;
   R⁵ is selected from H and C₁-C₆ alkyl.

In another aspect of the invention, the compounds useful according to the present invention include those having the formula XXIV:

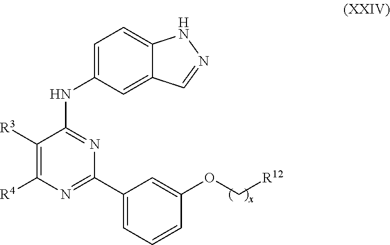

or pharmaceutically acceptable salt or stereoisomer thereof, wherein:
R¹² is selected from the group consisting of H, C₁-C₈ alkyl, C₂-C₈ alkenyl, C₂-C₈ alkynyl, —(C₁-C₆ alkyl)-O—(C₁-C₆ alkyl), amino, NR³¹R³², heteroaryl, C₃-C₇ cycloalkyl, a three to twelve membered heterocyclic or aromatic ring containing up to 3 heteroatoms, each of which may be optionally substituted by from 1 to 3 substituents independently selected from halo, oxo, C₁-C₆ alkyl, C₂-C₆ alkenyl, C₃-C₇ cycloalkyl, C₁-C₆ alkoxy, CN and C₁-C₃ perfluoro alkyl;
   x is selected from 0 to 2;
   each R³ and R⁴ is independently selected from the group consisting of H, C₁-C₈ alkyl, —CN, halo, —OH, —O—(C₁-C₆ alkyl), —O—(C₁-C₆ alkyl)-O—(C₁-C₆ alkyl), —NR³¹R³², CF₃, —O—(CH₂)ₐNR³¹R³², aryl, C₃-C₇ cycloalkyl, a three to twelve membered heterocyclic ring having up to 3 heteroatoms which is optionally substituted from 1 to 3 substituents independently selected from halo and C₁-C₆ alkyl;
R³¹ and R³² are independently selected from the group consisting of H, C₁-C₈ alkyl, C₂-C₈ alkenyl, C₂-C₈ alkynyl, C₃-C₇ cycloalkyl and —(C₁-C₆ alkyl)-O—(C₁-C₆ alkyl);
   or R³¹ and R³² may be taken together to form a three to twelve membered heterocyclic ring having up to 3 heteroatoms which is optionally substituted from 1 to 3 substituents independently selected from halo and C₁-C₆ alkyl;
a is selected from 1 to 6.

In another aspect of the invention, the compounds useful according to the present invention include those having the formula XXV:

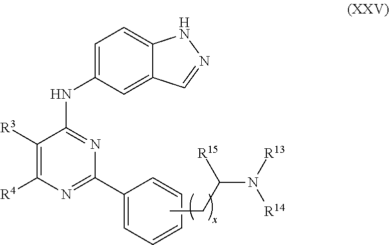

or pharmaceutically acceptable salt or stereoisomer thereof, wherein:
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), heteroaryl, C₃-C₇ cycloalkyl, a three to twelve membered heterocyclic or aromatic ring containing up to 3 heteroatoms, each of which may be optionally substituted by from 1 to 3 substituents independently selected from halo, oxo, C₁-C₆ alkyl, C₂-C₆ alkenyl, C₃-C₇ cycloalkyl, C₁-C₆ alkoxy, CN and C₁-C₃ perfluoro alkyl;
or R¹³ and R¹⁴ may be taken together form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, —OH, —NH₂, CN and C₁-C₃ perfluoro alkyl;
x is selected from 0 to 3;
   R¹⁵ is selected from the group consisting of H, C₁-C₈ alkyl, C₂-C₈ alkenyl, C₂-C₈ alkynyl, aryl, heteroaryl, heterocyclic ring, and C₃-C₇ cycloalkyl;
   each R³ and R⁴ is independently selected from the group consisting of H, C₁-C₈ alkyl, —CN, halo, —OH, —O—(C₁-C₆ alkyl), —O—(C₁-C₆ alkyl)-O—(C₁-C₆ alkyl), —NR³¹R³², CF₃, —O—(CH₂)ₐNR³¹R³², aryl, C₃-C₇ cycloalkyl, a three to twelve membered heterocyclic ring having up to 3 heteroatoms which is optionally substituted from 1 to 3 substituents independently selected from halo and C₁-C₆ alkyl;
R³¹ and R³² are independently selected from the group consisting of H, C₁-C₈ alkyl, C₂-C₈ alkenyl, C₂-C₈ alkynyl, and —(C₁-C₆ alkyl)-O—(C₁-C₆ alkyl);
   or R³¹ and R³² may be taken together to form a three to twelve membered heterocyclic ring having up to 3 heteroatoms which is optionally substituted from 1 to 3 substituents independently selected from halo and C₁-C₆ alkyl;
a is selected from 1 to 6.

In other aspects of the invention, the ROCK2 inhibiting compound may be selected from the ROCK2 compounds disclosed in PCT/US2006/011271, filed Mar. 27, 2006, which is incorporated by reference herein in its entirety. Thus, the ROCK2 inhibiting compound may have the formula XXXI:

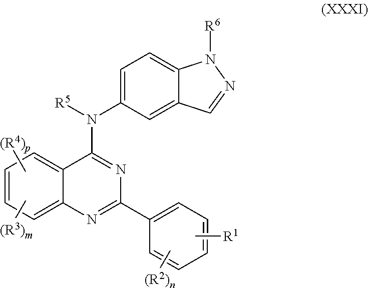

or pharmaceutically acceptable salt, wherein:
R¹ is selected from the group consisting of —O—(CH₂)_{y}—CO₂R¹², —O—(CH₂)_{y}—C(═O)NR¹³R¹⁴, —O—(CH₂)_{y}-heteroaryl, —O—(CH₂)_{y}-cycloalkyl, —O—C(═O)—(CH₂)_{y}—NR¹³R¹⁴, —O—(CH₂)_{z}—NR¹³R¹⁴, —NH—C(═O)—(CH₂)_{y}—NR³R¹⁴, —NH—C(═O)—X—R¹⁵, —NH—(CH₂)_{y}—NR¹³R¹⁴;
R¹² is selected from the group consisting of C₁-C₆ alkyl, —(C₁-C₆ alkyl)-O—(C₁-C₆ alkyl), —(C₁-C₆ alkyl)-NR¹⁶R¹⁷, —(C₁-C₆ alkyl)-C(═O)NR¹⁶R¹⁷, —(C₁-C₆ alkyl)-O—(C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted at one or more carbon atoms by from 1 to 3 substituents independently selected from halo, C₁-C₆ alkoxy, hydroxy, amino, cyano and C₁-C₃ perfluoro alkyl;
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), —(C₁-C₆ alkyl)-NR¹⁶R¹⁷, —(C₁-C₆ alkyl)-C(═O)NR¹⁶R¹⁷, aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R¹³ and R¹⁴ may be taken together form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, OH, NH₂, CN and C₁-C₃ perfluoro alkyl;

X is selected from a covalent bond, O, NH, and C₁-C₆ alkyl;
R¹⁵ is selected from the group consisting of heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, OH, NH₂, CN and C₁-C₃ perfluoro alkyl;
or R¹⁵ is selected from —(C₁-C₆ alkyl)-O—(C₁-C₆ alkyl), —(C₁-C₆ alkyl)-NR¹⁶R¹⁷, —CO₂R¹⁸, —O—(CH₂)ₓ—CO₂R¹⁸, and —C(═O)NR¹⁶R¹⁷;
   R¹⁶ and R¹⁷ independently selected from the group consisting of H, C₁-C₈ alkyl, C₂-C₈ alkenyl, C₁-C₈ alkynyl, —(C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, OH, NH₂, CN and C₁-C₃ perfluoro alkyl;
   or R¹⁶ and R¹⁷ may be taken together form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
   R¹⁸ is selected from the group consisting of H, aryl, aralkyl, heteroaryl, C₁-C₆ alkyl, —(C₁-C₆ alkyl)-O—(C₁-C₆ alkyl), —(C₁-C₆ alkyl)-NR¹⁶R¹⁷, —(C₁-C₆ alkyl)-O—(C₁-C₆ alkyl)-O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
x is selected from 0 to 6;
y is selected from 0 to 6;
z is selected from 2 to 6;
   each R² is independently selected from the group consisting of lower alkyl, CN, halo, hydroxy, lower alkoxy, amino, and perfluoro lower alkyl;
   each R³ is independently selected from the group consisting of lower alkyl, CN, halo, hydroxy, lower alkoxy, amino, and perfluoro lower alkyl;
   R⁴ is selected from —(CH₂)ₐ—NR⁴³R⁴⁴, —Y—R⁴², —O—(CH₂)ₐ—CO₂R⁴², —O—(CH₂)ₐ—C(═O)NR⁴³R⁴⁴, —O—(CH₂)ₐ-heteroaryl, —O—(CH₂)ₐ-cycloalkyl, —O—C(═O)—(CH₂)ₐ—NR⁴³R⁴⁴, —O—(CH₂)_{c}—NR⁴³R⁴⁴, —NH—C(═O)—(CH₂)ₐ—NR⁴³R⁴⁴, —NH—C(═O)—Y—R⁴⁵, —NH—C(═O)—(CH₂)ₐ—NR⁴³R⁴⁴;
R⁴² is selected from the group consisting of C₁-C₆ alkyl, —(C₁-C₆ alkyl)-O—(C₁-C₆ alkyl), —(C₁-C₆ alkyl)-NR⁴⁶R⁴⁷, —(C₁-C₆ alkyl)-C(═O)NR⁴⁶R⁴⁷, —(C₁-C₆ alkyl)-O—(C₁-C₆ alkyl)-O—(C₁-C₆ alkyl), each of which may be optionally substituted at one or more carbon atoms by from 1 to 3 substituents independently selected from halo, C₁-C₆ alkoxy, hydroxy, amino, cyano and C₁-C₃ perfluoro alkyl;
R⁴³ and R⁴⁴ are independently selected from the group consisting of H, C₁-C₈ alkyl, C₂-C₈ alkenyl, C₁-C₈ alkynyl, —(C₁-C₆ alkyl)-O—(C₁-C₆ alkyl), —(C₁-C₆ alkyl)-NR⁴⁶R⁴⁷, —(C₁-C₆ alkyl)-C(═O)NR⁴⁶R⁴⁷, aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R⁴³ and R⁴⁴ may be taken together form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
Y is selected from a covalent bond, 0, NH, and C₁-C₆ alkyl;
R⁴⁵ is selected from the group consisting of H, aryl, —(C₁-C₆ alkyl)-O—(C₁-C₆ alkyl), —(C₁-C₆ alkyl)-NR⁴⁶R⁴⁷, —CO₂R⁴⁸, —O—(CH₂)_{b}—CO₂R⁴⁸, and —C(═O)NR⁴⁶R⁴⁷,
   R⁴⁶ and R⁴⁷ independently selected from the group consisting of H, C₁-C₈ alkyl, C₂-C₈ alkenyl, C₁-C₈ alkynyl, —(C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
   or R⁴⁶ and R⁴⁷ may be taken together form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
   R⁴⁸ is selected from the group consisting of H, aryl, aralkyl, heteroaryl, C₁-C₆ alkyl, —(C₁-C₆ alkyl)-O—(C₁-C₆ alkyl), —(C₁-C₆ alkyl)-NR⁴⁶R⁴⁷, —(C₁-C₆ alkyl)-O—(C₁-C₆ alkyl)-O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
a is selected from 0 to 6;
b is selected from 0 to 6;
c is selected from 2 to 6;
   R⁵ is selected from the group consisting of H, C₁-C₆ alkyl, —(CH₂)_{d}—C(═O)—NR⁵³R⁵⁴, —C(═O)—(CH₂)_{d}—NR⁵³R⁵⁴, —C(═O)—X—R⁵⁵, and —C(═O)—(CH₂)_{d}—NR⁵³R⁵⁴;
R⁵³ and R⁵⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), —(C₁-C₆ alkyl)-NR⁵⁶R⁵⁷, —(C₁-C₆ alkyl)-C(═O)NR⁵⁶R⁵⁷, aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R⁵³ and R⁵⁴ may be taken together form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, hydroxy, amino, cyano and C₁-C₃ perfluoro alkyl;
R⁵⁵ is selected from the group consisting of H, aryl, —(C₁-C₆ alkyl)-O—(C₁-C₆ alkyl), —(C₁-C₆ alkyl)-NR⁵⁶R⁵⁷, —CO₂R⁵⁸, —O—(CH₂)ₑ—CO₂R⁵⁸, and —C(═O)NR⁵⁶R⁵⁷,
   R⁵⁶ and R⁵⁷ independently selected from the group consisting of H, C₁-C₈ alkyl, C₂-C₈ alkenyl, C₁-C₈ alkynyl, —(C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
   or R⁵⁶ and R⁵⁷ may be taken together form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
   R⁵⁸ is selected from the group consisting of H, aryl, aralkyl, heteroaryl, C₁-C₆ alkyl, —(C₁-C₆ alkyl)-O—(C₁-C₆ alkyl), —(C₁-C₆ alkyl)-NR⁵⁶R⁵⁷, —(C₁-C₆ alkyl)-O—(C₁-C₆ alkyl)-O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
   d is selected from 0 to 6;
   e is selected from 0 to 6;
      R⁶ is selected from the group consisting of H, C₁-C₆ alkyl, —(CH₂)ᵣ—C(═O)—NR⁶³R⁶⁴, —C(═O)—(CH₂)ᵣ—NR⁶³R⁶⁴, —C(═O)—X—R⁶⁵, and —C(═O)—(CH₂)ᵣ—NR⁶³R⁶⁴;
R⁶³ and R⁶⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), —(C₁-C₆ alkyl)-NR⁶⁶R⁶⁷, —(C₁-C₆ alkyl)-C(═O)NR⁶⁶R⁶⁷, aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R⁶³ and R⁶⁴ may be taken together form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, hydroxy, amino, cyano and C₁-C₃ perfluoro alkyl;
R⁶⁵ is selected from the group consisting of H, aryl, —(C₁-C₆ alkyl)-O—(C₁-C₆ alkyl), —(C₁-C₆ alkyl)-NR⁶⁶R⁶⁷, —CO₂R⁶⁸, —O—(CH₂)ₛ-CO₂R⁶⁸, and —C(═O)NR⁶⁶R⁶⁷,
   R⁶⁶ and R⁶⁷ independently selected from the group consisting of H, C₁-C₈ alkyl, C₂-C₈ alkenyl, C₁-C₈ alkynyl, —(C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
   or R⁶⁶ and R⁶⁷ may be taken together form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
   R⁶⁸ is selected from the group consisting of H, aryl, aralkyl, heteroaryl, C₁-C₆ alkyl, —(C₁-C₆ alkyl)-O—(C₁-C₆ alkyl), —(C₁-C₆ alkyl)-NR⁶⁶R⁶⁷, —(C₁-C₆ alkyl)-O—(C₁-C₆ alkyl)-O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
   r is selected from 0 to 6;
   s is selected from 0 to 6;
      n is selected from 0 to 4;
      m is selected from 0 to 3; and
      p is selected from 0 and 1.

In one embodiment of Formula XXXI, R⁴ and R⁵ are independently selected from H and C₁-C₆ alkyl. In another embodiment, R⁴ and R⁵ are H.

In an embodiment of the invention, the compound of formula XXXI has the formula XXXII:

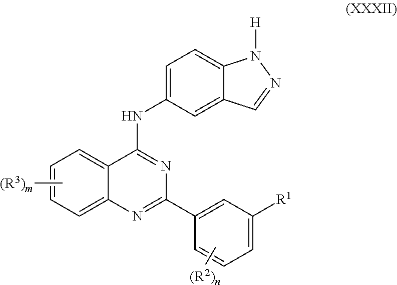

or a pharmaceutically acceptable salt thereof, wherein R¹, R², R³, n and m are as for the compound of the formula I.

In an embodiment of the invention, the compound of formula XXXI has the formula XXXIII:

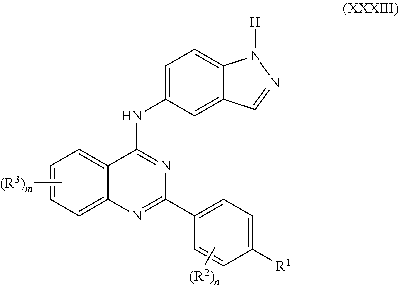

or a pharmaceutically acceptable salt thereof, wherein R¹, R², R³, n and m are as for the compound of the formula I.

In an embodiment of the invention, the compound of formula XXXI has the formula XXXIV:

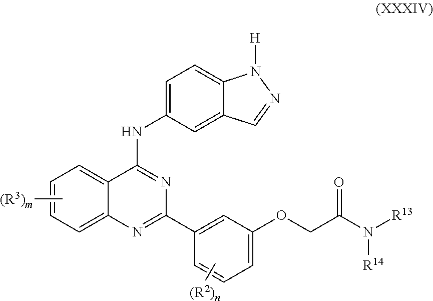

or a pharmaceutically acceptable salt thereof, wherein:
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), —(C₁-C₆ alkyl)-NR¹⁶R¹⁷, —(C₁-C₆ alkyl)-C(═O)NR¹⁶R¹⁷, aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R¹³ and R¹⁴ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, hydroxy, amino, cyano and C₁-C₃ perfluoro alkyl;
X is selected from a covalent bond, O, NH, and C₁-C₆ alkyl;
R¹⁶ and R¹⁷ are independently selected from the group consisting of H, C₁-C₈ alkyl, C₂-C₈ alkenyl, C₂-C₈ alkynyl, —(C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R¹⁶ and R¹⁷ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
each R² is independently selected from the group consisting of lower alkyl, CN, halo, hydroxy, lower alkoxy, amino, and perfluoro lower alkyl;
each R³ is independently selected from the group consisting of lower alkyl, CN, halo, hydroxy, lower alkoxy, amino, and perfluoro lower alkyl;
n is selected from 0 to 4; and
m is selected from 0 to 3.

In an embodiment of the invention, the compound of formula XXXI has the formula XXXIVₐ:

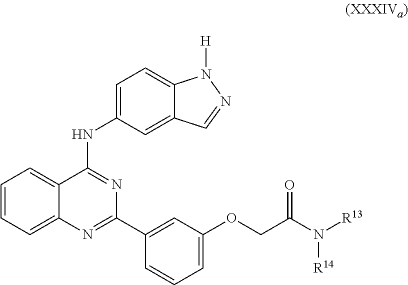

or a pharmaceutically acceptable salt thereof, wherein:
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), —(C₁-C₆ alkyl)-NR¹⁶R¹⁷, —(C₁-C₆ alkyl)-C(═O)NR¹⁶R¹⁷, aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R¹³ and R¹⁴ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
R¹⁶ and R¹⁷ are independently selected from the group consisting of H, C₁-C₈ alkyl, C₂-C₈ alkenyl, C₂-C₈ alkynyl, —(C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R¹⁶ and R¹⁷ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl.

In an embodiment of the invention, the compound of formula XXXI has the formula XXXV:

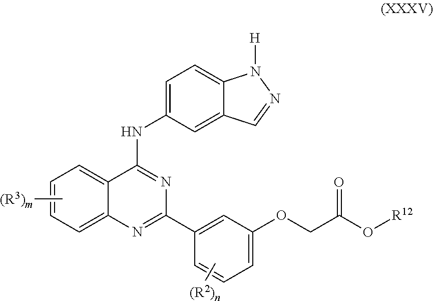

or a pharmaceutically acceptable salt thereof, wherein:
R¹² is selected from the group consisting of C₁-C₆ alkyl, —(C₁-C₆ alkyl)-O—(C₁-C₆ alkyl), —(C₁-C₆ alkyl)-NR¹⁶R¹⁷, —(C₁-C₆ alkyl)-C(═O)NR¹⁶R¹⁷, —(C₁-C₆ alkyl)-O—(C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted at one or more carbon atoms by from 1 to 3 substituents independently selected from halo, C₁-C₆ alkoxy, hydroxy, amino, cyano and C₁-C₃ perfluoro alkyl;
each R² is independently selected from the group consisting of lower alkyl, CN, halo, hydroxy, lower alkoxy, amino, and perfluoro lower alkyl;
each R³ is independently selected from the group consisting of lower alkyl, CN, halo, hydroxy, lower alkoxy, amino, and perfluoro lower alkyl;
n is selected from 0 to 4; and
m is selected from 0 to 3.

In an embodiment of the invention, the compound of formula XXXI has the formula XXXVₐ:

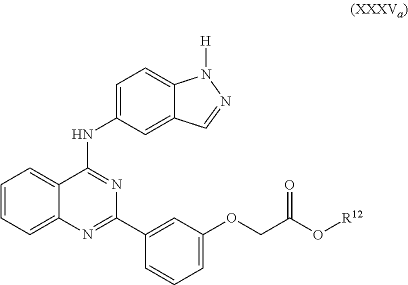

or a pharmaceutically acceptable salt thereof, wherein:
R¹² is selected from the group consisting of C₁-C₆ alkyl, —(C₁-C₆ alkyl)-O—(C₁-C₆ alkyl), —(C₁-C₆ alkyl)-NR¹⁶R¹⁷, —(C₁-C₆ alkyl)-C(═O)NR¹⁶R¹⁷, —(C₁-C₆ alkyl)-O—(C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted at one or more carbon atoms by from 1 to 3 substituents independently selected from halo, C₁-C₆ alkoxy, hydroxy, amino, cyano and C₁-C₃ perfluoro alkyl.

In another embodiment of the invention, the rho kinase inhibitor has the XXXVI:

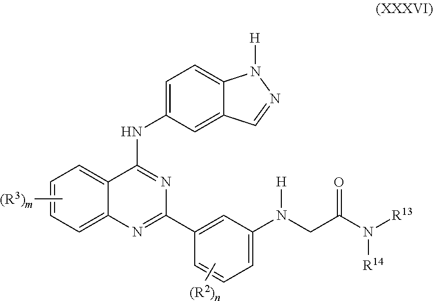

or a pharmaceutically acceptable salt thereof, wherein:
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), —(C₁-C₆ alkyl)-NR¹⁶R¹⁷, —(C₁-C₆ alkyl)-C(═O)NR¹⁶R¹⁷, aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R¹³ and R¹⁴ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, hydroxy, amino, cyano and C₁-C₃ perfluoro alkyl;
R¹⁶ and R¹⁷ are independently selected from the group consisting of H, C₁-C₈ alkyl, C₂-C₈ alkenyl, C₂-C₈ alkynyl, —(C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R¹⁶ and R¹⁷ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
each R² is independently selected from the group consisting of lower alkyl, CN, halo, hydroxy, lower alkoxy, amino, and perfluoro lower alkyl;
each R³ is independently selected from the group consisting of lower alkyl, CN, halo, hydroxy, lower alkoxy, amino, and perfluoro lower alkyl;
n is selected from 0 to 4; and
m is selected from 0 to 3.

In an embodiment of the invention, the compound of formula XXXVI has the formula XXXVIₐ:

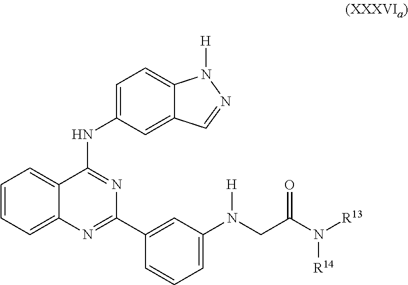

or a pharmaceutically acceptable salt thereof, wherein:
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), —(C₁-C₆ alkyl)-NR¹⁶R¹⁷, —(C₁-C₆ alkyl)-C(═O)NR¹⁶R¹⁷, aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R¹³ and R¹⁴ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, hydroxy, amino, cyano and C₁-C₃ perfluoro alkyl;
R¹⁶ and R¹⁷ are independently selected from the group consisting of H, C₁-C₈ alkyl, C₂-C₈ alkenyl, C₂-C₈ alkynyl, —(C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R¹⁶ and R¹⁷ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl.

In further aspects of the invention, the compound of formula XXXI is SLx-2119:

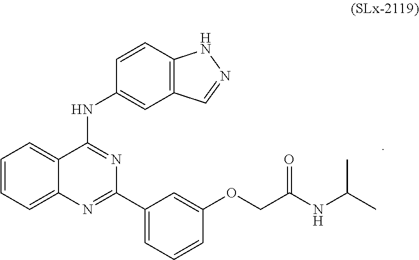 [SMILES: CC(C)NC(=O)COc1cccc(-c2nc(Nc3ccc4[nH]ncc4c3)c3ccccc3n2)c1]

In further aspects of the invention, the rho kinase inhibitor is selected from the group consisting of:
2-(3-(4-(1H-indazol-5-ylamino)quinazolin-2-yl)phenoxy)-N-isopropylacetamide,
2-(3-(4-(1H-indazol-5-ylamino)quinazolin-2-yl)phenoxy)-N-(2-methoxyethyl)acetamide,
2-(3-(4-(1H-indazol-5-ylamino)quinazolin-2-yl)phenoxy)-N-(pyridin-3-yl)acetamide,
2-(3-(4-(1H-indazol-5-ylamino)quinazolin-2-yl)phenoxy)-1-(4-methylpiperazin-1-yl)ethanone,
2-(3-(4-(1H-indazol-5-ylamino)quinazolin-2-yl)phenoxy)-1-morpholinoethanone,
2-(3-(4-(1H-indazol-5-ylamino)quinazolin-2-yl)phenoxy)-N-methylacetamide,
2-(3-(4-(1H-indazol-5-ylamino)quinazolin-2-yl)phenoxy)-N—((R)-pyrrolidin-3-yl)acetamide,
2-(3-(4-(1H-indazol-5-ylamino)quinazolin-2-yl)phenoxy)-N—((S)-pyrrolidin-3-yl)acetamide,
2-(3-(4-(1H-indazol-5-ylamino)quinazolin-2-yl)phenoxy)-N—((R)-tetrahydrofuran-3-yl)acetamide,
2-(3-(4-(1H-indazol-5-ylamino)quinazolin-2-yl)phenoxy)-1-(piperidin-1-yl)ethanone,
2-(3-(4-(1H-indazol-5-ylamino)quinazolin-2-yl)phenoxy)-N-tert-butylacetamide,
2-(3-(4-(1H-indazol-5-ylamino)quinazolin-2-yl)phenoxy)-N-ethylacetamide,
2-(3-(4-(1H-indazol-5-ylamino)quinazolin-2-yl)phenoxy)-N-(cyanomethyl)acetamide,
2-(3-(4-(1H-indazol-5-ylamino)quinazolin-2-yl)phenoxy)-N-cyclobutylacetamide,
2-(3-(4-(1H-indazol-5-ylamino)quinazolin-2-yl)phenoxy)-N-isobutylacetamide,
2-(3-(4-(1H-indazol-5-ylamino)quinazolin-2-yl)phenoxy)-N-(2,2,2-trifluoroethyl)acetamide,
2-(3-(4-(1H-indazol-5-ylamino)quinazolin-2-yl)phenoxy)-N-cyclohexylacetamide,
2-(3-(4-(1H-indazol-5-ylamino)quinazolin-2-yl)phenoxy)-N-neopentylacetamide,
2-(3-(4-(1H-indazol-5-ylamino)quinazolin-2-yl)phenoxy)-N-(prop-2-ynyl)acetamide,
N-(3-(4-(1H-indazol-5-ylamino)quinazolin-2-yl)phenyl)-4-methylpiperazine-1-carboxamide,
3-(3-(4-(1H-indazol-5-ylamino)quinazolin-2-yl)phenyl)-1,1-dimethylurea,
N-(3-(4-(1H-indazol-5-ylamino)quinazolin-2-yl)phenyl)-2-methoxyacetamide,
methyl 2-(3-(4-(1H-indazol-5-ylamino)quinazolin-2-yl)phenylamino)-2-oxoacetate,
1-(3-(4-(1H-indazol-5-ylamino)quinazolin-2-yl)phenyl)-3-(2-(dimethylamino)ethyl)urea,
N-(3-(4-(1H-indazol-5-ylamino)quinazolin-2-yl)phenyl)-2-morpholinoacetamide,
N-(3-(4-(1H-indazol-5-ylamino)quinazolin-2-yl)phenyl)-3-(4-isopropylpiperazin-1-yl)propanamide, and N-(3-(4-(1H-indazol-5-ylamino)quinazolin-2-yl)phenyl)piperidine-4-carboxamide, and N-(3-(4-(1H-indazol-5-ylamino)-6-(2-methoxyethoxy)quinazolin-2-yl)phenyl)butyramide.

In further aspects of the invention, the rho kinase inhibitor is

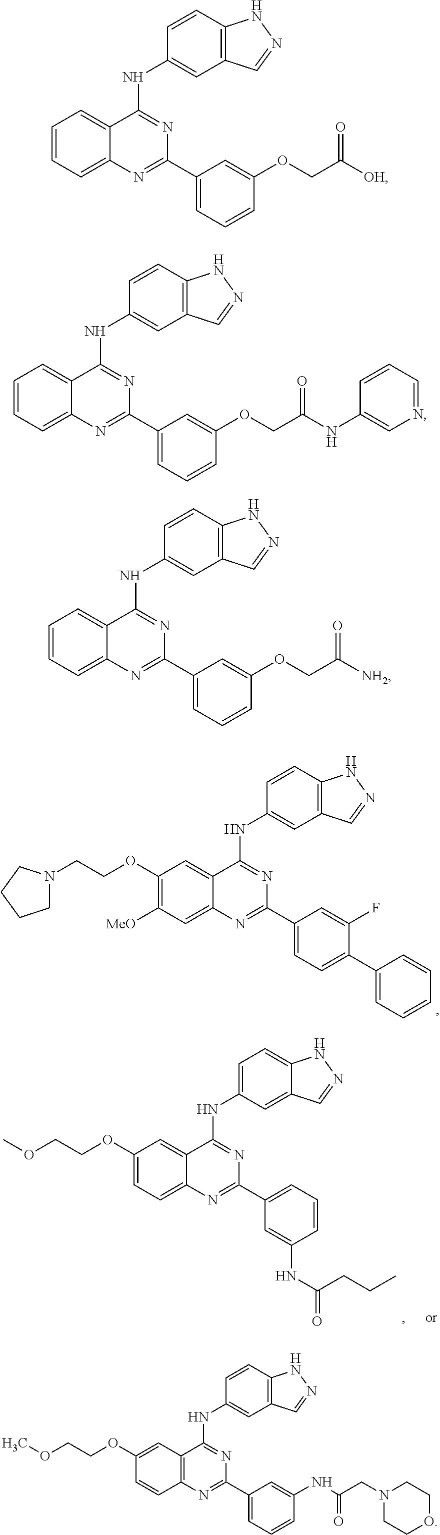

The term “heteroatom” as used herein means an atom of any element other than carbon or hydrogen. Preferred heteroatoms are nitrogen, oxygen, and sulfur.

The term “alkyl” refers to the radical of saturated aliphatic groups, including straight-chain alkyl groups and branched-chain alkyl groups. In preferred embodiments, a straight chain or branched chain alkyl has 10 or fewer carbon atoms in its backbone (e.g., C₁-C₁₀ for straight chain, C₃-C₁₀ for branched chain). Likewise, preferred cycloalkyls have from 3-10 carbon atoms in their ring structure, and more preferably have 3 to 6 carbons in the ring structure.

Unless the number of carbons is otherwise specified, “lower alkyl” as used herein means an alkyl group, as defined above, but having from one to six carbons, and more preferably from one to four carbon atoms. Likewise, “lower alkenyl” and “lower alkynyl” have similar chain lengths (C₂-C₆). Preferred alkyl groups are lower alkyls. In preferred embodiments, a substituent designated herein as alkyl is a lower alkyl.

The term “cycloalkyl” refers to saturated, carbocyclic groups having from 3 to 7 carbons in the ring. Preferred cycloalkyl groups include cyclopropyl, cyclobutyl, cyclopentyl and cyclohexyl.

The term “aralkyl”, as used herein, refers to an alkyl group substituted with an aryl group (e.g., an aromatic or heteroaromatic group).

The terms “alkenyl” and “alkynyl” refer to unsaturated aliphatic groups analogous in length and possible substitution to the alkyls described above, but that contain at least one double or triple bond respectively.

The term “aryl” as used herein includes 5- and 6-membered single-ring aromatic groups that may include from zero to four heteroatoms, for example, benzene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles”, “heteroaromatics” or “heteroaryl”. The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, aryls and/or heterocyclic groups.

The terms “heterocyclyl” or “heterocyclic group” refer to 3- to 10-membered ring structures, more preferably 5- or 6-membered rings, whose ring structures include one to four heteroatoms. Heterocycles can also be polycycles. Heterocyclic groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” refer to two or more rings (e.g., cycloalkyls, cycloalkenyls, aryls and/or heterocyclyls) in which two or more carbons are common to two adjoining rings, e.g., the rings are “fused rings”. Rings that are joined through non-adjacent atoms are termed “bridged” rings. Each of the rings of the polycyclic group can be substituted with such substituents as described above, for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

As used herein, the term “nitro” means —NO₂. The term “halogen” or “halo” designates —F, —Cl, —Br or —I. The term “hydroxyl” means —OH.

The terms “amine” and “amino” refer to both unsubstituted and substituted amines, e.g., a moiety that can be represented by the general formula:

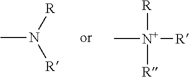

wherein R, R′ and R″ each independently represent H, alkyl, alkenyl, alkynyl, aralkyl, aryl, and heterocyclic groups, and most preferably H or lower alkyl.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. The term lower alkoxy refers to an alkoxy group having from 1 to 6 carbon atoms.

The term “oxo” as used herein refers to an oxygen atom that has a double bond to a another atom, particularly to carbon or sulfur.

As used herein, the definition of each expression, e.g. alkyl, m, n, R, etc., when it occurs more than once in any structure, is intended to be independent of its definition elsewhere in the same structure.

It will be understood that “substituted”, “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. Illustrative substituents include, for example, those described herein above.

Certain compounds of the present invention may exist in particular geometric or stereoisomeric forms. The present invention contemplates all such compounds, including cis- and trans-isomers, R- and S-enantiomers, diastereomers, the racemic mixtures thereof, and other mixtures thereof, as falling within the scope of the invention. Additional asymmetric carbon atoms may be present in a substituent such as an alkyl group. All such isomers, as well as mixtures thereof, are included in this invention.

Certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this context, refers to the relatively non-toxic, inorganic and organic acid addition salts of compounds of the present invention. Representative salts include the hydrochloride, hydrobromide, sulfate, bisulfate, phosphate, nitrate, acetate, stearate, laurate, benzoate, lactate, phosphate, tosylate, citrate, maleate, fumarate, succinate, tartrate, napthylate, and mesylate salts and the like. (See, for example, Berge et al. “Pharmaceutical Salts”, J. Pharm. Sci. (1977) 66:1-19, which is incorporated by reference).

In other cases, the compounds of the present invention may contain one or more acidic functional groups and, thus, are capable of forming pharmaceutically-acceptable salts with pharmaceutically-acceptable bases. Representative salts include alkali or alkaline earth salts such as lithium, sodium, potassium, calcium, magnesium salts and the like. Representative organic amines useful for the formation of base addition salts include ethylamine, diethylamine, ethylenediamine, ethanolamine, diethanolamine, piperazine and the like. (See, for example, Berge et al., supra).

In one aspect, the present invention provides compounds of Formula I that are inhibitors of Rho-kinase. Rho kinase (ROCK), a serine/threonine kinase, serves as a target protein for small GTP-binding protein Rho, and is an important mediator of numerous cellular functions, including focal adhesions, motility, smooth muscle contraction, and cytokinesis. In smooth muscle, ROCK plays an important role in Ca²⁺ sensitization and the control of vascular tone. It modulates the level of phosphorylation of the myosin II light chain of myosin II, mainly through inhibition of myosin phosphatase, and contributes to agonist-induced Ca²⁺ sensitization in smooth muscle contraction.

Rho kinase is found in two forms, ROCK 1 (ROCKβ; p160-ROCK) and ROCK 2 (ROCKα). In some embodiments, the compound of Formula I is selectively inhibits ROCK1. In some embodiments, the compound of Formula I selectively inhibits ROCK2. In some embodiments, the compound of Formula I is non-selective with respect to inhibition of ROCK1 and ROCK2.

Methods of determining kinase inhibition are well known in the art. For example, kinase activity of an enzyme and the inhibitory capacity of a test compound can be determined by measuring enzyme specific phosphorylation of a substrate. Commercial assays and kits can be employed. For example, kinase inhibition can be determined using an IMAP® assay (Molecular Devices). This assay method involves the use of a fluorescently-tagged peptide substrate. Phosphorylation of the tagged peptide by a kinase of interest promotes binding of the peptide to a trivalent metal-based nanoparticle via the specific, high affinity interaction between the phospho-group and the trivalent metal. Proximity to the nanoparticle results in increased fluorescence polarization. Inhibition of the kinase by a kinase inhibitor prevents phosphorylation of the substrate and thereby limits binding of the fluorescently-tagged substrate to the nanoparticle. Such an assay can be compatible with a microwell assay format, allowing simultaneous determination of IC₅₀ of multiple compounds.

In another aspect, the invention provides angiogenesis inhibitors for disease treatment. In certain embodiments, novel VEGFR2 antibodies or antigen binding fragments of such antibodies are employed, which are effective to inhibit VEGFR2-dependent signal transduction. As used herein, “inhibiting a receptor” means diminishing and/or inactivating the intrinsic kinase activity of the receptor to transduce a signal. A reliable assay for VEGFR2 inhibition is reduction of receptor phosphorylation.

The present invention is not limited by any particular mechanism of VEGFR2 inhibition. The mechanism followed by one antibody is not necessarily the same as that followed by another. Some possible mechanisms include preventing binding of the VEGF ligand to the extracellular binding domain of the VEGFR2, and preventing dimerization or oligomerization of receptors. Other mechanisms cannot, however, be ruled out.

Antibodies are proteins that recognize and bind to a specific antigen or substance. In preferred embodiments, the antibodies of the present invention bind KDR at least as strongly as the natural ligand. Affinity, represented by the equilibrium constant for the dissociation of an antigen with an antibody (Kd), measures the binding strength between an antigenic determinant and an antibody binding site. Avidity is the measure of the strength of binding between an antibody with its antigen. Avidity is related to both the affinity between an antigenic determinant and an antigen binding site on the antibody, and the number of binding sites (valence) per antibody. For example, a monovalent antibody has one binding site for a particular epitope. Typical values of K (the reciprocal of the dissociation constant K_{d}) are 10⁵ to 10¹¹ liters/mol. Any K weaker than 10⁴ liters/mol is considered to indicate binding which is nonspecific.

Antibodies of the invention inhibit activation of VEGFR2. One measure of VEGFR2 inhibition is reduced tyrosine kinase activity of the receptor. Tyrosine kinase inhibition can be determined using well-known methods, such as measuring the autophosphorylation level of the receptor. Inhibition of VEGFR2 can also be observed through inhibition or regulation of phosphorylation events of natural or synthetic VEGFR2 substrates and other components of the VEGFR2 signal transduction pathway. Phosphorylation can be detected, for example, using an antibody specific for phosphotyrosine in an ELISA assay or on a western blot. Some assays for tyrosine kinase activity are described in Panek et al., J. Pharmacol. Exp. Thera., 283: 1433-44 (1997) and Batley et al., Life Sci., 62: 143-50 (1998).

In vivo assays can also be utilized. For example, receptor tyrosine kinase inhibition can be observed by mitogenic assays using cell lines stimulated with receptor ligand in the presence and absence of inhibitor. For example, HUVEC cells (ATCC) stimulated with VEGF can be used to assay VEGFR inhibition. Another method involves testing for inhibition of growth of VEGF-expressing tumor cells, using for example, human tumor cells injected into a mouse. See, U.S. Pat. No. 6,365,157 (Rockwell et al.).

The invention provides anti-VEGFR2 antibodies, including nucleic acids encoding such antibodies and compositions comprising such antibodies. In one embodiment the invention provides an isolated antibody heavy chain variable region comprising a CDR-1H, CDR-2H, and CDR-3H sequence, wherein:

(i) the CDR-1H sequence is GFTFSWYX₁MX₂ (SEQ ID NO:185), wherein X₁ is V or I, X₂ is G or L,

(ii) the CDR-2H sequence is SIX₁X₂SGGX₃TX₄YADSVKG (SEQ ID NO:186), wherein X₁ is Y or G, X₂ is P or S, X₃ is A or F, X₄ is N or D, and

(iii) the CDR-3H sequence is GNYFDY (SEQ ID NO:3) or GLAAPRS (SEQ ID NO:11).

In one embodiment, the invention provides an isolated light chain variable region comprising a CDR-L1, CDR-L2, and CDR-L3, wherein

(i) the CDR-L1 sequence is X₁GX₂X₃LX₄X₅X₆X₇X₈S (SEQ ID NO: 187), wherein X₁ is S, Q, or T, X₂ is D, E, or Q, X₃ is K, S, N, I, or A, X₄ is G or R, X₅ is D, S, H, E, or N, X₆ is E, Y, Q, R, or N, X₇ is Y, F, or S, and X₈ is A or S, or SGSX₁SNX₂X₃X₄X₅X₆X₇X₈ (SEQ ID NO: 188), wherein X₁ is S, or T, X₂ is I or L, X₃ is E or G, X₄ is T, S, or N, X₅ is N or Y, X₆ is T, P, A, or Y, X₇ is V or L, and X₈ is N, I, or Y, or X₁GX₂SX₃DX₄GX₅YDYVS (SEQ ID NO: 189), wherein X₁ is A or T, X₂ is S or T, X₃ is H, S, or N, X₄ is I or V, and X₅ is S or A,

(ii) the CDR-L2 sequence is X₁X₂X₃X₄X₅PS (SEQ ID NO:190), wherein X₁ is Q, D, T, Y, S, or A, X₂ is D, N, S, T, or V, X₃ is D, N, S, T, or Y, X₄ is Q, K, N, or L, and X₅ is R or L, and

(iii) wherein the CDR-L3 sequence is QX₁WX₂X₃X₄X₅X₆X₇X₈ (SEQ ID NO:191), wherein X₁ is A or T, X₂ is D or G, X₃ is R or no amino acid, X₄ is S, F, or N, X₅ is S, T, or N, X₆ is S, T, or P, X₇ is A, V, L, I, or Y, and X₈ is V or L, or AX₁WDDX₂LX₃X₄X₅X₆ (SEQ ID NO:192, wherein X₁ is A, S, or T, X₂ is N or S, X₃ is N, I, or G, X₄ is G or S, X₅ is P, W, or V, and X₆ is V or L, or MYSTITX₁LL (SEQ ID NO:193), wherein X₁ is A or T.

In one embodiment, the invention provides an isolated light chain variable region comprising a CDR-L1, CDR-L2, and CDR-L3, wherein

(i) the CDR-L1 sequence is RASX₁X₂X₃X₄X₅X₆X₇YX₅X₉ (SEQ ID NO:194), wherein X₁ is Q, E, or H, X₂ is S, R, or N, X₃ is V, I, or L, X₄ is S, R, G or N, X₅ is S or N, X₆ is S, N, W, or D, X₇ is G or no amino acid, X₈ is L or F, and X₉ is A, G, M, or S, (ii) the CDR-L2 sequence is GASX₁RAT (SEQ ID NO:195), wherein X₁ is S, T, I, or N, and

(iii) the CDR-L3 sequence is QQX₁X₂X₃X₄X₅X₆X₇X₈ (SEQ ID NO:196), wherein X₁ is F or Y, X₂ is D, G, or Y, X₃ is S, T, or N, X₄ is S, L, or W, X₅ is P or no amino acid, X₆ is P or T, X₇ is L, I, V, P, W, or Y, and X₈ is T or S.

In an embodiment of the invention, an antibody is provided which comprises a heavy chain variable domain comprising one, two, three, four, five, or six of the light chain variable domain and heavy chain variable domain CDR sequences set forth above.

Non-limiting examples of VEGFR2-binding antibody sequences are provided. As described herein, from human Fab phage display libraries, two neutralizing antibodies were identified that bind to human VEGFR2, block binding of the ligand VEGFA to hVEGFR2, and inhibit the VEGFR2 phosphorylation and downstream signal transduction stimulated by VEGFA. Table 1 indicates amino acid sequences of the CDRs and variable domains of antibodies of the antibodies. The K_{d}s of Mab 101 and Mab 102 are about 6.6 mM and 1.7 nM, respectively.

TABLE 1
Antibody Amino Acid Sequences by SEQ ID NO

| | CDR- | CDR- | CDR- | V_{H} | CDR- | CDR- | CDR- | V_{L} |
|---|---|---|---|---|---|---|---|---|
| Mab | H1 | H2 | H3 | domain | L1 | L2 | L3 | domain |
| 101 | 1 | 2 | 3 | 4 | 5 | 6 | 7 | 8 |
| 102 | 9 | 10 | 11 | 12 | 13 | 14 | 15 | 16 |

The heavy chain of Mab 101 was reshuffled with κ light chain genes (K-library) and λ light chain genes (λ-library). 20 unique λ light chain variants were found by panning the λ-library against both human VEGFR2 and mouse VEGFR2. 22 unique κ light chain variants were found by panning the κ-library against both human VEGFR2 and mouse VEGFR2. Table 2 indicates amino acid sequences of the CDRs and variable domains of the light chains. The KDs of Mabs 105, 106, and 107 were increased about 10 fold (0.24 nM, 0.22 nM, and 0.12 nM, respectively).

TABLE 2
κ and λ light chains by SEQ ID NO

SEQ ID NO

| | light | CDR- | CDR- | CDR- | |
|---|---|---|---|---|---|
| Mab | chain | L1 | L2 | L3 | V_{L} |
| 103 | λ | 17 | 18 | 19 | 20 |
| 104 | λ | 21 | 22 | 23 | 24 |
| 105 | λ | 25 | 26 | 27 | 28 |
| 106 | λ | 29 | 30 | 31 | 32 |
| 107 | λ | 33 | 34 | 35 | 36 |
| 108 | λ | 37 | 38 | 39 | 40 |
| 109 | λ | 41 | 42 | 43 | 44 |
| 110 | λ | 45 | 46 | 47 | 48 |
| 111 | λ | 49 | 50 | 51 | 52 |
| 112 | λ | 53 | 54 | 55 | 56 |
| 113 | λ | 57 | 58 | 59 | 60 |
| 114 | λ | 61 | 62 | 63 | 64 |
| 115 | λ | 65 | 66 | 67 | 68 |
| 116 | λ | 69 | 70 | 71 | 72 |
| 117 | λ | 73 | 74 | 75 | 76 |
| 118 | λ | 77 | 78 | 79 | 80 |
| 119 | λ | 81 | 82 | 83 | 84 |
| 120 | λ | 85 | 86 | 87 | 88 |
| 121 | λ | 89 | 90 | 91 | 92 |
| 122 | λ | 93 | 94 | 95 | 96 |
| 123 | κ | 97 | 98 | 99 | 100 |
| 124 | κ | 101 | 102 | 103 | 104 |
| 125 | κ | 105 | 106 | 107 | 108 |
| 126 | κ | 109 | 110 | 111 | 112 |
| 127 | κ | 113 | 114 | 115 | 116 |
| 128 | κ | 117 | 118 | 119 | 120 |
| 129 | κ | 121 | 122 | 123 | 124 |
| 130 | κ | 125 | 126 | 127 | 128 |
| 131 | κ | 129 | 130 | 131 | 132 |
| 132 | κ | 133 | 134 | 135 | 136 |
| 133 | κ | 137 | 138 | 139 | 140 |
| 134 | κ | 141 | 142 | 143 | 144 |
| 135 | κ | 145 | 146 | 147 | 148 |
| 136 | κ | 149 | 150 | 151 | 152 |
| 137 | κ | 153 | 154 | 155 | 156 |
| 138 | κ | 157 | 158 | 159 | 160 |
| 139 | κ | 161 | 162 | 163 | 164 |
| 140 | κ | 165 | 166 | 167 | 168 |
| 141 | κ | 169 | 170 | 171 | 172 |
| 142 | κ | 173 | 174 | 175 | 176 |
| 143 | κ | 177 | 178 | 179 | 180 |
| 144 | κ | 181 | 182 | 183 | 184 |

The invention provides an isolated VEGFR2 antibody, and VEGFR2 binding fragments thereof, which comprises one, two, or three heavy chain CDRs and one, two, or three light chain CDRs, selected from the sequences set forth in Table 1 and Table 2. In an antibody of the invention, when more than one CDR is selected from the sequences presented in Table 1 and Table 2, the different CDRs need not be selected from the same monoclonal antibody presented in those tables, but can be selected from two or more antibody variable domains presented in the tables. Specific embodiments include, but are not limited to, the following. In an embodiment of the invention, the isolated VEGFR2 antibody comprises one, two, or three heavy chain CDRs having SEQ ID NO:1, SEQ ID NO:2, and SEQ ID NO:3. In an embodiment, of the invention, the antibody comprises one, two, or three light chain CDRs having SEQ ID NO:5, SEQ ID NO:6, and SEQ ID NO:7. In another embodiment, the antibody comprises one, two, or three light chain CDRs having sequences as set forth in Table 1 or 2. Non-limiting examples include a light chain variable region comprising one or more of SEQ ID NO:25, SEQ ID NO:26, and SEQ ID NO:27, one or more of SEQ ID NO:29, SEQ ID NO:30, and SEQ ID NO:31, or one or more of SEQ ID NO:33, SEQ ID NO:34, and SEQ ID NO:35. In certain embodiments, the VEGFR2 antibody comprises a heavy chain variable domain comprising SEQ ID NO:4 or SEQ ID NO: 12. In certain embodiments, the VEGFR2 antibody comprises a light chain variable domain comprising SEQ ID NO:8, SEQ ID NO:16, SEQ ID NO:27, SEQ ID NO:31, or SEQ ID NO:35. In certain embodiments, the antibodies comprise one of the above-mentioned heavy chain variable domains and one of the above-mentioned light chain variable domains. In certain embodiments, the VEGFR2 antibodies or binding fragments thereof comprise one or more CDRs or one or more variable domains with an amino acid sequence at least 85% at least 90%, at least 95%, at least 97%, at least 98%, or at least 99%, identical to the CDR and variable domain sequences set forth in Table 1 or 2.

“Identity” refers to the number or percentage of identical positions shared by two amino acid or nucleic acid sequences, taking into account the number of gaps, and the length of each gap, which need to be introduced for optimal alignment of the two sequences. “Substantially identical” means an amino acid sequence that which differs only by conservative amino acid substitutions, for example, substitution of one amino acid for another of the same class (e.g., valine for glycine, arginine for lysine, etc.) or by one or more non-conservative substitutions, deletions, or insertions located at positions of the amino acid sequence which do not destroy the function of the protein. Preferably, the amino acid sequence is at least 80%, more preferably at least 85%, and most preferably at least 90% similar to another amino acid sequence. Methods and computer programs for determining sequence similarity are publically available, including, but not limited to, the GCG program package (Devereux et al., Nucleic Acids Research 12: 387, 1984), BLASTP, BLASTN, FASTA (Altschul et al., J. Mol. Biol. 215:403 (1990), and the ALIGN program (version 2.0). The well-known Smith Waterman algorithm may also be used to determine similarity. The BLAST program is publicly available from NCBI and other sources (BLAST Manual, Altschul, et al., NCBI NLM NIH, Bethesda, Md. 20894; BLAST 2.0 at http://www.ncbi.nlm.nih.gov/blast/). In comparing sequences, these methods account for various substitutions, deletions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Certain embodiments of the invention involve the use of VEGFR2-binding antibody fragments. An Fv is the smallest fragment that contains a complete heavy and light chain variable domain, including all six hypervariable loops (CDRs). Lacking constant domains, the variable domains are noncovalently associated. The heavy and light chains may be connected into a single polypeptide chain (a “single-chain Fv” or “scFv”) using a linker that allows the V_{H} and V_{L} domains to associate to form an antigen binding site. In an embodiment of the invention, the linker is (Gly-Gly-Gly-Gly-Ser)₃. Since scFv fragments lack the constant domains of whole antibodies, they are considerably smaller than whole antibodies. scFv fragments are also free of normal heavy-chain constant domain interactions with other biological molecules which may be undesired in certain embodiments.

Fragments of an antibody containing V_{H}, V_{L}, and optionally C_{L}, C_{H}1, or other constant domains can also be used. Monovalent fragments of antibodies generated by papain digestion are referred to as Fab and lack the heavy chain hinge region. Fragments generated by pepsin digestion, referred to as F(ab′)₂, retain the heavy chain hinge and are divalent. Such fragments may also be recombinantly produced. Many other useful antigen-binding antibody fragments are known in the art, and include, without limitation, diabodies, triabodies, single domain antibodies, and other monovalent and multivalent forms.

The invention further provides multivalent antigen-binding proteins, which can be in the form, without limitation, of antibodies, antigen-binding fragments thereof, and proteins comprising all or part of antigen-binding portions of antibodies. Multivalent antigen-binding proteins may be monospecific, bispecific, or multispecific. The term specificity refers to the number of different types of antigenic determinants to which a particular molecule can bind. If an immunoglobulin molecule binds to only one type of antigenic determinant, the immunoglobulin molecule is monospecific. If the immunoglobulin molecule binds to different types of antigenic determinants then the immunoglobulin molecule is multispecific.

For example, a bispecific multivalent single chain antibody allows for the recognition of two different types of epitopes. Both epitopes may be on the same antigen (e.g., VEGFR2). Alternatively, one epitope may be on one antigen (e.g., VEGFR2), and the second epitope on a different antigen.

In one embodiment, a multivalent single chain antibody includes a variable light-chain fragment linked to a variable heavy-chain fragment (similar to an scFv), which is further linked by another peptide linker to at least one other antigen binding domain. Typically, the peptide linker is composed of about fifteen amino acid residues. In a preferred embodiment, the number of V_{L} and V_{H} domains is equivalent. For example, a bivalent single chain antibody can be represented as follows: V_{L}-L₁-V_{H}-L₂-V_{L}-L₃-V_{H} or V_{L}-L₁-V_{H}-L₂-V_{H}-L₃-V_{L} or V_{H}-L₁-V_{L}-L₂-V_{H}-L₃-V_{L} or V_{H}-L₁-V_{L}-L₂-V_{L}-L₃-V_{H}. Multivalent single chain antibodies which are trivalent or greater have one or more antibody fragments joined to a bivalent single chain antibody by additional peptide linkers. One example of a trivalent single chain antibody is: V_{L}-L₁-V_{H}-L₂-V_{L}-L₁-V_{H}-L₂-V_{L}-L_{I}-V_{H}.

Two single chain antibodies can be combined to form a diabody, also known as bivalent dimer. Diabodies have two chains. Each chain of the diabody includes a V_{H} domain connected to a V_{L} domain by a short linker of about 5-10 amino acid residues, e.g. (Gly-Gly-Gly-Gly-Ser), (Gly-Gly-Gly-Gly-Ser)₂. Such linkers are short enough to prevent intrachain pairing between domains on the same chain, thus driving interchain pairing between complementary domains on different chains and recreate two antigen-binding sites. The diabody structure is rigid and compact, with antigen-binding sites are at opposite ends of the molecule. Diabodies may be monospecfic or bispecific.

Three single chain antibodies can be combined to form a triabody, also known as a trivalent trimers. In some embodiments, triabodies are constructed with the carboxy terminus of a V_{L} or V_{H} domain directly fused to the amino terminus of a V_{H} or V_{L} domain, i.e., without any linker sequence. The triabody has three Fv heads with the polypeptides arranged in a cyclic, head-to-tail fashion. A possible conformation of the triabody molecule is planar with the three binding sites located in a plane at an angle of 120 degrees from one another. Triabodies may be monospecific, bispecific or trispecific.

It is understood that the anti-VEGFR2 antibodies of the invention, where used in a mammal for the purpose of prophylaxis or treatment, will be administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the antibodies.

In the methods of the present invention, a therapeutically effective amount of an antibody of the invention is administered to a mammal in need thereof. The term “administering” as used herein means delivering the antibodies of the present invention to a mammal by any method that may achieve the result sought. They may be administered, for example, intravenously or intramuscularly. Although human antibodies of the invention are particularly useful for administration to humans, they may be administered to other mammals as well. The term “mammal” as used herein is intended to include, but is not limited to, humans, laboratory animals, domestic pets and farm animals. “Therapeutically effective amount” means an amount of antibody of the present invention that, when administered to a mammal, is effective in producing the desired therapeutic effect, such as inhibiting kinase activity.

Compounds of the invention can be advantageously administered with second agents to patients in need thereof. For example, in certain embodiments, a rho-kinase inhibitor of the invention is administered with an angiogenesis inhibitor. In certain embodiments, a VEGFR2 inhibitor is administered with an anti-inflammatory agent or an immunosuppressant. In certain embodiments, a VEGFR2 inhibitor is administered with an antineoplastic agent.

In certain embodiments a Rho-kinase inhibitor and an angiogenesis inhibitor are administered to a subject in need thereof. In certain embodiments, the Rho-kinase inhibitor is a compound of Formula I. In certain embodiments, the Rho kinase inhibitor is selective for ROCK1 over ROCK2. In certain embodiments, the Rho-kinase inhibitor is selective for ROCK2 over ROCK1. In some embodiments, the ROCK2 selective inhibitor is

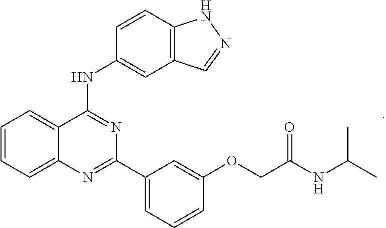 [SMILES: CC(C)NC(=O)COc1cccc(-c2nc(Nc3ccc4[nH]ncc4c3)c3ccccc3n2)c1]

Angiogenesis inhibitors include any substance that inhibits the growth of new blood vessels. For example, angiogenesis inhibitors include antagonists of VEGF, PlGF, and VEGF receptors, as well as the antibodies disclosed herein. By inhibitor is meant an inhibitor of a biological process or inhibitor of a target. In this regard, an angiogenesis inhibitor is an agent that reduces angiogenesis. A Rho-kinase inhibitor is an agent, such as a competitive inhibitor of ATP binding, that inhibits an activity or blocks an interaction of Rho-kinase. By antagonist is meant a substance that reduces or inhibits an activity or function in a cell associated with a target. For example, a VEGF antagonist reduces or blocks a function in a cell that is associated with VEGF. A VEGF antagonist may act on VEGF, by binding to VEGF and blocking binding to its receptors or may act on another cellular component involved in VEGF-mediated signal transduction. Similarly, a VEGFR2 antagonist is an agent that reduces or blocks VEGFR2-mediated signal transduction by binding to VEGFR2 and blocking ligand binding or interaction with a VEGFR2 substrate, or acts on another cellular component to reduce or block VEGFR2-mediated signal transduction. Thus, angiogenesis inhibitors include novel anti-VEGFR2 antibodies of the invention, and antagonists of, without limitation, VEGF, VEGFR1, VEGFR2, PDGF, PDGFR-β, neuropilin-1 (NRP1), and complement.

Antineoplastic agents include cytotoxic chemotherapeutic agents, targeted small molecules and biological molecules, and radiation. In certain embodiments of the invention, the anti-VEGFR2 antibodies of the invention are administered with irinotecan, etoposide, 5-fluorouracil, paclitaxel, or radiotherapy.

Anti-inflammatories and immunosuppressants include steroid drugs such as glucocorticoids (e.g., dexamethasone), FK506 (tacrolimus), ciclosporin, fingolimod, interferon, such as IFNβ or IFNγ, a tumor necrosis factor-alpha (TNF-α) binding protein such as infliximab (Remicade), etanercept (Enbrel), or adalimumab (Humira), and mycophenolic acid.

When a rho-kinase inhibitor is administered with a second agent, the rho-kinase inhibitor and the second agent can be adminstered sequentially or concomitantly. Sequentially means that one agent is administered for a time followed by administration of the second agent, which may be followed by administration of the first agent. When agents are administered sequentially, the level or one agent may not be maintained at a therapeutically effective level when the second agent is administered, and vice versa. Concomitantly means that the first and second agent are administered according to a schedule that maintains both agents at an substantially therapeutically effective level, even though the agents are not administered simultaneously. Each agent can be administered in single or multiple doses, and the doses can be administered on any schedule, including, without limitation, twice daily, daily, weekly, every two weeks, and monthly.

The invention also includes adjunctive administration. Adjunctive administration means that a second agent is administered to a patient in addition to a first agent that is already being administered to treat a disease or disease symptom. In some embodiments, adjunctive administration includes administering a second agent to a patient in which administration of the first agent did not treat, or did not sufficiently treat, the disease or disease symptom. In other embodiments, adjunctive administration includes administration of the second agent to a patient whose disease has been effectively treated by administration of the first agent.

Either agent can be administered adjunctively. In certain embodiments, a rho-kinase inhibitor is administered to a patient that is already receiving a second agent. In other embodiments, a second agent is administered to a patient that is already receiving a rho-kinase inhibitor. In some embodiments, a VEGF antagonist is administered with a second agent. In some embodiments, the effect of administering the first and second agents is synergistic. In some embodiments, administration of the first and second agents prevents or lengthens the time until relapse, compared to administration of either of the agents alone. In some embodiments, administration of the first and second agents allows for reduced dosage and/or frequency of administration of the first and second agent. In other embodiments, a second agent is administered to a patient that is already receiving the VEGF antagonist.

The invention provides methods and compounds for treating ocular disorders. In certain embodiments, the ocular disorder is characterized as having an angiogenic component. Excessive angiogenesis occurs in diseases such as cancer, diabetic blindness, age-related macular degeneration, rheumatoid arthritis, psoriasis, and more than 70 other conditions. In these conditions, new blood vessels feed diseased tissues, destroy normal tissues, and in the case of cancer, the new vessels allow tumor cells to escape into the circulation and lodge in other organs (tumor metastases). Excessive angiogenesis occurs when diseased cells produce abnormal amounts of angiogenic growth factors, overwhelming the effects of natural angiogenesis inhibitors. According to the invention, such disorders are treated by administering a Rho-kinase inhibitor, preferably a ROCK2 selective Rho-kinase inhibitor, and an angiogenesis inhibitor.

In one embodiment, the invention provides a method of treating age related macular degeneration (AMD), which occurs in “dry” and “wet” forms. The “wet” form of AMD causes vision loss due to abnormal blood vessel growth (neovascularization). Bleeding, leaking, and scarring from these retinal blood vessels eventually causes irreversible damage to the photoreceptors. The dry form results from atrophy of the retinal pigment epithelial layer, which causes vision loss through loss of photoreceptors (rods and cones) in the central part of the eye. In another embodiment, the invention provides a method of treating choroidal neovascularization (CNV). Choroidal neovascularization is a process in which new blood vessels grow in the choroid, through the Bruch membrane and invade the subretinal space, and is a symptom of, among other causes, age-related macular degeneration, myopia and ocular trauma. In another embodiment, the invention provides a method of treating diabetic macular edema (DME). In another embodiment, the invention provides a method of treating macular edema that is secondary to branch retinal vein occlusion (BRVO) or central retinal vein occlusion (CRVO). In other embodiments, the diseases to be treated include, without limitation, retinal neovascularization infectious and non-infectious, corneal neovascularization infectious and non-infectious, iris neovascularization, uveitis, neovascular glaucoma, and retinitis of prematurity (ROP). The method of treatment can be prophylactic, such as to stave off corneal neovascularization after corneal transplant, or to modulate the wound healing process in trabeculectomy surgery.

In one such embodiment, the disease or disorder is AMD, and a subject in need of treatment for AMD is administered an amount of a ROCK2 inhibitor effective to treat AMD. In another embodiment, the subject is administered a ROCK2 inhibitor and an angiogenesis inhibitor in amounts effective to treat AMD. In some embodiments, the angiogenesis inhibitor is a VEGFR2 antagonist. In certain such embodiments, the VEGFR2 antagonist binds to VEGF. In other such embodiments, the VEGFR2 antagonist binds to VEGFR2. Such VEGFR2-binding inhibitors include agents that bind to the extracellular domain of VEGFR2, including but not limited to antibodies and VEGFR2-binding fragments thereof, and agents that interact with the intracellular domain of VEGFR2 and block activation of VEGFR2-dependent signal transduction. VEGFR2 antagonists further include agents that interact with other cellular components to block VEGFR2-dependent signal transduction. In other embodiments of the invention, ocular diseases and disorders indicated above, are similarly treated.

According to the invention, a ROCK inhibitor and an angiogenesis inhibitor is administered to a subject in amounts effective amount to treat or preventing a pathologic condition characterized by excessive angiogenesis. Such conditions, involving for example, vascularization and/or inflammation, include atherosclerosis, rheumatoid arthritis (RA), hemangiomas, angiofibromas, and psoriasis. Other non-limiting examples of angiogenic disease are retinopathy of prematurity (retrolental fibroplastic), corneal graft rejection, corneal neovascularization related to complications of refractive surgery, corneal neovascularization related to contact lens complications, corneal neovascularization related to pterygium and recurrent pterygium, corneal ulcer disease, and non-specific ocular surface disease, insulin-dependent diabetes mellitus, multiple sclerosis, myasthenia gravis, Chron's disease, autoimmune nephritis, primary biliary cirrhosis, acute pancreatitis, allograph rejection, allergic inflammation, contact dermatitis and delayed hypersensitivity reactions, inflammatory bowel disease, septic shock, osteoporosis, osteoarthritis, cognition defects induced by neuronal inflammation, Osler-Weber syndrome, restenosis, and fungal, parasitic and viral infections, including cytomegaloviral infections.

Non-limiting examples of VEGF-binding agents include VEGF antibodies and VEGF traps (i.e., ligand binding domains of VEGF receptors. Two examples of antibodies (including VEGF-binding antibody fragments) are bevacizumab (Avastin), an antibody which binds to VEGF-A, and ranibizumab (Lucentis), an Fab derived from bevacizumab. In general, a VEGF trap is a protein that comprises VEGF binding domains of one or more VEGF receptor protein. VEGF-traps include, without limitation, soluble VEGFR-1, soluble neuropilin 1 (NRP1), soluble VEGFR-3 (which binds VEGF-C and VEGF-D), and aflibercept (Zaltrap; Eyelea; VEGF Trap R1R2), comprised of segments of the extracellular domains of human vascular endothelial growth factor receptors VEGFR1 and VEGFR2 fused to the constant region (Fc) of human IgG1. Conbercept (KH902) is a fusion protein which contains the extracellular domain 2 of VEGFR-1 (Flt-1) and extracellular domain 3, 4 of VEGFR-2 (KDR) fused to the Fc portion of human IgG1. Several VEGF traps containing KDR and FLT-1 Ig-like domains in various combinations are disclosed in U.S. Pat. No. 8,216,575. DARPins (an acronym for designed ankyrin repeat proteins) are genetically engineered antibody mimetic proteins typically exhibiting highly specific and high-affinity target protein binding. DARPin® MP0112 is a vascular endothelial growth factor (VEGF) inhibitor and has entered clinical trials for the treatment of wet macular degeneration and diabetic macular edema.

According to the invention, VEGF expression can be targeted. For example, VEGF inhibitor PTC299 targets VEGF post-transcriptionally by selectively binding the 5′- and 3′-untranslated regions (UTR) of VEGF messenger RNA (mRNA), thereby preventing translation of VEGF. Pegaptanib (Macugen) is an RNA aptamer directed against VEGF-165.

Non-limiting examples of agents that bind to the extracellular domain of VEGFR2 include the novel VEGFR2 antibodies and VEGFR2-binding fragments disclosed herein. Other examples are ramucirumab (IMC-1121B), IMC-1C11, and CDP791 (an engineered antibody fragment that comprises two antigen-binding fragments (di-Fab) of a humanized antibody covalently cross-linked at their hinge region and polyethylene glycol attached to the cross-linker.

Placental growth factor (PlGF) has been implicated in pathological angiogenesis. PlGF is structurally related to VEGF and is also a ligand for VEGFR-1. Consequently, VEGF traps comprising the extracellular domain of VEGFR1 (see above) are useful for targeting PlGF.

PDGF is composed of four polypeptide chains that form homodimers PDGF-AA, BB, CC, and DD as well as the heterodimer PDGF-AB. The PDGF receptors (PDGFR)-α and -β mediate PDGF functions. Specifically, PDGFRα binds to PDGF-AA, -BB, -AB, and -CC, whereas PDGFRβ interacts with -BB and -DD. Non-limiting examples of PDGF-binding agents include anti-PDGF antibodies and PDGF traps. Agents that target PDGF include Fovista™ (E10030, Ophthotech), a pegylated aptamer targeting PDGF-B, and AX102 (Sennino et al., 2007, Cancer Res. 75(15):7359-67), a DNA oligonucleotide aptamer that binds PDGF-B.

Agents that target PDGF receptors include ramucirumab (IMC-3G3, human IgG₁) an anti-PDGFRα antibody, crenolanib (CP-868596), a selective inhibitor of PDGFRα (IC₅₀=0.9 nM) and PDGFRβ (IC₅₀=1.8 nM), and nilotinib (Tasigna®), an inhibitor of PDGFRα and PDGFRβ and other tyrosine kinases.

Angiogenesis inhibitors include intracellular agents that block signal transduction mediated by, for example, VEGF, PDGF, ligands of VEGF or PDGF receptors, or complement. Intracellular agents that inhibit angiogenesis inhibitors include the following, without limitation. Sunitinib (Sutent; SU11248) is a panspecific small-molecule inhibitor of VEGFR1-VEGFR3, PDGFRα and PDGFRβ, stem cell factor receptor (cKIT), Flt-3, and colony-stimulating factor-1 receptor (CSF-1R). Axitinib (AG013736; Inlyta) is another small molecule tyrosine kinase inhibitor that inhibits VEGFR-1-VEGFR-3, PDGFR, and cKIT. Cediranib (AZD2171) is an inhibitor of VEGFR-1-VEGFR-3, PDGFRβ, and cKIT. Sorafenib (Nexavar) is another small molecular inhibitor of several tyrosine protein kinases, including VEGFR, PDGFR, and Rafkinases. Pazopanib (Votrient; (GW786034) inhibits VEGFR-1, -2 and -3, cKIT and PDGFR. Foretinib (GSK1363089; XL880) inhibits VEGFR2 and MET. CP-547632 is as a potent inhibitor of the VEGFR-2 and basic fibroblast growth factor (FGF) kinases. E-3810 ((6-(7-((1-aminocyclopropyl) methoxy)-6-methoxyquinolin-4-yloxy)-N-methyl-1-naphthamide) inhibits VEGFR-1, -2, and -3 and FGFR-1 and -2 kinases in the nanomolar range. Brivanib (BMS-582664) is a VEGFR-2 inhibitor that also inhibits FGF receptor signaling. CT-322 (Adnectin) is a small protein based on a human fibronectin domain and binds to and inhibits activation of VEGFR2. Vandetanib (Caprelas; Zactima; ZD6474) is an inhibitor of VEGFR2, EGFR, and RET tyrosine kinases. X-82 (Xcovery) is a small molecule indolinone inhibitor of signaling through the growth factor receptors VEGFR and PDGFR.

In another embodiment, the ocular disease is glaucoma. There are several types of glaucoma which can be treated, including, without limitation, the following types. The two most common, primary open-angle glaucoma and acute angle-closure glaucoma are characterized by high ocular pressure. Pigmentary glaucoma, neovascular glaucoma, and congenital glaucoma also are characterized by reduced fluid outflow and high intraocular pressure (IOP). Normal tension glaucoma, is thought to be due to another mechanism, in particular poor blood flow to the optic nerve. Secondary glaucoma can result from injury, infection, inflammation, tumor or cataracts, and is also associated with prolonged use of steroids, systemic hypertension, diabetic retinopathy, and central retinal vein occlusion.

The invention provides a method of treating glaucoma which comprises administering to a patient in need thereof, an effective amount of a Rho-kinase inhibitor. In certain embodiments, the Rho-kinase inhibitor is a compound of any one of Formulae I-XXV. The Rho-kinase inhibitor can be non-selective with respect to ROCK1 and ROCK2, or can be a selective ROCK1 inhibitor, or a selective ROCK2 inhibitor. Generally, it is preferred that the inhibitor inhibits ROCK1, i.e., inhibits both ROCK1 and ROCK2 or is selective for ROCK1. In the context of this invention, selective means the inhibitor demonstrates an IC₅₀ that is at least 2-fold, at least 5-fold, at least 10-fold, or at least 25-fold lower for one Rho kinase as compared to the IC₅₀ for the other Rho kinase. As discussed above, there are several types glaucomas, compounds selective for ROCK1 or ROCK2 can be beneficial for treating certain types. Also, certain glaucomas having a neovascular component can benefit from administration of a angiogenesis inhibitor in addition to a ROCK inhibitor.

According to the invention, a ROCK2 inhibitor and the angiogenesis inhibitor can be administered to a subject in the same pharmaceutical composition or in separate pharmaceutical compositions. The agents may be administered to a subject by the same or different routes of administration.

In an embodiment of the invention, the ROCK2 inhibitor and the angiogenesis inhibitor are both small molecules and are administered orally. In a preferred embodiment, the agents are administered once daily. When the agents are administered on the same scheduled, the ROCK2 inhibitor and the angiogenesis inhibitor may be combined in the same dosage form so that they are coadministered.

In one embodiment of the invention, the angiogenesis inhibitor is an antibody or an antigen binding fragment thereof which is administered by injection, preferably intravitreal injection and the ROCK2 inhibitor is a small molecule administered orally. In one such embodiment, the angiogenesis inhibitor is administered weekly or once or twice per month and the ROCK2 inhibitor is administered daily.

In some of the treatment methods described herein, the invention includes administration of a TGF-β antagonist. TGF-β plays an important role in scarring, including, without limitation, retinal scarring associated with macular degeneration, detached retina, and surgical interventions for glaucoma. Of the three TGF-βs expressed in mammals, TGF-β₂ is thought to be the most important isoform for retinal scarring, and there is a significant correlation between the levels of TGF-β₂ expression and the severity of retinal scarring.

According to the invention, when a ROCK inhibitor, or a VEGF antagonist, or a ROCK inhibitor and a VEGF antagonist are administered to a subject to treat an ocular disease, a TGF-β antagonist can be administered to the subject to reduce or prevent scarring. For example, in an embodiment of the invention, when a ROCK inhibitor is administered to treat an ocular disorder, a TGF-β antagonist is also administered. In another embodiment, when a VEGF antagonist is administered to a subject to treat an ocular disorder, a TGF-β antagonist is also administered. In another embodiment of the invention, when a ROCK inhibitor and a VEGF antagonist are administered to a subject to treat an ocular disorder, a TGF-β antagonist is also administered. In ocular diseases involving neovascularization, leakage of new blood vessels is followed by scar formation (e.g., discaform scar). The invention includes administration of a TGF-β antagonist as well as a VEGF antagonist and a ROCK2 inhibitor to a subject to treat neovascularization in ocular disease.

Useful TGF-β antagonists include, without imitation, the following: (i) anti-TGF-β antibodies and antigen binding fragments thereof, such as pan-TGF-β antibody GC-1008 (Genzyme), anti-TGF-β₁ antibody metelimumab (CAT-192) (Cambridge Antibody Technology), and antigen binding fragments of those antibodies, (ii) soluble TGF-β receptors or ligand binding fragments thereof, such as P144, a synthetic peptide encompassing amino acids 730-743 from the membraneproximal ligand-binding domain of TGF-β type III receptor (Esparza-López et al., 2001, J. Biol. Chem. 276(18):14588-96), and a type II TGF-β receptor—Fc (IgG₁) fusion (Smith, J. et al., 1999, Circulation Res. 84:1212-22), (iii) peptides that bind to TGF-β receptors that block one or more isoforms of TGF-β, such as the 25 amino acid peptides from TGF-β₁, TGF-β₂, and TGF-β₃ disclosed by Huang et al., 1997, J. Biol. Chem. 272:27155-59, that bind to TGF-β receptors, and (iv) antisense agents that inhibit TGF-β synthesis, such as trabedersen (Antisense Pharma GmbH), an oligonucleotide that inhibits the synthesis of TGF-β₂. Additional antagonists are disclosed in WO2006/052568, WO 02/094833, WO 04/048382, WO 04/048381, WO 04/050659, WO 04/021989, WO 04/026871, and WO 04/026307.

Th17 cells are novel subset of helper CD4⁺ T cells that secrete IL-17, IL-21 and IL-22. The pro-inflammatory activity of Th17 cells can be beneficial to the host during infection, but uncontrolled Th17 function has been linked and actively involved in several autoimmune pathologies and development of acute graft-versus-host disease (GVHD), a disease characterized by selective epithelial damage to target organs that is mediated by mature T cells present in the stem cell or bone marrow inoculums. Indeed, high levels of IL-17 are detected in the sera and biopsies of rheumatoid arthritis (RA) and systemic lupus erythematosus (SLE) patients which correlates with destruction of synovial tissue and disease activity. The pathological role of IL-17 in arthritic joints is associated with its stimulation of pro-inflammatory cytokine production and increased recruitment of T cells and innate immune cells. Moreover, numbers of Th17 cells are significantly increased in the peripheral blood of RA patients as well as elevated concentrations of IL-17 were seen in supernatants of their PBMCs after stimulation with anti-CD3/CD28 antibodies ex vivo. In addition, in multiple sclerosis (MS) patients, myelin reactive Th17 cells are also enriched and produce high amounts of IL-22 and IFN-γ. Further, a significant higher number of IL-17⁺ cells is detected in disease-affected gut areas compared to healthy areas of the same subjects with Crohn's disease (CD).

According to the invention, targeting Th17 (IL-17-secreting) cells by rho-kinase inhibition provides a method for treating Th17 cell-mediated diseases, including but not limited to autoimmune disorders such as RA, MS, SLE, Psoriasis, and Crohn's disease, and GVHD in humans. In an embodiment of the invention, the Rho-kinase inhibitor is a compound of Formula I. In some embodiments, the rho-kinase inhibitor inhibits ROCK1 and ROCK2. In some embodiments, the rho-kinase inhibitor selectively inhibits ROCK2. In some embodiments, selective inhibition of ROCK2 reduces or prevents toxicities associated with complete inhibition of ROCK activity.

The development and function of Th17 cells depends on activation of specific intracellular signaling pathways. The steroid receptor-type nuclear receptor RORγt is selectively expressed in Th17 cells and appears to be required for IL-17 production. The induction of RORγt has been observed to be mediated by IL-6, IL-21 and IL-23 via a STAT3-dependent mechanism. STAT3 also binds directly to the IL-17 and IL-21 promoters. In addition to RORγt and STAT3, the interferon regulatory factor 4 (IRF4) is required for the differentiation of Th17 cells since IRF4 KO mice failed to mount Th17 response and were resistant to development of autoimmune responses. Recent studies have demonstrated that phosphorylation of IRF4 by Rho-kinase 2 (ROCK2) regulates IL-17 and IL-21 production and development of autoimmunity in mice.

In certain embodiments, a dose of a compound or a composition is administered to a subject every day, every other day, every couple of days, every third day, once a week, twice a week, three times a week, or once every two weeks. In other embodiments, two, three or four doses of a compound or a composition is administered to a subject every day, every couple of days, every third day, once a week or once every two weeks. In some embodiments, a dose(s) of a compound or a composition is administered for 2 days, 3 days, 5 days, 7 days, 14 days, or 21 days. In certain embodiments, a dose of a compound or a composition is administered for 1 month, 1.5 months, 2 months, 2.5 months, 3 months, 4 months, 5 months, 6 months or more.

Methods of administration include but are not limited to parenteral, intradermal, intravitrial, intramuscular, intraperitoneal, intravenous, subcutaneous, intranasal, epidural, oral, sublingual, intranasal, intracerebral, intravaginal, transdermal, transmucosal, rectally, by inhalation, or topically, particularly to the ears, nose, eyes, or skin. The mode of administration is left to the discretion of the practitioner. In most instances, administration will result in the release of a compound into the bloodstream. For treatment of ocular disease, intravitrial administration of biological agents is preferred.

In specific embodiments, it may be desirable to administer a compound locally. This may be achieved, for example, and not by way of limitation, by local infusion, topical application, by injection, by means of a catheter, or by means of an implant, said implant being of a porous, non-porous, or gelatinous material, including membranes, such as sialastic membranes, or fibers. In such instances, administration may selectively target a local tissue without substantial release of a compound into the bloodstream.

Pulmonary administration can also be employed, e.g., by use of an inhaler or nebulizer, and formulation with an aerosolizing agent, or via perfusion in a fluorocarbon or synthetic pulmonary surfactant. In certain embodiments, a compound is formulated as a suppository, with traditional binders and vehicles such as triglycerides.

In another embodiment, a compound is delivered in a vesicle, in particular a liposome (See Langer, 1990, Science 249:1527-1533; Treat et al., in Liposomes in the Therapy of Infectious Disease and Bacterial infection, Lopez-Berestein and Fidler (eds.), Liss, New York, pp. 353-365 (1989); Lopez Berestein, ibid., pp. 317-327; see generally ibid.).

In another embodiment, a compound is delivered in a controlled release system (See, e.g., Goodson, in Medical Applications of Controlled Release, supra, vol. 2, pp. 115-138 (1984)). Examples of controlled-release systems are discussed in the review by Langer, 1990, Science 249:1527-1533 may be used. In one embodiment, a pump may be used (See Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (See Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; See also Levy et al., 1985, Science 228:190; During et al., 1989, Ann. Neurol. 25:351; Howard et al., 1989, J. Neurosurg. 71:105).

The above-described administration schedules are provided for illustrative purposes only and should not be considered limiting. A person of ordinary skill in the art will readily understand that all doses are within the scope of the invention.

It is to be understood and expected that variations in the principles of invention herein disclosed may be made by one skilled in the art and it is intended that such modifications are to be included within the scope of the present invention.

Throughout this application, various publications are referenced. These publications are hereby incorporated into this application by reference in their entireties to more fully describe the state of the art to which this invention pertains. The following examples further illustrate the invention, but should not be construed to limit the scope of the invention in any way.

### EXAMPLES

Abbreviations used in the following examples and preparations include:
Ac₂O acetic anhydride
AcOH acetic acid
Bn Benzyl
Celite® diatomaceous earth
DCM dichloromethane
DIEA di-isopropylethylamine
DMAP 4-dimethylamino pyridine
DME 1,2-dimethoxylethane
DMF dimethylformamide
DMSO dimethyl sulfoxide
EDC 1-(3-dimethylaminopropyl)-3-ethylcarbodimide hydrochloride
EtOAc ethyl acetate
EtOH ethyl alcohol or ethanol
Et₂O ethyl ether
Et₃N triethylamine
g grams
HOBt 1-hydroxybenzotriazole
HPLC high pressure liquid chromatography
h hour(s)
MeCN acetonitrile
min minute(s)
MeOH methyl alcohol or methanol
mL milliliter
mmol millimoles
MS mass spectrometry
NMR nuclear magnetic resonance
iPrOH iso-propanol
PyBOP® benzotriazol-1-yl-oxytripyrrolidinophosphonium
rt room temperature
s singlet
t triplet
THF tetrahydrofuran

Mass spectrometry was conducted by: SynPep Co., 6905 Ct. Dublin, Calif. 94568, or it was recorded on an LC-MS: Waters 2695 Separations Module with a Waters ZQ2000 single quadrapole MS detector. Unless stated all mass spectrometry was run in ESI mode. 1H NMR spectra were recorded on a Varian 400 MHz machine using Mercury software. In sofar the synthesis of the following examples of compounds of the present inventation is not explicitely described in such example, the synthesis is as described herein in general terms and the appropriate starting material can be easily selected for synthesizing the compound of the example.

### Example 1

### 2-Bromo-N-isoopropylacetamide

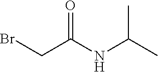

A solution of iso-propyl amine (5.0 g, 7.20 mL, 84.6 mmol) in 63 mL of DCM was cooled to −10° C. To this was added a solution of a-bromoacetylbromide (8.53 g, 3.68 mL, 42.3 mmol) in 10.5 mL of DCM. The reaction mixture was stirred for 10 min. The iso-propylammonium hydrobromide was filtered from the mixture and the filtrate then concentrated in vacuo to give the title compound as a white solid (5.30 g, 70%).

### Example 2

### N-isopropyl-2-(3-(4,4,5,5-tetramethyl-1,3,2-dioxaborolan-2-yl)phenoxy)acetamide

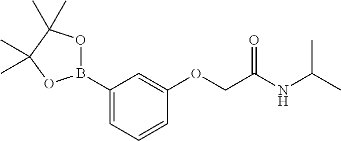 [SMILES: CC(C)NC(=O)COc1cccc(B2OC(C)(C)C(C)(C)O2)c1]

A mixture of 3-(4,4,5,5-tetramethyl-1,3,2-dioxaborolan-2-yl)phenol (0.50 g, 2.27 mmol), 2-bromo-N-isoopropylacetamide (0.61 g, 3.41 mmol), and K₂CO₃ (0.47 g, 3.41 mmol) in DMF (3 mL) was stirred at rt followed by addition of ice water. The precipitate was filtered and washed with water and dried to provide the title compound (0.32 g, 44%).

### Example 3

### tert-Butyl 5-((2-chloropyrimidin-4-yl)amino)-1H-indazole-1-carboxylate

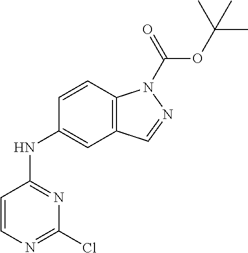 [SMILES: CC(C)(C)OC(=O)n1ncc2cc(Nc3ccnc(Cl)n3)ccc21]

A mixture of 2,4-dichloropyrimidine (1.99 g, 13.4 mmol), tert-butyl 5-amino-1H-indazole-1-carboxylate (3.4 g, 14.7 mmol), DIEA (3 mL), and DMF (13 mL) was stirred at 65° C. for 7 h, concentrated in vacuo, and titurated with Et₂O. The precipitate was filtered and washed with IPA and dried to provide the title compound (1.83 g, 40%).

### Example 4

### tert-Butyl 5-((2-(3-(2-(isopropylamino)-2-oxoethoxy)phenyl)pyrimidin-4-yl)amino)-1H-indazole-1-carboxylate

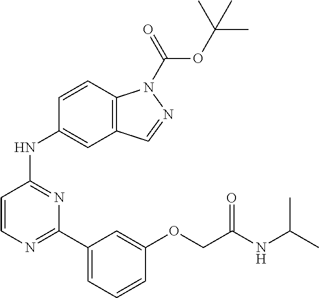 [SMILES: CC(C)NC(=O)COc1cccc(-c2nccc(Nc3ccc4c(cnn4C(=O)OC(C)(C)C)c3)n2)c1]

A mixture of tert-butyl 5-((2-chloropyrimidin-4-yl)amino)-1H-indazole-1-carboxylate (100 mg, 0.29 mmol), N-isopropyl-2-(3-(4,4,5,5-tetramethyl-1,3,2-dioxaborolan-2-yl)phenoxy)acetamide (130 mg, 0.41 mmol), Pd(dppf)Cl₂.CH₂Cl₂ (20 mg, 0.02 mmol), and K₂CO₃ (80 mg, 0.58 mmol) in dioxane/water (10 and 2 mL) was heated in microwave for 30 min. The reaction was worked up and purified by chromatography to provide the title compound (176 mg, 35%).

### Example 5

### 2-(3-(4-((1H-indazol-5-yl)amino)pyrimidin-2-yl)phenoxy)-N-isopropylacetamide HCl salt

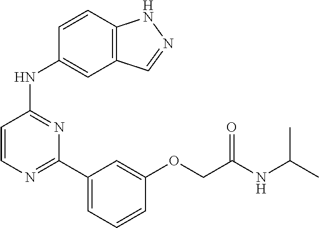 [SMILES: CC(C)NC(=O)COc1cccc(-c2nccc(Nc3ccc4[nH]ncc4c3)n2)c1]

tert-Butyl 5-((2-(3-(2-(isopropylamino)-2-oxoethoxy)phenyl)pyrimidin-4-yl)amino)-1H-indazole-1-carboxylate was taken up in 4 M HCl in dioxane and stirred at rt for 2 h. The volatiles were removed in vacuo to give the title compound as HCl salt. ¹H NMR (400 MHz, DMSO-d₆) δ12.6 (d, J=6.8 Hz, 6H), 3.93 (m, 1H), 4.51 (s, 2H), 6.75 (d, J=6 Hz, 1H), 7.14 (d, J=7.6 Hz, 1H), 7.44-7.59 (m, 3H), 7.87-7.91 (m, 3H), 8.09 (s, 1H), 8.18 (s, 1H), 8.31 (d, J=6.4 Hz, 1H), 10.19 (d, J=0.8 Hz, 1H), 13.10 (s, 1H). MS (ES+) m/e 403 (M+H)⁺.

### Example 6

### tert-butyl 2-(3-(4,4,5,5-tetramethyl-1,3,2-dioxaborolan-2-yl)phenoxy)acetate

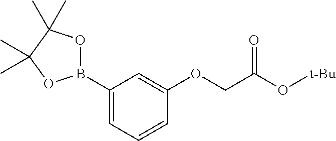 [SMILES: CC(C)(C)OC(=O)COc1cccc(B2OC(C)(C)C(C)(C)O2)c1]

### 3-(4,4,5,5-tetramethyl-1,3,2-dioxaborolan-2-yl)phenol, tert-butyl 2-bromoacetate

A mixture of 3-(4,4,5,5-tetramethyl-1,3,2-dioxaborolan-2-yl)phenol (4 g, 18.2 mmol), tert-butyl 2-bromoacetate (5.7 g, 27.3 mmol) and K₂CO₃ (3.44 g, 27.3 mmol) in CH₃CN (100 mL) was stirred at 70° C. overnight. The reaction mixture was poured into water and extracted with EtOAc. The organic layer was dried over Na₂SO₄ and removed to give a residue. The residue was purified by column chromatograph to give the title compound (4g, 67%) as a white solid.

### Example 7

### N-(2-chloropyrimidin-4-yl)-1H-indazol-5-amine

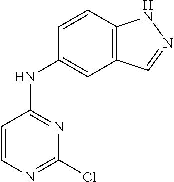 [SMILES: Clc1nccc(Nc2ccc3[nH]ncc3c2)n1]

A mixture of compound 2,4-dichloropyrimidine (14.8 g, 0.1 mol), 1H-indazol-5-amine (14.6 g, 110 mmol) and Et₃N (15 g, 150 mmol) in EtOH (200 mL) was stirred at 80° C. for 3 hrs. The reaction mixture was cooled and filtered. The filtered cake was collected and dried to give compound 5 (15 g, 60%) as a solid.

### Example 8

### tert-butyl 5-((tert-butoxycarbonyl)(2-chloropyrimidin-4-yl)amino)-1H-indazole-1-carboxylate

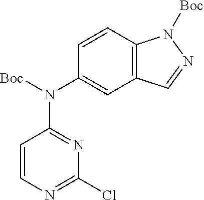 [SMILES: CC(C)(C)OC(=O)N(c1ccc2c(cnn2C(=O)OC(C)(C)C)c1)c1ccnc(Cl)n1]

To a mixture of N-(2-chloropyrimidin-4-yl)-1H-indazol-5-amine (7.35 g, 30 mmol), Boc₂O (18.9 g, 90 mmol) in CH₂Cl₂ (150 mL) was added DMAP (3.6 g, 30 mmol) at 0° C. during 5 min. After 0.5 hr, the reaction was completed. The reaction mixture was washed with water, dried over Na₂SO₄ and removed to give a residue, which was purified by gel column chromatograph to give the title compound (6g, 67%) as a white solid.

### Example 9

### tert-butyl 5-((2-(3-(2-(tert-butoxy)-2-oxoethoxy)phenyl)pyrimidin-4-yl)(tert-butoxycarbonyl)amino)-1 H-indazole-1-carboxylate

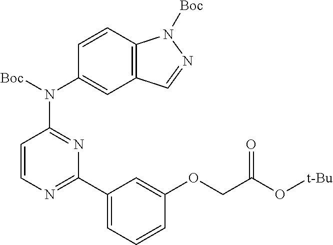 [SMILES: CC(C)(C)OC(=O)COc1cccc(-c2nccc(N(C(=O)OC(C)(C)C)c3ccc4c(cnn4C(=O)OC(C)(C)C)c3)n2)c1]

A mixture of tert-butyl 5-((tert-butoxycarbonyl)(2-chloropyrimidin-4-yl)amino)-1H-indazole-1-carboxylate (4 g, 8.9 mmol), tert-butyl 2-(3-(4,4,5,5-tetramethyl-1,3,2-dioxaborolan-2-yl)phenoxy)acetate (3.3 g, 10 mmol), KOAc (35 g, 360 mmol), Pd(dppf)₂Cl₂ (400 mg) and Boc₂O (3.9 g, 18 mmol) in dioxane/water (10/1, 100 mL) was stirred at 100° C. for 3 days. The reaction mixture was poured into water and extracted with EtOAc. The organic layer was dried over Na₂SO₄ and removed to give a residue, which was purified by gel column chromatograph to give the title compound (3g, 54%) as a solid.

### Example 10

### 2-(3-(4-((1H-indazol-5-yl)amino)pyrimidin-2-yl)phenoxy)acetic acid

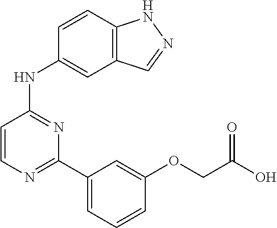 [SMILES: O=C(O)COc1cccc(-c2nccc(Nc3ccc4[nH]ncc4c3)n2)c1]

A mixture of compound tert-butyl 5-((2-(3-(2-(tert-butoxy)-2-oxoethoxy)phenyl)pyrimidin-4-yl)(tert-butoxycarbonyl)amino)-1H-indazole-1-carboxylate (2 g) and CF₃COOH (20 mL) in DCM (20 mL) was stirred at 25° C. for 2 hrs. The solvent was removed to give the title compound (1.5 g) as a yellow solid.

### Example 11

### (S)-tert-butyl 3-(2-(3-(4-((1H-indazol-5-yl)amino)pyrimidin-2-yl)phenoxy)acetamido)pyrrolidine-1-carboxylate

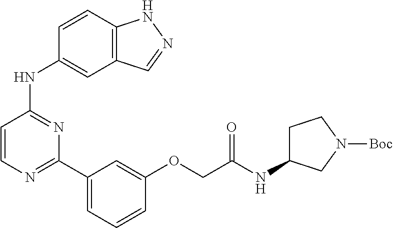 [SMILES: CC(C)(C)OC(=O)N1CC[C@H](NC(=O)COc2cccc(-c3nccc(Nc4ccc5[nH]ncc5c4)n3)c2)C1]

A mixture of 2-(3-(4-((1H-indazol-5-yl)amino)pyrimidin-2-yl)phenoxy)acetic acid (600 mg, 1.66 mmol), 3-amino-pyrrolidine-1-carboxylic acid tert-butyl ester (300 mg, 1.62 mmol), HATU (760 mg, 2 mmol) and Et₃N (250 mg, 2 mmol) in DMF (18 mL) was stirred at 25° C. overnight. The reaction mixture was poured into water and extracted with EtOAc. The organic layer was dried over Na₂SO₄ and concentrated to give a residue, which was purified by HPLC to provide the title compound (300 mg, 50%) as a solid.

### Example 12

### (R)-2-(3-(4-((1H-indazol-5-yl)amino)pyrimidin-2-yl)phenoxy)-N-(pyrrolidin-3-yl)acetamide HCl salt

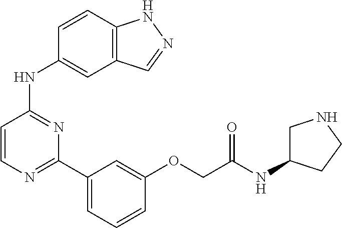 [SMILES: O=C(COc1cccc(-c2nccc(Nc3ccc4[nH]ncc4c3)n2)c1)N[C@@H]1CCNC1]

¹H NMR (400 MHz, DMSO-d₆) δ1.84-1.96 (m, 1H), 2.07-2.19 (m, 1H), 3.07-3.38 (m, 4H), 4.40-4.42 (m, 1H), 4.65 (s, 2H), 7.36 (d, J=6.4 Hz, 1H), 7.56 (t, J=8.0 Hz, 1H), 7.65 (d, J=8.0 Hz, 1H), 7.90-7.94 (m, 2H), 8.14-8.29 (m, 2H), 8.66 (d, J=6.8 Hz, 1H), 9.39-9.80 (m, 3H), 11.87 (s, 1H). MS (ES+) m/e 430 (M+H)⁺.

### Example 13

### (R)-2-(3-(4-((1H-indazol-5-yl)amino)pyrimidin-2-yl)phenoxy)-N-(pyrrolidin-3-yl)acetamide HCl salt

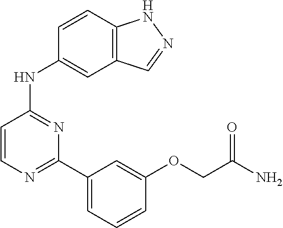 [SMILES: NC(=O)COc1cccc(-c2nccc(Nc3ccc4[nH]ncc4c3)n2)c1]

A mixture of compound 2-(3-(4-((1H-indazol-5-yl)amino)pyrimidin-2-yl)phenoxy)acetic acid (500 mg, 1.39 mmol), NH₄Cl (125 mg, 2 mmol), HATU (720 mg, 1.89 mmol) and Et₃N (200 mg, 2 mmol) in DMF (15 mL) was stirred at 25° C. overnight. The reaction mixture was poured into water and extracted with EtOAc. The organic layer was dried over Na₂SO₄ and concentrated to give a residue, which was purified by HPLC to provide the title compound (60.1 mg, 12%) as a solid. ¹H NMR (400 MHz, DMSO-d₆) δ4.52 (s, 2H), 6.78 (d, J=6.4 Hz, 1H), 7.19 (dd, J=9.6 and 1.6 Hz, 1H), 7.40-7.62 (m, 5H), 7.84-7.87 (m, 2H), 8.11 (s, 1H), 8.15 (s, 1H), 8.33 (d, J=6.4 Hz, 1H), 10.35 (s, 1H). MS (ES+) m/e 361 (M+H)⁺.

### Example 14

### 2-(3-(4-((1H-indazol-5-yl)amino)pyrimidin-2-yl)phenoxy)-1-(1,1-dioxidothiomorpholino)ethanone TFA salt

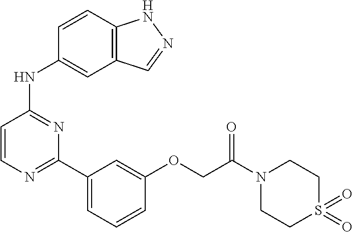 [SMILES: O=C(COc1cccc(-c2nccc(Nc3ccc4[nH]ncc4c3)n2)c1)N1CCS(=O)(=O)CC1]

A mixture of compound 2-(3-(4-((1H-indazol-5-yl)amino)pyrimidin-2-yl)phenoxy)acetic acid (500 mg, 1.39 mmol), thiomorpholine 1,1-dioxide (375 mg, 2.2 mmol), HATU (720 mg, 1.89 mmol) and Et3N (200 mg, 2 mmol) in DMF (15 mL) was stirred at 25° C. overnight. The reaction mixture was poured into water and extracted with EtOAc. The organic layer was dried over Na2SO4 and concentrated to give a residue, which was purified by pre-HPLC to give the title compound (54.6 mg, 10%) as a solid. 1H NMR (400 MHz, DMSO-d6) δ3.11 (b, 2H), 3.30 (b, 2H), 3.85 (b, 4H), 5.00 (s, 2H), 6.76 (d, J=6.4 Hz, 1H), 6.91 (s, 1H), 7.09 (s, 1H), 7.16 (dd, J=8.0 and 2.4 Hz, 1H), 7.45 (t, J=8.0 Hz, 1H), 7.47-7.50 (m, 2H), 7.81 (s, 1H), 7.86 (d, J=8.0 Hz, 1H), 8.08 (s, 1H), 8.12 (b, 1H), 8.33 (d, J=6.4 Hz, 1H), 10.26 (s, 1H). MS (ES+) m/e 479 (M+H)⁺.

### Example 15

### N-(3-(4,4,5,5-tetramethyl-1,3,2-dioxaborolan-2-yl)phenyl)morpholine-4-carboxamide

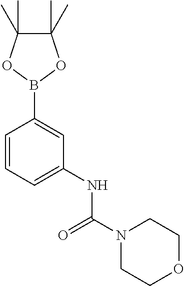 [SMILES: CC1(C)OB(c2cccc(NC(=O)N3CCOCC3)c2)OC1(C)C]

To a mixture of triphosgene (600 mg, 2 mmol) in THF (10 mL) was dropped a solution of 3-(4,4,5,5-tetramethyl-1,3,2-dioxaborolan-2-yl)aniline (836 mg, 4 mmol) and Et₃N (1.2 g, 12 mmol) in THF (10 mL) during 10 min at 40° C. After 0.5 hr, morpholine (435 mg, 5 mmol) was added to the reaction mixture. After 15 min, the reaction mixture was quenched with saturated NH₄Cl, extracted with EtOAc. The organic layer was dried over Na₂SO₄ and concentrated to give a residue, which was purified by gel column chromatography to give the title compound (530 mg, 50%) as a white solid.

### Example 16

### tert-butyl 5-((tert-butoxycarbonyl)(2-(3-(morpholine-4-carboxamido)phenyl)pyrimidin-4-yl)amino)-1H-indazole-1-carboxylate

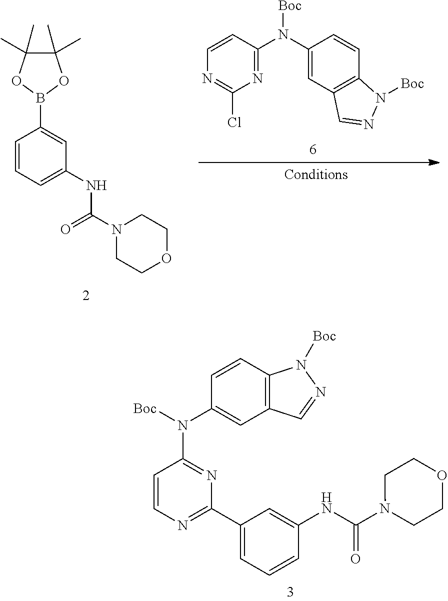 [SMILES: CC1(C)OB(c2cccc(NC(=O)N3CCOCC3)c2)OC1(C)C ; CC(C)(C)OC(=O)N(c1ccc2c(cnn2C(=O)OC(C)(C)C)c1)c1ccnc(Cl)n1 ; CC(C)(C)OC(=O)N(c1ccc2c(cnn2C(=O)OC(C)(C)C)c1)c1ccnc(-c2cccc(NC(=O)N3CCOCC3)c2)n1]

A mixture of tert-butyl 5-((tert-butoxycarbonyl)(2-chloropyrimidin-4-yl)amino)-1H-indazole-1-carboxylate (300 mg, 0.7 mmol), N-(3-(4,4,5,5-tetramethyl-1,3,2-dioxaborolan-2-yl)phenyl)morpholine-4-carboxamide (250 mg, 0.8 mmol), CsF (510 mg, 3 mmol), Pd(PPh₃)₄ (120 mg) and Boc₂O (432 mg, 2 mmol) in dioxane/water (10/1, 10 mL) was stirred at 130° C. under microwave for 20 min. Three pots were combined. The reaction mixture was diluted with water and extracted with EtOAc. The organic layer was dried over Na₂SO₄ and concentrated to give a residue, which was purified by column chromatograph to give the title compound (360 mg).

### Example 17

### N-(3-(4-((1H-indazol-5-yl)amino)pyrimidin-2-yl)phenyl)morpholine-4-carboxamide HCl salt

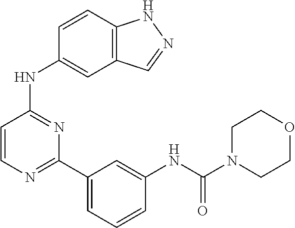 [SMILES: O=C(Nc1cccc(-c2nccc(Nc3ccc4[nH]ncc4c3)n2)c1)N1CCOCC1]

A mixture of tert-butyl 5-((tert-butoxycarbonyl)(2-(3-(morpholine-4-carboxamido)phenyl)pyrimidin-4-yl)amino)-1H-indazole-1-carboxylate (360 mg, crude) in MeOH/HCl (4M, 20 mL) was stirred at 25° C. overnight. The reaction mixture was concentrated to give title compound (68 mg) as an HCl salt. ¹H NMR (400 MHz, DMSO-d₆) δ3.49 (s, 4H), 3.62 (s, 4H), 7.14 (b, 1H), 7.50 (t, J=7.6 Hz, 1H), 7.60-7.65 (m, 3H), 7.88 (d, J=6.8 Hz, 1H), 8.22 (s, 1H), 8.28 (d, J=5.6 Hz, 1H), 8.46 (b, 1H), 8.68 (s, 1H), 9.00 (s, 1H), 11.77 (s, 1H). MS (ES+) m/e 416 (M+H)⁺.

### Example 18

### Methyl 3-(4-((1H-indazol-5-yl)amino)pyrimidin-2-yl)benzoate

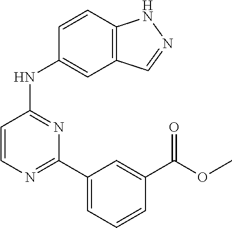 [SMILES: COC(=O)c1cccc(-c2nccc(Nc3ccc4[nH]ncc4c3)n2)c1]

A mixture of N-(2-chloropyrimidin-4-yl)-1H-indazol-5-amine (3.7 g, 15 mmol), 3-methoxycarbonylphenylboronic acid (3.3 g, 18 mmol), K₂CO₃ (4.14 g, 30 mmol) and Pd(dppf)₂Cl₂ (700 mg) in dioxane/water (4/1, 75 mL) was stirred at 100° C. for 16 hrs. The reaction mixture was concentrated to give the title compound (7 g, crude) which was carried out directly for next step reaction without further purification.

### Example 19

### 3-(4-((1H-indazol-5-yl)amino)pyrimidin-2-yl)benzoic acid

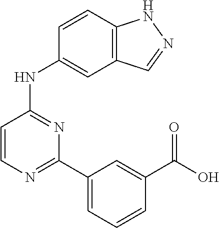 [SMILES: O=C(O)c1cccc(-c2nccc(Nc3ccc4[nH]ncc4c3)n2)c1]

To a mixture of methyl 3-(4-((1H-indazol-5-yl)amino)pyrimidin-2-yl)benzoate (7 g, crude) in dioxane (120 mL) was added NaOH (2M, 120 mL). The reaction mixture was refluxed for 1.5 h and was then cooled to rt and extracted with EtOAc (100 mL). The aqueous phase was separated and acidified with HCl (6M). The mixture was filtered and the filter cake was collected, dried to give the title compound (1.3 g, crude) used for the next step reaction directly.

### Example 20

### 3-(4-((1H-indazol-5-yl)amino)pyrimidin-2-yl)-N-(1,1-dioxidotetrahydro-2H-thiopyran-4-yl)benzamide TFA salt

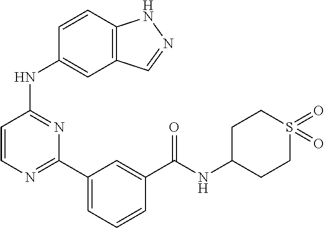 [SMILES: O=C(NC1CCS(=O)(=O)CC1)c1cccc(-c2nccc(Nc3ccc4[nH]ncc4c3)n2)c1]

To the mixture of 3-(4-((1H-indazol-5-yl)amino)pyrimidin-2-yl)benzoic acid (400 mg, 1.2 mmol), 4-aminotetrahydro-2H-thiopyran 1,1-dioxide (300 mg, 1.4 mmol), HATU (720 mg, 1.89 mmol) and Et₃N (200 mg, 2 mmol) in DMF (15 mL) was stirred at 25° C. overnight. The reaction mixture was poured into water and extracted with EtOAc. The organic layer was dried over Na₂SO₄ and concentrated to give a residue, which was purified by HPLC to give the title compound (81 mg, 20%) as a solid. ¹H NMR (400 MHz, CD₃OD) δ2.19-2.24 (m, 2H), 2.31-2.33 (m, 2H), 3.12-3.15 (m, 2H), 3.34-3.38 (m, 2H), 4.23-4.28 (m, 1H), 6.93 (d, J=6.8 Hz, 1H), 7.58-7.76 (m, 3H), 8.12 (s, 1H), 8.14 (s, 1H), 8.24 (d, J=7.2 Hz, 1H), 8.31 (d, J=8.0 Hz, 1H), 8.65 (s, 1H). MS (ES+) m/e 463 (M+H)⁺.

### Example 21

### tert-Butyl 4-(3-(4-((1H-indazol-5-yl)amino)pyrimidin-2-yl)benzamido)piperidine-1-carboxylate

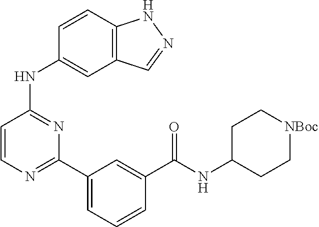 [SMILES: CC(C)(C)OC(=O)N1CCC(NC(=O)c2cccc(-c3nccc(Nc4ccc5[nH]ncc5c4)n3)c2)CC1]

A mixture of 3-(4-((1H-indazol-5-yl)amino)pyrimidin-2-yl)benzoic acid (400 mg, 1.2 mmol), 4-amino-piperidine-1-carboxylic acid tert-butyl ester (300 mg, 1.5 mmol), HATU (720 mg, 1.89 mmol) and Et₃N (200 mg, 2 mmol) in DMF (15 mL) was stirred at 25° C. overnight. The reaction mixture was poured into water and extracted with EtOAc. The organic layer was dried over Na₂SO₄ and concentrated to give a residue, which was purified by HPLC to provide the title compound (200 mg, 30%) as a solid.

### Example 22

### 3-(4-((1H-indazol-5-yl)amino)pyrimidin-2-yl)-N-(piperidin-4-yl)benzamide HCl salt

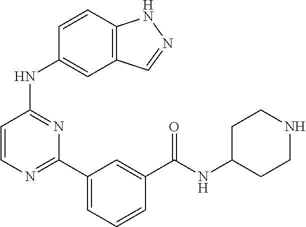 [SMILES: O=C(NC1CCNCC1)c1cccc(-c2nccc(Nc3ccc4[nH]ncc4c3)n2)c1]

A mixture of tert-butyl 4-(3-(4-((1H-indazol-5-yl)amino)pyrimidin-2-yl)benzamido)piperidine-1-carboxylate (200 mg) in HCl in MeOH (5 mL) was stirred at 25° C. for 3 h. The solvent was removed to provide the title compound (150 mg) as a yellow solid. ¹H NMR (400 MHz, DMSO-d₆) δ1.79-1.87 (m, 2H), 1.98-2.01 (m, 2H), 3.01-3.04 (m, 2H), 3.32-3.36 (m, 2H), 3.64-3.69 (m, 1H), 7.10-7.15 (m, 1H), 7.66 (d, J=8.0 Hz, 1H), 7.75 (t, J=8 Hz, 1H), 8.16 (s, 1H), 8.20 (s, 1H), 8.36 (d, J=6.8 Hz, 1H), 8.42 (d, J=7.6 Hz, 1H), 8.78 (d, J=7.2 Hz, 1H), 8.90 (s, 1H), 8.96 (s, 1H), 11.73 (s, 1H). MS (ES+) m/e 414 (M+H)⁺.

### Example 23

### N-(2-chloro-5-methylpyrimidin-4-yl)-1H-indazol-5-amine

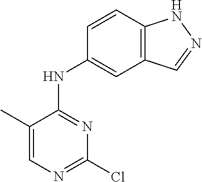 [SMILES: Cc1cnc(Cl)nc1Nc1ccc2[nH]ncc2c1]

To a mixture of compound 2,4-dichloro-5-methylpyrimidine (5 g, 30.8 mmol) and 5-aminoindazole (4.1 g, 30.8 mmol) in anhydrous ethanol (100 mL) was added Na₂CO₃ (16 g, 154 mmol). The resulting mixture was heated at 80° C. overnight under N₂. After cooling to room temperature, the mixture was diluted with water and extracted with EtOAc. The organic phase was dried over anhydrous Na₂SO₄ and concentrated in vacuo followed by by column chromatography on silica gel (eluted with DCM:MeOH=50:1) to provide the title compound (7 g, yield 87%) as a brown solid.

### Example 24

### tert-butyl 5-((tert-butoxycarbonyl)(2-chloro-5-methylpyrimidin-4-yl)amino)-1H-indazole-1-carboxylate

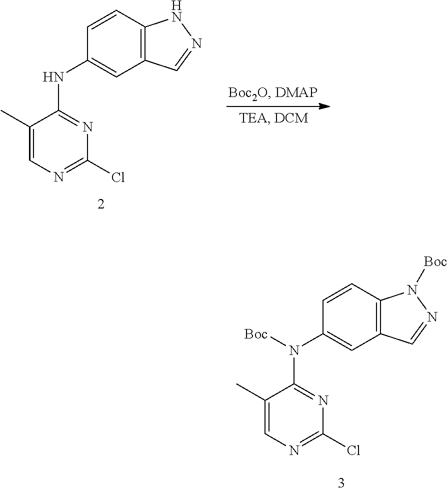 [SMILES: Cc1cnc(Cl)nc1Nc1ccc2[nH]ncc2c1 ; Cc1cnc(Cl)nc1N(C(=O)OC(C)(C)C)c1ccc2c(cnn2C(=O)OC(C)(C)C)c1]

To a solution of N-(2-chloro-5-methylpyrimidin-4-yl)-1H-indazol-5-amine (5 g, 19.3 mmol) in anhydrous DCM (100 mL) was added Boc₂O (12.6 g, 57.9 mmol), TEA (5.85 g, 57.9 mmol) and DMAP (1.17 g, 9.56 mmol). The resulting mixture was stirred at room temperature for 4 h. The mixture was diluted with water and extracted with DCM. The organic phase was dried over anhydrous Na₂SO₄, and the solvent was removed in vacuo followed by purification by column chromatograph on silica gel (eluted with PE:EA=10:1) to give compound the title compound (5 g, yield 56%) as a white solid.

### Example 25

### tert-butyl 5-((tert-butoxycarbonyl)(2-(3-(2-(cyclopropylamino)-2-oxoethoxy)phenyl)-5-methylpyrimidin-4-yl)amino)-1H-indazole-1-carboxylate

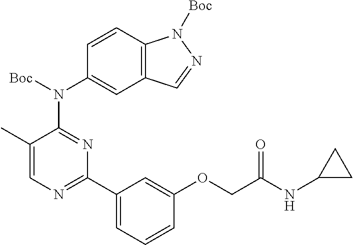 [SMILES: Cc1cnc(-c2cccc(OCC(=O)NC3CC3)c2)nc1N(C(=O)OC(C)(C)C)c1ccc2c(cnn2C(=O)OC(C)(C)C)c1]

To a mixture of compound tert-butyl 5-((tert-butoxycarbonyl)(2-chloro-5-methylpyrimidin-4-yl)amino)-1H-indazole-1-carboxylate (1.9 g, 4.14 mmol), compound N-cyclopropyl-2-(3-(4,4,5,5-tetramethyl-1,3,2-dioxaborolan-2-yl)phenoxy)acetamide (1.57 g, 4.97 mmol) and CsF (1.89 g, 12.42 mmol) in 1,4-dioxane (20 mL) and water (5 mL) was added Pd(PPh₃)₄ (239 mg, 0.21 mmol). The resulting mixture was heated at 100° C. overnight under N₂. After cooling to room temperature, the mixture was diluted with water and extracted with EtOAc, the organic phase was dried over anhydrous Na₂SO₄ and concentrated under reduced pressure to give a residue, which was purified by column chromatography on silica gel (eluted with PE:EA=5:1) to provide the title compound (1.6 g, yield 62%) as a yellow solid.

### Example 26

### 2-(3-(4-((1H-indazol-5-yl)amino)-5-methylpyrimidin-2-yl)phenoxy)-N-cyclopropylacetamide hydrochloride

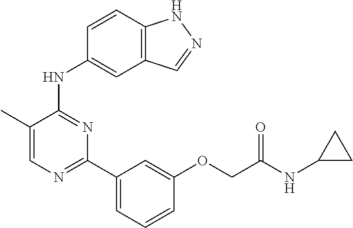 [SMILES: Cc1cnc(-c2cccc(OCC(=O)NC3CC3)c2)nc1Nc1ccc2[nH]ncc2c1]

To a solution of compound tert-butyl 5-((tert-butoxycarbonyl)(2-(3-(2-(cyclopropylamino)-2-oxoethoxy)phenyl)-5-methylpyrimidin-4-yl)amino)-1H-indazole-1-carboxylate (500 mg, 0.81 mmol) in EtOAc (2 mL) was added HCl/EtOAc (10 mL) and stirred at room temperature overnight. The formed precipitated was filtered and washed with EtOAc, dried in vacuo to afford the title compound (300 mg, yield 89%) as a yellow solid. ¹H NMR (400 MHz, CD₃OD) δ0.50-0.54 (m, 2H), 0.70-0.75 (m, 2H), 2.42 (s, 3H), 2.66-2.71 (m, 1H), 4.51 (s, 2H), 7.27 (dd, J=8.4 and 2.4 Hz, 1H), 7.49 (t, J=8.0 Hz, 1H), 7.64-7.68 (m, 2H), 7.72 (s, 1H), 8.04 (s, 2H), 8.20 (s, 1H), 8.28 (s, 1H). MS (ES+) m/e 451 (M+H)⁺.

### Example 27

### tert-butyl 5-((tert-butoxycarbonyl)(2-(3-(2-((1-(tert-butoxycarbonyl)piperidin-4-yl)amino)-2-oxoethoxy)phenyl)-5-methylpyrimidin-4-yl)amino)-1H-indazole-1-carboxylate

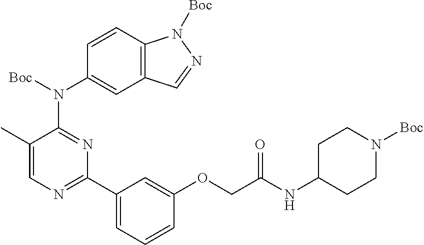 [SMILES: Cc1cnc(-c2cccc(OCC(=O)NC3CCN(C(=O)OC(C)(C)C)CC3)c2)nc1N(C(=O)OC(C)(C)C)c1ccc2c(cnn2C(=O)OC(C)(C)C)c1]

To a mixture of tert-butyl 5-((tert-butoxycarbonyl)(2-chloro-5-methylpyrimidin-4-yl)amino)-1H-indazole-1-carboxylate (1.4 g, 3.05 mmol), tert-butyl 4-(2-(3-(4,4,5,5-tetramethyl-1,3,2-dioxaborolan-2-yl)phenoxy)acetamido)piperidine-1-carboxylate (1.68 g, 3.66 mmol) and CsF (1.39 g, 9.15 mmol) in 1,4-dioxane (20 mL) and water (5 mL) was added Pd(PPh₃)₄ (176 mg, 0.15 mmol). The resulting mixture was heated at 100° C. overnight under N₂. After cooling to room temperature, the mixture was diluted with water and extracted with EtOAc, the organic phase was dried over anhydrous Na₂SO₄ and concentrated under reduced pressure to give a residue, which was purified by column chromatography on silica gel (eluted with PE:EA=1:1) to afford the title compound (1.1 g, yield 47%) as a yellow solid.

### Example 28

### 2-(3-(4-((1H-indazol-5-yl)amino)-5-methylpyrimidin-2-yl)phenoxy)-N-(piperidin-4-yl)acetamide hydrochloride

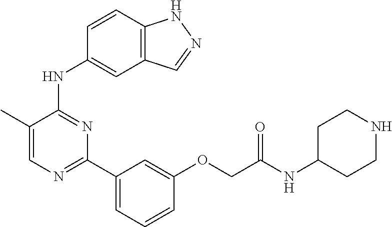 [SMILES: Cc1cnc(-c2cccc(OCC(=O)NC3CCNCC3)c2)nc1Nc1ccc2[nH]ncc2c1]

To a solution of tert-butyl 5-((tert-butoxycarbonyl)(2-(3-(2-((1-(tert-butoxycarbonyl)piperidin-4-yl)amino)-2-oxoethoxy)phenyl)-5-methylpyrimidin-4-yl)amino)-1H-indazole-1-carboxylate (700 mg, 0.92 mmol) in EtOAc (2 mL) was added HCl/EtOAc (10 mL) and stirred at room temperature overnight. The formed precipitated was filtered and washed with EtOAc, dried in vacuo to afford the title compound (230 mg, yield 54%) as a yellow solid. ¹H NMR (400 MHz, CD₃OD) δ1.73-1.84 (m, 2H), 2.01-2.05 (m, 2H), 2.42 (s, 3H), 3.02-3.09 (m, 2H), 3.39-3.43 (m, 2H), 3.99-4.05 (m, 1H), 4.57 (s, 2H), 7.30 (dd, J=8.4 and 2.4 Hz, 1H), 7.51 (t, J=8.4 Hz, 1H), 7.67-7.74 (m, 4H), 8.05 (s, 1H), 8.20 (s, 1H), 8.24 (s, 1H). MS (ES+) m/e 494 (M+H)⁺.

### Example 29

### tert-butyl 2-(3-bromophenoxyl)acetate

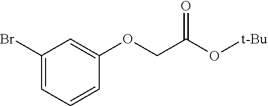 [SMILES: CC(C)(C)OC(=O)COc1cccc(Br)c1]

To a solution of 3-bromophenol (5 g, 28.9 mmol) in MeCN (100 mL) was added t-butyl bromoacetate (6.76 g, 34.7 mmol) and K₂CO₃ (5.98 g, 43.3 mmol). The resulting mixture was heated at 80° C. overnight under nitrogen. After cooling to room temperature, the mixture was diluted with water and extracted with EtOAc. The organic layer was washed with brine and dried over Na₂SO₄, filtrated and concentrated to give the residue, which was purified by column chromatography on silica gel (PE:EA=50:1) to give the title compound (7 g, yield 84%) as a oil liquid.

### Example 30

### 2-(3-bromophenoxyl)acetyl chloride

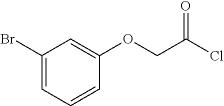 [SMILES: O=C(Cl)COc1cccc(Br)c1]

To a solution of tert-butyl 2-(3-bromophenoxyl)acetate (4.6 g, 16 mmol) in anhydrous DCM (50 mL) was added TFA (18 g, 0.16 mol) and stirred at room temperature overnight. After TLC showed the reaction was completed, the mixture was concentrated under reduced pressure to get a crude acid. The acid was dissolved in anhydrous DCM (50 mL), oxalyl chloride (2.44 g, 19.2 mmol) and DMF (0.2 mL) were added into the solution. The mixture was stirred at room temperature for 4 h. The mixture was concentrated under reduced pressure to provide a white solid, which was used for next step reaction without further purification.

### Example 31

### 2-(3-bromophenoxy)-N-cyclopropylacetamide

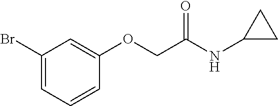 [SMILES: O=C(COc1cccc(Br)c1)NC1CC1]

To a solution of 2-(3-bromophenoxyl)acetyl chloride (2.3 g, 9.24 mmol) in anhydrous DCM (30 mL) was added triethylamine (2.8 g, 27.7 mmol) and cyclopropylamine (632 mg, 11.1 mmol) at 0° C. Then the resulting mixture was stirred at room temperature overnight. The reaction was diluted with water and extracted with EtOAc. The organic layer was dried over anhydrous Na₂SO₄ and concentrated under reduced pressure to give a residue, which was purified by column chromatography on silica gel (eluted with PE:EA=5:1) to give the title compound (1.95 g, yield 78%) as a white solid.

### Example 32

### N-cyclopropyl-2-(3-(4,4,5,5-tetramethyl-1,3,2-dioxaborolan-2-yl)phenoxy)acetamide

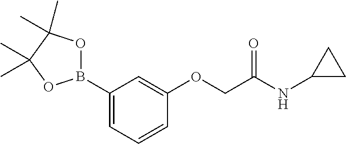 [SMILES: CC1(C)OB(c2cccc(OCC(=O)NC3CC3)c2)OC1(C)C]

To a mixture of 2-(3-bromophenoxy)-N-cyclopropylacetamide (1.95 g, 7.25 mmol), bis(pinacolato)diboron (2.76 g, 10.87 mmol) and KOAc (2.13 g, 21.7 mmol) in DMSO (20 mL) was added Pd(dppf)Cl₂-DCM (265 mg, 0.36 mmol). The resulting mixture was heated at 100° C. overnight under N₂. After cooling to room temperature, the mixture was diluted with water and extracted with EtOAc, the organic phase was dried over anhydrous Na₂SO₄ and concentrated under reduced pressure to give a residue, which was purified by column chromatography on silica gel (eluted with PE:EA=10:1) to provide the title compound (1.4 g, yield 60%) as a white solid.

### Example 33

### tert-butyl 4-(2-(3-bromophenoxyl)acetamido)piperidine-1-carboxylate

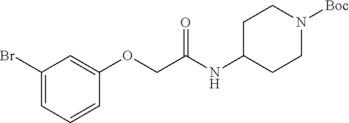 [SMILES: CC(C)(C)OC(=O)N1CCC(NC(=O)COc2cccc(Br)c2)CC1]

To a solution of 2-(3-bromophenoxyl)acetyl chloride (2.1 g, 8.43 mmol) in anhydrous DCM (30 mL) was added triethylamine (2.56 g, 25.3 mmol) and 4-amino-1-Boc-piperidine (2.02 g, 10.1 mmol) at 0° C. Then the resulting mixture was stirred at room temperature overnight. The reaction was diluted with water and extracted with EtOAc. The organic layer was dried over anhydrous Na₂SO₄ and concentrated under reduced pressure to give a residue, which was purified by column chromatography on silica gel (eluted with PE:EA=5:1) to give the tile compound (2.8 g, yield 80%) as a white solid.

### Example 34

### tert-butyl 4-(2-(3-(4,4,5,5-tetramethyl-1,3,2-dioxaborolan-2-yl)phenoxy)acetamido)piperidine-1-carboxylate

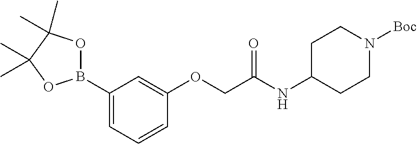 [SMILES: CC(C)(C)OC(=O)N1CCC(NC(=O)COc2cccc(B3OC(C)(C)C(C)(C)O3)c2)CC1]

To a mixture of tert-butyl 4-(2-(3-bromophenoxyl)acetamido)piperidine-1-carboxylate (2.8 g, 6.79 mmol), bis(pinacolato)diboron (2.59 g, 10.2 mmol), and KOAc (1.99 g, 20.4 mmol) in DMSO (30 mL) was added Pd(dppf)Cl₂-DCM (249 mg, 0.34 mmol). The resulting mixture was heated to 100° C. overnight under N₂. After cooling to room temperature, the mixture was diluted with water and extracted with EtOAc, the organic phase was dried over anhydrous Na₂SO₄ and concentrated under reduced pressure to give a residue, which was purified by column chromatography on silica gel (eluted with PE:EA=5:1) to provide the title compound (1.7 g, yield 54%) as a white solid.

### Example 35

### N-(2-chloropyrimidin-4-yl)-6-fluoro-1H-indazol-5-amine

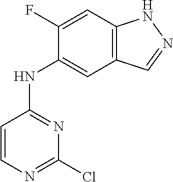 [SMILES: Fc1cc2[nH]ncc2cc1Nc1ccnc(Cl)n1]

To a mixture of 2,4-dichloropyrimidine (730 mg, 4.89 mmol) and 6-fluoro-5-aminoindazole (740 mg, 4.89 mmol) in anhydrous ethanol (15 mL) was added Na₂CO₃ (1.56 g, 14.7 mmol). The resulting mixture was heated at 80° C. overnight under N₂. After cooling to room temperature, the mixture was diluted with water and extracted with EtOAc, the organic phase was dried over anhydrous Na₂SO₄ and concentrated under reduced pressure to give a residue, which was purified by column chromatography on silica gel (eluted with PE:EA=3:1) to provide the title compound (750 mg, yield 58%) as a brown solid.

### Example 36

### tert-butyl 5-((tert-butoxycarbonyl)(2-chloropyrimidin-4-yl)amino)-6-fluoro-1H-indazole-1-carboxylate

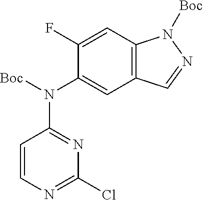 [SMILES: CC(C)(C)OC(=O)N(c1ccnc(Cl)n1)c1cc2cnn(C(=O)OC(C)(C)C)c2cc1F]

To a solution of N-(2-chloropyrimidin-4-yl)-6-fluoro-1H-indazol-5-amine (750 mg, 2.85 mmol) in anhydrous DCM (10 mL) was added Boc₂O (1.86 g, 8.55 mmol), TEA (864 mg, 8.55 mmol) and DMAP (173 mg, 1.42 mmol). The resulting mixture was stirred at room temperature for 4 hrs. The mixture was diluted with water and extracted with DCM. The organic phase was dried over anhydrous Na₂SO₄, and the solvent was removed in vacuum to give a residue, which was purified by column chromatograph on silica gel (eluted with PE:EA=20:1) to give the title compound (800 g, yield 60%) as a white solid.

### Example 37

### tert-butyl 5-((tert-butoxycarbonyl)(2-(3-(2-(isopropylamino)-2-oxoethoxy)phenyl)pyrimidin-4-yl)amino)-6-fluoro-1H-indazole-1-carboxylate

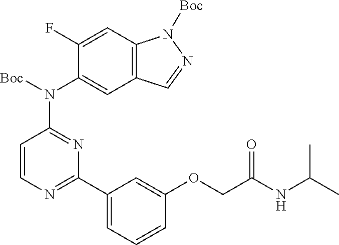 [SMILES: CC(C)NC(=O)COc1cccc(-c2nccc(N(C(=O)OC(C)(C)C)c3cc4cnn(C(=O)OC(C)(C)C)c4cc3F)n2)c1]

To a mixture of tert-butyl 5-((tert-butoxycarbonyl)(2-chloropyrimidin-4-yl)amino)-6-fluoro-1H-indazole-1-carboxylate (920 mg, 1.98 mmol) in 1,4-dioxane (16 mL) and water (4 mL) was added N-isopropyl-2-(3-(4,4,5,5-tetramethyl-1,3,2-dioxaborolan-2-yl)phenoxy)acetamide (760 mg, 2.38 mmol), Pd(PPh₃)₄ (115 mg, 0.1 mmol) and CsF (906 mg, 5.96 mmol). The resulting mixture was heated at 100° C. overnight under N₂. After cooling to room temperature, the mixture was diluted with water and extracted with EtOAc, the organic phase was dried over anhydrous Na₂SO₄ and concentrated under reduced pressure to give a residue, which was purified by column chromatography on silica gel (eluted with PE:EA=3:1) to provide the title compound (600 mg, yield 48%) as a yellow solid.

### Example 38

### 2-(3-(4-((6-fluoro-1H-indazol-5-yl)amino)pyrimidin-2-yl)phenoxy)-N-isopropylacetamide hydrochloride

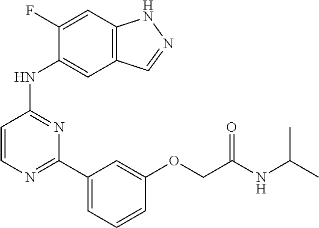 [SMILES: CC(C)NC(=O)COc1cccc(-c2nccc(Nc3cc4cn[nH]c4cc3F)n2)c1]

To a mixture of tert-butyl 5-((tert-butoxycarbonyl)(2-(3-(2-(isopropylamino)-2-oxoethoxy)phenyl)pyrimidin-4-yl)amino)-6-fluoro-1H-indazole-1-carboxylate (520 mg, 0.84 mmol) in EtOAc (2 mL) was added HCl/EtOAc (10 mL) and stirred at room temperature overnight. The formed precipitate was filtered and washed with EtOAc, dried in vacuo to afford the title compound (200 mg, yield 56%) as a yellow solid. 1H NMR (400 MHz, DMSO-d₆) δ1.04 (d, J=6.4 Hz, 6H), 3.88-3.93 (m, 1H), 4.51 (s, 2H), 7.00 (b, 1H), 7.22 (d, J=7.6 Hz, 1H), 7.49 (t, J=7.6 Hz, 1H), 7.56 (d, J=10.4 Hz, 1H), 7.75-7.79 (m, 2H), 7.93 (d, J=7.2 Hz, 1H), 8.09 (d, J=6.4 Hz, 1H), 8.16 (s, 1H), 8.36 (d, J=6.4 Hz, 1H), 11.09 (s, 1H). MS (ES+) m/e 457 (M+H)⁺.

### Example 39

### N-(2-chloro-5-methoxypyrimidin-4-yl)-1H-indazol-5-amine

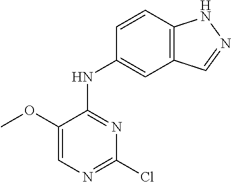 [SMILES: COc1cnc(Cl)nc1Nc1ccc2[nH]ncc2c1]

To the mixture of 2,4-dichloro-5-methoxypyrimidine (3.56 g, 0.02 mol) in EtOH (80 mL) was added 1H-indazol-5-amine (2.66 g, 0.02 mol), and then DIEA (7.8 g, 0.06 mol). The resulting mixture was stirred at 45° C. overnight. The reaction mixture was cooled to room temperature and filtered. The cake was rinsed by MTBE and collected to give the title compound (4.4 g, yield 81%) as brown solid.

### Example 40

### tert-butyl 5-((tert-butoxycarbonyl)(2-chloro-5-methoxypyrimidin-4-yl)amino)-1H-indazole-1-carboxylate

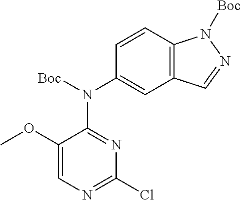 [SMILES: COc1cnc(Cl)nc1N(C(=O)OC(C)(C)C)c1ccc2c(cnn2C(=O)OC(C)(C)C)c1]

To the mixture of N-(2-chloro-5-methoxypyrimidin-4-yl)-1H-indazol-5-amine (4.2 g, 15.3 mmol) in DCM (50 mL) were added TEA (4.6 g, 45.9 mmol), (Boc)₂O (8.32 g, 38.2 mmol), and DMAP (0.2 g). The resulting mixture was stirred at room temperature for 1 h and concentrated followed by purification by column chromatograph to provide the title compound (5.5 g, yield 76%) as light yellow solid.

### Example 41

### tert-butyl 5-((tert-butoxycarbonyl)(2-(3-(2-(isopropylamino)-2-oxoethoxy)phenyl)-5-methoxypyrimidin-4-yl)amino)-1H-indazole-1-carboxylate

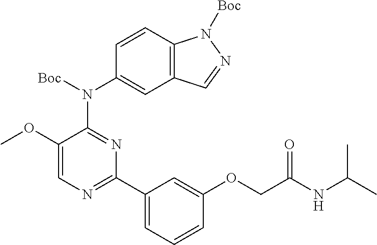 [SMILES: COc1cnc(-c2cccc(OCC(=O)NC(C)C)c2)nc1N(C(=O)OC(C)(C)C)c1ccc2c(cnn2C(=O)OC(C)(C)C)c1]

To the solution of tert-butyl 5-((tert-butoxycarbonyl)(2-chloro-5-methoxypyrimidin-4-yl)amino)-1H-indazole-1-carboxylate (1.5 g, 3.16 mmol) in the solvents (dioxane:water=10:1, 33 ml) were added N-isopropyl-2-(3-(4,4,5,5-tetramethyl-1,3,2-dioxaborolan-2-yl) phenoxy)acetamide (1.2 g, 3.79 mmol), (Boc)₂O (1.38 g, 6.32 mmol), CsF (1.4 g, 9.48 mmol), and then Pd(PPh₃)₄ (0.11 g, 0.095 mmol) under N₂. The resulting mixture was stirred at 90° C. for 24 hrs. The mixture was purified by column chromatograph to give the title compound (0.98 g, yield 49%) as white solid.

### Example 42

### 2-(3-(4-((1H-indazol-5-yl)amino)-5-hydroxypyrimidin-2-yl)phenoxy)-N-isopropylacetamide

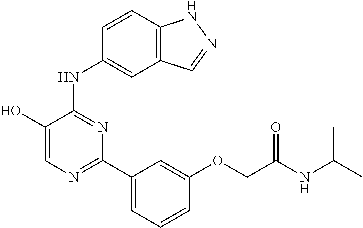 [SMILES: CC(C)NC(=O)COc1cccc(-c2ncc(O)c(Nc3ccc4[nH]ncc4c3)n2)c1]

The mixture of tert-butyl 5-((tert-butoxycarbonyl)(2-(3-(2-(isopropylamino)-2-oxoethoxy)phenyl)-5-methoxypyrimidin-4-yl)amino)-1H-indazole-1-carboxylate (1.2 g, 2 mmol) and pyridine hydrochloride (7.8 g) was stirred at 140° C. for 1 hr. The reaction cooled to room temperature. Water (20 mL) was added followed by NH₃.H₂O to adjust the pH to 6-7. The mixture was filtered. The cake was collected and dried to give the title compound (0.46 g, yield 55%) as gray solid.

### Example 43

### 2-(3-(4-((1H-indazol-5-yl)amino)-5-(2-(dimethylamino)ethoxy)pyrimidin-2-yl)phenoxy)-N-isopropylacetamide TFA salt

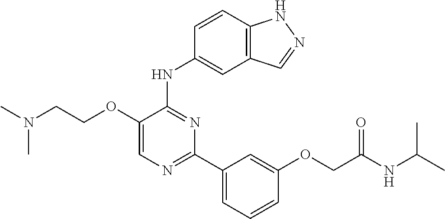 [SMILES: CC(C)NC(=O)COc1cccc(-c2ncc(OCCN(C)C)c(Nc3ccc4[nH]ncc4c3)n2)c1]

To the mixture of 2-(3-(4-((1H-indazol-5-yl)amino)-5-hydroxypyrimidin-2-yl)phenoxy)-N-isopropylacetamide (450 mg, 1.07 mmol) in THF (45 mL) were added 2-(dimethylamino) ethanol (115 mg, 1.29 mmol), and then PPh₃ (563 mg, 2.15 mmol) was added. The resulting mixture was stirred at room temperature for 0.5 hr. Then DEAD (374 mg, 2.15 mmol) was added. The resulting mixture was heated at reflux overnight. The solvent was removed under reduced pressure followed by addition of 10 mL EtOAc and 10 mL water. TFA was added to adjust pH=4-5. The aqueous solution was concentrated and purified by Prep HPLC to give the title compound (0.1 g, yield 10%) as yellow solid. ¹H NMR (400 MHz, DMSO-d₆) δ1.07 (d, J=6.4 Hz, 6H), 2.94 (d, J=4.0 Hz, 6H), 3.64-3.65 (m, 2H), 3.92-3.94 (m, 1H), 4.48 (s, 2H), 4.52-4.53 (m, 2H), 7.02 (dd, J=9.2 and 1.6 Hz, 1H), 7.37 (t, J=7.2 Hz, 1H), 7.60-7.89 (m, 5H), 8.12-8.19 (m, 3H), 8.90 (s, 1H), 9.60 (b, 1H). MS (ES+) m/e 490 (M+H)⁺.

### Example 44

### N-(2,6-dichloropyrimidin-4-yl)-1H-indazol-5-amine

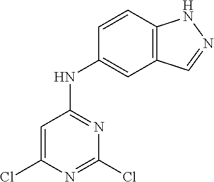 [SMILES: Clc1cc(Nc2ccc3[nH]ncc3c2)nc(Cl)n1]

A solution of 2,4,6-trichloropyrimidine (3.67 g, 20 mmol), 1H-indazol-5-amine (2.66 g, 20 mmol) and TEA (3.03 g, 30 mmol) in EtOH (75 mL) was heated at reflux overnight. After removing the solvent, the residue was re-crystallized in MeOH to provide the title compound (4.2 g, yield 50%) as a solid.

### Example 45

### N-(2-chloro-6-(4-methylpiperazin-1-yl)pyrimidin-4-yl)-1H-indazol-5-amine

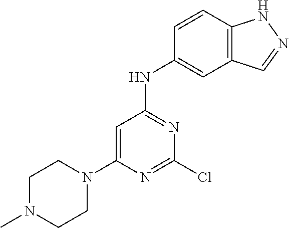 [SMILES: CN1CCN(c2cc(Nc3ccc4[nH]ncc4c3)nc(Cl)n2)CC1]

A solution of N-(2,6-dichloropyrimidin-4-yl)-1H-indazol-5-amine (4.2 g, 15 mmol), 1-methylpiperazine (2.0 g, 20 mmol) and TEA (3.03 g, 30 mmol) in MeOH (75 mL) was refluxed overnight. After removing the solvent, the residue was re-crystallized in DCM to give the title compound (3 g, yield 58%) as a solid.

### Example 46

### tert-butyl 5-((tert-butoxycarbonyl)(2-chloro-6-(4-methylpiperazin-1-yl)pyrimidin-4-yl)amino)-1H-indazole-1-carboxylate

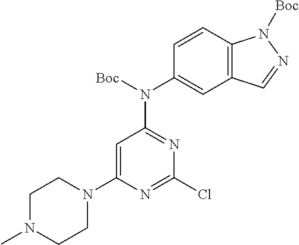 [SMILES: CN1CCN(c2cc(N(C(=O)OC(C)(C)C)c3ccc4c(cnn4C(=O)OC(C)(C)C)c3)nc(Cl)n2)CC1]

To a solution of N-(2-chloro-6-(4-methylpiperazin-1-yl)pyrimidin-4-yl)-1H-indazol-5-amine (1.5 g, 4.4 mmol) in DCM (20 mL) were added TEA (0.93 g, 9.2 mmol), (Boc)₂O (3 g, 13.9 mmol) and DMAP (1.1 g, 9.2 mmol). The resulting mixture was stirred at room temperature for 3 hrs. After removing the solvent, the residue was purified by column chromatography on silica gel (eluting with petroleum ether:ethyl acetate=50:1-10:1) to give the title (1.2 g, yield 50%) as a solid.

### Example 47

### tert-butyl 5-((tert-butoxycarbonyl)(2-(3-(2-(isopropylamino)-2-oxoethoxy)phenyl)-6-(4-methylpiperazin-1-yl)pyrimidin-4-yl)amino)-1H-indazole-1-carboxylate

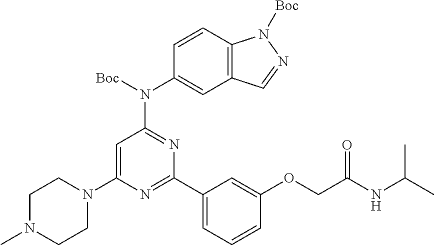 [SMILES: CC(C)NC(=O)COc1cccc(-c2nc(N3CCN(C)CC3)cc(N(C(=O)OC(C)(C)C)c3ccc4c(cnn4C(=O)OC(C)(C)C)c3)n2)c1]

To a solution of tert-butyl 5-((tert-butoxycarbonyl)(2-chloro-6-(4-methylpiperazin-1-yl)pyrimidin-4-yl)amino)-1H-indazole-1-carboxylate (0.600 g, 1.1 mmol), Pd(dppf)Cl₂ (50 mg), CsF (0.501 g, 3.3 mmol) and N-isopropyl-2-(3-(4,4,5,5-tetramethyl-1,3,2-dioxaborolan-2-yl)phenoxy)acetamide (0.640 g, 2 mmol) in dioxane/water (10:1, 10 mL) was stirred at 100° C. overnight. After removing the solvent, the residue was purified by HPLC to give the title compound (250 mg, yield 32%).

### Example 48

### 2-(3-(4-((1H-indazol-5-yl)amino)-6-(4-methylpiperazin-1-yl)pyrimidin-2-yl)phenoxy)-N-isopropylacetamide TFA salt

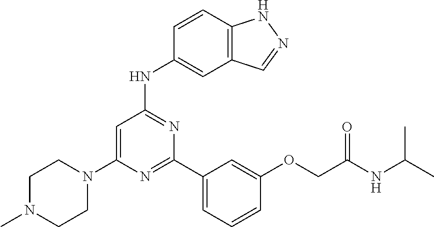 [SMILES: CC(C)NC(=O)COc1cccc(-c2nc(Nc3ccc4[nH]ncc4c3)cc(N3CCN(C)CC3)n2)c1]

A solution of tert-butyl 5-((tert-butoxycarbonyl)(2-(3-(2-(isopropylamino)-2-oxoethoxy)phenyl)-6-(4-methylpiperazin-1-yl)pyrimidin-4-yl)amino)-1H-indazole-1-carboxylate (250 mg, 0.36 mmol) in DCM (10 mL) and TFA (3.0 mL) was stirred at room temperature for 3 hrs. The mixture was concentrated in vacuo to provide the title compound (150 mg, yield 70%) as a solid. ¹H NMR (400 MHz, DMSO-d₆) δ1.02 (d, J=6.4 Hz, 6H), 2.79 (s, 3H), 3.04 (b, 2H), 3.20-3.27 (m, 2H), 3.47-3.50 (m, 2H), 3.85-3.93 (m, 1H), 4.45 (s, 2H), 4.71-4.75 (m, 2H), 6.56 (s, 1H), 7.01 (dd, J=8.4 and 2.4 Hz, 1H), 7.36 (t, J=8.0 Hz, 1H), 7.41-7.52 (m, 4H), 7.88 (d, J=7.6 Hz, 1H), 7.98 (s, 2H), 9.53 (s, 1H), 10.00 (s, 1H). MS (ES+) m/e 501 (M+H)⁺.

### Example 49

### N-(2-chloropyrimidin-4-yl)-6-methyl-1H-indazol-5-amine

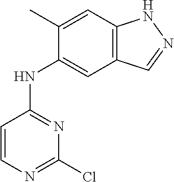 [SMILES: Cc1cc2[nH]ncc2cc1Nc1ccnc(Cl)n1]

A solution of 2,4-dichloropyrimidine (0.69 g, 4.6 mmol), 6-methyl-1H-indazol-5-ylamine hydrochloride (0.85 g, 4.6 mmol) and TEA (1.4 g, 13.8 mmol) in EtOH (16 mL) was heated at reflux overnight. The volatiles were removed to give the crude title compound (1.7 g), which was used for the next step reaction without purification.

### Example 50

### tert-butyl 5-((tert-butoxycarbonyl)(2-chloropyrimidin-4-yl)amino)-6-methyl-1H-indazole-1-carboxylate

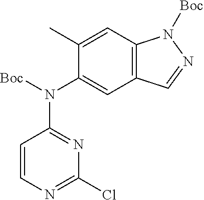 [SMILES: Cc1cc2c(cnn2C(=O)OC(C)(C)C)cc1N(C(=O)OC(C)(C)C)c1ccnc(Cl)n1]

To a solution of N-(2-chloropyrimidin-4-yl)-6-methyl-1H-indazol-5-amine (1.7 g, crude) in DCM (20 mL) were added TEA (0.93 g, 9.2 mmol), (Boc)₂O (3 g, 13.9 mmol) and DMAP (1.1 g, 9.2 mmol). The resulting mixture was stirred at room temperature for 3 hrs. After removal of the solvent, the residue was purified by column chromatography on silica gel (eluting with petroleum ether:ethyl acetate=50:1-10:1) to give the title compound (0.45 g, yield 21.1% for 2 steps) as a solid.

### Example 51

### tert-butyl 5-((tert-butoxycarbonyl)(2-(3-(2-(isopropylamino)-2-oxoethoxy)phenyl)pyrimidin-4-yl)amino)-6-methyl-1H-indazole-1-carboxylate

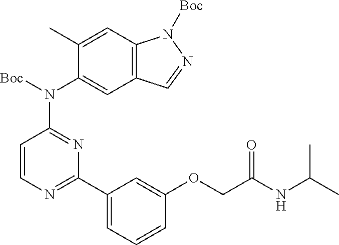 [SMILES: Cc1cc2c(cnn2C(=O)OC(C)(C)C)cc1N(C(=O)OC(C)(C)C)c1ccnc(-c2cccc(OCC(=O)NC(C)C)c2)n1]

A solution of tert-butyl 5-((tert-butoxycarbonyl)(2-chloropyrimidin-4-yl)amino)-6-methyl-1H-indazole-1-carboxylate (0.45 g, 1.67 mmol), Pd(dppf)Cl₂ (50 mg), Na₂CO₃ (0.35 g, 3.34 mmol) and N-isopropyl-2-(3-(4,4,5,5-tetramethyl-1,3,2-dioxaborolan-2-yl)phenoxy)acetamide (0.64 g, 2 mmol) in dioxane/water (10:1, 10 mL) was stirred at 100° C. overnight. After removing the solvent, the residue was purified by HPLC to give the title compound (180 mg, yield 17%).

### Example 52

### N-isopropyl-2-(3-(4-((6-methyl-1H-indazol-5-yl)amino)pyrimidin-2-yl)phenoxy)acetamide TFA salt

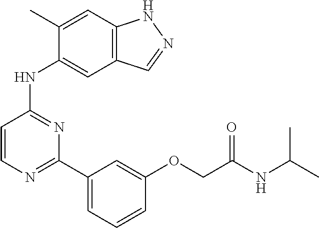 [SMILES: Cc1cc2[nH]ncc2cc1Nc1ccnc(-c2cccc(OCC(=O)NC(C)C)c2)n1]

To a solution of tert-butyl 5-((tert-butoxycarbonyl)(2-(3-(2-(isopropylamino)-2-oxoethoxy)phenyl)pyrimidin-4-yl)amino)-6-methyl-1H-indazole-1-carboxylate (180 mg, 0.29 mmol) in DCM (10 mL) was added TFA (1.5 mL) was added. The mixture was stirred at room temperature overnight and concentrated in vacuo to provide the title compound (170 mg, yield 96%). MS (ES+) m/e 417 (M+H)⁺.

### Example 53

### N-(2-chloropyrimidin-4-yl)-N-methyl-1H-indazol-5-amine

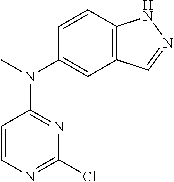 [SMILES: CN(c1ccc2[nH]ncc2c1)c1ccnc(Cl)n1]

To a solution of 2,4-dichloropyrimidine (1.0 g, 6.7 mmol) in EtOH (20 mL) was added (1H-Indazol-5-yl)-methyl-amine (1.0 g, 6.8 mmol) and TEA (2.02 g, 20 mmol). The resulting mixture was refluxed overnight. The solvent was removed to give the crude title compound (2.5 g), which was used for the next step reaction without purification.

### Example 54

### tert-butyl 5-((2-chloropyrimidin-4-yl)(methyl)amino)-1H-indazole-1-carboxylate

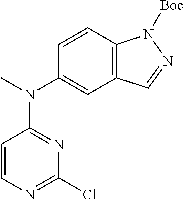 [SMILES: CN(c1ccc2c(cnn2C(=O)OC(C)(C)C)c1)c1ccnc(Cl)n1]

To a solution of N-(2-chloropyrimidin-4-yl)-N-methyl-1H-indazol-5-amine (2.5 g, crude) in DCM (50 mL) were added TEA (2.0 g, 20 mmol), (Boc)₂O (4.2 g, 19.2 mmol), and DMAP (1.0 g) was added. The resulting mixture was stirred at room temperature for 1 hr. The mixture was concentrated in vacuo and purified by column chromatograph to give the title compound (0.95 g, yield 38.8% for 2 steps) as a solid.

### Example 55

### tert-butyl 5-((2-(3-(2-(isopropylamino)-2-oxoethoxy)phenyl)pyrimidin-4-yl)(methyl)amino)-1H-indazole-1-carboxylate

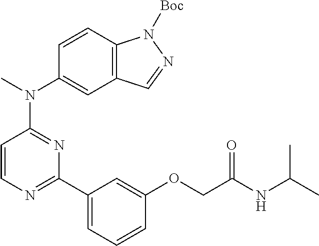 [SMILES: CC(C)NC(=O)COc1cccc(-c2nccc(N(C)c3ccc4c(cnn4C(=O)OC(C)(C)C)c3)n2)c1]

To a solution of tert-butyl 5-((2-chloropyrimidin-4-yl)(methyl)amino)-1H-indazole-1-carboxylate (0.6 g, 1.67 mmol) in the solvent (dioxane:water=4:1, 20 mL) were added N-isopropyl-2-[3-(4,4,5,5-tetramethyl-[1,3,2]dioxaborolan-2-yl)-phenoxy]-acetamide (0.6 g, 1.88 mmol), (Boc)₂O (1.09 g, 5 mmol), K₃PO₄ (1.06 g, 5 mmol), t-Bu₃P (0.4 g, 2 mmol) and Pd₂(dba)₃ (0.1 g) under N₂. The resulting mixture was stirred at 100° C. for 24 hrs and concentrated in vacuo to provide the crude material which was carried out for the next step reaction without further purification.

### Example 56

### 2-(3-(4-((1H-indazol-5-yl)(methyl)amino)pyrimidin-2-yl)phenoxy)-N-isopropylacetamide TFA salt

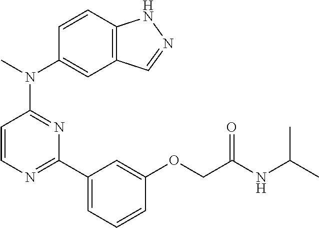 [SMILES: CC(C)NC(=O)COc1cccc(-c2nccc(N(C)c3ccc4[nH]ncc4c3)n2)c1]

To tert-butyl 5-((2-(3-(2-(isopropylamino)-2-oxoethoxy)phenyl)pyrimidin-4-yl)(methyl)amino)-1H-indazole-1-carboxylate as above was added HCl/MeOH (4M, 5 mL) and the mixture was stirred for 2.0 hr. The solvent was removed in vacuo and the residue was purified by HPLC to give the title compound (120 mg) as a TFA salt. ¹H NMR (400 MHz, CD3OD) δ1.20 (d, J=6.8 Hz, 6H), 3.85 (s, 3H), 4.07-4.13 (m, 1H), 4.62 (s, 2H), 6.37-6.45 (m, 1H), 7.39 (dd, J=10.4 and 2.0 Hz, 1H), 7.43 (d, J=2.0 Hz, 1H), 7.60 (t, J=7.6 Hz, 1H), 7.79 (d, J=8.8 Hz, 1H), 7.86-7.89 (m, 2H), 7.91 (s, 1H), 8.05 (b, 1H), 8.18 (s, 1H). MS (ES+) m/e 417 (M+H)⁺.

### Example 57

### 2-chloro-N-(1H-pyrazol-4-yl)pyrimidin-4-amine

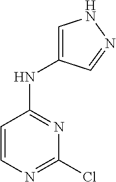 [SMILES: Clc1nccc(Nc2cn[nH]c2)n1]

The mixture of 2,4-dichloro-pyrimidine (200 mg, 2.41 mmol), 1H-pyrazol-4-ylamine (431 mg, 2.89 mmol), and TEA (730 mg, 7.23 mmol) in i-PrOH (8 mL) was stirred at 50° C. overnight. After cooling, the reaction mixture was concentrated. The crude product was used directly for the next step without purification.

### Example 58

### tert-butyl 4-((tert-butoxycarbonyl)(2-chloropyrimidin-4-yl)amino)-1H-pyrazole-1-carboxylate

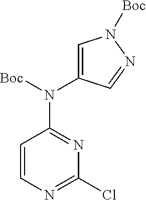 [SMILES: CC(C)(C)OC(=O)N(c1cnn(C(=O)OC(C)(C)C)c1)c1ccnc(Cl)n1]

To a mixture of (2-chloro-pyrimidin-4-yl)-(1H-pyrazol-4-yl)-amine (470 mg, 2.41 mmol), TEA (730 mg, 7.23 mmol) and DMAP (607 mg, 4.82 mmol) in dry DCM (15 mL), Boc₂O (1040 mg, 4.82 mmol) was added slowly. The reaction mixture was stirred at room temperature for 3 h, and concentrated. The residue was purified by column chromatography on silica gel to give the title compound (200 mg, 0.5 mmol, 21% yield) as a white solid.

### Example 59

### tert-butyl 4-((2-(3-(2-(isopropylamino)-2-oxoethoxy)phenyl)pyrimidin-4-yl)amino)-1H-pyrazole-1-carboxylate

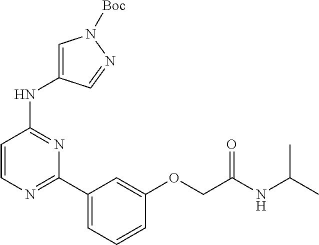 [SMILES: CC(C)NC(=O)COc1cccc(-c2nccc(Nc3cnn(C(=O)OC(C)(C)C)c3)n2)c1]

To a mixture of 4-[tert-butoxycarbonyl-(2-chloro-pyrimidin-4-yl)-amino]-pyrazole-1-carboxylic acid tert-butyl ester (118.5 mg, 0.3 mmol), N-isopropyl-2-(3-(4,4,5,5-tetramethyl-1,3,2-dioxaborolan-2-yl) phenoxy) acetamide (134 mg, 0.42 mmol), Na₂CO₃ (64 mg, 0.6 mmol), and Boc₂O (130 mg, 0.6 mmol) in EtOH (3 mL) and H₂O (0.3 mL), Pd(dppf)₂Cl₂ (21 mg, 0.03 mmol) was added. The mixture was stirred at 1300 under N₂ protection under microwave for 30 minutes. After cooling, the mixture was concentrated. The residue was purified by flash column chromatograph on silica gel, and then purified by P-HPLC to give the title compound (30 mg, 0.066 mmol, 22% yield) as a white solid.

### Example 60

### 2-(3-(4-((1H-pyrazol-4-yl)amino)pyrimidin-2-yl)phenoxy)-N-isopropylacetamide TFA salt

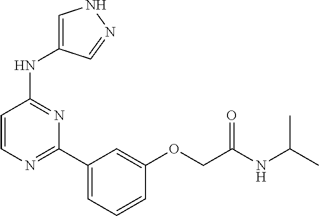 [SMILES: CC(C)NC(=O)COc1cccc(-c2nccc(Nc3cn[nH]c3)n2)c1]

To a solution of tert-butyl 4-((2-(3-(2-(isopropylamino)-2-oxoethoxy)phenyl) pyrimidin-4-yl)amino)-1H-pyrazole-1-carboxylate (167 mg, 0.369 mmol) in DCM (20 mL) TFA (2 mL) was added. The mixture was stirred at room temperature for 5 hrs, and then concentrated to give the title compound (170 mg, 0.364 mmol, 98% yield) as a yellow solid. 1H NMR (400 MHz, CD3OD) δ 8.20-8.18 (d, J=7.2 Hz, 1H), 8.07 (s 2H), 7.80-7.78 (d, 2H), 7.64-7.60 (t, J=8.8 Hz, 1H), 7.40-7.40 (dd, J=9.6, 2.4 Hz, 1H), 6.91-6.89 (d, J=7.2 Hz, 1H), 4.63 (s, 2H), 4.13-4.09 (m, 1H) 1.18-1.16 (d, J=6.4 Hz 6H). MS (ES+) m/e 353 (M+H)⁺.

### Example 61

### 5-((2-chloropyrimidin-4-yl)oxy)-1H-indazole

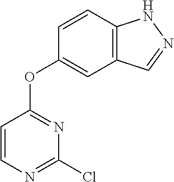 [SMILES: Clc1nccc(Oc2ccc3[nH]ncc3c2)n1]

The mixture of 2,4-dichloro-pyrimidine (184 mg, 1.232 mmol), 1H-indazol-5-ol (150 mg, 1.12 mmol), and TEA (340 mg, 3.36 mmol) in EtOH (5 mL) was stirred at 800 overnight. After cooling, the reaction mixture was concentrated. The crude product was used directly for the next step without purification.

### Example 62

### tert-butyl 5-((2-chloropyrimidin-4-yl)oxy)-1H-indazole-1-carboxylate

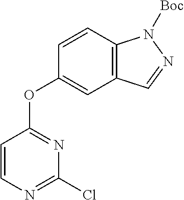 [SMILES: CC(C)(C)OC(=O)n1ncc2cc(Oc3ccnc(Cl)n3)ccc21]

To a stirred mixture of 5-((2-chloropyrimidin-4-yl)oxy)-1H-indazole (275 mg, 1.12 mmol), TEA (340 mg, 3.36 mmol) and DMAP (28 mg, 0.224 mmol) in dry DCM (5 mL), Boc₂O (484 mg, 2.24 mmol) was added slowly. The reaction mixture was stirred at room temperature for 2 hrs, and then concentrated. The residue was purified by column chromatography on silica gel to give the title compound (200 mg, 0.57 mmol, 50% yield) as a white solid.

### Example 63

### tert-butyl 5-((2-(3-(2-(isopropylamino)-2-oxoethoxy)phenyl)pyrimidin-4-yl)oxy)-1H-indazole-1-carboxylate

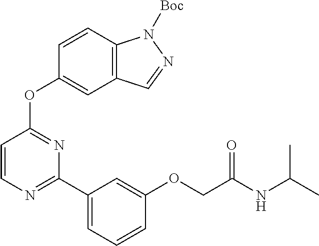 [SMILES: CC(C)NC(=O)COc1cccc(-c2nccc(Oc3ccc4c(cnn4C(=O)OC(C)(C)C)c3)n2)c1]

To a mixture of tetr-butyl-5-((2-chloropyrimidin-4-yl)oxy)-1H-indazole-1-carboxylate (104 mg, 0.3 mmol), N-isopropyl-2-(3-(4,4,5,5-tetramethyl-1,3,2-dioxaborolan-2-yl) phenoxy) acetamide (134 mg, 0.42 mmol), t-Bu₃P (61 mg, 0.3 mmol), K₃PO₄.3H₂O (160 mg, 0.6 mmol), and Boc₂O (130 mg, 0.6 mmol) in dioxane (3 mL) and H₂O (0.4 mL), Pd₂(dba)₃ (27 mg, 0.03 mmol) was added. The mixture was stirred at 800 under N₂ protection overnight. After cooling, the mixture was concentrated. The residue was purified by reverse-phase HPLC to give the title compound (58 mg, 0.115 mmol, 38% yield) as a white solid.

### Example 64

### 2-(3-(4-((1H-indazol-5-yl)oxy)pyrimidin-2-yl)phenoxy)-N-isopropylacetamide HCl salt

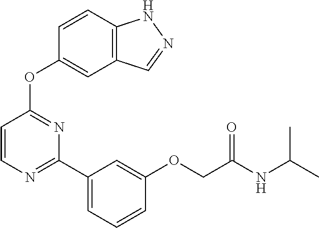 [SMILES: CC(C)NC(=O)COc1cccc(-c2nccc(Oc3ccc4[nH]ncc4c3)n2)c1]

The solution of tert-butyl 5-((2-(3-(2-(isopropylamino)-2-oxoethoxy)phenyl) pyrimidin-4-yl)oxy)-1H-indazole-1-carboxylate (340 mg, 0.675 mmol) HCl (g)/EtOAc (40 mL) was stirred at room temperature for 3 hrs, and then concentrated to provide the title compound (272 mg, 0.621 mmol, 92% yield) as a white solid. ¹H NMR (400 MHz, CD3OD) δ 8.87-8.85 (d, J=6.4 Hz, 1H), 8.25 (s 1H), 7.78-7.71 (m, 2H), 7.69-7.65 (m, 2H), 7.50-7.43 (m, 3H), 7.30-727 (dd, J=8.0, 2.4 Hz, 1H), 4.48 (s, 2H), 4.06-4.00 (m, 1H), 1.14-1.12 (d, J=6.4 Hz 6H). MS (ES+) m/e 404 (M+H)⁺.

### Example 65

### 2-(3-(4-((1H-indazol-5-yl)amino)pyrimidin-2-yl)phenoxy)-1-(piperazin-1-yl)ethanone HCl salt

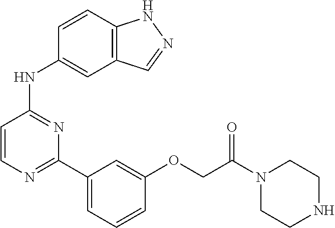 [SMILES: O=C(COc1cccc(-c2nccc(Nc3ccc4[nH]ncc4c3)n2)c1)N1CCNCC1]

The title compound was prepared using essentially the same procedure described for example 12. ¹H NMR (400 MHz, CD3OD) δ 3.24 (b, 4H), 3.82 (b, 4H), 5.00 (s, 2H), 6.93 (b, 1H), 7.33 (dd, J=7.6 and 1.6 Hz, 1H), 7.56 (t, J=8.4 Hz, 1H), 7.68 (d, J=8.4 Hz, 2H), 7.79 (b, 2H), 8.15 (s, 1H), 8.22 (d, J=7.2 Hz, 2H). (ES+) m/e 430 (M+H)⁺.

### Example 66

### 2-(3-(4-amino-6-chloropyrimidin-2-yl)phenoxy)-N-isopropylacetamide

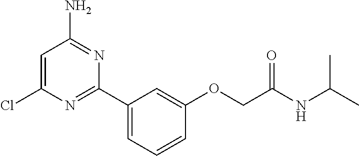 [SMILES: CC(C)NC(=O)COc1cccc(-c2nc(N)cc(Cl)n2)c1]

### Example 67

### 2-(3-(6-amino-2-chloropyrimidin-4-yl)phenoxy)-N-isopropylacetamide

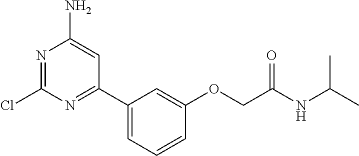 [SMILES: CC(C)NC(=O)COc1cccc(-c2cc(N)nc(Cl)n2)c1]

To a mixture of 4-amino-2,6-dichloropyrimidine (1.016 g, 6.72 mmol), N-isopropyl-2-(3-(4,4,5,5-tetramethyl-1,3,2-dioxaborolan-2-yl)phenoxy)acetamide (2.032 g, 6.37 mmol) and CsF (2.858 g, 18.803 mmol) in 1,4-dioxane (31.2 mL) and H₂O (6.3 mL) was added Pd(PPh₃)₄ (0.362 g, 0.313 mmol). The resulting mixture was stirred at 100° C. overnight under N₂. After cooling to room temperature, the mixture was concentrated under reduced pressure to give a residue, which was purified by column chromatography on silica gel (eluted with PE:EtOAc=2:1) to obtain compound 2-(3-(4-amino-6-chloropyrimidin-2-yl)phenoxy)-N-isopropylacetamide (670 mg, yield 33%) as a white powder and 2-(3-(6-amino-2-chloropyrimidin-4-yl)phenoxy)-N-isopropylacetamide (460 mg, yield 22%) as a white powder.

### Example 68

### 2-(3-(4-amino-6-(4-methylpiperazin-1-yl)pyrimidin-2-yl)phenoxy)-N-isopropylacetamide

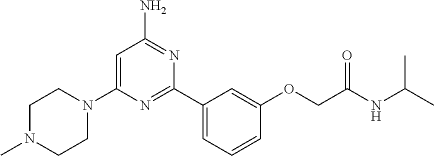 [SMILES: CC(C)NC(=O)COc1cccc(-c2nc(N)cc(N3CCN(C)CC3)n2)c1]

To a solution of compound 2-(3-(4-amino-6-chloropyrimidin-2-yl)phenoxy)-N-isopropylacetamide (0.97 g, 3.031 mmol) in n-BuOH (15 mL) was added 1-methylpiperazine (1.5 g, 15 mmol) and stirred at 120° C. overnight under N₂. After cooling to room temperature, the mixture was concentrated under reduced pressure to give a residue, which was purified by column chromatography on silica gel (eluted with EtOAc:MeOH=20:1) to obtain the title compound (460 mg, yield 39%) as a light yellow powder.

### Example 69

### 2-(3-(6-amino-2-(4-methylpiperazin-1-yl)pyrimidin-4-yl)phenoxy)-N-isopropylacetamide

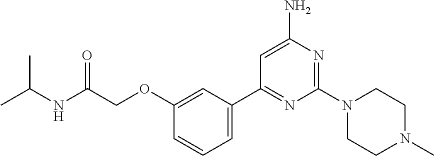 [SMILES: CC(C)NC(=O)COc1cccc(-c2cc(N)nc(N3CCN(C)CC3)n2)c1]

To a solution of compound 2-(3-(6-amino-2-chloropyrimidin-4-yl)phenoxy)-N-isopropylacetamide (0.436 g, 1.363 mmol) in n-BuOH (10 mL) was added 1-methylpiperazine (0.682 g, 6.815 mmol) and stirred at 120° C. overnight under N₂. After cooling to room temperature, the mixture was concentrated under reduced pressure to give a residue, which was purified by column chromatography on silica gel (eluted with EtOAc:MeOH=20:1) to obtain the title compound (0.273 g, yield 52%) as a light yellow powder.

### Example 70

### N-isopropyl-2-(3-(4-(4-methylpiperazin-1-yl)-6-(pyridin-4-ylamino)pyrimidin-2-yl)phenoxy)acetamide

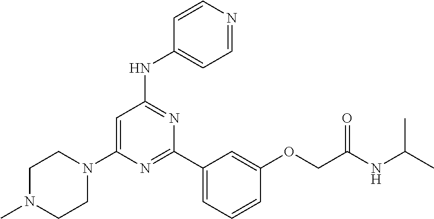 [SMILES: CC(C)NC(=O)COc1cccc(-c2nc(Nc3ccncc3)cc(N3CCN(C)CC3)n2)c1]

To a mixture of compound 2-(3-(4-amino-6-(4-methylpiperazin-1-yl)pyrimidin-2-yl)phenoxy)-N-isopropylacetamide (460 mg, 1.199 mmol), 4-iodopyridine (319.5 mg, 1.559 mmol), Pd₂(dba)₃ (109.8 mg, 0.12 mmol) and X-Phos (57 mg, 0.12 mmol) in anhydrous 1,4-dioxane (15 mL) was added Cs₂CO₃ (1.17 g, 3.3 mmol ). The resulting mixture was heated to 120° C. overnight under N₂. After cooling to room temperature, the mixture was diluted with 1,4-dioxane and filtered through celite pad. The filtrate was concentrated and the residue was washed with EtOAc and dried in vacuo to obtain the title compound (173 mg, yield 31.3%) as a white solid. ¹H NMR (400 MHz, CD3OD) δ 1.17 (d, J=6.4 Hz, 6H), 2.36 (s, 3H), 2.55 (t, J=5.2 Hz, 4H), 3.72 (t, J=4.8 Hz, 4H), 4.06-4.16 (m, 1H), 4.56 (s, 2H), 5.97 (s, 1H), 7.19 (dd, J=8.0 and 2.4 Hz, 1H), 7.39 (t, J=8.0 Hz, 1H), 7.78 (d, J=6.4 Hz, 2H), 7.98 (s, 1H), 8.02 (d, J=8.0 Hz, 1H), 8.32 (d, J=5.6 Hz, 1H). m/e 462 (M+H)⁺.

### Example 71

### N-isopropyl-2-(3-(2-(4-methylpiperazin-1-yl)-6-(pyridin-4-ylamino)pyrimidin-4-yl)phenoxy)acetamide

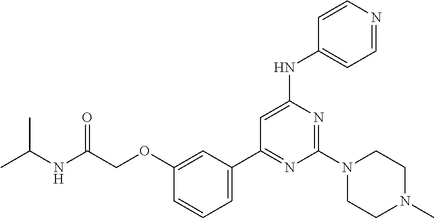 [SMILES: CC(C)NC(=O)COc1cccc(-c2cc(Nc3ccncc3)nc(N3CCN(C)CC3)n2)c1]

To a mixture of compound 2-(3-(6-amino-2-(4-methylpiperazin-1-yl)pyrimidin-4-yl)phenoxy)-N-isopropylacetamide (493 mg, 1.286 mmol), 4-iodopyridine (290 mg, 1.415 mmol), Pd₂(dba)₃ (117.8 mg, 0.129 mmol) and X-Phos (61.4 mg, 0.129 mmol) in anhydrous 1,4-dioxane (15 mL) was added Cs₂CO₃ (1258 mg, 3.858 mmol). The resulting mixture was heated to 120° C. overnight under N₂. After cooling to room temperature, the mixture was diluted with 1,4-dioxane and filtered through celite pad. The filtrate was concentrated and the residue was washed with EtOAc and dried in vacuo to obtain the title compound (184 mg, yield 31.0%) as a white solid. 1H NMR (400 MHz, CD3OD) δ 1.17 (d, J=6.4 Hz, 6H), 2.36 (s, 3H), 2.57 (t, J=4.8 Hz, 4H), 3.65 (b, 4H), 4.08-4.14 (m, 1H), 4.54 (s, 2H), 6.57 (s, 1H), 7.08 (dd, J=8.0 and 2.4 Hz, 1H), 7.39 (t, J=8.4 Hz, 1H), 7.62 (d, J=7.6 Hz, 1H), 7.69-7.73 (m, 3H), 8.31 (d, J=6.4 Hz, 1H). m/e 462 (M+H)⁺.

### Example 72

### tert-Butyl 4-((3-bromophenoxy)methyl)piperidine-1-carboxylate

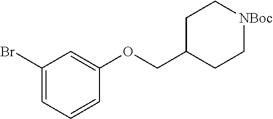 [SMILES: CC(C)(C)OC(=O)N1CCC(COc2cccc(Br)c2)CC1]

A solution of tert-butyl 4-(hydroxymethyl)piperidine-1-carboxylate (1.5 g, 7.0 mmol), 3-bromophenol (1.5 g, 7.0 mmol) and PPh₃ (2.7 g, 10.5 mmol) was stirred in dry THF (30 mL) was stirred at 0° C. under a nitrogen atmosphere. To this mixture was added DEAD (1.8 g, 10.5 mmol) dropwise over a period of 5 min, and the reaction was monitored by TLC. After complete disappearance of the starting material, the solvent was evaporated under reduced pressure and the resulting oil purified by column chromatography (PE/EA, 9/1) to provide the title compound (2.4 g crude) which was used directly without further purification. m/e 372 (M+H)⁺.

### Example 73

### tert-butyl 4-((3-(4,4,5,5-tetramethyl-1,3,2-dioxaborolan-2-yl)phenoxy)methyl) piperidine-1-carboxylate

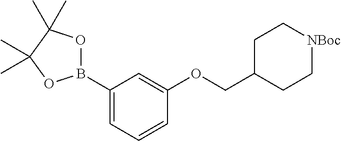 [SMILES: CC(C)(C)OC(=O)N1CCC(COc2cccc(B3OC(C)(C)C(C)(C)O3)c2)CC1]

A solution of tert-butyl 4-((3-bromophenoxy)methyl)piperidine-1-carboxylate (2.4 g, 6.5 mmol), Pin₂B₂ (2.5 g, 9.7 mmol), Pd(dppf)Cl₂ (250 mg) and potassium acetate (1.9 g, 19.4 mmol) in 50 mL of dioxane was degassed and flushed with N₂, heated at 80° C. for 14 h. The mixture was concentrated to give a residue, which was diluted with EtOAc (300 L), filtered, concentrated and was purified by chromatography (EA:PE, 1:10) to give the title compound (600 mg, 22%) as a yellow solid. 1H NMR (300 MHz, CDCl₃) δ 7.38 (1H, m), 7.27 (2H, m), 6.96 (1H, m), 3.82 (2H, d, J=6.0 Hz), 2.76 (1H, m), 1.73 (4H, m), 1.84 (4H, m).

### Example 74

### tert-Butyl 5-(tert-butoxycarbonyl(2-(3-((1-(tert-butoxycarbonyl)piperidin-4-yl) methoxy)phenyl)pyrimidin-4-yl)amino)-1H-indazole-1-carboxylate

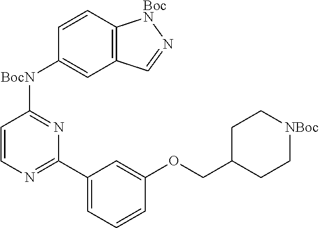 [SMILES: CC(C)(C)OC(=O)N1CCC(COc2cccc(-c3nccc(N(C(=O)OC(C)(C)C)c4ccc5c(cnn5C(=O)OC(C)(C)C)c4)n3)c2)CC1]

A mixture of tert-butyl 5-(tert-butoxycarbonyl(2-chloropyrimidin-4-yl)amino)-1H-indazole-1-carboxylate (642 mg, 1.44 mmol), tert-butyl 4-((3-(4,4,5,5-tetramethyl-1,3,2-dioxaborolan-2-yl)phenoxy)methyl) piperidine-1-carboxylate (600 mg, 1.44 mmol), KOAc (564 mg, 5.76 mmol), Boc₂O (604 mg, 2.88 mmol) and Pd(dppf)Cl₂ (70 mg) in dioxane/water (30 mL/3 mL) was degassed and flushed with N₂, heated at 100° C. 16 h. The mixture was concentrated to give a residue, which was diluted with DCM, filtered, concentrated and purified by chromatography (PE/EA, 5/1) to give the title compound (300 mg, crude) as a yellow oil. m/e 701 (M+H)⁺.

### Example 75

### N-(2-(3-(piperidin-4-ylmethoxy)phenyl)pyrimidin-4-yl)-1H-indazol-5-amine

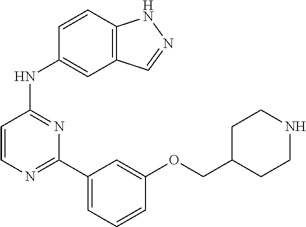 [SMILES: c1cc(OCC2CCNCC2)cc(-c2nccc(Nc3ccc4[nH]ncc4c3)n2)c1]

tert-Butyl-5-(tert-butoxycarbonyl(2-(3-((1-(tert-butoxycarbonyl)piperidin-4-yl) methoxy)phenyl)pyrimidin-4-yl)amino)-1H-indazole-1-carboxylate (250 mg, 0.36 mmol) was dissolved in 30 mL HCl/Et₂O (saturated). It was stirred at room temperature over night. The mixture was concentrated to give a residue, which was diluted with water, and extracted by EA. The water phase was adjusted to 11 using saturated NaHCO₃ solution. It was concentrated and the residue was further purified by preparative TLC to provide the title compound as a yellow solid (50 mg, 35%). ¹H NMR (300 MHz, CD₃OD) δ 8.24 (2H, m), 8.04 (1H, s), 7.90 (2H, m), 7.56 (2H, m), 7.38 (1H, t, J=6.0 Hz), 7.04 (1H, dd, J=9.0 Hz, J=3.0 Hz), 6.65 (1H, d, J=6.0 Hz), 3.95 (2H, d, J=6.0 Hz), 3.42 (2H, d, J=3.0 Hz), 2.15 (2H, t, J=3.0 Hz), 2.12 (1H, m), 2.07 (2H, q, J=3.0 Hz), 1.69 (2H, q, J=3.0 Hz); m/e 401 (M+H)⁺.

### Example 76

### tert-Butyl 4-(3-bromophenoxy) piperidine-1-carboxylate

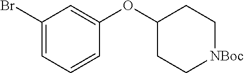 [SMILES: CC(C)(C)OC(=O)N1CCC(Oc2cccc(Br)c2)CC1]

A solution of tert-butyl 4-hydroxypiperidine-1-carboxylate (1.4 g, 7.0 mmol), 3-bromophenol (1.2 g, 7.0 mmol) and PPh₃ (2.7 g, 10.4 mmol) was stirred in dry THF (35 mL) at 0° C. under a nitrogen atmosphere. To this mixture was added DEAD (1.8 g, 10.4 mmol) dropwise over a period of 5 min, and the reaction was monitored by TLC. After complete disappearance of starting material, the solvent was evaporated under reduced pressure and the resulting oil purified by column chromatography (PE/EA, 9/1) to provide the title compound (1.3 g, 52%). m/e 357 (M+H)⁺.

### Example 77

### tert-Butyl-4-(3-(4,4,5,5-tetramethyl-1,3,2-dioxaborolan-2-yl)phenoxy)piperidine-1-carboxylate

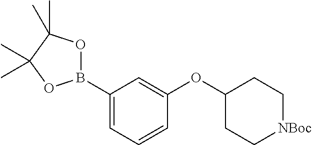 [SMILES: CC(C)(C)OC(=O)N1CCC(Oc2cccc(B3OC(C)(C)C(C)(C)O3)c2)CC1]

A solution of tert-butyl 4-(3-bromophenoxy) piperidine-1-carboxylate (1.3 g, 3.5 mmol), Pin₂B₂ (1.4 g, 5.3 mmol), Pd(dppf)Cl₂ (135 mg) and potassium acetate (1.0 g, 10.6 mmol) in 20 mL of dioxane was degassed, flushed with N₂, and heated at 80° C. for 14 h. The mixture was concentrated to give a residue, which was diluted with EtOAc (200 mL), filtered, concentrated and purified by chromotography (EA:PE, 1:10) to give the title compound (500 mg, 36%) as a yellow solid. ¹H NMR (300 MHz, CDCl₃) δ 7.38 (1H, m), 7.32 (1H, m), 7.27 (1H, m), 7.25 (1H, m), 7.38 (1H, m), 4.51 (1H, m), 3.69 (2H, m), 3.36 (2H, m), 1.88 (2H, m), 1.74 (2H, m), 1.34 (2H, m).

### Example 78

### tert-Butyl-5-(tert-butoxycarbonyl(2-(3-(1-(tert-butoxycarbonyl)piperidin-4-yloxy)phenyl)pyrimidin-4-yl)amino)-1H-indazole-1-carboxylate

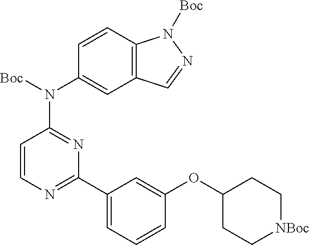 [SMILES: CC(C)(C)OC(=O)N1CCC(Oc2cccc(-c3nccc(N(C(=O)OC(C)(C)C)c4ccc5c(cnn5C(=O)OC(C)(C)C)c4)n3)c2)CC1]

A mixture of tert-butyl 5-(tert-butoxycarbonyl(2-chloropyrimidin-4-yl)amino)-1H-indazole-1-carboxylate (221 mg, 0.5 mmol), tert-butyl 4-(3-(4,4,5,5-tetramethyl-1,3,2-dioxaborolan-2-yl)phenoxy)piperidine-1-carboxylate (200 mg, 0.5 mmol), KOAc (196 mg, 2.0 mmol), Boc₂O (210 mg, 1.0 mmol) and Pd(dppf)Cl₂ (40 mg) in dioxane/water (30 mL/3 mL) was degassed and flushed with N₂, heated at 100° C. 16 h. The mixture was concentrated to give a residue, which was diluted with DCM, filtered, concentrated and purified by chromatography (PE/EA, 5/1) to provide the title compound (180 mg, crude) as a yellow oil. m/e 687 (M+H)⁺.

### Example 79

### N-(2-(3-(Piperidin-4-yloxy)phenyl)pyrimidin-4-yl)-1H-indazol-5-amine

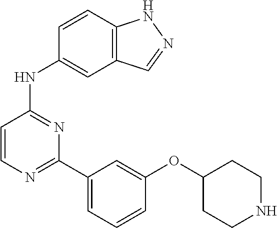 [SMILES: c1cc(OC2CCNCC2)cc(-c2nccc(Nc3ccc4[nH]ncc4c3)n2)c1]

tert-Butyl-5-(tert-butoxycarbonyl(2-(3-(1-(tert-butoxycarbonyl)piperidin-4-yloxy)phenyl)pyrimidin-4-yl)amino)-1H-indazole-1-carboxylate (170 mg, 0.25 mmol) was dissolved in 30 mL HCl/Et₂O (saturated). It was stirred at room temperature overnight. The mixture was concentrated to give a residue, which was diluted with water, and extracted by EA. The water phase was adjusted to 11 using saturated NaHCO₃ solution. It was concentrated and the residue was further purified by preparative TLC to provide the title compound (20 mg, 21%) as a yellow solid. ¹H NMR (300 MHz, CD₃OD) δ 8.26 (1H, d, J=6.0 Hz), 8.18 (1H, s), 8.04 (1H, s), 7.96 (1H, s), 7.93 (1H, t, J=3.0 Hz), 7.56 (2H, m), 7.42 (1H, t, J=9.0 Hz), 7.14 (1H, dd, J=9.0 Hz, J=3.0 Hz), 6.87 (1H, d, J=6.0 Hz), 5.80 (1H, m), 3.38 (2H, m), 3.30 (2H, m), 2.16 (2H, m), 2.11 (2H, m); m/e 387 (M+H)⁺.

### Example 80

### 2-(3-Bromo-4-fluorophenoxy)-N-cyclopropylacetamide

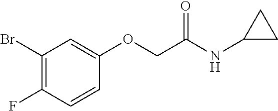 [SMILES: O=C(COc1ccc(F)c(Br)c1)NC1CC1]

A solution of 2-chloro-N-cyclopropylacetamide (1.7 g, 13.1 mmol), 3-bromo-4-fluorophenol (2.5 g, 13.1 mmol) and K₂CO₃ (2.7 g, 19.6 mmol) in 20 mL of acetone was heated at 60° C. for 16 h. The mixture was filtered and concentrated to give a residue, which was purified by column chromatography (PE/EA, 3/1) to give the title compound (2.5 g, 66%) as a yellow solid. m/e 289 (M+H)⁺.

### Example 81

### N-Cyclopropyl-2-(4-fluoro-3-(4,4,5,5-tetramethyl-1,3,2-dioxaborolan-2-yl)phenoxy)acetamide

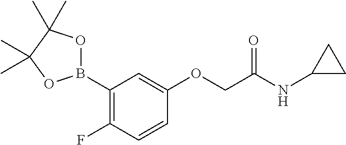 [SMILES: CC1(C)OB(c2cc(OCC(=O)NC3CC3)ccc2F)OC1(C)C]

A solution of 2-(3-bromo-4-fluorophenoxy)-N-cyclopropylacetamide (2.5 g, 8.7 mmol), Pin₂B₂ (3.3 g, 13.0 mmol), Pd(dppf)Cl₂ (330 mg) and potassium acetate (2.6 g, 26.0 mmol) in 35 mL of dioxane was degassed and flushed with N₂, heated at 80° C. for 14 h. The mixture was concentrated to give a residue, which was diluted with EtOAc (200 mL), filtered, concentrated and purified by chromatography (EA:PE, 1:5) to give the title compound (600 mg, 21%) as a yellow solid. m/e 336 (M+H)⁺.

### Example 82

### 2-(3-(4-(1H-indazol-5-ylamino)pyrimidin-2-yl)-4-fluorophenoxy)-N-cyclopropylacetamide

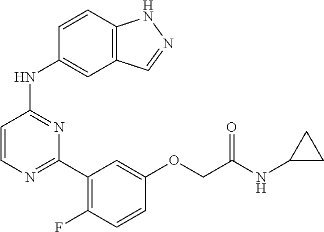 [SMILES: O=C(COc1ccc(F)c(-c2nccc(Nc3ccc4[nH]ncc4c3)n2)c1)NC1CC1]

A mixture of N-(2-chloropyrimidin-4-yl)-1H-indazol-5-amine (150 mg, 0.61 mmol), N-cyclopropyl-2-(4-fluoro-3-(4,4,5,5-tetramethyl-1,3,2-dioxaborolan-2-yl)phenoxy)acetamide (205 mg, 0.61 mmol), KOAc (240 mg, 2.45 mmol) and Pd(dppf)Cl₂ (60 mg) in dioxane/water (20 mL/3 mL) was degassed and flushed with N₂, heated at 100° C. 16 h. The mixture was concentrated to give a residue, which was diluted with DCM, filtered, concentrated and purified by chromatography (DCM/MeOH, 20/1) followed by further purification by preparative TLC to give the title compound (35 mg, 14%) as a yellow solid. 1H NMR (300 MHz, CD₃OD) δ 8.26 (1H, d, J=6.0 Hz), 8.17 (1H, s), 8.03 (1H, s), 7.52 (3H, m), 7.13 (2H, m), 6.70 (1H, d, J=6.0 Hz), 4.53 (2H, s), 2.72 (1H, t, J=3.0 Hz), 0.75 (2H, t, J=3.0 Hz), 0.57 (2H, t, J=3.0 Hz); m/e 419 (M+H)⁺.

### Example 83

### 2-(3-(4-(1H-indazol-5-ylamino)-5-methylpyrimidin-2-yl)-4-fluorophenoxy)-N-cyclopropylacetamide

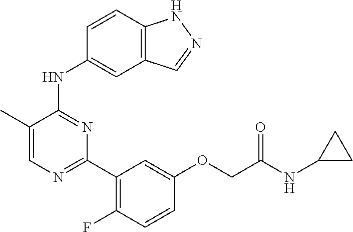 [SMILES: Cc1cnc(-c2cc(OCC(=O)NC3CC3)ccc2F)nc1Nc1ccc2[nH]ncc2c1]

A mixture of N-(2-chloro-5-methylpyrimidin-4-yl)-1H-indazol-5-amine (209 mg, 0.80 mmol), N-cyclopropyl-2-(4-fluoro-3-(4,4,5,5-tetramethyl-1,3,2-dioxaborolan-2-yl)phenoxy)acetamide (270 mg, 0.80 mmol), KOAc (316 mg, 3.22 mmol) and Pd(dppf)Cl₂ (60 mg) in dioxane/water (20 mL/3 mL) was degassed and flushed with N₂, heated at 100° C. 16 h. The mixture was concentrated to give a residue, which was diluted with DCM, filtered, concentrated and purified by chromatography (DCM/MeOH, 20/1) followed by further purification by preparative TLC to give the title compound (35 mg, 10%) as a yellow solid.

¹H NMR (300 MHz, CD₃OD) δ 8.16 (1H, s), 8.12 (1H, s), 8.02 (1H, s), 7.70 (1H, dd, J=9.0 Hz, J=3.0 Hz), 7.54 (1H, d, J=9.0 Hz), 7.44 (1H, m), 7.10 (2H, m), 4.46 (2H, s), 2.69 (1H, m), 2.31 (3H, s), 0.73 (2H, t, J=3.0 Hz), 0.55 (2H, t, J=3.0 Hz); m/e 433 (M+H)⁺.

### Example 84

### 2-(3-Bromo-5-fluorophenoxy)-N-cyclopropylacetamide

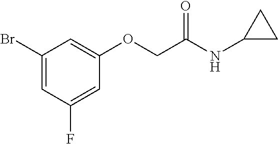 [SMILES: O=C(COc1cc(F)cc(Br)c1)NC1CC1]

A solution of 2-chloro-N-cyclopropylacetamide (2.8 g, 20.9 mmol), 3-bromo-5-fluorophenol (4.0 g, 20.9 mmol) and K₂CO₃ (4.3 g, 31.4 mmol) in 40 mL of acetone was heated at 60° C. for 16 h. The mixture was filtered and concentrated to give a residue, which was purified by column chromatography (PE/EA, 3/1) to give the title compound (4.3 g, 71%) as a yellow solid. m/e 288 (M+H)⁺.

### Example 85

### N-cyclopropyl-2-(3-fluoro-5-(4,4,5,5-tetramethyl-1,3,2-dioxaborolan-2-yl)phenoxy)acetamide

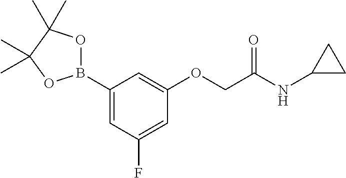 [SMILES: CC1(C)OB(c2cc(F)cc(OCC(=O)NC3CC3)c2)OC1(C)C]

A solution of 2-(3-bromo-5-fluorophenoxy)-N-cyclopropylacetamide (4.3 g, 14.9 mmol), Pin₂B₂ (5.7 g, 22.4 mmol), Pd(dppf)Cl₂ (600 mg) and potassium acetate (4.4 g, 44.8 mmol) in 50 mL of dioxane was degassed and flushed with N₂, heated at 80° C. for 14 h. The mixture was concentrated to give a residue, which was diluted with EtOAc (200 mL), filtered, concentrated and purified by chromatography (EA:PE, 1:5) to give the title compound (3.2 g, 64%) as a yellow solid. m/e 336 (M+H)⁺.

### Example 86

### 2-(3-(4-(1H-indazol-5-ylamino)pyrimidin-2-yl)-5-fluorophenoxy)-N-cyclopropylacetamide

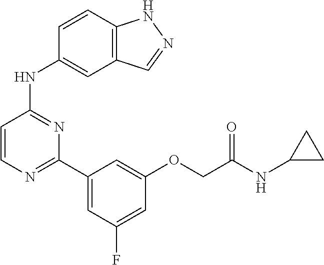 [SMILES: O=C(COc1cc(F)cc(-c2nccc(Nc3ccc4[nH]ncc4c3)n2)c1)NC1CC1]

A mixture of N-(2-chloropyrimidin-4-yl)-1H-indazol-5-amine (340 mg, 1.4 mmol), N-cyclopropyl-2-(3-fluoro-5-(4,4,5,5-tetramethyl-1,3,2-dioxaborolan-2-yl)phenoxy)acetamide (650 mg, 1.9 mmol), CsF (835 mg, 5.5 mmol) and Pd(dppf)Cl₂ (200 mg) in dioxane/water (20 mL/3 mL) was degassed and flushed with N₂, heated at 100° C. 16 h. The mixture was concentrated to give a residue, which was diluted with DCM, filtered, concentrated and purified by chromatography (DCM/MeOH, 20/1) to give the title compound (80 mg, 14%) as a yellow solid. ¹H NMR (300 MHz, CD₃OD) δ 8.26 (1H, d, J=6.0 Hz), 8.08 (1H, s), 8.06 (1H, s), 7.78 (1H, s), 7.65 (1H, dd, J=9.0 Hz, J=3.0 Hz), 7.58 (2H, s), 6.89 (1H, dt, J=9.0 Hz, J=3.0 Hz), 6.66 (1H, d, J=6.0 Hz), 4.58 (2H, s), 2.74 (1H, m), 0.76 (2H, t, J=3.0 Hz), 0.58 (2H, t, J=3.0 Hz); m/e 419 (M+H)⁺.

### Example 87

### 2-(3-(4-(1H-indazol-5-ylamino)-5-methylpyrimidin-2-yl)-5-fluorophenoxy)-N-cyclopropylacetamide

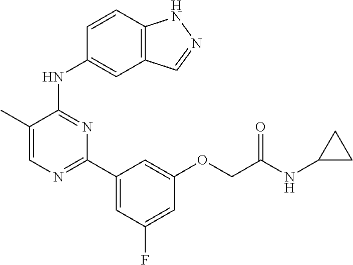 [SMILES: Cc1cnc(-c2cc(F)cc(OCC(=O)NC3CC3)c2)nc1Nc1ccc2[nH]ncc2c1]

A mixture of N-(2-chloro-5-methylpyrimidin-4-yl)-1H-indazol-5-amine (360 mg, 1.4 mmol), N-cyclopropyl-2-(3-fluoro-5-(4,4,5,5-tetramethyl-1,3,2-dioxaborolan-2-yl)phenoxy)acetamide (650 mg, 1.9 mmol), CsF (835 mg, 5.5 mmol) and Pd(dppf)Cl₂ (200 mg) in dioxane/water (20 mL/3 mL) was degassed and flushed with N₂, heated at 100° C. 16 h. The mixture was concentrated to give a residue, which was diluted with DCM, filtered, concentrated and purified by chromatography (DCM/MeOH, 20/1) to give the title compound (60 mg, 10%) as a yellow solid. ¹H NMR (300 MHz, CD₃OD) δ 8.14 (1H, s), 8.04 (2H, m), 7.70 (2H, m), 7.57 (2H, m), 6.81 (1H, dt, J=9.0 Hz, J=3.0 Hz), 4.51 (2H, s), 2.72 (1H, m), 2.29 (3H, s), 0.72 (2H, t, J=3.0 Hz), 0.57 (2H, t, J=3.0 Hz); m/e 433 (M+H)⁺.

### Example 88

### tert-Butyl 3-((3-bromophenoxy)methyl)piperidine-1-carboxylate

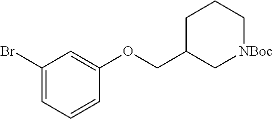 [SMILES: CC(C)(C)OC(=O)N1CCCC(COc2cccc(Br)c2)C1]

A solution of tert-butyl 3-(hydroxymethyl)piperidine-1-carboxylate (935 mg, 4.35 mmol), 3-bromophenol (753 mg, 4.35 mmol) and PPh₃ (1.71 mg, 6.53 mmol) was stirred in dry THF (30 mL) at 0° C. under a nitrogen atmosphere. To this mixture was added dropwise DEAD (1.14 g, 6.53 mmol) over a period of 5 min, and the reaction was monitored by TLC. After complete disappearance of starting material, the mixture was poured to EA (50 mL), washed with brine (3×20 mL), dried over Na₂SO₄ and filtered. The filtrate was evaporated under reduced pressure and the resulting oil was purified by column chromatography (PE/EA, 10/1) to get the title compound (0.8 g, crude). m/e 370 (M+H)⁺.

### Example 89

### tert-Butyl 3-((3-(4,4,5,5-tetramethyl-1,3,2-dioxaborolan-2-yl)phenoxy)methyl) piperidine-1-carboxylate

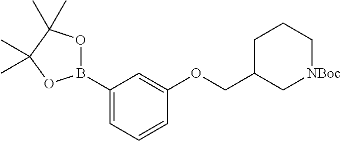 [SMILES: CC(C)(C)OC(=O)N1CCCC(COc2cccc(B3OC(C)(C)C(C)(C)O3)c2)C1]

A solution of tert-butyl 3-((3-bromophenoxy)methyl)piperidine-1-carboxylate (0.7 g, 1.9 mmol), Pin₂B₂ (0.72 g, 2.8 mmol), Pd(dppf)Cl₂ (154 mg) and potassium acetate (556 mg, 5.67 mmol) in dioxane (50 mL) was degassed and flushed with N₂, heated at 80° C. for 14 h. The mixture was concentrated to give a residue, which was diluted with EtOAc (100 mL), washed with brine (3×30 mL), dried over Na₂SO₄ and filtered. The filtrate was evaporated under reduced pressure and the resulting oil was purified by column chromatography (EA:PE, 1:5) to give the title compound (0.9 g, crude). m/e 418 (M+H)⁺.

### Example 90

### tert-Butyl-5-(tert-butoxycarbonyl(2-(3-((1-(tert-butoxycarbonyl)piperidin-3-yl) methoxy)phenyl)pyrimidin-4-yl)amino)-1H-indazole-1-carboxylate

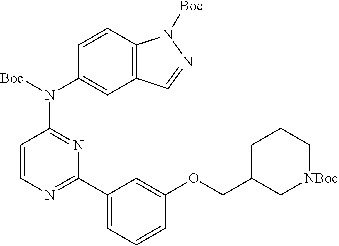 [SMILES: CC(C)(C)OC(=O)N1CCCC(COc2cccc(-c3nccc(N(C(=O)OC(C)(C)C)c4ccc5c(cnn5C(=O)OC(C)(C)C)c4)n3)c2)C1]

A mixture of tert-butyl 5-(tert-butoxycarbonyl(2-chloropyrimidin-4-yl)amino)-1H-indazole-1-carboxylate (240 mg, 0.54 mmol), tert-butyl 3-((3-(4,4,5,5-tetramethyl-1,3,2-dioxaborolan-2-yl)phenoxy)methyl) piperidine-1-carboxylate (270 mg, 0.65 mmol), CsF (788 mg, 5.4 mmol), Boc₂O (353 mg, 1.62 mmol) and Pd(dppf)Cl₂ (88 mg) in dioxane/water (20 mL/2 mL) was degassed and flushed with N₂, heated at 100° C. for 16 h. The mixture was concentrated to give a residue, which was diluted with EtOAc (100 mL), washed with brine (30 mL×3), dried over Na₂SO₄ and filtered. The filtrate was evaporated under reduced pressure and the resulting oil was purified by column chromatography (DCM:MeOH, 50:1) to give the title compound (0.1 g) as a yellow oil. m/e 701 (M+H)⁺.

### Example 91

### N-(2-(3-(piperidin-3-ylmethoxy)phenyl)pyrimidin-4-yl)-1H-indazol-5-amine

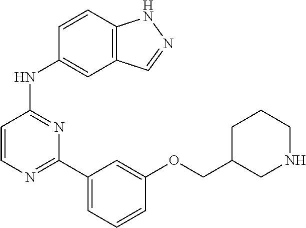 [SMILES: c1cc(OCC2CCCNC2)cc(-c2nccc(Nc3ccc4[nH]ncc4c3)n2)c1]

tert-Butyl-5-(tert-butoxycarbonyl(2-(3-((1-(tert-butoxycarbonyl)piperidin-3-yl) methoxy)phenyl)pyrimidin-4-yl)amino)-1H-indazole-1-carboxylate (100 mg, 0.14 mmol) was dissolved in HFIP (3 mL), the solution was stirred at 150° C. for 1 h with M.W. The mixture was concentrated to give a residue, which was purified by pre-TLC (DCM:MeOH, 4:1) to give the title compound as a yellow solid (40 mg, 70%). ¹H NMR (400 MHz, CD₃OD) δ 8.26 (d, J=8.4 Hz, 1H), 8.21 (s, 1H), 8.07 (s, 1H), 7.91-7.88 (m, 2H), 7.61-7.52 (m, 2H), 7.44 (t, J=8.0 Hz, 1H), 7.14-7.12 (m, 1H), 6.74 (d, J=6.4 Hz, 1H), 4.11-4.07 (m, 1H), 3.97-3.93 (m, 1H), 3.58-3.54 (m, 1H), 3.41-3.31 (m, 1H), 3.00-2.89 (m, 2H), 2.36-2.27 (m, 1H), 2.04-1.97 (m, 1H), 1.85-1.78 (m, 1H), 1.56-1.45 (m, 1H); m/e 401 (M+H)⁺.

### Example 92

### tert-Butyl 3-(3-bromophenoxyl)pyrrolidine-1-carboxylate

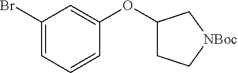 [SMILES: CC(C)(C)OC(=O)N1CCC(Oc2cccc(Br)c2)C1]

A solution of tert-butyl 3-hydroxypyrrolidine-1-carboxylate (1.0 g, 5.8 mmol), 3-bromophenol (1.08 g, 5.8 mmol) and PPh₃ (2.28 g, 8.7 mmol) in dry THF (35 mL) was stirred at 0° C. under a nitrogen atmosphere. To this mixture was added DEAD (1.51 g, 8.7 mmol) dropwise over a period of 5 min, and the reaction was monitored by TLC. After complete disappearance of starting material, the mixture was poured to EA (50 mL), washed with brine (20 mL×3), dried over Na₂SO₄ and filtered. The filtrate was evaporated under reduced pressure and the resulting oil was purified by column chromatography (PE/EA, 10/1) to afford the title compound (0.8 g, crude). m/e 342 (M+H)⁺.

### Example 93

### tert-Butyl 3-(3-(4,4,5,5-tetramethyl-1,3,2-dioxaborolan-2-yl)phenoxy)pyrrolidine-1-carboxylate

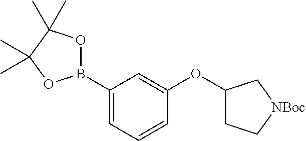 [SMILES: CC(C)(C)OC(=O)N1CCC(Oc2cccc(B3OC(C)(C)C(C)(C)O3)c2)C1]

A solution of tert-butyl 3-(3-bromophenoxyl)pyrrolidine-1-carboxylate (0.8 g, 2.3 mmol), Pin₂B₂ (1.91 g, 7.5 mmol), Pd(dppf)Cl₂ (408 mg) and potassium acetate (1.47 g, 15 mmol) in dioxane (50 mL) was degassed and flushed with N₂, heated at 80° C. for 14 h. The mixture was concentrated to give a residue, which was diluted with EtOAc (100 mL), washed with brine (3×30 mL), dried over Na₂SO₄ and filtered. The filtrate was evaporated under reduced pressure and the resulting oil was purified by column chromatography (EA:PE, 1:5) to give the title compound as a yellow oil (450 mg, crude). m/e 390 (M+H)⁺.

### Example 94

### tert-Butyl-5-(tert-butoxycarbonyl(2-(3-(1-(tert-butoxycarbonyl)pyrrolidin-3-yloxy)phenyl)pyrimidin-4-yl)amino)-1H-indazole-1-carboxylate

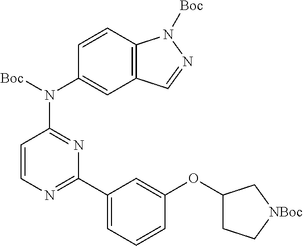 [SMILES: CC(C)(C)OC(=O)N1CCC(Oc2cccc(-c3nccc(N(C(=O)OC(C)(C)C)c4ccc5c(cnn5C(=O)OC(C)(C)C)c4)n3)c2)C1]

A mixture of tert-butyl 5-(tert-butoxycarbonyl(2-chloropyrimidin-4-yl)amino)-1H-indazole-1-carboxylate (466 mg, 1.05 mmol), tert-butyl 3-(3-(4,4,5,5-tetramethyl-1,3,2-dioxaborolan-2-yl)phenoxy)pyrrolidine-1-carboxylate (488 mg, 1.25 mmol), CsF (1.53 g, 10.5 mmol), Boc₂O (687 mg, 3.15 mmol) and Pd(dppf)Cl₂ (172 mg) in dioxane/water (30 mL/3 mL) was degassed and flushed with N₂, heated at 100° C. for 16 h. The mixture was concentrated to give a residue, which was diluted with EtOAc (100 mL), washed with brine (3×30 mL), dried over Na₂SO₄ and filtered. The filtrate was evaporated under reduced pressure and the resulting oil was purified by column chromatography (PE/EA, 5/1) to give the title compound as a yellow solid (200 mg, 28.5%). m/e 673 (M+H)⁺.

### Example 95

### N-(2-(3-(pyrrolidin-3-yloxy)phenyl)pyrimidin-4-yl)-1H-indazol-5-amine

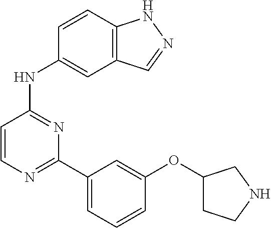 [SMILES: c1cc(OC2CCNC2)cc(-c2nccc(Nc3ccc4[nH]ncc4c3)n2)c1]

tert-Butyl-5-(tert-butoxycarbonyl(2-(3-(1-(tert-butoxycarbonyl)pyrrolidin-3-yloxy)phenyl)pyrimidin-4-yl)amino)-1H-indazole-1-carboxylate (135 mg, 0.25 mmol) was dissolved in 2 mL of con. HCl. It was stirred for 5 minutes at room temperature. 10 mL of water was added and then adjust pH 9-10 by IN NaOH. Extracted with EA (30 mL×3), dried over Na₂SO₄ and filtered. The filtrate was evaporated under reduced pressure and the resulting oil was purified by pre-TLC (DCM/MeOH, 5/1) to give the title compound as a yellow solid (45 mg, 62.5%). ¹H NMR (400 MHz, CD₃OD) δ 8.27 (d, J=6.0 Hz, 1H), 8.17 (s, 1H), 8.05 (s, 1H), 7.98 (d, J=7.6 Hz, 1H), 7.90-7.86 (m, 1H), 7.59-7.53 (m, 2H), 7.44 (t, J=8.0 Hz, 1H), 7.11 (dd, J=2.0 Hz, J=2.4 Hz, 1H), 6.67 (d, J=6.0 Hz, 1H), 5.28-5.26 (m, 1H), 3.62-3.31 (m, 4H), 2.40-2.25 (m, 2H); m/e 373 (M+H)⁺.

### Example 96

### 2-(5-Bromo-2-fluorophenoxy)-N-cyclopropylacetamide

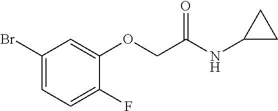 [SMILES: O=C(COc1cc(Br)ccc1F)NC1CC1]

A solution of 2-chloro-N-cyclopropylacetamide (1.0 g, 7.5 mmol), 5-bromo-2-fluorophenol (1.44 g, 7.5 mmol) and K₂CO₃ (1.55 g, 11.25 mmol) in 30 mL of acetone was heated at 60° C. for 16 h. The mixture was filtered and concentrated to give a residue, which was purified by column chromatography (PE/EA, 2/1) to give the title as a yellow solid (1.2 g, 55.3%). m/e 288 (M+H)⁺.

### Example 97

### N-cyclopropyl-2-(2-fluoro-5-(4,4,5,5-tetramethyl-1,3,2-dioxaborolan-2-yl)phenoxy)acetamide

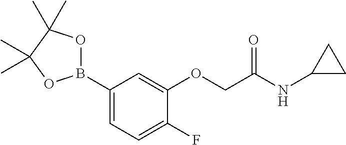 [SMILES: CC1(C)OB(c2ccc(F)c(OCC(=O)NC3CC3)c2)OC1(C)C]

A solution of 2-(5-bromo-2-fluorophenoxy)-N-cyclopropylacetamide (0.83 g, 2.88 mmol), Pin₂B₂ (1.1 g, 4.33 mmol), Pd(dppf)Cl₂ (120 mg) and potassium acetate (0.85 g, 8.64 mmol) in 35 mL of dioxane was degassed and flushed with N₂, heated at 80° C. for 14 h.

The mixture was concentrated to give a residue, which was diluted with EtOAc (100 mL), washed with brine (3×30 mL), dried over Na₂SO₄ and filtered. The filtrate was evaporated under reduced pressure and the resulting oil was purified by column chromatography (PE/EA, 2/1) to give the title compound as an oil (600 mg, 21%). m/e 336 (M+H)⁺.

### Example 98

### 2-(5-(4-(1H-indazol-5-ylamino)pyrimidin-2-yl)-2-fluorophenoxy)-N-cyclopropylacetamide

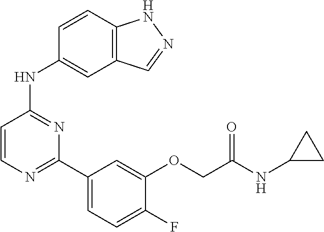 [SMILES: O=C(COc1cc(-c2nccc(Nc3ccc4[nH]ncc4c3)n2)ccc1F)NC1CC1]

A mixture of N-(2-chloropyrimidin-4-yl)-1H-indazol-5-amine (147 mg, 0.6 mmol), N-cyclopropyl-2-(2-fluoro-5-(4,4,5,5-tetramethyl-1,3,2-dioxaborolan-2-yl)phenoxy)acetamide (300 mg, 0.89 mmol), KOAc (235 mg, 2.4 mmol) and Pd(dppf)Cl₂ (70 mg) in dioxane/water (20 mL/3 mL) was degassed and flushed with N₂, heated at 100° C. for 16 h. The mixture was concentrated to give a residue, which was diluted with DCM (30 mL) and filtered. The filtrate was concentrated and purified by column chromatography (DCM/MeOH, 10/1) to give the title compound as a yellow solid (35 mg, 14%). ¹H NMR (400 MHz, DMSO) δ 13.01 (s, 1H), 9.65 (s, 1H), 8.33 (d, J=5.6 Hz, 1H), 8.21-8.20 (m, 1H), 8.09-7.96 (m, 3H), 7.62-7.55 (m, 2H), 7.39-7.34 (m, 1H), 6.68 (d, J=6.0 Hz, 1H), 4.64 (s, 2H), 2.70-2.65 (m, 1H), 0.63-0.59 (m, 2H), 0.48-0.43 (m, 2H); m/e 419 (M+H)⁺.

### Example 99

### 2-(5-(4-(1H-indazol-5-ylamino)-5-methylpyrimidin-2-yl)-2-fluorophenoxy)-N-cyclopropylacetamide

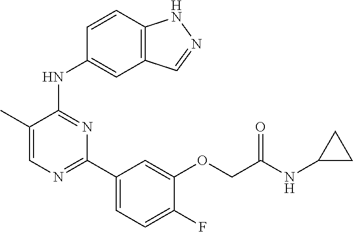 [SMILES: Cc1cnc(-c2ccc(F)c(OCC(=O)NC3CC3)c2)nc1Nc1ccc2[nH]ncc2c1]

A mixture of N-(2-chloro-5-methylpyrimidin-4-yl)-1H-indazol-5-amine (100 mg, 0.38 mmol), N-cyclopropyl-2-(2-fluoro-5-(4,4,5,5-tetramethyl-1,3,2-dioxaborolan-2-yl)phenoxy)acetamide (130 mg, 0.38 mmol), KOAc (151 mg, 1.55 mmol) and Pd(dppf)Cl₂ (50 mg) in dioxane/water (20 mL/3 mL) was degassed and flushed with N₂, heated at 100° C. for 16 h. The mixture was concentrated to give a residue, which was diluted with DCM (30 mL) and filtered. The filtrate was concentrated and purified by column chromatography (DCM/MeOH, 10/1) to give the title compound as a yellow solid (10 mg, 2.9%). ¹H NMR (400 MHz, DMSO) δ 13.01 (s, 1H), 8.62 (s, 1H), 8.21 (s, 1H), 8.17 (d, J=4.0 Hz, 1H), 8.11-8.09 (m, 2H), 7.97-7.94 (m, 1H), 7.85-7.82 (m, 1H), 7.71-7.68 (m, 1H), 7.59-7.56 (m, 1H), 7.31-7.26 (m, 1H), 4.58 (s, 2H), 2.68-2.63 (m, 1H), 2.25 (s, 3H), 0.66-0.58 (m, 2H), 0.47-0.43 (m, 2H); m/e 433 (M+H)⁺.

### Example 100

### N-(6-chloro-2-(pyrrolidin-1-yl)pyrimidin-4-yl)-1H-indazol-5-amine

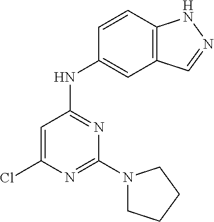 [SMILES: Clc1cc(Nc2ccc3[nH]ncc3c2)nc(N2CCCC2)n1]

A mixture of N-(2,6-dichloropyrimidin-4-yl)-1H-indazol-5-amine (0.88 g, 3.17 mol), pyrrolidine (225 mg, 3.17 mmol) and DIPEA (818 mg, 6.34 mmol) in BuOH (30 mL) was stirred at 120° C. for 12 h. The mixture was concentrated to give a residue, which was purified by pre-HPLC to give the title compound as a white solid (0.8 g, 80%). m/e 315 (M+H)⁺.

### Example 101

### tert-butyl-5-(6-(3-(2-(cyclopropylamino)-2-oxoethoxy)phenyl)-2-(pyrrolidin-1-yl) pyrimidin-4-ylamino)-1H-indazole-1-carboxylate

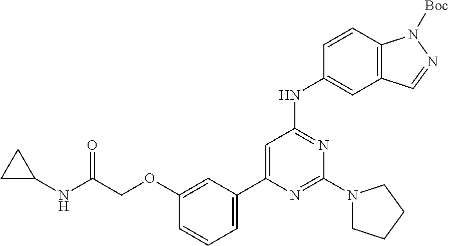 [SMILES: CC(C)(C)OC(=O)n1ncc2cc(Nc3cc(-c4cccc(OCC(=O)NC5CC5)c4)nc(N4CCCC4)n3)ccc21]

A mixture of N-(6-chloro-2-(pyrrolidin-1-yl)pyrimidin-4-yl)-1H-indazol-5-amine (300 mg, 0.95 mmol), N-cyclopropyl-2-(3-(4,4,5,5-tetramethyl-1,3,2-dioxaborolan-2-yl)phenoxy)acetamide (452 mg, 1.43 mmol), CsF (1.38 g, 1.55 mmol) and Pd(dppf)Cl₂ (150 mg) in dioxane/water (30 mL/3 mL) was degassed and flushed with N₂, heated at 100° C. for 16 h. The mixture was concentrated to give a residue, which was diluted with DCM (50 mL) and filtered. The filtrate was concentrated and purified by column chromatography (DCM/MeOH, 10/1) to give the title compound as a yellow solid (90 mg, 16%). m/e 570 (M+H)⁺.

### Example 102

### 2-(3-(6-(1H-indazol-5-ylamino)-2-(pyrrolidin-1-yl)pyrimidin-4-yl)phenoxy)-N-cyclopropylacetamide

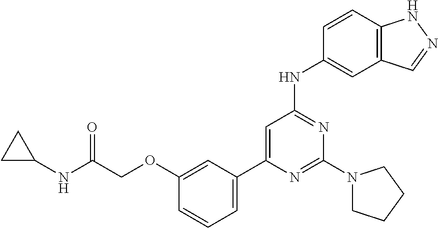 [SMILES: O=C(COc1cccc(-c2cc(Nc3ccc4[nH]ncc4c3)nc(N3CCCC3)n2)c1)NC1CC1]

tert-Butyl 5-(6-(3-(2-(cyclopropylamino)-2-oxoethoxy)phenyl)-2-(pyrrolidin-1-yl) pyrimidin-4-ylamino)-1H-indazole-1-carboxylate (90 mg, 0.16 mmol) was dissolved in HFIP (2 mL), the solution was stirred at 150° C. for 1 h with M.W. The mixture was concentrated to give a residue, which was purified by pre-TLC (DCM:MeOH, 4:1) to give the title compound as a yellow solid (35 mg, 46.7%). ¹H NMR (400 MHz, DMSO) δ 12.92 (s, 1H), 9.27 (s, 1H), 8.32 (s, 1H), 8.19 (d, J=4.4 Hz, 1H), 7.99 (s, 1H), 7.60 (s, 1H), 7.56-7.47 (m, 3H), 7.39 (t, J=8.0 Hz, 1H), 7.02 (dd, J=2.0 Hz, J=2.0 Hz, 1H), 6.47 (s, 1H), 4.50 (s, 2H), 3.63-3.60 (m, 4H), 2.73-2.66 (m, 1H), 1.95-1.98 (m, 4H), 0.66-0.61 (m, 2H), 0.51-0.47 (m, 2H); m/e 470 (M+H)⁺.

### Example 103

### 3-(3-bromophenyl)-N-cyclopropylpropanamide

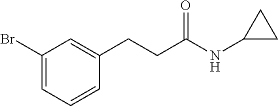 [SMILES: O=C(CCc1cccc(Br)c1)NC1CC1]

3-(3-Bromophenyl) propanoic acid (3.0 g, 13.1 mmol) was added to a solution of SOCl2 (10 ml) and was stirred for 2 hours at 70° C. The mixture was concentrated under reduced pressure. The residue was dissolved in CH2C12 (20 ml), then was added dropwise into the mixture of cyclopropanamine (1.17 g, 19.6 mmol) and triethylamine (4.0 g, 39.3 mmol) at 0° C., then the reaction mixture was stirred overnight at ambient temperature. The reaction mixture was quenched with IN HCl and the organic layer was washed with brine, dried, concentrated to residue. The residue was purified by chromatography (PE/EA: 1/1 to 1/2) to give the title compound as white solid (2.8 g, 79%). ¹H NMR (400 MHz, CDCl₃) 67.36 (m, 2H), 7.15 (m, 2H), 5.53 (s, 1H), 2.94 (t, 2H), 2.68 (s, 1H), 2.41 (t, 2H), 0.77 (m, 2H), 0.44 (m, 2H). m/e 268 (M+H)⁺.

### Example 104

### N-Cyclopropyl-3-(3-(4,4,5,5-tetramethyl-1,3,2-dioxaborolan-2-yl)phenyl)propanamide

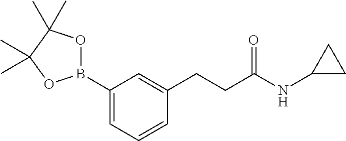 [SMILES: CC1(C)OB(c2cccc(CCC(=O)NC3CC3)c2)OC1(C)C]

PdCl₂(dppf) (420 mg, 0.5 mmol) was added into the mixture of 3-(3-bromophenyl)-N-cyclopropylpropanamide (2.8 g, 10.3 mmol), 4,4,4′,4′,5,5,5′,5′-octamethyl-2,2′-bi(1,3,2-dioxaborolane) (3.9 g, 15.5 mmol) and KOAc (2.5 g, 25.7 mmol) in dioxane (80 ml). The mixture was stirred overnight at 100° C. under nitrogen. The reaction mixture was then concentrated in vacuo, and the residue was purified by chromatography (PE/EA: 5/1 to 1/1) to give the title compound as an off-white solid (3.0 g, 92%). ¹H NMR (300 MHz, CDCl₃) δ 7.60-7.67 (m, 2H), 7.27-7.33 (m, 2H), 5.47 (s, 1H), 2.92-2.97 (m, 2H), 2.41 (t, 2H), 2.04 (s, 1H), 1.34 (s, 12H), 0.70-0.72 (m, 2H), 0.38-0.40 (m, 2H); m/e 316 (M+H)⁺.

### Example 105

### tert-Butyl 5-(tert-butoxycarbonyl(2-(3-(3-(cyclopropylamino)-3-oxopropyl)phenyl) pyrimidin-4-yl)amino)-1H-indazole-1-carboxylate

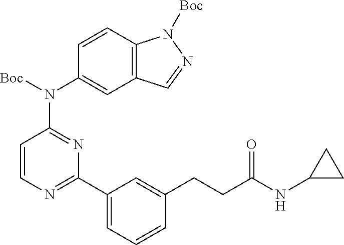 [SMILES: CC(C)(C)OC(=O)N(c1ccc2c(cnn2C(=O)OC(C)(C)C)c1)c1ccnc(-c2cccc(CCC(=O)NC3CC3)c2)n1]

PdCl₂(dppf) (165 mg, 0.21 mmol) was added into the mixture of N-cyclopropyl-3-(3-(4,4,5,5-tetramethyl-1,3,2-dioxaborolan-2-yl)phenyl)propanamide (380 mg, 1.2 mmol), Boc2O (650 mg, 3.0 mmol), CsF (600 mg, 4.0 mmol) and tert-butyl 5-(tert-butoxycarbonyl(2-chloropyrimidin-4-yl)amino)-1H-indazole-1-carboxylate (446 mg, 1.0 mmol) in dioxane/H₂O (30 ml, 10/1) under N₂ flow. The mixture was stirred for 24 h at 100° C. under nitrogen. The reaction mixture was extracted with EA (60 ml) and washed with brine, dried, concentrated in vacuo, and the residue was purified by chromatography (PE/EA: 5/1 to 1/5) to give the title compound product (240 mg) as a yellow oil. m/e 599 (M+H)⁺.

### Example 106

### 3-(3-(4-(1H-indazol-5-ylamino)pyrimidin-2-yl)phenyl)-N-cyclopropylpropanamide

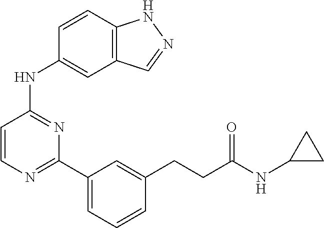 [SMILES: O=C(CCc1cccc(-c2nccc(Nc3ccc4[nH]ncc4c3)n2)c1)NC1CC1]

tert-Butyl-5-(tert-butoxycarbonyl(2-(3-(3-(cyclopropylamino)-3-oxopropyl)phenyl)pyrimidin-4-yl)amino)-1H-indazole-1-carboxylate (220 mg, 0.367 mmol) was added to a mixture of saturated HCl in ethyl ether (30 mL). The mixture was stirred for 3 hours at ambient temperature. Then the mixture was filtered and the yellow solid was added to HCl (5 ml), then was stirred for 10 minutes and diluted with H2O (50 ml), filtered. The obtained off-white crystals as hydrochloride salt was added to saturated NaHCO₃ (10 ml) and was stirred for 2 h. The mixture was filtered and the solid was washed with H₂O (10 ml), dried to give the title compound (50 mg, 34%) as an off-white solid. ¹H NMR (300 MHz, DMSO) δ13.00 (s, 1H), 9.62 (s, 1H), 8.34-8.32 (m, 3H), 8.22 (m, 1H), 8.06 (s, 1H), 7.85 (s, 1H), 7.55 (m, 1H), 7.40 (m, 3H), 6.66 (d, 1H), 2.90 (m, 2H), 2.60 (m, 1H), 2.40 (m, 2H), 0.57 (m, 2H), 0.33 (m, 2H). m/e 399 (M+H)⁺.

### Example 107

### 2-(3-bromophenylthio)-N-cyclopropylacetamide

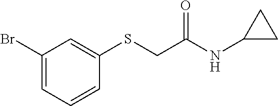 [SMILES: O=C(CSc1cccc(Br)c1)NC1CC1]

A solution of 2-chloro-N-cyclopropylacetamide (1.33 g, 10 mmol), 3-bromobenzenethiol (1.6 g, 8.5 mmol) and K₂CO₃ (4.8 g, 35 mmol) in 30 mL of acetone was heated at 70° C. overnight. The mixture was filtered and concentrated to give a residue, which was purified by column chromatography (PE/EA, 1/1) to give the title compound (2.4 g, 96%) as a white solid. ¹H NMR 6 (300 MHz, CDCl3) 7.39 (1H, m), 7.31 (1H, m), 7.14 (2H, m), 6.71 (1H, s), 3.58 (2H, s), 2.64-2.77 (1H, m), 0.73-0.84 (2H, m), 0.41 (2H, m); m/e 286 (M+H)⁺.

### Example 108

### N-cyclopropyl-2-(3-(4,4,5,5-tetramethyl-1,3,2-dioxaborolan-2-yl)phenylthio)acetamide

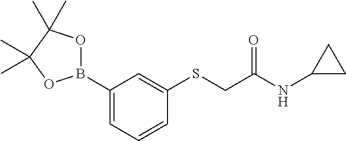 [SMILES: CC1(C)OB(c2cccc(SCC(=O)NC3CC3)c2)OC1(C)C]

A solution of 2-(3-bromophenylthio)-N-cyclopropylacetamide (2.43 g, 9.1 mmol), Pin₂B₂ (3.5 g, 13.7 mmol), Pd(dppf)Cl₂ (730 mg) and potassium acetate (2.67 g, 27.3 mmol) in 30 mL of dioxane was degassed and flushed with N₂, heated at 95° C. for 12 h. The mixture was concentrated to give a residue, which was diluted with EtOAc (200 mL), filtered, concentrated and purified by chromatography (EA:PE, 1:1) to give the title compound (2.4 g, 82%) as a yellow oil. m/e 286 (M+H)⁺.

### Example 109

### tert-Butyl 5-(tert-butoxycarbonyl(2-(3-(2-(cyclopropylamino)-2-oxoethylthio)phenyl)pyrimidin-4-yl)amino)-1H-indazole-1-carboxylate

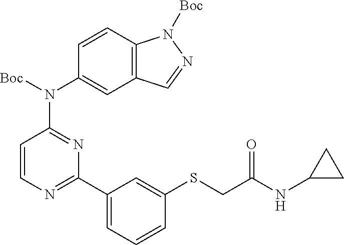 [SMILES: CC(C)(C)OC(=O)N(c1ccc2c(cnn2C(=O)OC(C)(C)C)c1)c1ccnc(-c2cccc(SCC(=O)NC3CC3)c2)n1]

A mixture of N-cyclopropyl-2-(3-(4,4,5,5-tetramethyl-1,3,2-dioxaborolan-2-yl)phenylthio)acetamide (380 mg, 1.1 mmol), N-(2-chloropyrimidin-4-yl)-1H-indazol-5-amine (246 mg, 1.0 mmol), CsF (730 mg, 5.0 mmol), Boc₂O (650 mg, 3.0 mmol), and Pd(dppf)Cl₂ (1700 mg) in dioxane/water (27 mL/3 mL) was degassed and flushed with N₂, heated at 100° C. 24 h. The mixture was concentrated to give a residue, which was diluted with DCM, filtered, concentrated and purified by chromatography (DCM/MeOH, 20/1) to give the crude title compound (200 mg) as a yellow solid. m/e 617 (M+H)⁺.

### Example 110

### 2-(3-(4-(1H-indazol-5-ylamino)pyrimidin-2-yl)phenylthio)-N-cyclopropylacetamide

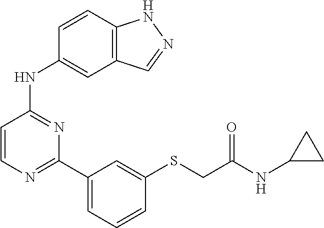 [SMILES: O=C(CSc1cccc(-c2nccc(Nc3ccc4[nH]ncc4c3)n2)c1)NC1CC1]

A mixture of tert-butyl 5-(tert-butoxycarbonyl(2-(3-(2-(cyclopropylamino)-2-oxoethylthio)phenyl)pyrimidin-4-yl)amino)-1H-indazole-1-carboxylate (209 mg, 0.80 mmol) in con. HCl (3 mL) was stirred for 10 minutes followed by addition of ice. The reaction mixture was adjusted to pH 10 with NaHCO3 solution and extracted with CH₂Cl₂/MeOH (1/1, 20 ml). Filtered and the filtrate was concentrated to give a residue, which was purified by chromatography (DCM/MeOH, 20/1) followed by further purification by pre-TLC to give the title compound (25 mg, 18%) as a yellow solid. ¹H NMR (300 MHz, CD₃OD) δ 8.36 (1H, m), 8.24 (1H, d, J=6 Hz), 8.14 (2H, m), 8.05 (1H, s), 7.56 (2H, s), 7.49 (1H, m), 7.41 (1H, d, J=6 Hz), 6.63 (1H, d, J=6 Hz), 3.60 (2H, s), 2.58 (1H, m), 0.61 (2H, m), 0.36 (2H, m); m/e 417 (M+H)⁺.

### Example 111

### 2-(3-(4-aminopyrimidin-2-yl)phenoxy)-N-isopropylacetamide

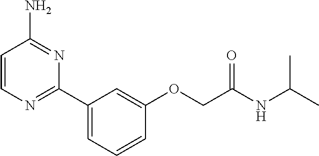 [SMILES: CC(C)NC(=O)COc1cccc(-c2nccc(N)n2)c1]

A mixture of 2-chloropyrimidin-4-amine (0.50 g, 3.8 mmol), N-isopropyl-2-(3-(4,4,5,5-tetramethyl-1,3,2-dioxaborolan-2-yl)phenoxy)acetamide (1.46 g, 4.6 mmol), CsF (1.75 g, 11.4 mmol), and Pd(PPh₃)₄ (0.2 g, 0.2 mmol) in a mixture of dioxane (8 mL) and H₂O (2 mL) was stirred at 100° C. overnight under N₂. After cooling to room temperature, the mixture was concentrated under reduced pressure to give a residue, which was purified by column chromatography on silica gel (eluted with PE:EtOAc=1:1) to provide the title compound (400 mg, yield 36%) as colourless oil.

### Example 112

### N-isopropyl-2-(3-(4-(pyridin-4-ylamino)pyrimidin-2-yl)phenoxy)acetamide

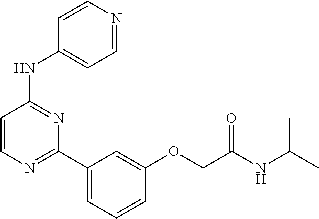 [SMILES: CC(C)NC(=O)COc1cccc(-c2nccc(Nc3ccncc3)n2)c1]

A mixture of compound 2-(3-(4-aminopyrimidin-2-yl)phenoxy)-N-isopropylacetamide

(300 mg, 1.1 mmol), 4-bromopyridine (258 mg, 1.3 mmol), Cs₂CO₃ (1026 mg, 3.3 mmol), Pd₂(dba)₃ (96 mg, 0.1 mmol), and X-Phos (51 mg, 0.1 mmol) in anhydrous dioxane (30 mL) was stirred at 120° C. overnight under N₂. After cooling to room temperature, the mixture was filtered, the filtrate was concentrated, the residue was washed with EtOAc then was filtered to provide the title compound (200 mg, yield 52%) as white solid. 1H NMR (400 MHz, CD₃OD) δ 10.09 (s, 1H), 8.47-7.92 (m, 8H), 7.45 (t, J=7.6 Hz, 1H), 7.12-6.82 (m, 2H), 4.52 (s, 2H), 3.99-3.92 (m, 1H), 1.06 (d, J=6.8 Hz, 6H); m/e 364 (M+H)⁺.

### Example 113

### 2-chloro-4-(4-(pyridin-4-yl)piperidin-1-yl)pyrimidine

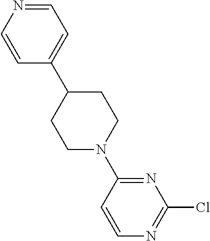 [SMILES: Clc1nccc(N2CCC(c3ccncc3)CC2)n1]

2,4-Dichloropyrimide (745 mg, 5 mmol), 1,2,3,4,5,6-hexahydro-[4,4′]bipyridinyl (811 mg, 5 mmol), and TEA (758 mg, 7.5 mmol) in EtOH (15 mL) was stirred at reflux overnight. After removing the solvent, the residue was purified by column chromatography on silica gel (eluting with petroleum ether:ethyl acetate=5:1-1:1) to give the title compound (500 mg, yield 36.4%) as a white solid.

### Example 114

### N-isopropyl-2-(3-(4-(4-(pyridin-4-yl)piperidin-1-yl)pyrimidin-2-yl)phenoxy)acetamide

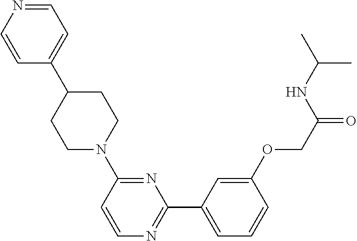 [SMILES: CC(C)NC(=O)COc1cccc(-c2nccc(N3CCC(c4ccncc4)CC3)n2)c1]

A mixture of 2-chloro-4-(4-(pyridin-4-yl)piperidin-1-yl)pyrimidine (500 mg, 1.82 mmol), Pd(dppf)₂Cl₂ (50 mg), Na₂CO₃ (579 mg, 5.46 mmol) and N-isopropyl-2-(3-(4,4,5,5-tetramethyl-1,3,2-dioxaborolan-2-yl)phenoxy)acetamide (871 mg, 2.73 mmol) in dioxane/water (10:1, 10 mL) was stirred at 100° C. overnight. After removing the solvent, the residue was purified by P-HPLC to give the title compound (300 mg, yield 35.2%) as a solid. ¹H NMR (400 MHz, CD₃OD) δ 8.80 (d, J=6.8 Hz, 2H), 8.23 (d, J=7.6 Hz, 1H), 8.10 (d, J=6.4 Hz, 2H), 7.83-7.81 (m, 2H), 7.59 (t, J=8.4 Hz, 1H), 7.37-7.20 (m, 2H), 5.54 (d, J=13.2 Hz, 1H), 4.62 (s, 2H), 4.52 (d, J=14 Hz, 1H), 4.12-4.06 (m, 1H), 3.61-3.47 (m, 4H), 2.26-1.89 (m, 4H), 1.17 (d, J=7.6 Hz, 6H); m/e 432 (M+H)⁺.

### Example 115

### 4,6-dichloro-2-iodopyrimidine

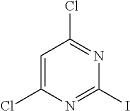

To a solution of compound 4,6-dichloropyrimidin-2-amine (39 g, 237.82 mmol) in CH3CN (300 mL), CH₂12 (1000 mL) was added then t-BuONO (129.3 g, 1.25 mol) was added and the mixture was heated to reflux overnight. The mixture was concentrated under reduced pressure and the residue was purified by column chromatography to give compound the title compound (30 g, yield 46%) as a yellow solid.

### Example 116

### 2-(3-(4,6-dichloropyrimidin-2-yl)phenoxy)-N-isopropylacetamide

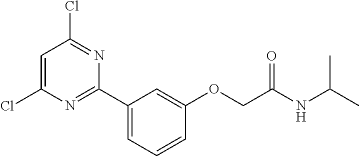 [SMILES: CC(C)NC(=O)COc1cccc(-c2nc(Cl)cc(Cl)n2)c1]

To a mixture of compound 4,6-dichloro-2-iodopyrimidine (13.92 g, 50.64 mmol), N-isopropyl-2-(3-(4,4,5,5-tetramethyl-1,3,2-dioxaborolan-2-yl)phenoxy)acetamide (18 g, 56.39 mmol), Na₂CO₃ (13.88 g, 130.96) in DME (150 mL) and water (50 mL), Pd(PPh₃)₄ (5.04 g, 4.36 mmol) was added and the mixture was heated to reflux overnight under N₂. Then the reaction mixture was poured into water (100 mL), extracted with EtOAc (150 mL×2) and the organic phase was washed by brine, dried with Na₂SO₄ and concentrated under reduced pressure and the residue was purified by column chromatography to give the title compound (9.05 g, yield 52%) as white solid.

### Example 117

### 2-(3-(4-((1H-indazol-5-yl)amino)-6-chloropyrimidin-2-yl)phenoxy)-N-isopropylacetamide

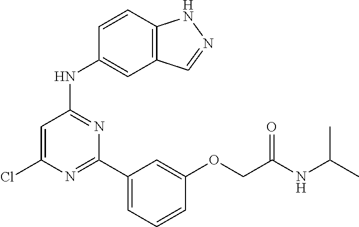 [SMILES: CC(C)NC(=O)COc1cccc(-c2nc(Cl)cc(Nc3ccc4[nH]ncc4c3)n2)c1]

To a solution of compound 2-(3-(4,6-dichloropyrimidin-2-yl)phenoxy)-N-isopropylacetamide (5.7 g, 16.75 mmol) in iPrOH (110 mL), DIPEA (6.5 g, 48.82 mmol) and 1H-indazol-5-amine (2.23 g, 17.25 mmol) were added and the reaction mixture was heated to reflux overnight. The reaction mixture was concentrated under reduced pressure and the residue was purified by column chromatography to give the title compound (3.22 g, yield 44%). ¹H NMR (400 MHz, DMSO-d₆) δ 13.05 (s, 1H), 9.90 (s, H), 8.09 (b, 2H), 7.95-7.89 (m, 3H), 7.59-7.41 (m, 3H), 7.12 (d, J=8.0 Hz, 1H), 6.66 (s, 1H), 4.51 (s, 2H), 4.00-3.94 (m, 1H), 1.08 (d, J=6.4 Hz, 6H); m/e 437 (M+H)⁺.

### Example 118

### 2-(3-(4-((1H-indazol-5-yl)amino)-6-(piperazin-1-yl)pyrimidin-2-yl)phenoxy)-N-isopropylacetamide

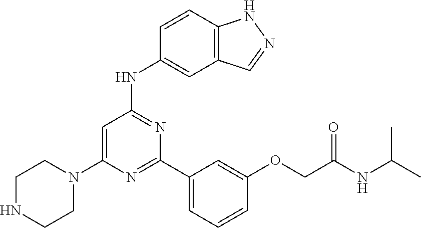 [SMILES: CC(C)NC(=O)COc1cccc(-c2nc(Nc3ccc4[nH]ncc4c3)cc(N3CCNCC3)n2)c1]

To a stirred solution of 2-(3-(4-((1H-indazol-5-yl)amino)-6-chloropyrimidin-2-yl)phenoxy)-N-isopropylacetamide (300 mg, 0.687 mmol) in iprOH (20 mL), Et₃N (3 mL), and piperazine (592 mg, 6.87 mmol) were added at room temperature. The mixture was stirred overnight at 110° C. Then reaction mixture was concentrated under reduced pressure and the residue was purified by pre-HPLC to provide the title compound (114 mg, yield 34%). ¹H NMR (400 MHz, DMSO-d₆) δ 12.91 (s, 1H), 9.01 (s, 1H), 8.08 (s, 1H), 8.02 (s, 1H), 7.94-7.89 (m, 3H), 7.51-7.35 (m, 3H), 7.04 (dd, J=8.0 and 2.0 Hz, 1H), 5.82 (s, 1H), 4.50 (s, 2H), 3.98-3.92 (m, 1H), 3.48 (b, 4H), 2.76 (b, 4H), 1.07 (d, J=6.8 Hz, 6H); m/e 487 (M+H)⁺.

### Example 119

### 2-(3-(4-((1H-indazol-5-yl)amino)-6-morpholinopyrimidin-2-yl)phenoxy)-N-isopropylacetamide

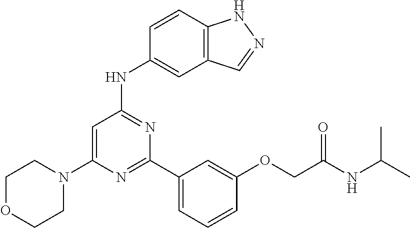 [SMILES: CC(C)NC(=O)COc1cccc(-c2nc(Nc3ccc4[nH]ncc4c3)cc(N3CCOCC3)n2)c1]

The title compound was synthesized using the same procedure as that for 2-(3-(4-((1H-indazol-5-yl)amino)-6-(piperazin-1-yl)pyrimidin-2-yl)phenoxy)-N-isopropylacetamide (example 118) (112 mg, yield 34%). ¹H NMR (400 MHz, DMSO-d₆) δ 12.93 (s, 1H), 9.09 (s, 1H), 8.08 (s, 1H), 8.03 (s, 1H), 7.95-7.91 (m, 3H), 7.49-7.38 (m, 3H), 7.05 (d, J=8.0 Hz, 1H), 5.84 (s, 1H), 4.50 (s, 2H), 3.94 (b, 1H), 3.69 (s, 4H), 3.52 (s, 4H), 1.06 (d, J=6.8 Hz, 6H); m/e 488 (M+H)⁺.

### Example 120

### 2-(3-(4-((1H-indazol-5-yl)amino)-6-(4-methyl-1,4-diazepan-1-yl)pyrimidin-2-yl)phenoxy)-N-isopropylacetamide

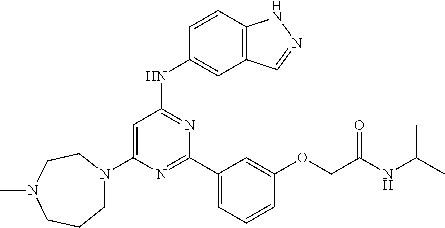 [SMILES: CC(C)NC(=O)COc1cccc(-c2nc(Nc3ccc4[nH]ncc4c3)cc(N3CCCN(C)CC3)n2)c1]

The title compound was synthesized using the same procedure as that for 2-(3-(4-((1H-indazol-5-yl)amino)-6-(piperazin-1-yl)pyrimidin-2-yl)phenoxy)-N-isopropylacetamide (example 118) (100 mg, yield 65%). ¹H NMR (400 MHz, DMSO-d₆) δ 12.94 (s, 1H), 9.00 (s, 1H), 8.13 (s, 1H), 8.05 (s, 1H), 7.97-7.91 (m, 3H), 7.53-7.05 (m, 4H), 5.75 (s, 1H), 4.53 (s, 2H), 4.02-3.62 (m, 5H), 2.64 (b, 2H), 2.50 (b, 2H), 2.26 (s, 3H), 1.92 (b, 2H), 1.09 (d, J=6.8 Hz, 6H); m/e 515 (M+H)⁺.

### Example 121

### 2-(3-(4-((1H-indazol-5-yl)amino)-6-(1,4-diazepan-1-yl)pyrimidin-2-yl)phenoxy)-N-isopropylacetamide

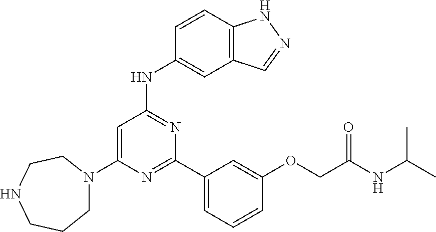 [SMILES: CC(C)NC(=O)COc1cccc(-c2nc(Nc3ccc4[nH]ncc4c3)cc(N3CCCNCC3)n2)c1]

The title compound was synthesized using the same procedure as that for 2-(3-(4-((1H-indazol-5-yl)amino)-6-(piperazin-1-yl)pyrimidin-2-yl)phenoxy)-N-isopropylacetamide (example 118) (110 mg, yield 32%). ¹H NMR (400 MHz, DMSO-d₆) δ 12.90 (s, 1H), 8.96 (s, 1H), 8.11 (s, 1H), 8.02 (s, 1H), 7.92-7.03 (m, 5H), 5.74 (s, 1H), 4.50 (s, 2H), 3.96-3.68 (m, 3H), 2.85 (b, 2H), 2.66 (b, 2H), 2.31 (b, 2H), 1.77 (b, 2H), 1.07 (d, J=6.8 Hz, 6H); m/e 501 (M+H)⁺.

### Example 122

### 2-(3-(4-((1H-indazol-5-yl)amino)-6-(dimethylamino)pyrimidin-2-yl)phenoxy)-N-isopropylacetamide

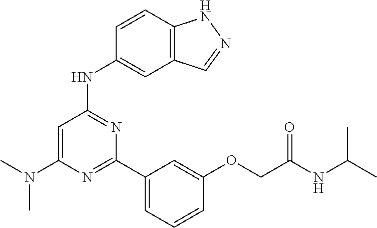 [SMILES: CC(C)NC(=O)COc1cccc(-c2nc(Nc3ccc4[nH]ncc4c3)cc(N(C)C)n2)c1]

The title compound was synthesized using the same procedure as that for 2-(3-(4-((1H-indazol-5-yl)amino)-6-(piperazin-1-yl)pyrimidin-2-yl)phenoxy)-N-isopropylacetamide (example 118) (101 mg, yield 33%). ¹H NMR (400 MHz, DMSO-d₆) δ 12.90 (s, 1H), 9.00 (s, 1H), 8.10 (s, 1H), 8.02 (s, 1H), 7.97-7.88 (m, 3H), 7.48 (s, 2H), 7.38 (t, J=8.0 Hz, 1H), 7.03 (d, J=7.2 Hz, 1H), 5.73 (s, 1H), 4.50 (s, 2H), 3.98-3.93 (m, 1H), 3.07 (s, 6H), 1.08 (d, J=6.8 Hz, 6H); m/e 446 (M+H)⁺.

### Example 123

### 2-(3-(4-((1H-indazol-5-yl)amino)-6-(piperidin-1-yl)pyrimidin-2-yl)phenoxy)-N-isopropylacetamide

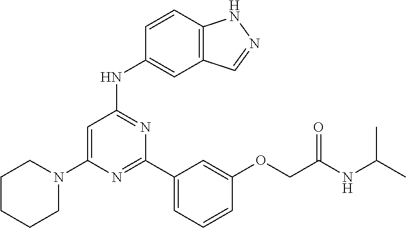 [SMILES: CC(C)NC(=O)COc1cccc(-c2nc(Nc3ccc4[nH]ncc4c3)cc(N3CCCCC3)n2)c1]

The title compound was synthesized using the same procedure as that for 2-(3-(4-((1H-indazol-5-yl)amino)-6-(piperazin-1-yl)pyrimidin-2-yl)phenoxy)-N-isopropylacetamide (example 118) (110 mg, yield 33%). ¹H NMR (400 MHz, DMSO-d₆) δ 12.91 (s, 1H), 8.98 (s, 1H), 8.09 (s, 1H), 8.02 (s, 1H), 7.94-7.89 (m, 3H), 7.49-7.03 (m, 5H), 5.85 (s, 1H), 5.00 (s, 2H), 3.96-3.93 (m, 1H), 3.58 (b, 4H), 1.55 (b, 6H), 1.07 (d, J=6.8 Hz, 6H); m/e 486 (M+H)⁺.

### Example 124

### 2-(3-(4-((1H-indazol-5-yl)amino)-6-((2-methoxyethyl)(methyl)amino)pyrimidin-2-yl)phenoxy)-N-isopropylacetamide

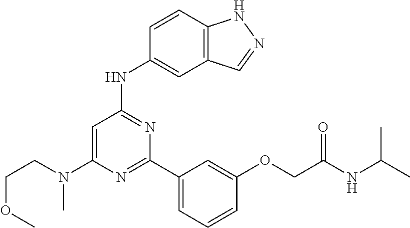 [SMILES: COCCN(C)c1cc(Nc2ccc3[nH]ncc3c2)nc(-c2cccc(OCC(=O)NC(C)C)c2)n1]

The title compound was synthesized using the same procedure as that for 2-(3-(4-((1H-indazol-5-yl)amino)-6-(piperazin-1-yl)pyrimidin-2-yl)phenoxy)-N-isopropylacetamide (example 118) (110 mg, yield 33%). ¹H NMR (400 MHz, DMSO-d₆) δ 13.00 (s, 1H), 9.07 (s, 1H), 8.15 (s, 1H), 8.08 (s, 1H), 8.05-7.92 (m, 3H), 7.53 (s, 1H), 7.42 (t, J=8.0 Hz, 1H), 7.07 (d, J=7.6 Hz, 1H), 5.79 (s, 1H), 4.53 (s, 2H), 4.03-3.95 (m, 1H), 2.78 (b, 2H), 3.59-3.56 (m, 2H), 3.29 (s, 3H), 3.07 (s, 3H), 1.10 (d, J=6.8 Hz, 1H); m/e 490 (M+H)⁺.

### Example 125

### 2-(3-(4-((1H-indazol-5-yl)amino)-6-((2-(dimethylamino)ethyl)(methyl)amino)pyrimidin-2-yl)phenoxy)-N-isopropylacetamide

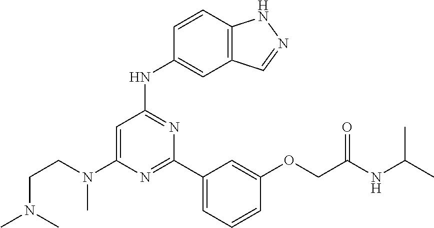 [SMILES: CC(C)NC(=O)COc1cccc(-c2nc(Nc3ccc4[nH]ncc4c3)cc(N(C)CCN(C)C)n2)c1]

The title compound was synthesized using the same procedure as that for 2-(3-(4-((1H-indazol-5-yl)amino)-6-(piperazin-1-yl)pyrimidin-2-yl)phenoxy)-N-isopropylacetamide (example 118) (100 mg, yield 29%). ¹H NMR (400 MHz, DMSO-d₆) δ 12.91 (s, 1H), 8.99 (s, 1H), 8.09 (s, 1H), 8.02 (s, 1H), 7.96-7.86 (m, 3H), 7.49-7.03 (m, 4H), 5.71 (s, 1H), 4.46 (s, 2H), 4.00-3.92 (m, 1H), 3.67 (b, 2H), 3.30 (b, 2H), 3.01 (s, 3H), 2.15 (s, 6H), 1.07 (d, J=6.8 Hz, 6H); m/e 503 (M+H)⁺.

### Example 126

### 2-(3-(4-chloro-6-(2-(dimethylamino)ethoxy)pyrimidin-2-yl)phenoxy)-N-isopropylacetamide

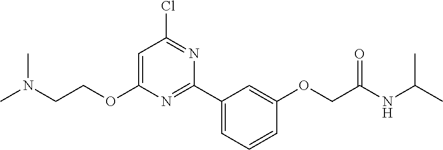 [SMILES: CC(C)NC(=O)COc1cccc(-c2nc(Cl)cc(OCCN(C)C)n2)c1]

To a solution of compound 2-(3-(4,6-dichloropyrimidin-2-yl)phenoxy)-N-isopropylacetamide (1 g, 2.9 mmol) in toluene (24 mL) were added NaOH (232 mg, 5.8 mmol) and 2-(dimthylamino)ethanol (261 mg, 2.9 mmol). The resulting mixture was stirred for 3 hrs at 110° C. Then the reaction mixture was diluted with water and extracted with DCM. The organic layer was dried over Na₂SO₄, filtered and concentrated under reduced pressure to give a residue which was purified by column chromatograph on silica gel (eluted with DCM:MeOH=100:1) to give compound the title compound (550 mg, yield 48%) as a solid.

### Example 127

### 2-(3-(4-((1H-indazol-5-yl)amino)-6-(2-(dimethylamino)ethoxy)pyrimidin-2-yl)phenoxy)-N-isopropylacetamide

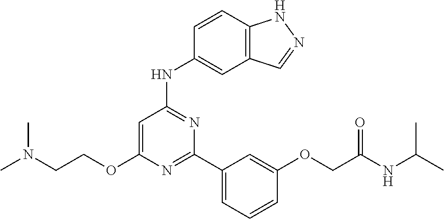 [SMILES: CC(C)NC(=O)COc1cccc(-c2nc(Nc3ccc4[nH]ncc4c3)cc(OCCN(C)C)n2)c1]

To a solution of compound 2-(3-(4-chloro-6-(2-(dimethylamino)ethoxy)pyrimidin-2-yl)phenoxy)-N-isopropylacetamide (100 mg, 0.25 mmol) in EtOH (1 mL) were added 1H-indozal-5-amine (101.5 mg, 0.76 mmol) and TFA (0.25 mL). The resulting mixture was heated to 80° C. overnight. The mixture was concentrated and purified by chromatography on silica gel column and purified by Prep-TLC again to give the title compound (100 mg, yield 16%) as a light yellow solid. ¹H NMR (400 MHz, MeOD) δ 8.05-7.96 (m, 4H), 7.54-7.49 (m, 2H), 7.40-7.08 (m, 2H), 5.94 (s, 1H), 4.55 (s, 4H), 4.14-4.06 (m, 1H), 2.79-2.76 (m, 2H), 2.33 (s, 6H), 1.16 (d, J=6.8 Hz, 6H); m/e 490 (M+H)⁺.

### Example 128

### 3-(4,4,5,5-tetramethyl-1,3,2-dioxaborolan-2-yl)phenyl morpholine-4-carboxylate

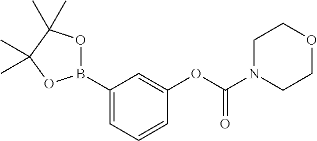 [SMILES: CC1(C)OB(c2cccc(OC(=O)N3CCOCC3)c2)OC1(C)C]

To a mixture of 3-(4,4,5,5-tetramethyl-1,3,2-dioxaborolan-2-yl)phenol (500 mg, 2.27 mmol), DMAP (277.3 mg) and Et₃N (450.46 mg, 4.54, mmol) in DCM (10 ml) was added dropwise a solution of morpholine-4-carbonyl chloride (339.5 mg, 2.27 mmol) in DCM (10 ml) at 0° C. Water was added to the mixture and extracted with DCM (40 mL×2). The organic phase was dried with Na₂SO₄ and concentrated under reduced pressure to give the title compound (600 mg, yield 79%) which was used to next step directly.

### Example 129

### 3-(4-((1H-indazol-5-yl)amino)pyrimidin-2-yl)phenyl morpholine-4-carboxylate

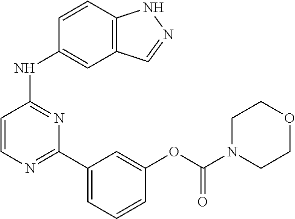 [SMILES: O=C(Oc1cccc(-c2nccc(Nc3ccc4[nH]ncc4c3)n2)c1)N1CCOCC1]

To a stirred solution of tert-butyl 5-((tert-butoxycarbonyl)(2-chloropyrimidin-4-yl)amino)-1H-indazole-1-carboxylate (100 mg, 0.2243 mmol) in EtOH (3 mL) and H₂O (0.3 ml) were added Na₂CO₃ (47.54 mg, 0.4485 mmol), (Boc)₂O (93.29 mg, 0.4485 mmol) and 3-(4,4,5,5-tetramethyl-1,3,2-dioxaborolan-2-yl)phenyl morpholine-4-carboxylate (149.44 mg, 0.4485 mmol) at room temperature. The mixture was degassed by budding nitrogen through the solution Pd(PPh₃)₂Cl₂ (15.07 mg, 0.02243 mmol) was added and the mixture was heated under microwave irradiation for 20 minutes at 110° C. The mixture was dried and concentrated under reduced pressure and the residue was purified by column chromatograph on silica gel (DCM:MeOH=50:1) to give the title compound (50 mg, yield 53%). ¹H NMR (400 MHz, DMSO-d₆) δ 13.02 (s, 1H), 9.66 (s, 1H), 8.33-8.18 (m, 4H), 7.55-7.51 (m, 3H), 7.25 (d, J=8.0 Hz, 1H), 6.67 (d, J=5.6 Hz, 1H), 3.64 (b, 6H), 3.45 (b, 2H); m/e 417 (M+H)⁺.

### Example 130

### 3-(4,4,5,5-tetramethyl-1,3,2-dioxaborolan-2-yl)phenyl dimethylcarbamate

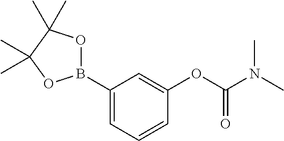

To a mixture of 3-(4,4,5,5-tetramethyl-1,3,2-dioxaborolan-2-yl)phenol (500 mg, 2.23 mmol), DMAP (277.4 mg) and Et₃N (450.46 mg, 4.46, mmol) in DCM (15 mL) was added dropwise a solution of dimethylcarbamic chloride (238.6 mg, 2.23 mmol) in DCM (15 ml) at 0° C. and the reaction mixture was stirred overnight at room temperature. Water was added to the mixture and extracted with DCM (40 mL×2). The organic phase was dried with Na₂SO₄ and concentrated under reduced pressure to give the title compound (500 mg, yield 76%) which was used to next step directly.

### Example 131

### 3-(4-((1H-indazol-5-yl)amino)pyrimidin-2-yl)phenyl dimethylcarbamate

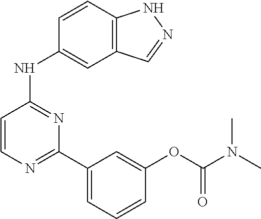 [SMILES: CN(C)C(=O)Oc1cccc(-c2nccc(Nc3ccc4[nH]ncc4c3)n2)c1]

To a stirred solution of tert-butyl 5-((tert-butoxycarbonyl)(2-chloropyrimidin-4-yl)amino)-1H-indazole-1-carboxylate (100 mg, 0.2243 mmol) in EtOH (3 mL) and H₂O (0.3 ml) was added Na₂CO₃ (47.54 mg, 0.4485 mmol), (Boc)₂O (93.29 mg, 0.4485 mmol) and 3-(4,4,5,5-tetramethyl-1,3,2-dioxaborolan-2-yl)phenyl dimethylcarbamate (130.58 mg, 0.4485 mmol) at room temperature. The mixture was degassed by budding nitrogen through the solution, Pd(PPh₃)₂Cl₂ (15.07 mg, 0.02243 mmol) was added and the mixture was heated under microwave irradiation for 20 min at 110° C. The mixture was concentrated under reduced pressure and the residue was purified by column chromatograph on silica gel (DCM:MeOH=50:1) to give the title compound (30 mg, yield 37%). ¹H NMR (400 MHz, DMSO-d₆) δ 13.02 (s, 1H), 9.66 (s, 1H), 8.34-8.04 (m, 5H), 7.55-7.47 (m, 3H), 7.21 (d, J=8.0 Hz, 1H), 6.67 (d, J=5.6 Hz, 1H), 3.07 (s, 3H), 2.94 (s, 3H); m/e 375 (M+H)⁺.

### Example 132

### 3-((3-(4,4,5,5-tetramethyl-1,3,2-dioxaborolan-2-yl)phenoxy)methyl)pyridine

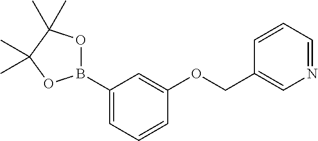 [SMILES: CC1(C)OB(c2cccc(OCc3cccnc3)c2)OC1(C)C]

To a solution of compound 3-(4,4,5,5-tetramethyl-1,3,2-dioxaborolan-2-yl)phenol (1.5 g, 6.8 mmol) in DMF (20 mL) was added NaH (0.82 g, 20.4 mmol) potionwise at 0° C. with stirring. After 30 minutes, compound 3-(chloromethyl)pyridine hydrochloride (1.4 g, 8.9 mmol) was added portionwise at 0° C., and the resulting mixture was allowed to warm to 20° C. and stirred for 16 hrs. It was quenched with water, extracted with EtOAc (100 mL×3), and the extracts were washed with brine, dried over Na₂SO₄, concentrated under reduced pressure, and the residue was purified by chromatography on silica gel column (eluted with PE:EA=10:1 to 2:1) to give the title compound (1 g, yield 50%) as a white solid.

### Example 133

### tert-butyl 1H-indazol-5-yl(2-(3-(pyridin-3-ylmethoxy)phenyl)pyrimidin-4-yl)carbamate

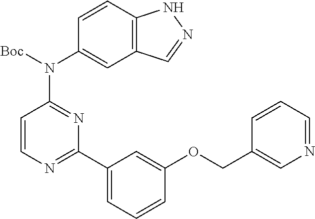 [SMILES: CC(C)(C)OC(=O)N(c1ccc2[nH]ncc2c1)c1ccnc(-c2cccc(OCc3cccnc3)c2)n1]

A mixture of compound 3-((3-(4,4,5,5-tetramethyl-1,3,2-dioxaborolan-2-yl)phenoxy)methyl)pyridine (1 g, 3.2 mmol), compound tert-butyl 5-((tert-butoxycarbonyl)(2-chloropyrimidin-4-yl)amino)-1H-indazole-1-carboxylate (670 mg, 1.5 mmol), K₂CO₃ (414 mg, 3 mmol) and Pd(dppf)₂Cl₂ (109 mg, 0.15 mmol) in dioxane (20 mL) and H₂O (5 mL) was heated at 90° C. for 16 hrs under N₂ atmosphere. Then it was concentrated and the residue was purified by chromatography on silica gel column (eluted with DCM:MeOH=100:1 to 20:1) to give the title compound (370 mg, yield 50%) as a light red oil.

### Example 134

### tert-butyl 1H-indazol-5-yl(2-(3-(pyridin-3-ylmethoxy)phenyl)pyrimidin-4-yl)carbamate

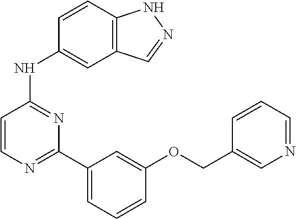 [SMILES: c1cncc(COc2cccc(-c3nccc(Nc4ccc5[nH]ncc5c4)n3)c2)c1]

(370 mg, 0.75 mmol) in DCM (5 mL) was added TFA (5 mL), and the resulting solution was stirred at 20° C. for 3 hrs. It was concentrated and the residue was dissolved in DCM/MeOH (10:1, 100 mL), washed with aqueous K₂CO₃ and brine, dried over Na₂SO₄, concentrated and the residue was purified by chromatography on silica gel column (eluted with DCM:MeOH=100:1 to 20:1) and recrystallized from MeOH to afford the title compound (110 mg, yield 37%) as a light yellow solid. ¹H NMR (400 MHz, DMSO-d₆) δ 13.04 (s, 1H), 9.67 (s, 1H), 8.71 (s, 1H), 8.57 (s, 1H), 8.35 (d, J=5.6 Hz, 1H), 8.05 (s, 1H), 8.00-7.89 (m, 4H), 7.56-6.68 (m, 6H), 5.25 (s, 2H).

13.02 (s, 1H), 9.66 (s, 1H), 8.33-8.18 (m, 4H), 7.55-7.51 (m, 3H), 7.25 (d, J=8.0 Hz, 1H), 6.67 (d, J=5.6 Hz, 1H), 3.64 (b, 6H), 3.45 (b, 2H); m/e 417 (M+H)⁺.

### Example 135

### 4-((3-(4,4,5,5-tetramethyl-1,3,2-dioxaborolan-2-yl)phenoxy)methyl)pyridine

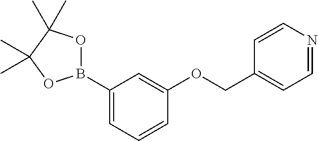 [SMILES: CC1(C)OB(c2cccc(OCc3ccncc3)c2)OC1(C)C]

To a solution of compound 3-(4,4,5,5-tetramethyl-1,3,2-dioxaborolan-2-yl)phenol (1.5 g, 6.8 mmol) in DMF (20 mL) was added NaH (0.82 g, 20.4 mmol) potionwise at 0° C. with stirring. After 30 minutes, compound 4-(chloromethyl)pyridine (1.4 g, 8.9 mmol) was added portionwise at 0° C., and the resulting mixture was allowed to warm to 20° C. and stirred for 16 hrs. It was quenched with water, extracted with EtOAc (100 mL×3), and the extracts were washed with brine, dried over Na₂SO₄, concentrated, and the residue was purified by chromatography on silica gel column (eluted with PE:EA=10:1 to 2:1) to give the title compound (1 g, yield 50%) as a white solid.

### Example 136

### N-(2-(3-(pyridin-4-ylmethoxy)phenyl)pyrimidin-4-yl)-1H-indazol-5-amine

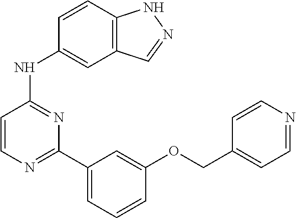 [SMILES: c1cc(OCc2ccncc2)cc(-c2nccc(Nc3ccc4[nH]ncc4c3)n2)c1]

A mixture of compound 4-((3-(4,4,5,5-tetramethyl-1,3,2-dioxaborolan-2-yl)phenoxy)methyl)pyridine (139 mg, 0.44 mmol) (6 batches), compound tert-butyl 5-((tert-butoxycarbonyl)(2-chloropyrimidin-4-yl)amino)-1H-indazole-1-carboxylate (100 mg, 0.22 mmol), Na₂CO₃ (47 mg, 0.44 mmol), Boc₂O (96 mg, 0.44 mmol) and Pd(PPh₃)₂Cl₂ (15.4 mg, 0.022 mmol) in EtOH (2 mL) and H₂O (0.2 mL) was heated in microwave reactor at 110° C. for 20 minutes under N₂ atmosphere. Then it was cooled, diluted with water, extracted with DCM, the extracts were concentrated and the residue was purified by chromatography on silica gel column (eluted with DCM:MeOH=100:1 to 20:1) and recrystallized from MeOH to afford the title compound (110 mg, yield 23%) as a light yellow solid. ¹H NMR (400 MHz, MeOD) δ 8.51 (d, J=5.6 Hz, 2H), 8.25 (d, J=6.0 Hz, 1H), 8.15 (s, 1H), 8.05-7.94 (m, 3H), 7.57-7.16 (m, 6H), 6.65 (d, J=6.0 Hz, 1H), 5.26 (s, 2H)); m/e 395 (M+H)⁺.

### Example 137

### 2-(3-(2-methoxyethoxyl)phenyl)-4,4,5,5-tetramethyl-1,3,2-dioxaborolane

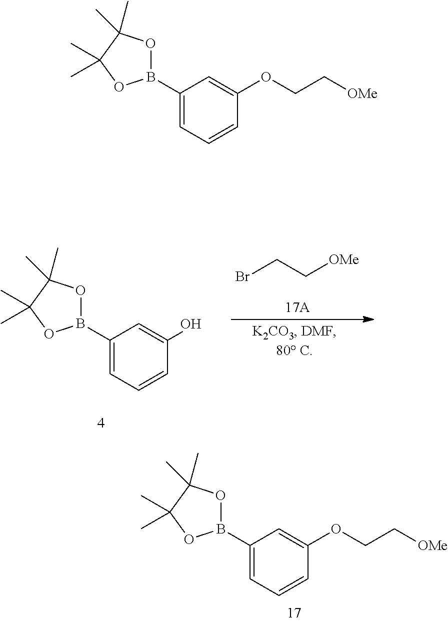 [SMILES: COCCOc1cccc(B2OC(C)(C)C(C)(C)O2)c1 ; CC1(C)OB(c2cccc(O)c2)OC1(C)C ; COCCBr ; COCCOc1cccc(B2OC(C)(C)C(C)(C)O2)c1]

To a mixture of 3-(4,4,5,5-tetramethyl-1,3,2-dioxaborolan-2-yl)phenol (800 mg, 3.636 mmol) in DCM (20 ml) were added KI (1.2 g, 7.273 mmol), K₂CO₃ (1.307 g 10.091 mmol) and 1-bromo-2-methoxyethane (1.01 g, 7.273 mmol) at room temperature. The mixture was stirred overnight at 80° C. The mixture was extracted with DCM (30 mL×2) and the organic phase was dried with Na₂SO₄ and concentrated under reduced pressure and the residue was purified by column chromatography to give the title compound (550 mg, yield 50%).

### Example 138

### N-(2-(3-(2-methoxyethoxyl)phenyl)pyrimidin-4-yl)-1H-indazol-5-amine

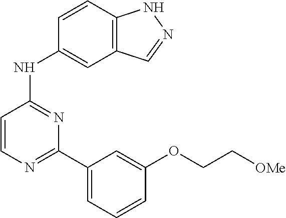 [SMILES: COCCOc1cccc(-c2nccc(Nc3ccc4[nH]ncc4c3)n2)c1]

To a stirred solution of tert-butyl 5-((tert-butoxycarbonyl)(2-chloropyrimidin-4-yl)amino)-1H-indazole-1-carboxylate (100 mg, 0.2243 mmol) in EtOH (3 mL) and H₂O (0.3 ml) was added Na₂CO₃ (47.54 mg, 0.4485 mmol), (Boc)₂O (93.29 mg, 0.4485 mmol) and 2-(3-(2-methoxyethoxyl)phenyl)-4,4,5,5-tetramethyl-1,3,2-dioxaborolane (124.76 mg, 0.4485 mmol) at room temperature. The mixture was degassed by budding nitrogen through the solution, then Pd(PPh₃)₂Cl₂ (15.07 mg, 0.02243 mmol) was added and the mixture was heated under microwave irradiation for 20 minutes at 110° C. The mixture was concentrated under reduced pressure and the residue was purified by column chromatograph on silica gel (DCM:MeOH=50:1) to give the title compound (40 mg, yield 49%). ¹H NMR (400 MHz, DMSO-d₆) δ 13.01 (s, 1H), 9.61 (s, 1H), 8.32 (d, J=5.6 Hz, 1H), 8.21 (s, 1H), 8.01-7.91 (m, 3H), 7.55-7.36 (m, 3H), 7.04 (d, J=7.6 Hz, 1H), 6.65 (d, J=5.6 Hz, 1H), 4.16 (b, 2H), 3.68 (b, 2H); m/e 362 (M+H)⁺.

### Example 139

### tert-butyl 1H-indazol-5-yl(2-(3-methoxyphenyl)pyrimidin-4-yl)carbamate

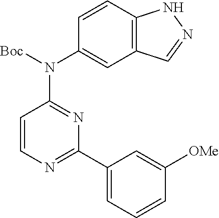 [SMILES: COc1cccc(-c2nccc(N(C(=O)OC(C)(C)C)c3ccc4[nH]ncc4c3)n2)c1]

To a stirred solution of tert-butyl 5-((tert-butoxycarbonyl)(2-chloropyrimidin-4-yl)amino)-1H-indazole-1-carboxylate (100 mg, 0.2243 mmol) in EtOH (3 mL) and H₂O (0.3 ml) were added Na₂CO₃ (47.54 mg, 0.4485 mmol), (Boc)₂O (93.29 mg, 0.4485 mmol) and (3-methoxyphenyl)boronic acid (68.17 mg, 0.4485 mmol) at room temperature. The mixture was degassed by budding nitrogen through the solution, then Pd(PPh₃)₂Cl₂ (15.07 mg, 0.02243 mmol) was added and the mixture was heated under microwave irradiation for 20 min at 110° C. The mixture was concentrated under reduced pressure and the residue was purified by column chromatograph on silica gel to give the title compound (80 mg, yield 85%).

### Example 140

### N-(2-(3-methoxyphenyl)pyrimidin-4-yl)-1H-indazol-5-amine

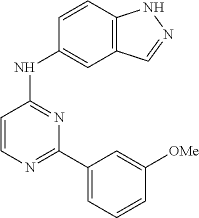 [SMILES: COc1cccc(-c2nccc(Nc3ccc4[nH]ncc4c3)n2)c1]

To a stirred solution of tert-butyl 1H-indazol-5-yl(2-(3-methoxyphenyl)pyrimidin-4-yl)carbamate (1 g, 2.39 mmol) in DCM (20 mL) was added TFA (10 ml) at room temperature and the mixture was stirred overnight at room temperature. Then the mixture was concentrated under reduced pressure and the residue was purified by pre_HPLC to give the title compound (200 mg, yield 26%) as white solid. ¹H NMR (400 MHz, DMSO-d₆) δ 13.05 (s, 1H), 10.02 (s, 1H), 8.32 (d, J=6.0 Hz, 1H), 8.17 (s, 1H), 8.05 (s, 1H), 7.89-7.42 (m, 3H), 7.09 (d, J=7.6 Hz, 1H), 6.72 (d, J=6.0 Hz, 1H), 3.83 (s, 3H); m/e 318 (M+H)⁺.

### Example 141

### 4-(2-(3-bromophenoxyl)ethyl)morpholine

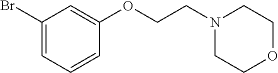 [SMILES: Brc1cccc(OCCN2CCOCC2)c1]

To a solution of 3-bromophenol (7.8 g, 45.2 mmol) in DMF (120 mL) was added 4-(2-chloroethyl)morpholine (8.54 g, 45.2 mmol), K₂CO₃ (414 mg, 3 mmol) and KI (7.5 mg, 45.2 mmol), and the resulting mixture was heated at 20° C. and stirred for 16 hrs. It was quenched with water, extracted with EtOAc (200 mL×3), and the extracts were washed with brine, dried over Na₂SO₄, concentrated, and the residue was purified by chromatography on silica gel column (eluted with DCM:MeOH=100:1 to 20:1) to give compound the title compound (7 g, yield 54%) as a red liquid.

### Example 142

### 4-(2-(3-(4,4,5,5-tetramethyl-1,3,2-dioxaborolan-2-yl)phenoxy)ethyl)morpholine

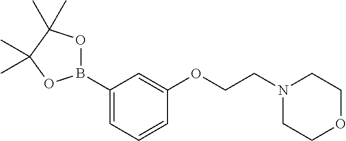 [SMILES: CC1(C)OB(c2cccc(OCCN3CCOCC3)c2)OC1(C)C]

A mixture of compound 4-(2-(3-bromophenoxyl)ethyl)morpholine (1.1 g, 3.8 mmol), BIPN (1.47 mg, 5.8 mmol), KOAc (0.83 mg, 8.5 mmol) and Pd(dppf)₂Cl₂ (0.28 mg, 0.38 mmol) in dioxane (15 mL) was heated at 90° C. for 16 hrs under N₂ atmosphere. Then it was concentrated and the residue was purified by chromatography on silica gel column (eluted with DCM:MeOH=100:1 to 20:1) to give compound the title compound (1 g, yield 78%) as a light red oil.

### Example 143

### tert-butyl 1H-indazol-5-yl(2-(3-(2-morpholinoethoxyl)phenyl)pyrimidin-4-yl)carbamate

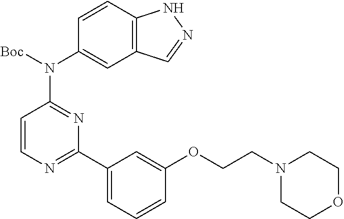 [SMILES: CC(C)(C)OC(=O)N(c1ccc2[nH]ncc2c1)c1ccnc(-c2cccc(OCCN3CCOCC3)c2)n1]

A mixture of compound 4-(2-(3-(4,4,5,5-tetramethyl-1,3,2-dioxaborolan-2-yl)phenoxy)ethyl)morpholine (1 g, 3.2 mmol), compound tert-butyl 5-((tert-butoxycarbonyl)(2-chloropyrimidin-4-yl)amino)-1H-indazole-1-carboxylate (670 mg, 1.5 mmol), K₂CO₃ (414 mg, 3 mmol) and Pd(dppf)₂Cl₂ (109 mg, 0.15 mmol) in dioxane (20 mL) and H₂O (5 mL) was heated at 90° C. for 16 hrs under N₂ atmosphere. Then it was concentrated and the residue was purified by chromatography on silica gel column (eluted with DCM:MeOH=100:1 to 20:1) to give the tile compound (380 mg, yield 50%) as a light red oil.

### Example 144

### N-(2-(3-(2-morpholinoethoxyl)phenyl)pyrimidin-4-yl)-1H-indazol-5-amine

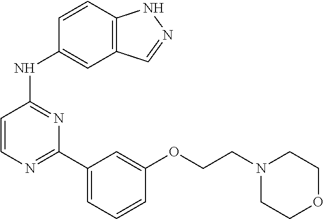 [SMILES: c1cc(OCCN2CCOCC2)cc(-c2nccc(Nc3ccc4[nH]ncc4c3)n2)c1]

To a solution of compound tert-butyl 1H-indazol-5-yl(2-(3-(2-morpholinoethoxyl)phenyl)pyrimidin-4-yl)carbamate (380 mg, 0.75 mmol) in DCM (5 mL) was added TFA (5 mL), and the resulting solution was stirred at 20° C. for 3 hrs. It was concentrated and the residue was dissolved in DCM/MeOH (10:1, 100 mL), washed with aqueous K₂CO₃ and brine, dried over Na₂SO₄, concentrated and the residue was purified by chromatography on silica gel column (eluted with DCM:MeOH=80:1 to 15:1) and recrystallized from EtOAc and PE to afford the tile compound (120 mg, yield 39%) as a light yellow solid. ¹H NMR (400 MHz, DMSO-d₆) δ 13.04 (s, 1H), 9.65 (s, 1H), 8.36 (m, 3H), 8.08 (s, 1H), 7.94 (s, 1H), 7.57-7.38 (m, 3H), 7.06 (d, J=8.0 Hz, 1H), 6.68 (d, J=5.6 Hz, 1H), 4.17 (t, J=5.6 Hz, 2H), 3.58 (s, 4H), 2.73 (t, J=5.6 Hz, 2H), 2.67 (s, 4H); m/e 417 (M+H)⁺.

### Example 145

### tert-butyl 5-((2-(3-acetylphenyl)pyrimidin-4-yl)amino)-1H-indazole-1-carboxylate

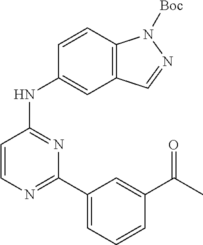 [SMILES: CC(=O)c1cccc(-c2nccc(Nc3ccc4c(cnn4C(=O)OC(C)(C)C)c3)n2)c1]

A mixture of tert-butyl 5-((tert-butoxycarbonyl)(2-chloropyrimidin-4-yl)amino)-1H-indazole-1-carboxylate (100 mg, 0.22 mmol), 3-acetylphenyl)boronic acid (73.7 mg, 0.44 mmol), Na₂CO₃ (47.6 mg, 0.44 mmol), (Boc)₂O (98 mg, 0.44 mmol), Pd(PPh₃)Cl₂ (16 mg, 0.022 mmol) in EtOH:H₂O (3.3 mL, 10:1) was heated under microwave irradiation for 20 min at 110° C. After reaction, it was evaporated, EA and water was added, separated the organic layer, washed with saturated brine, dried over Na₂SO₄, concentrated and purified by silica gel to give the title compound (64 mg, yield 67%).

### Example 146

### tert-butyl 5-((2-(3-(1-aminoethyl)phenyl)pyrimidin-4-yl)amino)-1H-indazole-1-carboxylate

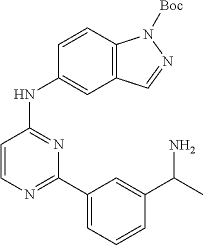 [SMILES: CC(N)c1cccc(-c2nccc(Nc3ccc4c(cnn4C(=O)OC(C)(C)C)c3)n2)c1]

To a stirred solution of tert-butyl 5-((2-(3-acetylphenyl)pyrimidin-4-yl)amino)-1H-indazole-1-carboxylate (500 mg, 1.2 mmol), AcONH₄ (924 mg, 12 mmol) in MeOH (10 mL) was added NaBH₃CN (91 mg, 1.44 mmol), the mixture was stirred 6 hrs at reflux. After reaction, it was evaporated, diluted with water, filtered to give the title compound as a white solid (300 mg, crude).

### Example 147

### N-(2-(3-(1-aminoethyl)phenyl)pyrimidin-4-yl)-1H-indazol-5-amine

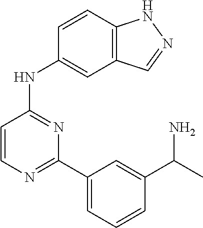 [SMILES: CC(N)c1cccc(-c2nccc(Nc3ccc4[nH]ncc4c3)n2)c1]

A solution of tert-butyl 5-((2-(3-(1-aminoethyl)phenyl)pyrimidin-4-yl)amino)-1H-indazole-1-carboxylate (300 mg, crude) in HCl/MeOH (20 mL) was stirred at 40° C. for 6 h. After reaction, it was evaporated, then water was added, adjusted the PH to 9 with saturated Na₂CO₃, filtered to give the crude product, which was purified by Pre-HPLC to provide the title compound (100 mg) as a white solid. ¹H NMR (400 MHz, DMSO-d₆) δ 13.03 (s, 1H), 9.65 (s, 1H), 8.44 (s, 1H), 8.35 (d, J=5.6 Hz, 2H), 8.18 (d, J=7.2 Hz, 1H), 8.05 (s, 1H), 7.55-7.40 (m, 4H), 6.67 (d, J=5.6 Hz, 1H), 4.12-4.07 (m, 1H), 2.08 (b, 2H), 1.31 (d, J=6.4 Hz, 3H); m/e 331 (M+H)⁺.

### Example 148

### tert-butyl 5-((2-(4-acetylphenyl)pyrimidin-4-yl)amino)-1H-indazole-1-carboxylate

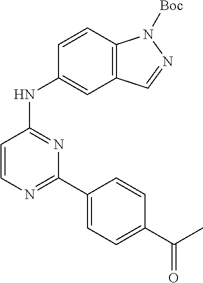 [SMILES: CC(=O)c1ccc(-c2nccc(Nc3ccc4c(cnn4C(=O)OC(C)(C)C)c3)n2)cc1]

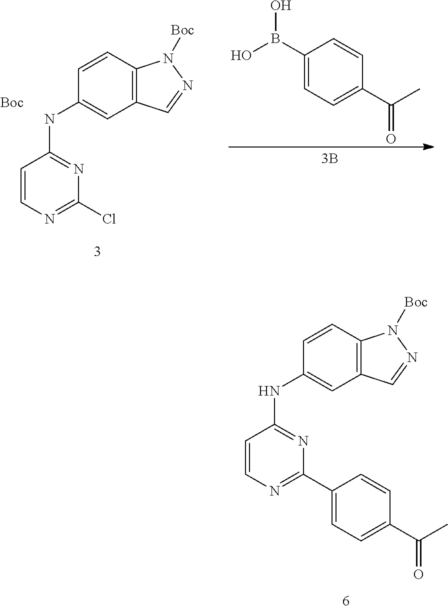 [SMILES: CC(C)(C)OC(=O)n1ncc2cc(Nc3ccnc(Cl)n3)ccc21 ; CC(=O)c1ccc(B(O)O)cc1 ; CC(=O)c1ccc(-c2nccc(Nc3ccc4c(cnn4C(=O)OC(C)(C)C)c3)n2)cc1 ; "Boc"]

A mixture of tert-butyl 5-((tert-butoxycarbonyl)(2-chloropyrimidin-4-yl)amino)-1H-indazole-1-carboxylate (1.5 g, crude), (4-acetylphenyl)boronic acid (1.12 g, 6.8 mmol), Na₂CO₃ (721 mg, 6.8 mmol), (Boc)₂O (1.48 g, 6.8 mmol), Pd(PPh₃)Cl₂ (239 mg, 0.34 mmol) in EtOH:H₂O (16.5 mL, 10:1) was heated under microwave irradiation for 20 min at 110° C. After reaction, it was evaporated, EA and water was added, separated the organic layer, washed with saturated brine, dried over Na₂SO₄, concentrated to give the title compound (2.6 g, crude).

### Example 149

### tert-butyl 5-((2-(4-(1-aminoethyl)phenyl)pyrimidin-4-yl)amino)-1H-indazole-1-carboxylate

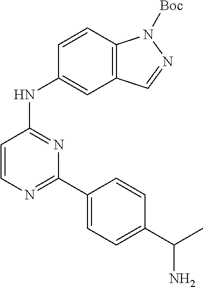 [SMILES: CC(N)c1ccc(-c2nccc(Nc3ccc4c(cnn4C(=O)OC(C)(C)C)c3)n2)cc1]

To a stirred solution of tert-butyl 5-((2-(4-acetylphenyl)pyrimidin-4-yl)amino)-1H-indazole-1-carboxylate (2.6 g, 6.1 mmol), AcONH₄ (4.7 g, 61 mmol) in MeOH (60 mL) was added NaBH₃CN (461 mg, 7.32 mmol), the mixture was stirred for 10 h at reflux. After reaction, the solvent was evaporated, then water was added, filtered to give the title compound (2.1 g, crude).

### Example 150

### N-(2-(4-(1-aminoethyl)phenyl)pyrimidin-4-yl)-1H-indazol-5-amine

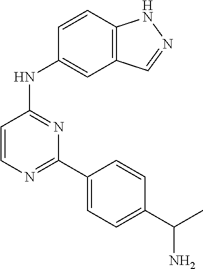 [SMILES: CC(N)c1ccc(-c2nccc(Nc3ccc4[nH]ncc4c3)n2)cc1]

A solution of tert-butyl 5-((2-(4-(1-aminoethyl)phenyl)pyrimidin-4-yl)amino)-1H-indazole-1-carboxylate (2.1 g, crude) in HCl/MeOH (60 mL) was stirred at 40° C. for 6 h. After reaction, the solvent was evaporated, then water was added, adjusted the pH to 9 with saturated Na₂CO₃, filtered to give the crude product, which was purified by Pre-HPLC to give the title compound (113.5 mg) as a white solid. ¹H NMR (400 MHz, DMSO-d₆) δ 13.02 (s, 1H), 9.58 (s, 1H), 8.34-8.27 (m, 4H), 8.20 (s, 1H), 8.08 (s, 1H), 7.56 (s, 2H), 7.49 (d, J=6.0 Hz, 1H), 4.05-4.03 (m, 1H), 1.27 (d, J=6.4 Hz, 3H); m/e 331 (M+H)⁺.

### Example 151

### 2-(3-(2,2-diethoxyethoxyl)phenyl)-4,4,5,5-tetramethyl-1,3,2-dioxaborolane

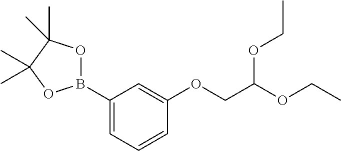 [SMILES: CCOC(COc1cccc(B2OC(C)(C)C(C)(C)O2)c1)OCC]

To a mixture of compound 3-(4,4,5,5-tetramethyl-1,3,2-dioxaborolan-2-yl)phenol (550 mg, 2.5 mmol), 2-bromo-1,1-diethoxyethane (985 mg, 5 mmol), Cs₂CO₃ (2.43 g, 7.5 mmol) in DMF (25 mL) was added KI (106 mg, 1 mmol), then the mixture was stirred overnight at 110° C. After reaction, water was added, then extracted with EA, washed with saturated brine, dried over Na₂SO₄, concentrated to give the title compound (360 mg, crude) as a light yellow oil.

### Example 152

### tert-butyl 5-((2-(3-(2,2-diethoxyethoxyl)phenyl)pyrimidin-4-yl)amino)-1H-indazole-1-carboxylate

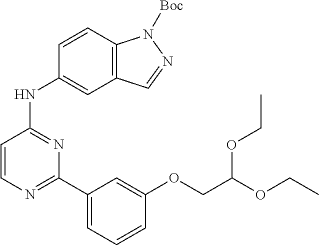 [SMILES: CCOC(COc1cccc(-c2nccc(Nc3ccc4c(cnn4C(=O)OC(C)(C)C)c3)n2)c1)OCC]

A mixture of 2-(3-(2,2-diethoxyethoxyl)phenyl)-4,4,5,5-tetramethyl-1,3,2-dioxaborolane (100 mg, crude), tert-butyl 5-((tert-butoxycarbonyl)(2-chloropyrimidin-4-yl)amino)-1H-indazole-1-carboxylate (70 mg, 0.15 mmol), Na₂CO₃ (65 mg, 0.6 mmol), (Boc)₂O (130 mg, 0.6 mmol), Pd(PPh₃)Cl₂ (20 mg, 0.03 mmol) in EtOH:H₂O (4.4 mL, 10:1) was heated under microwave irradiation for 20 min at 110° C. After reaction, it was evaporated, EA and water was added, separated the organic layer, washed with saturated brine, dried over Na₂SO₄, concentrated and purified by Pre-TLC to give the title compound (30 mg).

### Example 153

### 2-(3-(4-((1H-indazol-5-yl)amino)pyrimidin-2-yl)phenoxy)acetaldehyde

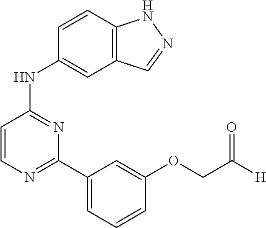 [SMILES: O=CCOc1cccc(-c2nccc(Nc3ccc4[nH]ncc4c3)n2)c1]

To a solution of tert-butyl 5-((2-(3-(2,2-diethoxyethoxyl)phenyl)pyrimidin-4-yl)amino)-1H-indazole-1-carboxylate (510 mg, 0.98 mmol) in THF (20 mL) was added dropwise 3M HCl (10 mL), then the mixture was stirred at reflux for 7 h. After reaction, it was evaporated, then water was added, adjusted the pH to 9 with saturated Na₂CO₃, filtered to give the title compound (420 mg, crude).

### Example 154

### N-(2-(3-(2-(isopropylamino)ethoxy)phenyl)pyrimidin-4-yl)-1H-indazol-5-amine

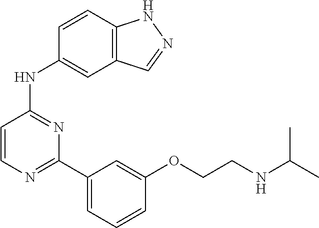 [SMILES: CC(C)NCCOc1cccc(-c2nccc(Nc3ccc4[nH]ncc4c3)n2)c1]

To a stirred solution of 2-(3-(4-((1H-indazol-5-yl)amino)pyrimidin-2-yl)phenoxy)acetaldehyde (420 mg, crude), isopropylamine (245 mg, 4.16 mmol) in MeOH (15 mL) was added NaBH₃CN (131 mg, 2.08 mmol), the mixture was stirred 7 h at reflux. After reaction, it was evaporated and purified by Pre-HPLC to give the title compound (90 mg) as a white solid. ¹H NMR (400 MHz, DMSO-d₆) δ 13.02 (s, 1H), 9.65 (s, 1H), 8.34 (d, J=6.0 Hz, 1H), 8.27 (s, 1H), 8.10 (s, 1H), 7.94 (d, J=3.2 Hz, 2H), 7.57-7.38 (m, 3H), 7.05 (d, J=8.0 Hz, 1H), 6.68 (d, J=6.0 Hz, 1H), 4.106-4.079 (m, 1H), 2.91 (s, 2H), 2.80-2.77 (m, 1H), 1.61 (b, 1H), 1.00 (d, J=6.4 Hz, 6H); m/e 389 (M+H)⁺.

### Example 155

### N-(2,6-dichloropyrimidin-4-yl)-1H-indazol-5-amine

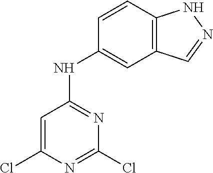 [SMILES: Clc1cc(Nc2ccc3[nH]ncc3c2)nc(Cl)n1]

To a stirred solution of 2,4,6-trichloropyrimidine (5.5 g, 30 mmol) in EtOH (100 mL) were added TEA (1.5 g, 45 mmol) and compound 1H-indazol-5-amine (3.99 g, 30 mmol) at room temperature. The mixture was refluxed overnight. After removing the solvent the residue was re-crystallized in MeOH to give the title compound as a solid (3.4 g, yield 40%).

### Example 156

### tert-butyl 4-(6-((1H-indazol-5-yl)amino)-2-chloropyrimidin-4-yl)piperazine-1-carboxylate

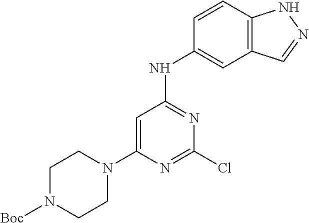 [SMILES: CC(C)(C)OC(=O)N1CCN(c2cc(Nc3ccc4[nH]ncc4c3)nc(Cl)n2)CC1]

To a stirred solution of N-(2,6-dichloropyrimidin-4-yl)-1H-indazol-5-amine (1 g, 3.5 mmol) in EtOH (10 mL) was added TEA (1.4 g, 7 mmol), and compound tert-butyl piperazine-1-carboxylate (0.67 g, 3.5 mmol) at room temperature. The mixture was refluxed overnight. After reaction, water was added, separated the organic layer and saturated brine, dried over Na₂SO₄ and concentrated under reduced pressure to give the title compound (1.2 g) which was used directly for next step reaction without further purification.

### Example 157

### tert-butyl 5-((tert-butoxycarbonyl)(6-(4-(tert-butoxycarbonyl)piperazin-1-yl)-2-chloropyrimidin-4-yl)amino)-1H-indazole-1-carboxylate

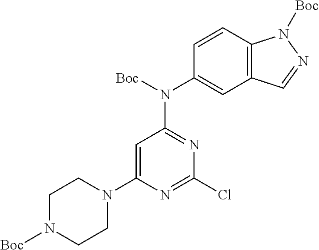 [SMILES: CC(C)(C)OC(=O)N1CCN(c2cc(N(C(=O)OC(C)(C)C)c3ccc4c(cnn4C(=O)OC(C)(C)C)c3)nc(Cl)n2)CC1]

To a stirred solution of tert-butyl 4-(6-((1H-indazol-5-yl)amino)-2-chloropyrimidin-4-yl)piperazine-1-carboxylate (1.2 g, crude) in DCM (10 mL) was added (Boc)₂O (3 g, 14 mmol), TEA (1.4 g, 14 mmol) and DMAP (0.5 g, 3.5 mmol) at room temperature. The mixture was stirred at room temperature for 30 min. After reaction, water was added, separated the organic layer, washed with citric acid monohydrate and saturated brine, dried over Na₂SO₄ and concentrated under reduced pressure. The residue was purified by column chromatograph on silica gel to give the title compound (0.6 g).

### Example 158

### tert-butyl 5-((tert-butoxycarbonyl)(6-(4-(tert-butoxycarbonyl)piperazin-1-yl)-2-(3-methoxyphenyl)pyrimidin-4-yl)amino)-1H-indazole-1-carboxylate

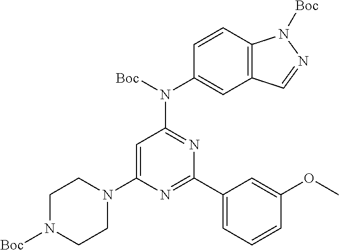 [SMILES: COc1cccc(-c2nc(N3CCN(C(=O)OC(C)(C)C)CC3)cc(N(C(=O)OC(C)(C)C)c3ccc4c(cnn4C(=O)OC(C)(C)C)c3)n2)c1]

A mixture of tert-butyl 5-((tert-butoxycarbonyl)(6-(4-(tert-butoxycarbonyl)piperazin-1-yl)-2-chloropyrimidin-4-yl)amino)-1H-indazole-1-carboxylate (600 mg, 0.95 mmol), (3-methoxyphenyl)boronic acid (160 mg, 1.05 mmol), Na₂CO₃ (201 mg, 1.9 mmol), Pd(dppf)Cl₂ (70 mg, 0.095 mmol) in dioxane:H₂O (6.6 mL, 10:1) was heated under microwave irradiation for 20 min at 140° C. After reaction, it was evaporated, EA and water was added, separated the organic layer, washed with saturated brine, dried over Na₂SO₄ concentrated and purified by silica gel to give the title compound (250 mg, yield 37.5%).

### Example 159

### N-(2-(3-methoxyphenyl)-6-(piperazin-1-yl)pyrimidin-4-yl)-1H-indazol-5-amine

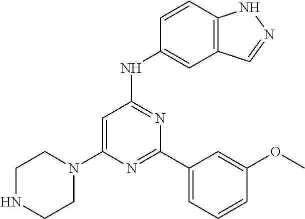 [SMILES: COc1cccc(-c2nc(Nc3ccc4[nH]ncc4c3)cc(N3CCNCC3)n2)c1]

To a solution of tert-butyl 5-((tert-butoxycarbonyl)(6-(4-(tert-butoxycarbonyl)piperazin-1-yl)-2-(3-methoxyphenyl)pyrimidin-4-yl)amino)-1H-indazole-1-carboxylate (250 mg) in DCM (5 mL) was added TFA (1 mL). The mixture was stirred at room temperature for 30 min. it was evaporated, then water was added, adjusted the pH to 9 with saturated Na₂CO₃, filtered to give the title compound (115 mg, yield 80%) as a white solid. 1H NMR (400 MHz, DMSO-d₆) δ 12.98 (s, 1H), 9.30 (s, 1H), 8.09 (s, 1H), 8.00 (s, 1H), 7.53-7.37 (m, 6H), 7.03 (d, J=8.8 Hz, 1H), 6.50 (s, 1H), 3.82 (s, 3H), 3.73 (b, 4H), 3.39 (s, 1H), 2.71 (b, 4H); m/e 402 (M+H)⁺.

### Example 160

### N-(2-chloro-6-(2-(dimethylamino)ethoxy)pyrimidin-4-yl)-1H-indazol-5-amine

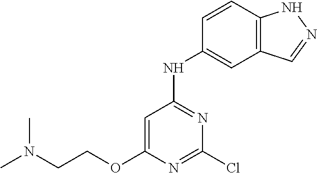 [SMILES: CN(C)CCOc1cc(Nc2ccc3[nH]ncc3c2)nc(Cl)n1]

To a stirred solution of N-(2,6-dichloropyrimidin-4-yl)-1H-indazol-5-amine (2 g, 7 mmol) in EtOH (20 mL) was added TEA (2.8 g, 7 mmol), and 2-(dimethylamino)ethanol (0.64 g, 7 mmol) at room temperature. The mixture was refluxed overnight. After reaction, water was added, separated the organic layer and saturated brine, dried over Na₂SO₄ and concentrated under reduced pressure to give the title compound (1.5 g). The residue was used into next step.

### Example 161

### N-(6-(2-(dimethylamino)ethoxy)-2-(3-methoxyphenyl)pyrimidin-4-yl)-1H-indazol-5-amine

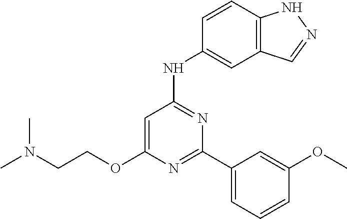

A mixture of N-(2-chloro-6-(2-(dimethylamino)ethoxy)pyrimidin-4-yl)-1H-indazol-5-amine (1.5 g, 4.5 mmol), (3-methoxyphenyl)boronic acid (661 mg, 5 mmol), Na₂CO₃ (954 mg, 9 mmol), Pd(dppf)Cl₂ (300 mg, 0.45 mmol) in dioxane:H₂O (22 mL, 10:1) was heated under microwave irradiation for 30 min at 140° C. After reaction, it was evaporated, EA and water was added, separated the organic layer, washed with saturated brine, dried over Na₂SO₄ and concentrated which was purified by Pre-HPLC to give the title compound as white solid (125 mg). 1H NMR (400 MHz, DMSO-d₆) δ 13.00 (s, 1H), 9.67 (s, 1H), 8.18 (s, 1H), 8.04 (s, 1H), 7.56-7.41 (m, 5H), 7.08-6.86 (m, 2H), 4.46-4.43 (m, 1H), 3.83 (s, 3H), 2.66-2.63 (m, 2H), 2.22 (s, 6H); m/e 405 (M+H)⁺.

### Example 162

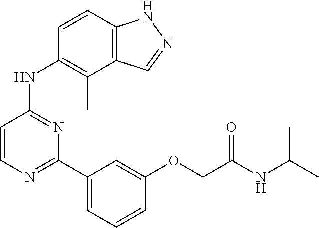 [SMILES: Cc1c(Nc2ccnc(-c3cccc(OCC(=O)NC(C)C)c3)n2)ccc2[nH]ncc12]

The following compounds were synthesized using the procedures described above:

TABLE 1

| Example | | | calc | Observed |
|---|---|---|---|---|
| No. | Structure | Formula | M + H | M + H |
| 163 | 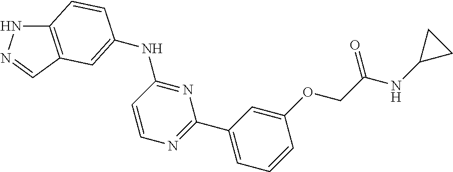 [SMILES: O=C(COc1cccc(-c2nccc(Nc3ccc4[nH]ncc4c3)n2)c1)NC1CC1] | C₂₂H₂₀N₆O₂ | 401 | 401 |
| 164 | 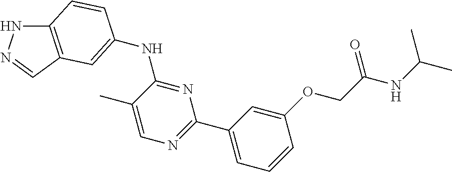 [SMILES: Cc1cnc(-c2cccc(OCC(=O)NC(C)C)c2)nc1Nc1ccc2[nH]ncc2c1] | C₂₃H₂₄N₆O₂ | 417 | 417 |
| 165 | 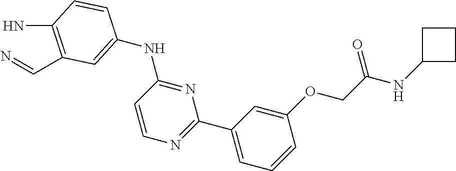 [SMILES: N=Cc1cc(Nc2ccnc(-c3cccc(OCC(=O)NC4CCC4)c3)n2)ccc1N] | C₂₃H₂₂N₆O₂ | 415 | 415 |
| 166 | 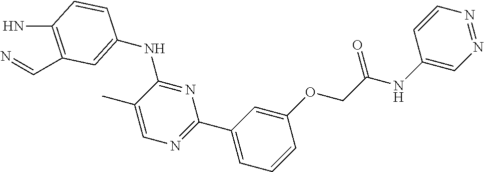 [SMILES: Cc1cnc(-c2cccc(OCC(=O)Nc3ccnnc3)c2)nc1Nc1ccc(N)c(C=N)c1] | C₂₄H₂₀N₈O₂ | 453 | 453 |
| 167 | 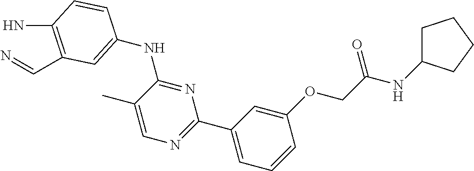 [SMILES: Cc1cnc(-c2cccc(OCC(=O)NC3CCCC3)c2)nc1Nc1ccc(N)c(C=N)c1] | C₂₅H₂₆N₆O₂ | 443 | 443 |
| 168 | 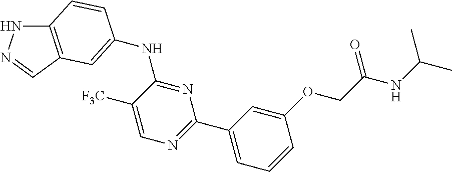 [SMILES: CC(C)NC(=O)COc1cccc(-c2ncc(C(F)(F)F)c(Nc3ccc4[nH]ncc4c3)n2)c1] | C₂₃H₂₁F₃N₆O₂ | 471 | 471 |
| 169 | 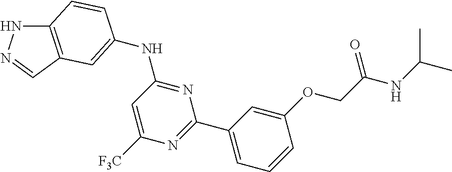 [SMILES: CC(C)NC(=O)COc1cccc(-c2nc(Nc3ccc4[nH]ncc4c3)cc(C(F)(F)F)n2)c1] | C₂₃H₂₁F₃N₆O₂ | 471 | 471 |
| 170 | 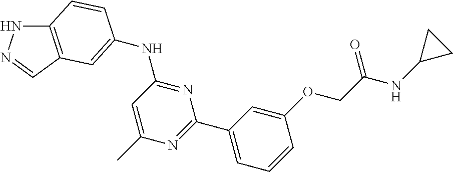 [SMILES: Cc1cc(Nc2ccc3[nH]ncc3c2)nc(-c2cccc(OCC(=O)NC3CC3)c2)n1] | C₂₃H₂₂N₆O₂ | 415 | 415 |
| 171 | 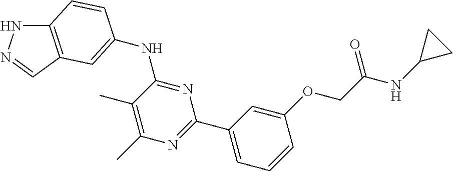 [SMILES: Cc1nc(-c2cccc(OCC(=O)NC3CC3)c2)nc(Nc2ccc3[nH]ncc3c2)c1C] | C₂₄H₂₄N₆O₂ | 429 | 429 |
| 172 | 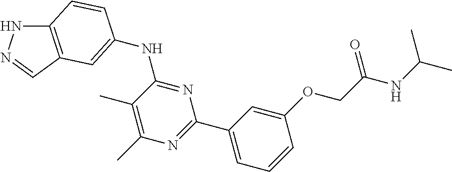 [SMILES: Cc1nc(-c2cccc(OCC(=O)NC(C)C)c2)nc(Nc2ccc3[nH]ncc3c2)c1C] | C₂₄H₂₆N₆O₂ | 431 | 431 |
| 173 | 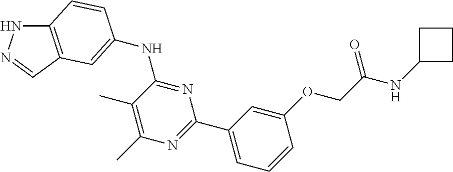 [SMILES: Cc1nc(-c2cccc(OCC(=O)NC3CCC3)c2)nc(Nc2ccc3[nH]ncc3c2)c1C] | C₂₅H₂₆N₆O₂ | 443 | 443 |
| 174 | 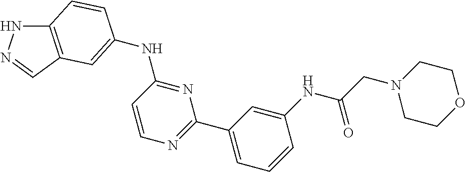 [SMILES: O=C(CN1CCOCC1)Nc1cccc(-c2nccc(Nc3ccc4[nH]ncc4c3)n2)c1] | C₂₃H₂₃N₇O₂ | 430 | 430 |
| 175 | 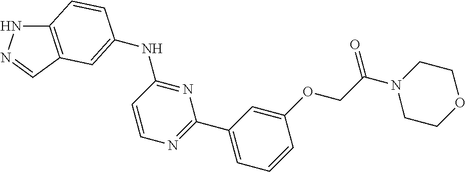 [SMILES: O=C(COc1cccc(-c2nccc(Nc3ccc4[nH]ncc4c3)n2)c1)N1CCOCC1] | C₂₃H₂₂N₆O₃ | 431 | 431 |
| 176 | 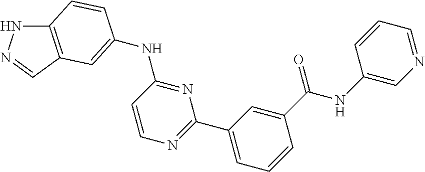 [SMILES: O=C(Nc1cccnc1)c1cccc(-c2nccc(Nc3ccc4[nH]ncc4c3)n2)c1] | C₂₃H₁₇N₇O | 408 | 408 |
| 177 | 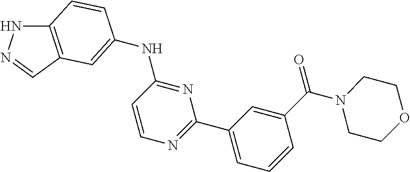 [SMILES: O=C(c1cccc(-c2nccc(Nc3ccc4[nH]ncc4c3)n2)c1)N1CCOCC1] | C₂₂H₂₀N₆O₂ | 401 | 401 |
| 178 | 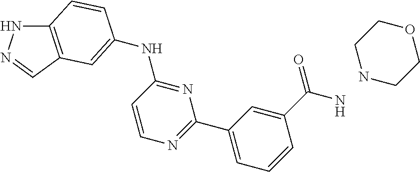 | C₂₂H₂₁N₇O₂ | 416 | 416 |
| 179 | 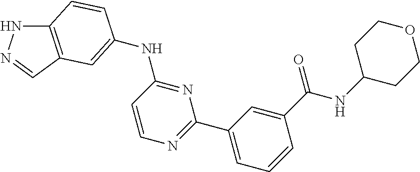 [SMILES: O=C(NC1CCOCC1)c1cccc(-c2nccc(Nc3ccc4[nH]ncc4c3)n2)c1] | C₂₃H₂₂N₆O₂ | 415 | 415 |
| 180 | 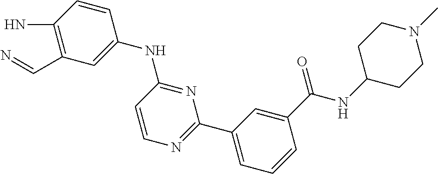 [SMILES: CN1CCC(NC(=O)c2cccc(-c3nccc(Nc4ccc(N)c(C=N)c4)n3)c2)CC1] | C₂₄H₂₅N₇O | 428 | 428 |

### Example 181

### ROCK1 and ROCK2 Compound Selectivity

Dose response curves for Rho-kinase inhibition were derived from a Millipore immuno-based 96 well plate assay (Millipore catalog number CSA001). Purified active ROCK1 and ROCK2 were obtained from Invitrogen (catalog numbers ROCKI, PV3691 and ROCK2, PV3759). The kit components include assay plates, which are pre-coated with recombinant MYPT1, which contains a specifically phosphorylatable Thr696. The inhibitory activities of compounds are measured according to the manufactures protocol. Briefly, decreasing concentrations of test compounds or the known ROCK inhibitor Y-27963, are added, from 50 uM to 0.003 uM to reaction buffer containing 5 mM MgCl2, and 10 mUnits of ROCK1 or ROCK2 in assay dilution buffer. This mixture is overlayed into the 96 well plate and the reaction is initiated with the addition of 2.5 uM ATP. The assay proceeds at 300 Celsius for 30 minutes with gentle shaking at 120 rpm. The assay is terminated by washing of the plate 3 times with Tris-buffered saline and tween wash buffer. Anti-phospho-MYPT1 (Thr696) antibody is added to each well to detect the phosphorylated substrate and incubated for 1 hour at room temperature after which HRP conjugated anti-rabbit IgG secondary is added for 1 hour at room temperature. After washing the assay is developed using a substrate reagent and the absorbance is read at 450 nm on a Tecan Infinite M1000 reflecting the relative remaining ROCK phosphorylation activity.

Data showing inhibition of ROCK1 and ROCK2, and selectivity of certain compounds for ROCK2 inhibition, is presented in Table 3.

TABLE 3
IC₅₀ and Ki for ROCK1 and ROCK2

| | ROCK1 IC₅₀ | ROCK2 IC₅₀ | ROCK1 Ki | ROCK2 Ki |
|---|---|---|---|---|
| Compound | (μM) | (μM) | (μM) | (μM) |
| Ex. 12 | 1.77 | 0.54 | 0.07 | 0.02 |
| Ex. 26 | 53.45 | 0.72 | 2.01 | 0.03 |
| Ex. 28 | 6.69 | 1.96 | 0.26 | 0.08 |
| SLx-2119 | 13.11 | 1.02 | 0.50 | 0.04 |
| Y-27263 | 1.13 | 1.63 | 0.04 | 0.06 |
| Ex. 14 | | 4.25 | | 0.17 |
| Ex. 48 | 0.33 | 0.47 | 0.01 | 0.02 |
| Ex. 13 | 22.53 | 4.64 | 0.87 | 0.18 |

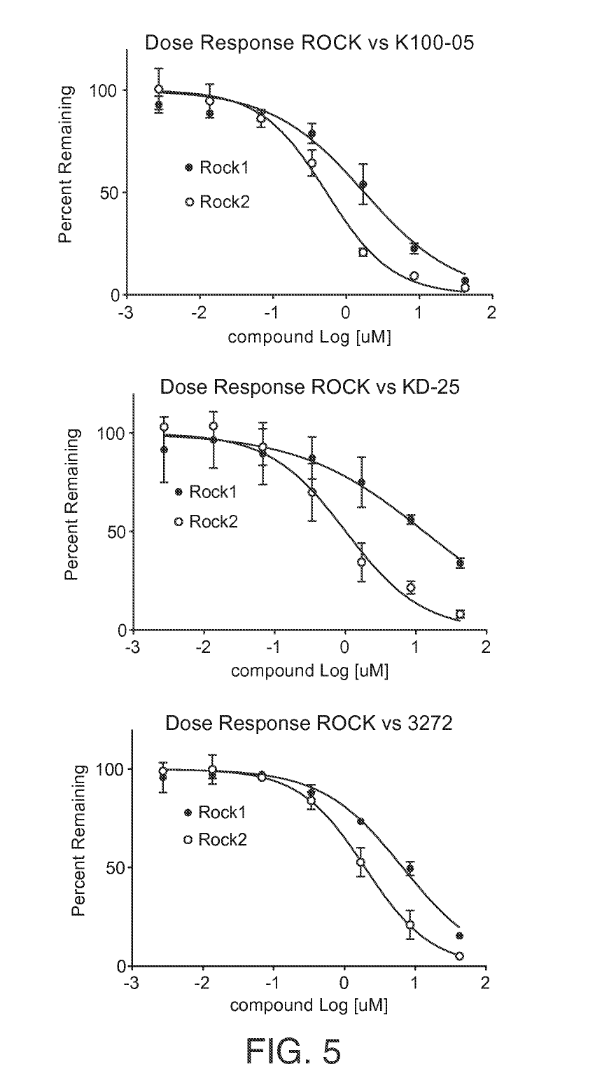

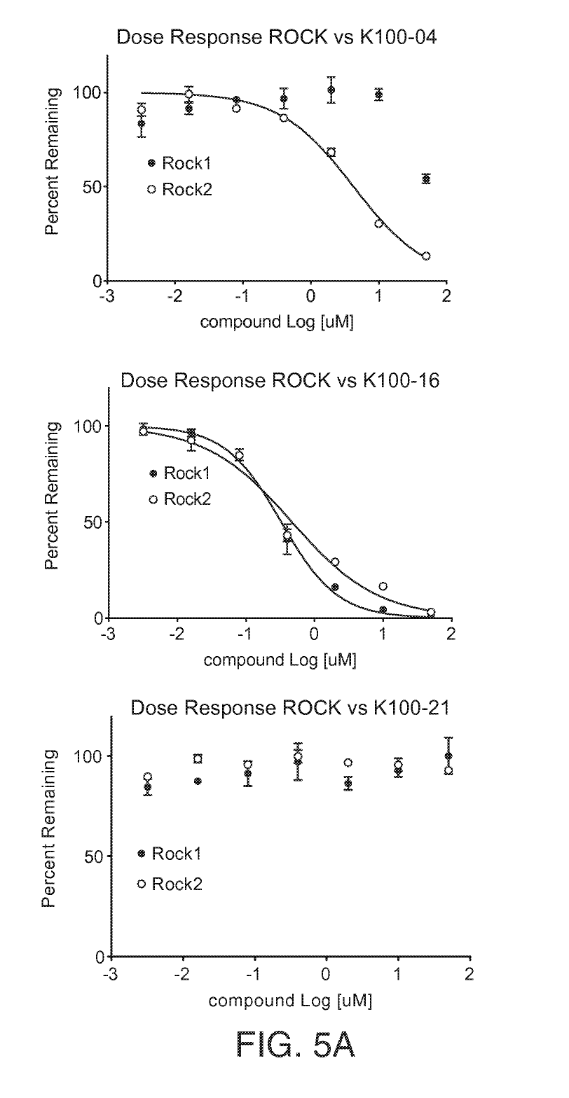

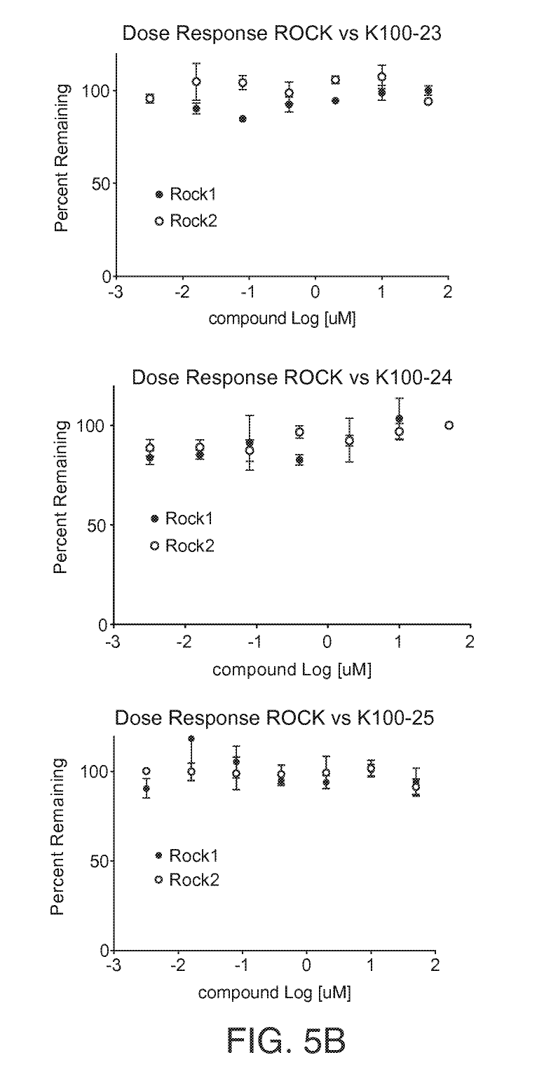

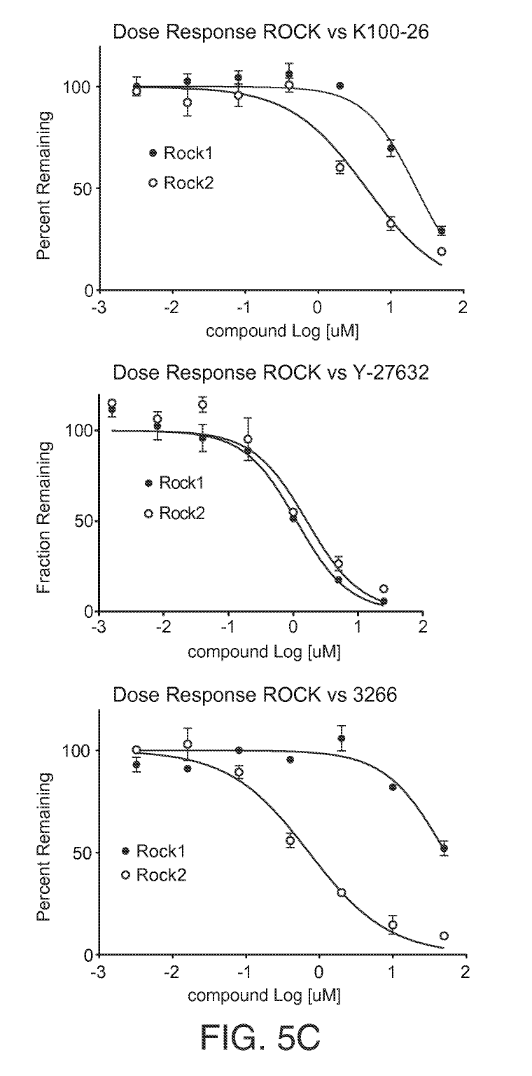

Dose response curves for inhibition of ROCK1 vs ROCK2 is shown in FIG. 5.

### Example 182

### ROCK1 and ROCK2 Compound Selectivity

Dose response curves for Rho-kinase inhibition were derived from a Invitrogen Z′-LYTE™ Kinase Assay Kit (Invitrogen catalog number PV3793). Purified active ROCK1 and ROCK2 were obtained from Invitrogen (catalog numbers ROCK1, PV3691 and ROCK2, PV3759). The kit components include a coumarin and fluorescein labeled peptide based on myosin light chain 2 (KKRPQRRYSNVF), a proprietary protease containing development reagent and a proprietary Stop buffer used to terminate the development reaction. The inhibitory activities of compounds are measured according to the manufactures protocol. Briefly, decreasing concentrations of test compounds or the known ROCK inhibitor Y-27963, are added, from 10 uM to 2.56×10⁻⁵ uM to reaction buffer containing 50 mM HEPES pH 7.5, 10 mM MgCl₂, 5 mM EGTA, and 0.05% Brij-35 and of ROCK1 at 0.18 ug/mL or ROCK2 at 0.8 ug/mL in assay dilution buffer. This mixture is overlayed into a white 96-well half area plate and the reaction is initiated with the addition of 5 uM ATP for ROCK1 or 12 uM ATP for ROCK2. The assay proceeds at room temperature for 1 hour followed by the addition of development reagent, and further incubation for 1 hour at room temperature. STOP reagent is then added and the reaction and immediately the coumarin and fluorescein emission signals are read on a Tecan Infinite M1000 fluorescence plate reader (excitation: 400 nm; emission 445 and 520 nm, respectively). By comparing the emission ratios of the test samples against control samples, percent phosphorylation values are calculated and the concentration of inhibitor that produces 1/2 inhibition of kinase activity (IC₅₀) is determined using Prism. Table 4 provides IC₅₀ concentrations for compounds of the above examples. Several of the compounds also demonstrated activity in a preliminary assay that measured inhibition of myosin light chain phosphorylation (pMLC). For compounds marked ND, activity was not determinable under the test conditions employed.

TABLE 4
ROCK Inhibition

| | ROCK2 | ROCK1 | pMLC | | ROCK2 | ROCK1 | pMLC |
|---|---|---|---|---|---|---|---|
| Ex. | IC₅₀ | IC₅₀ | inhibi- | Ex. | IC₅₀ | IC₅₀ | inhibi- |
| No. | (nM) | (nM) | tion | No. | (nM) | (nM) | tion |
| 14 | | | + | 91 | | | + |
| 12 | | | ND | 75 | | | ++ |
| 43 | 25 | 750 | ++ | 79 | | | + |
| 48 | 1000 | 300 | ++ | 95 | | | ++ |
| 38 | | | + | 110 | | | |
| 52 | | | | 82 | | | |
| 56 | | | ND | 86 | | | |
| 126 | | | ND | 98 | | | |
| 112 | | | ND | 83 | | | |
| 114 | | | ND | 87 | | | |
| 60 | | | ND | 99 | | | |
| 74 | | | ND | 106 | | | |
| 60 | | | ND | 102 | | | |
| 65 | | | ND | 5 | 70 | >3000 | |
| 71 | | | + | 26 | 30 | 3500 | |
| 117 | | | + | 163 | 80 | 5900 | |
| 118 | 40 | 6600 | + | 164 | 60 | 5000 | |
| 119 | | | + | 165 | 50 | 1700 | |
| 120 | | | | 166 | 20 | 2200 | |
| 121 | 13 | 1390 | + | 28 | 70 | 2500 | |
| 122 | | | ND | 167 | 60 | 3400 | |
| 123 | | | ND | 168 | 30 | >10000 | |
| 124 | | | + | 169 | >10000 | >10000 | |
| 125 | | | + | 170 | 70 | 4100 | |
| 127 | | | ++ | 171 | 80 | 7500 | |
| 129 | 10 | 1100 | + | 172 | 120 | >10000 | |
| 131 | 39 | 2000 | + | 173 | 30 | >10000 | |
| 134 | | 830 | + | 174 | 191 | 2800 | |
| 136 | | 170 | + | 17 | 30 | 1200 | |
| 138 | 8 | 1980 | + | 175 | 500 | | |
| 140 | 55 | 1908 | + | 176 | 13 | 3500 | |
| 144 | 58 | 1927 | + | 177 | 4900 | >10000 | |
| 147 | >5500 | >2000 | | 178 | 700 | 4400 | |
| 150 | 1000 | >3000 | ++ | 179 | 310 | 2400 | |
| 154 | 40 | >10000 | ++ | 22 | 340 | 10000 | |
| 159 | 200 | 100 | ++ | 180 | 380 | >10000 | |
| 161 | 3200 | 1000 | | 20 | 400 | >10000 | |

### Example 183

### ROCK2 Selective Inhibitor, KD025, Inhibits IL-17/IL-21 Secretion and Proliferation in Human CD4⁺ T Cells In Vitro

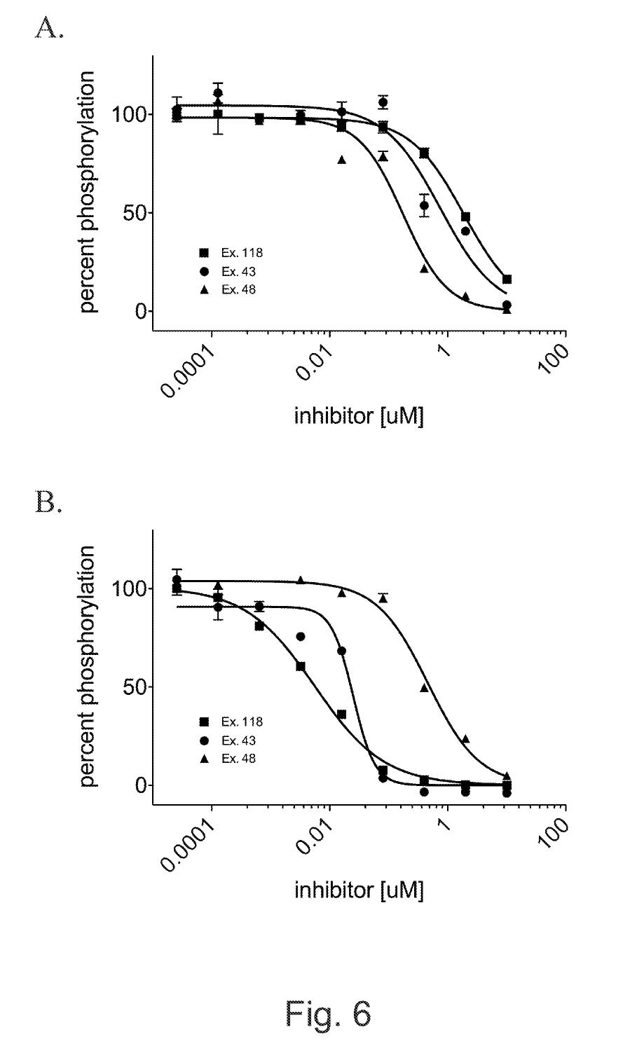

Activation of resting T cells, resulting in cytokine secretion and proliferation, involves two distinct signals from antigen-presenting cells (APCs), mimicked by co-stimulation of the T cell receptor (TCR)/CD3 complex and the CD28 receptor. Using freshly purified CD4⁺ human T cells and stimulatory antibodies against CD3 and CD28 to stimulate IL-17 and IL-21 secretion in response to TCR activation, it was found that the treatment with ROCK2 selective inhibitor, KD025, significantly inhibited IL-17 and IL-21 secretion in a dose-dependent manner. Under the same conditions, the inhibition of IFN-γ secretion was less robust and significant only at high dose (10 μM) of the inhibitor (FIG. 6A). Consistent with the inhibitory effect on cytokine secretion, the treatment of T cells with KD025 down-regulated their ability to proliferate in response to TCR stimulation in vitro (FIG. 6B).

### Example 184

### ROCK2 siRNA, but not ROCK1 siRNA Inhibits, IL-17 and IL-21 Secretion

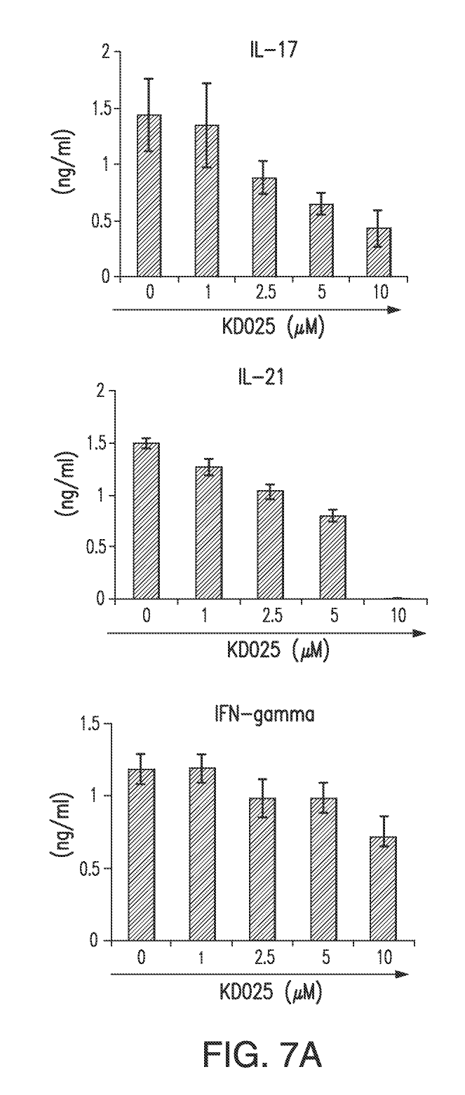

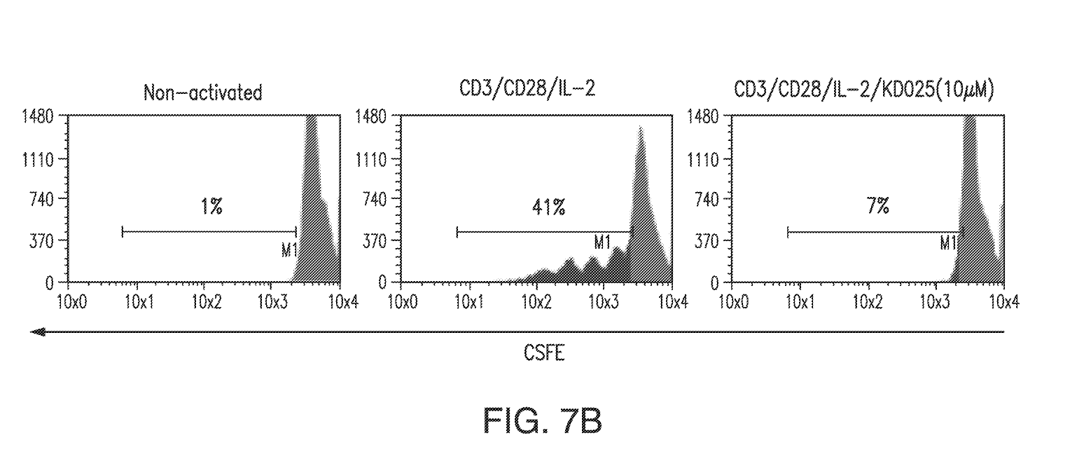

To confirm the role of ROCK2 in regulation of IL-17 and IL-21 secretion in human T cells we specifically silenced ROCK1 and ROCK2 expression by RNA interference. Specific ROCK1 and ROCK2 small interfering RNA (siRNA) reduced the protein expression levels by 72% and 84% respectively. Silencing of ROCK2, but not of ROCK1 significantly reduced the IL-17 and IL-21, with minimal effect on IFN-γ secretion in human T cells (FIG. 7).

### Example 185

### KD025 Inhibits STAT3 Phosphorylation

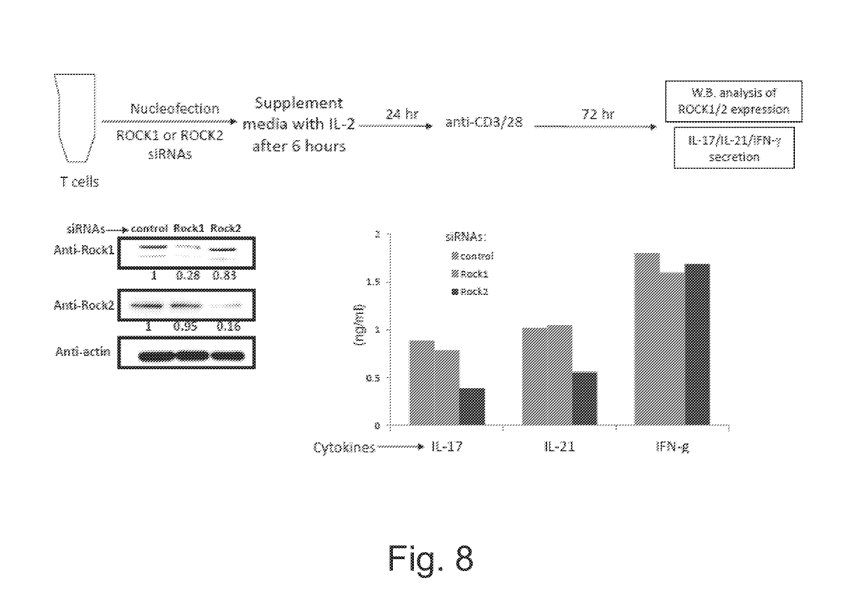

STAT3 plays a critical role in Th17 differentiation via regulation of RORγt expression and direct binding to the IL-17 and IL-21 promoters. In addition, recent studies have demonstrated that RhoA-dependent STAT3 stimulation requires ROCK activity and leads to activation of STAT3 phosphorylation on amino acid Y705. Using two different experimental designs, KD025 was demonstrated to significantly down-regulates the phosphorylation of STAT3. In one experiment, T cells were pre-treated with KD025 and then stimulation with anti-CD3/CD28 antibodies. Pre-treatment with KD025 resulted in reduced phosphorylation of STAT3 (FIG. 8A). In a different experiment, cells we cultured under Th17-skewing conditions for 5 days and then treated with the ROCK2 selective inhibitor for 3 hours. STAT3 phosphorylation was reduced by treatment with the ROCK2 inhibitor (FIG. 8B).

### Example 186

### KD025 Down-Regulates IL-17, IL-21 and IFN-γ Secretion (a) and the Increased Frequency of IFN-γ and IL-17-Expressing Cells (b) in RA Patients Ex Vivo

Rheumatoid arthritis (RA) is a chronic autoimmune inflammatory disease leading to the destruction of joint architecture. The pathogenic events that involved in RA development are not fully understood, although the pivotal role of pro-inflammatory cytokines, such as TNF-α, IL-1β, IFN-β, IL-6 and more recent IL-17 in the induction and maintenance of RA pathogenesis is well documented. Moreover, the frequency of Th17 cells in peripheral blood of RA patients is significantly increased compared to healthy controls and correlates with disease activity score (DAS).

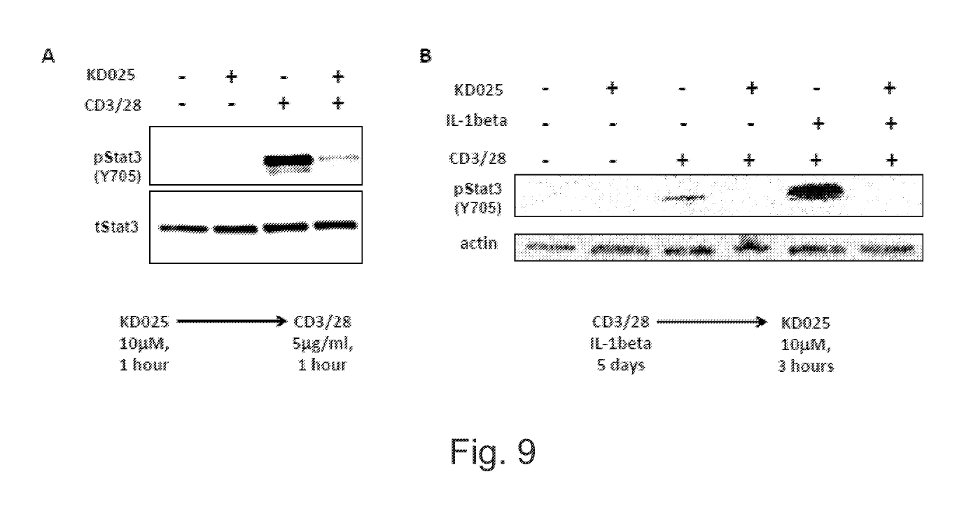

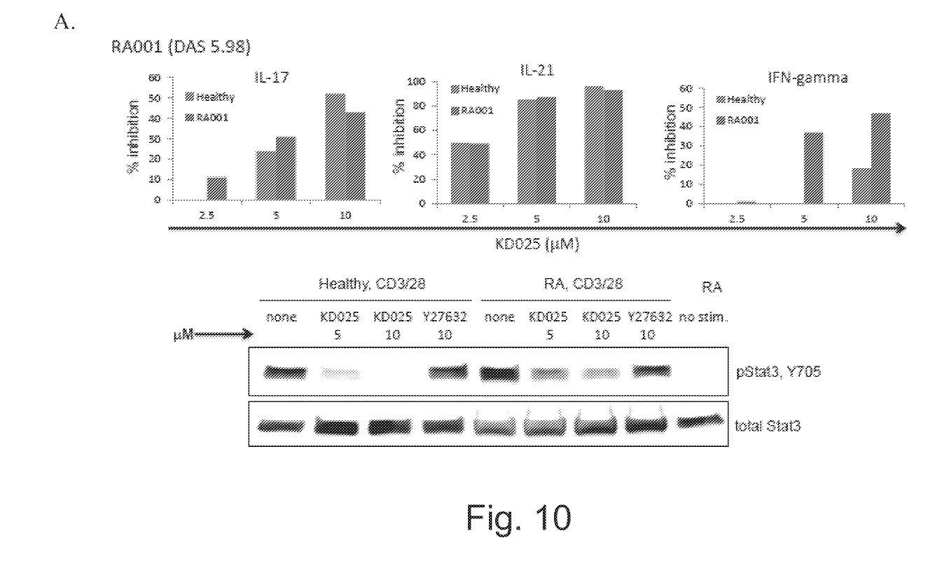

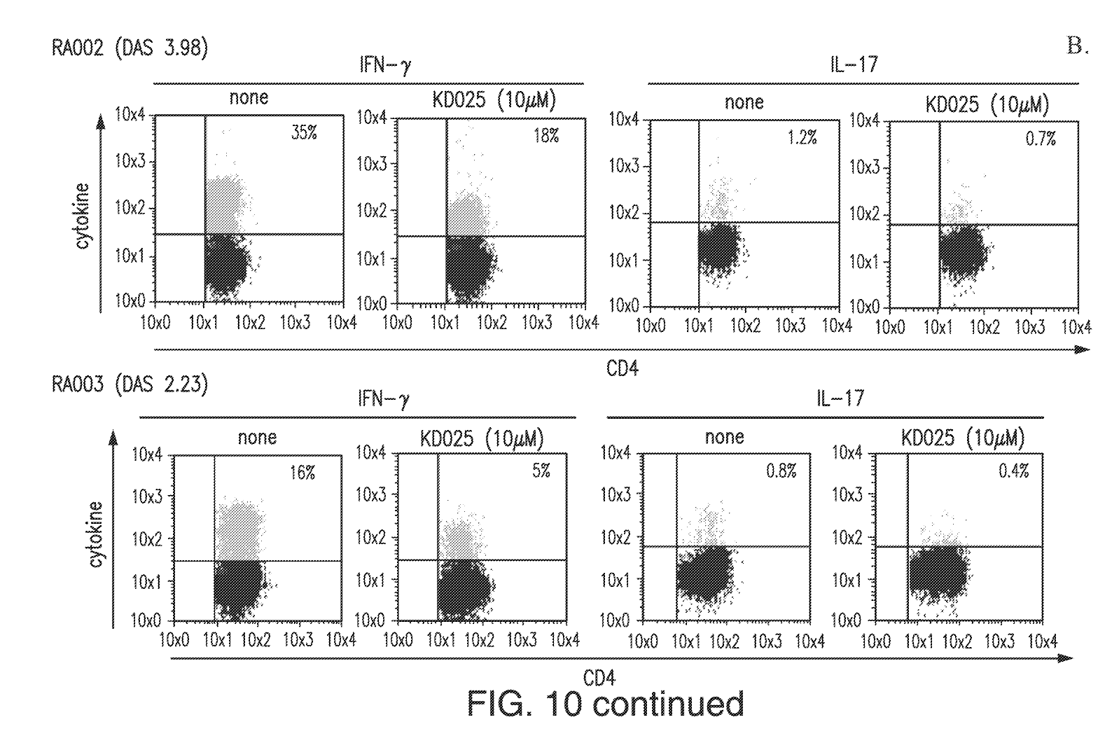

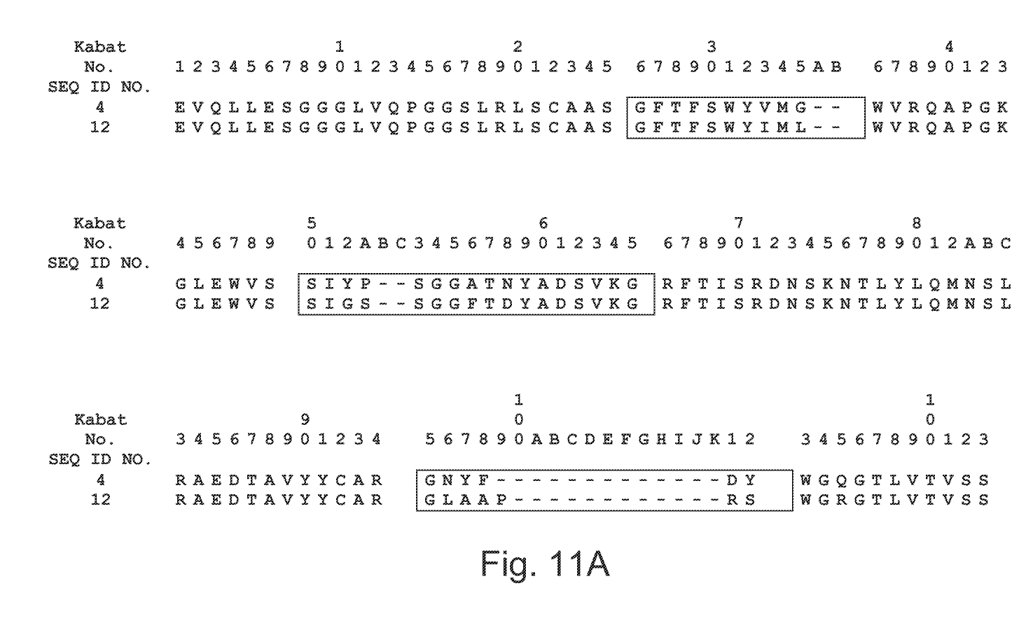

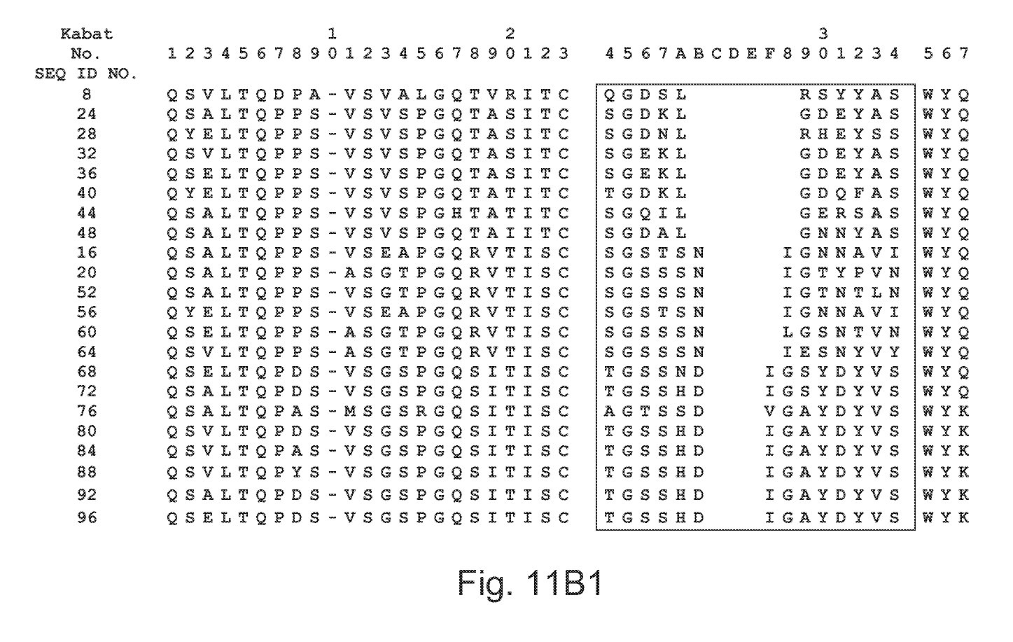

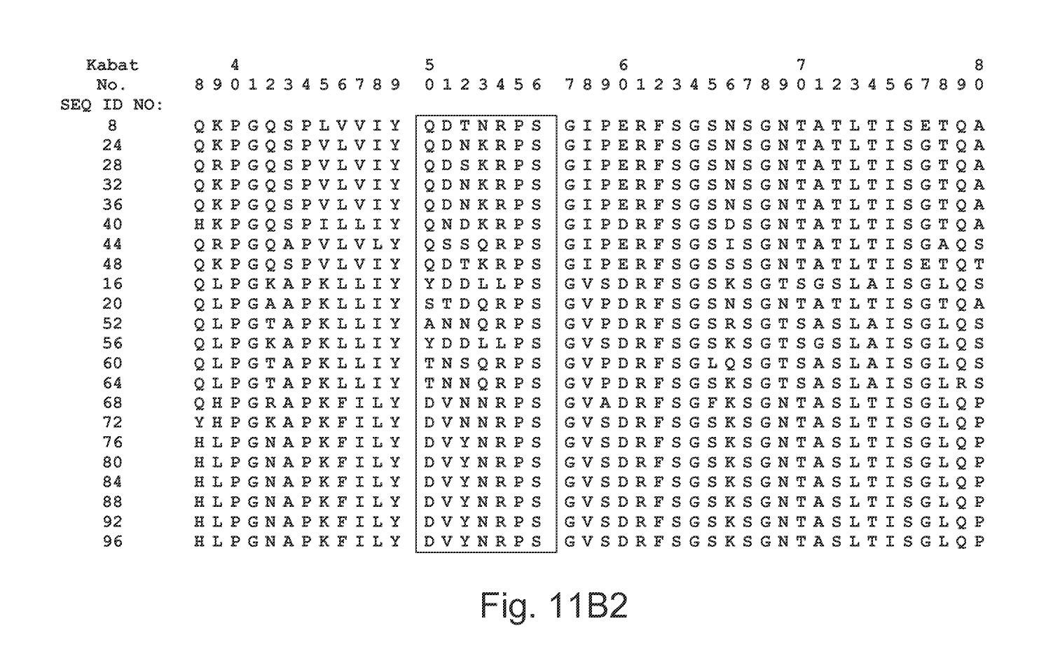

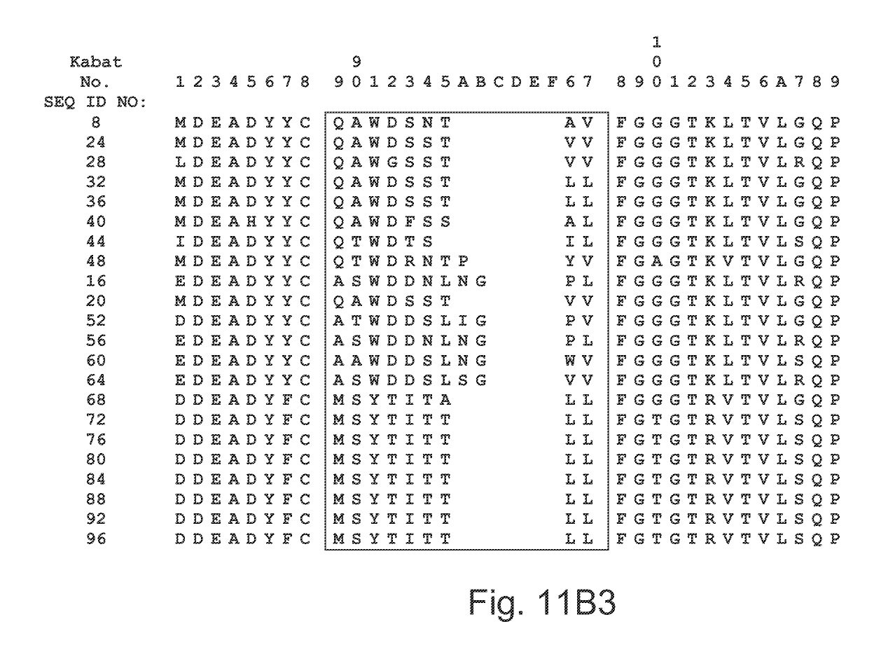

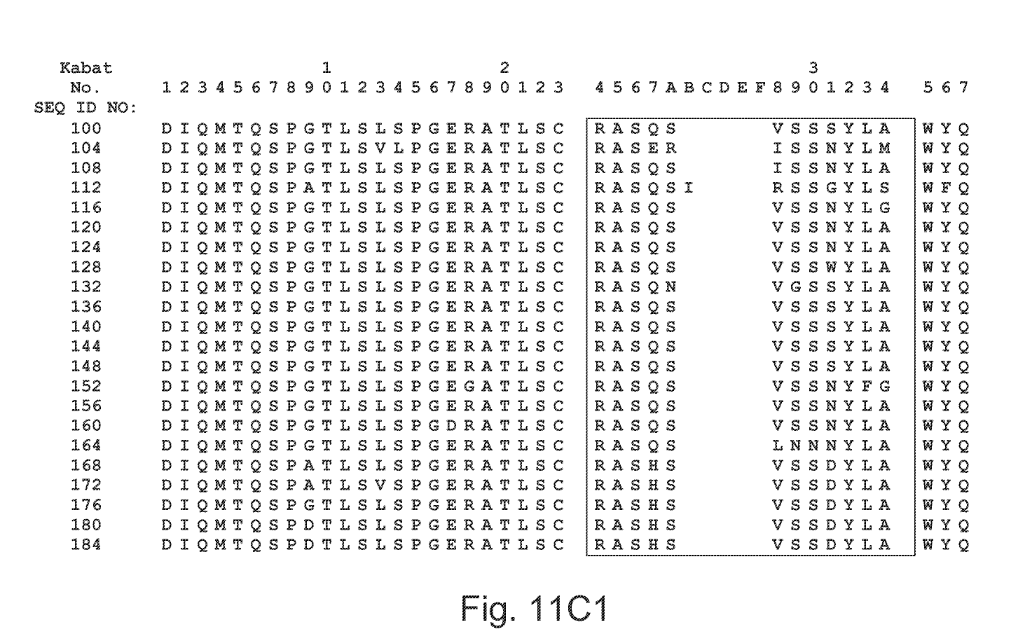

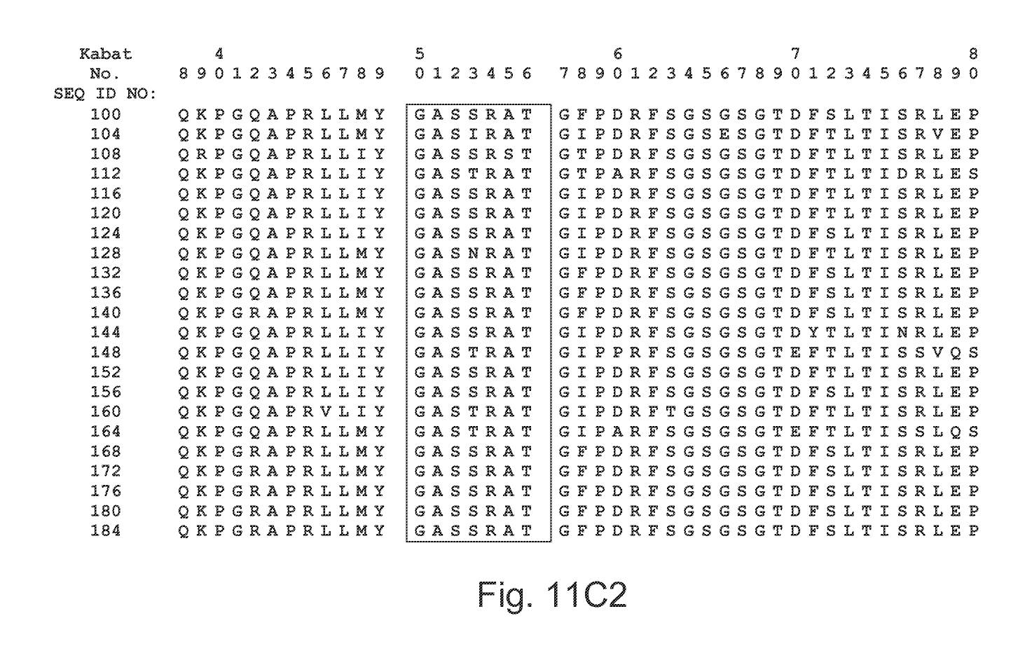

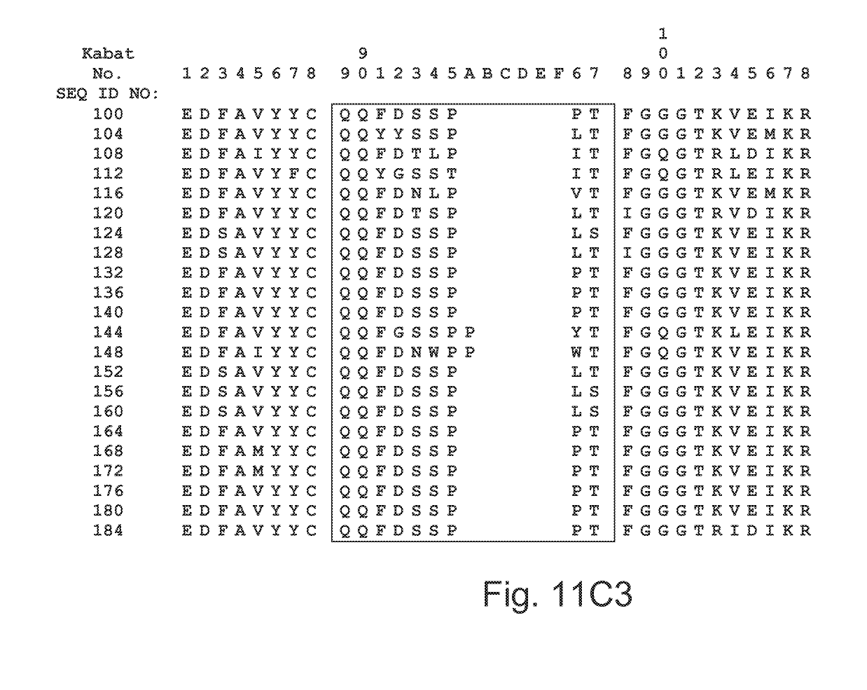

CD4⁺ T cells were purified from peripheral blood of RA patients at different stages of the disease or from healthy controls and stimulated using anti-CD3/CD28 antibodies in presence of KD025 ex vivo. The ROCK2 selective inhibitor significantly down-regulated the ability of CD4⁺ T cell to secrete IL-17, IL-21 and IFN-γ in response to TCR stimulation in a STAT3-dependent manner (FIG. 9A). In contrast to healthy controls, the degree of inhibition of IFN-γ secretion was comparable to inhibition levels of IL-17 and IL-21. Moreover, culture of CD4⁺ T cells from 2 different RA patients in presence of KD025 significantly reduced the frequencies of both IL-17 and IFN-γ-producing cells as was demonstrated by intracellular staining (FIG. 9B).

### Example 187

### Antibodies that Neutralize Human VEGFR2

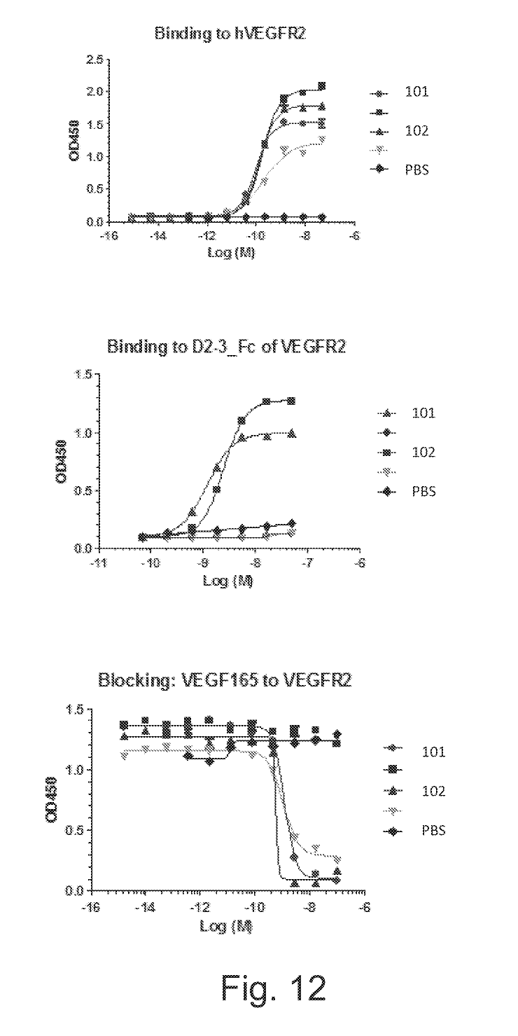

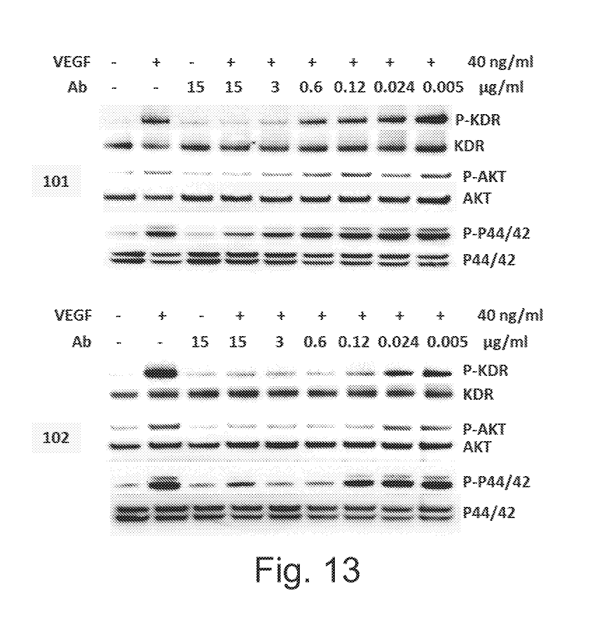

Two antibodies that bind to and neutralize human VEGFR2, identified in Table 1, were isolated from human Fab phage display libraries. The antibodies block binding of the ligand VEGFA to hVEGFR2 (FIG. 12). The antibodies also bind to porcine aortic endothelial (PAE) cells expressing KDR, and inhibit VEGFA-stimulated phosphorylation of VEGFR2, AKT, and MAPK. (FIG. 13). Table 1 indicates amino acid sequences of the CDRs and variable domains of the antibodies. The K_{d}S of Mab 101 and Mab 102 are about 6.6 mM and 1.7 nM, respectively.

The heavy chain of Mab 101 was reshuffled with κ light chain genes (K-library) and λ light chain genes (λ-library). 20 unique λ light chain variants were found by panning the λ-library against both human VEGFR2 and mouse VEGFR2. 22 unique κ light chain variants were found by panning the κ-library against both human VEGFR2 and mouse VEGFR2. Table 2 indicates amino acid sequences of the CDRs and variable domains of the light chains. The K_{d}S of Mabs 105, 106, and 107 were increased about 10 fold (0.24 nM, 0.22 nM, and 0.12 nM, respectively) (Table 3). These antibodies, and antibody Mab101 from which they are derived, bind to domains 2 and 3 of VEGFR and to constructs containing those domains.

TABLE 5
Antibody Binding Data

| | ka | kd | KD |
|---|---|---|---|
| Antibody | 10⁴ M⁻¹s⁻¹ | 10⁻⁴ s⁻¹ | nM |
| 107 | 55.8 | 0.934 | 0.167 |
| 109 | 30.6 | 3.80 | 1.24 |
| 104 | 79.2 | 1.13 | 0.165 |
| 110 | 44.9 | 3.10 | 0.69 |
| 108 | 71.9 | 1.75 | 0.244 |
| 105 | 24.3 | 0.591 | 0.243 |
| 101 | 29.8 | 5.93 | 1.81 |

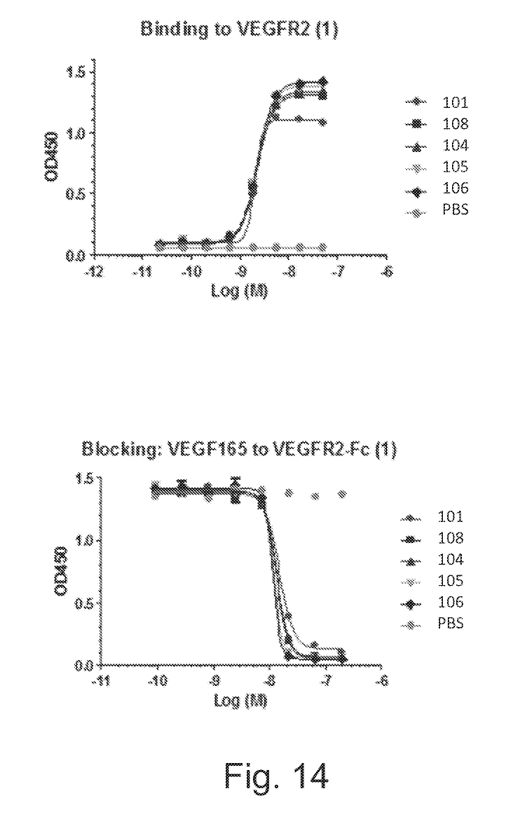

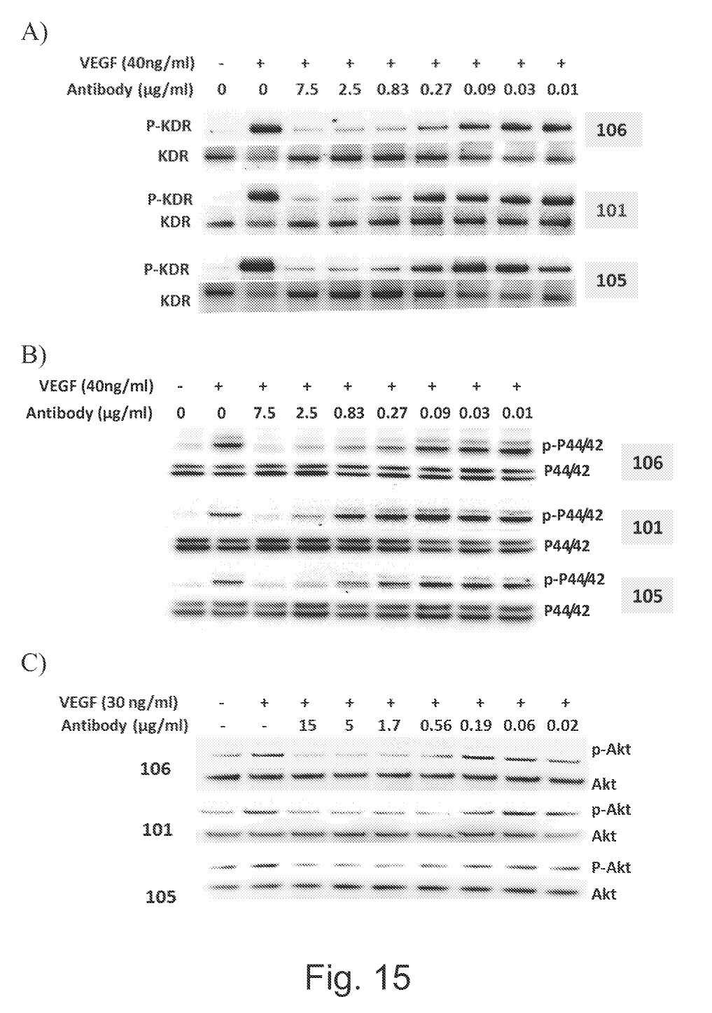

Like the parent antibody, these antibodies bind to VEGFR2 and block binding of VEGFA to VEGFR2 (FIG. 14), and inhibit VEGFA-stimulated phosphorylation of VEGFR2, AKT, and MAPK (FIG. 15).

Several of the antibodies, including Mabs 138, 139, 140, and 146, also cross react with mouse VEGFR2.

TABLE 6
Cross Reactivity

| | hVEGFR2 | | | mVEGFR2 | | |
|---|---|---|---|---|---|---|
| | ka | kd | KD | ka | kd | KD |
| Antibody | 10⁴M⁻¹s⁻¹ | 10⁻⁴s⁻¹ | nM | 10⁴M⁻¹s⁻¹ | 10⁻⁴s⁻¹ | nM |
| 138 | 19.7 | 1.42 | 0.72 | 23.4 | 5.90 | 2.55 |
| 139 | 14.6 | 1.75 | 1.20 | 13.0 | 3.17 | 2.44 |
| 106 | 35.6 | 0.512 | 0.144 | | | |

Mabs 138, 139, and 140 inhibited VEGFA-stimulated phosphorylation of VEFGR2 and downstream signal transduction molecules, including MAPK.

### Example 188

### Treatment of AMD

A study is conducted of 50 men and women having subfoveal choroidal neovascularization (CNV) lesion secondary to AMD. The study employs Mab 106, which is administered weekly by intravitreal injection for three consecutive weeks. The study also employs a compound of Example 43, which inhibits ROCK2 and is ROCK2 selective. The compound of Example 43 is administered either by i) weekly intravitreal injection, or ii) daily administration by in eye drops. Various amounts of each drug are administered at the designated intervals with certain control subjects receiving only the compound of Example 43, or only Mab 106, or placebo. Subjects are observed over six months to determine intraocular inflammation, macular thickness, and the status of macular perfusion. Visual acuity is also measured over the time period.

For each concentration of Mab 106, intraocular inflammation and macular perfusion is less when the compound of Example 43 is administered. Over the time period of the study, loss of visual acuity is reduced in subjects that receive both agents and some subjects experience improvement.

### Example 189

### Treatment of Proliferative Diabetic Retinopathy

Patients with proliferative diabetic retinopathy or clinically significant diabetic macular edema requiring surgical intervention are treated preoperatively with pegaptanib (a pegylated anti-VEGF aptamer with specificity for VEGF 165) with or without concomitant treatment with a compound of Example 154. The agents are administered by intravitreal injection. Subjects are evaluated over 8 weeks following surgery as to post operative rebleed, macular edema post injection, and pre- and post-injection levels of VEGF₁₆₅. It is expected that compared to pegaptanib alone, administration of compound 154 results in reduced bleeding and macular edema.

We claim:

1. A method of treating an ocular disorder having an angiogenic component in a subject, which comprises administering to the subject an effective amount of a rho kinase inhibitor and an angiogenesis inhibitor.

2. The method of claim 1, wherein the ocular disorder is age related macular degeneration (AMD), choroidal neovascularization (CNV), diabetic macular edema (DME), iris neovascularization, uveitis, neovascular glaucoma, or retinitis of prematurity (ROP).

3. The method of claim 1, wherein the rho kinase inhibitor is ROCK2 selective.

4. The method of claim 1, wherein the rho kinase inhibitor is a compound of Formula XVI: 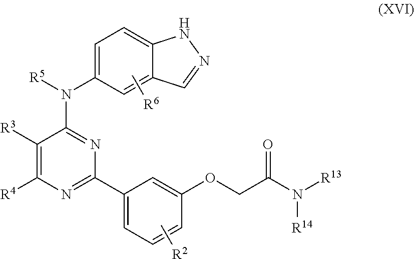
or pharmaceutically acceptable salt thereof, wherein:
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), heteroaryl, C₃-C₇ cycloalkyl, a three to twelve membered heterocyclic or aromatic ring containing up to 3 heteroatoms, each of which may be optionally substituted by from 1 to 3 substituents independently selected from halo, oxo, C₁-C₆ alkyl, C₂-C₆, alkenyl, C₃-C₇ cycloalkyl, C₁-C₆ alkoxy, CN and C₁-C₃ perfluoro alkyl;
or R¹³ and R¹⁴ may be taken together form a three to twelve membered heterocyclic ring having up to 3 heteroatoms which is optionally substituted by from 1 to 3 substituents independently selected from halo, C₁-C₆ alkyl, C₂-C₆, alkenyl, C₁-C₆ alkoxy, C₃-C₇ cycloalkyl, oxo, —OH, —NH₂, CN and C₁-C₃ perfluoro alkyl;
R² is selected from H and halo;
each R³ and R⁴ is independently selected from the group consisting of H, C₁-C₈ alkyl, —CN, halo, —OH, —O—(C₁-C₆ alkyl), —O—(C₁-C₆ alkyl)-O—(C₁-C₆ alkyl), —NR³¹R³², C₁-C₃ perfluoro alkyl, —O—(CH₂)ₐNR³¹R³², —NR³¹—(CH₂)ₐNR³³R³⁴, —NR³¹—(CH₂)ₐOR³³, aryl, C₃-C₇ cycloalkyl, a three to twelve membered heterocyclic ring having up to 3 heteroatoms which is optionally substituted from 1 to 3 substituents independently selected from halo, C₁-C₆ alkyl, and —(C₁-C₆ alkyl)-O—(C₁-C₆ alkyl);
R³¹ and R³² are independently selected from the group consisting of H, C₁-C₈ alkyl, C₂-C₈ alkenyl, C₂-C₈ alkynyl, and —(C₁-C₆ alkyl)-O—(C₁-C₆ alkyl);
or R³¹ and R³² may be taken together to form a three to twelve membered heterocyclic ring having up to 3 heteroatoms which is optionally substituted from 1 to 3 substituents independently selected from halo and C₁-C₆ alkyl;
R³³ and R³⁴ are independently selected from the group consisting of H and C₁-C₈ alkyl;
a is selected from 0 to 6;
R⁵ is selected from H and C₁-C₆ alkyl;
R⁶ is selected from the group consisting of H, halo, and C₁-C₆ alkyl.

5. The method of claim 1, wherein the rho kinase inhibitor is a compound of Formula XVII: 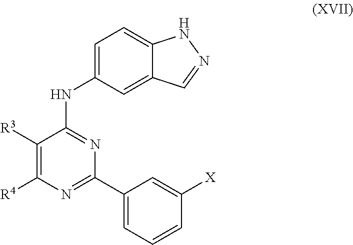
or pharmaceutically acceptable salt thereof, wherein:
X is selected from the group consisting of —NH—C(═O)—CHR¹³R¹⁴; —NH—C(═O)—(CH₂)_{b}—NR¹³R¹⁴; —C(═O)NR¹³R¹⁴;
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), aryl, heteroaryl, C₃-C₇ cycloalkyl, a three to twelve membered heterocyclic ring containing up to 3 heteroatoms, each of which may be optionally substituted by from 1 to 3 substituents independently selected from halo, oxo, C₁-C₆ alkyl, C₂-C₆, alkenyl, C₃-C₇ cycloalkyl, C₁-C₆ alkoxy, CN and C₁-C₃ perfluoro alkyl;
each R³ and R⁴ is independently selected from the group consisting of H, C₁-C₈ alkyl, —CN, halo, —OH, —O—(C₁-C₆ alkyl), —O—(C₁-C₆ alkyl)-O—(C₁-C₆ alkyl), —NR³¹R³², C₁-C₃ perfluoro alkyl, —O—(CH₂)ₐNR³¹R³², aryl, C₃-C₇ cycloalkyl, a three to twelve membered heterocyclic ring having up to 3 heteroatoms which is optionally substituted from 1 to 3 substituents independently selected from halo and C₁-C₆ alkyl;
R³¹ and R³² are independently selected from the group consisting of H, C₁-C₈ alkyl, C₂-C₈ alkenyl, C₂-C₈ alkynyl, and —(C₁-C₆ alkyl)-O—(C₁-C₆ alkyl);
a is selected from 0 to 6;
b is selected from 0 to 1.

6. The method of claim 1, wherein the rho kinase inhibitor is a compound of Formula XVIII: 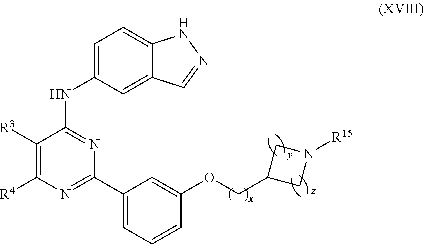
or pharmaceutically acceptable salt thereof, wherein:
each R³ and R⁴ is independently selected from the group consisting of H, C₁-C₈ alkyl, —CN, halo, —OH, —O—(C₁-C₆ alkyl), —O—(C₁-C₆ alkyl)-O—(C₁-C₆ alkyl), —NR³¹R³², C₁-C₃ perfluoro alkyl, —O—(CH₂)ₐNR³¹R³², aryl, C₃-C₇ cycloalkyl, a three to twelve membered heterocyclic ring having up to 3 heteroatoms which is optionally substituted from 1 to 3 substituents independently selected from halo and C₁-C₆ alkyl;
R³¹ and R³² are independently selected from the group consisting of H, C₁-C₈ alkyl, C₂-C₈ alkenyl, C₂-C₈ alkynyl, and —(C₁-C₆ alkyl)-O—(C₁-C₆ alkyl);
a is selected from 0 to 6;
R¹⁵ is selected from the group consisting of H, C₁-C₈ alkyl, —CN, halo, —OH, —O—(C₁-C₆ alkyl), —O—(C₁-C₆ alkyl)-O—(C₁-C₆ alkyl), —C(═O)—O—C(R)₃₃₁, C₁-C₃ perfluoro alkyl, C₃-C₇ cycloalkyl, a three to twelve membered heterocyclic or aromatic ring having up to 3 heteroatoms which is optionally substituted from 1 to 3 substituents independently selected from halo and C₁-C₆ alkyl;
x is selected from 1 to 3;
y is selected from 0 to 3;
z is selected from 0 to 3;
wherein y or z are independently selected and one of which is at least 1.

7. The method of claim 1, wherein the rho kinase inhibitor is a compound of Formula XIX: 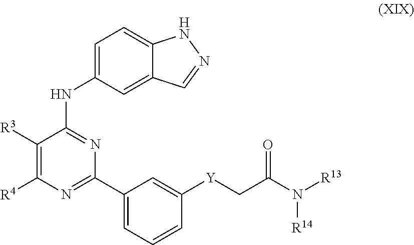
or pharmaceutically acceptable salt thereof, wherein:
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), heteroaryl, C₃-C₇ cycloalkyl, a three to twelve membered heterocyclic or aromatic ring containing up to 3 heteroatoms, each of which may be optionally substituted by from 1 to 3 substituents independently selected from halo, oxo, C₁-C₆ alkyl, C₂-C₆, alkenyl, C₃-C₇ cycloalkyl, C₁-C₆ alkoxy, CN and C₁-C₃ perfluoro alkyl;
or R¹³ and R¹⁴ may be taken together form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, —OH, —NH₂, CN and C₁-C₃ perfluoro alkyl;
Y is selected from the group consisting of S, CH₂, and —CR³¹R³²—
R² is selected from H and halo;
each R³ and R⁴ is independently selected from the group consisting of H, C₁-C₈ alkyl, —CN, halo, —OH, —O—(C₁-C₆ alkyl), —O—(C₁-C₆ alkyl)-O—(C₁-C₆ alkyl), —NR³¹R³², C₁-C₃ perfluoro alkyl, —O—(CH₂)ₐNR³¹R³², —NR³¹—(CH₂)ₐNR³³R³⁴, —NR³¹—(CH₂)ₐOR³³, aryl, C₃-C₇ cycloalkyl, a three to twelve membered heterocyclic ring having up to 3 heteroatoms which is optionally substituted from 1 to 3 substituents independently selected from halo, C₁-C₆ alkyl, and —(C₁-C₆ alkyl)-O—(C₁-C₆ alkyl);
R³¹ and R³² are independently selected from the group consisting of H, halo, C₁-C₈ alkyl, C₂-C₈ alkenyl, C₂-C₈ alkynyl, and —(C₁-C₆ alkyl)-O—(C₁-C₆ alkyl);
or R³¹ and R³² may be taken together to form a three to twelve membered cycloalkyl or heterocyclic ring having up to 3 heteroatoms which is optionally substituted from 1 to 3 substituents independently selected from halo and C₁-C₆ alkyl;
R³³ and R³⁴ are independently selected from the group consisting of H and C₁-C₈ alkyl;
a is selected from 0 to 6.

8. The method of claim 1, wherein the rho kinase inhibitor is a compound of Formula XX: 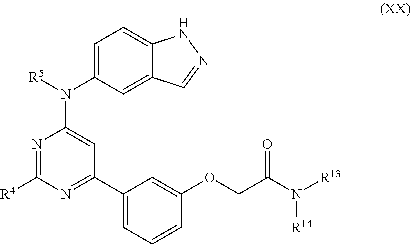
or pharmaceutically acceptable salt thereof, wherein:
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), heteroaryl, C₃-C₇ cycloalkyl, a three to twelve membered heterocyclic or aromatic ring containing up to 3 heteroatoms, each of which may be optionally substituted by from 1 to 3 substituents independently selected from halo, oxo, C₁-C₆ alkyl, C₂-C₆, alkenyl, C₃-C₇ cycloalkyl, C₁-C₆ alkoxy, CN and C₁-C₃ perfluoro alkyl;
or R¹³ and R¹⁴ may be taken together form a three to twelve membered heterocyclic ring having up to 3 heteroatoms which is optionally substituted by from 1 to 3 substituents independently selected from halo, C₁-C₆ alkyl, C₂-C₆, alkenyl, C₁-C₆ alkoxy, C₃-C₇ cycloalkyl, oxo, —OH, —NH₂, CN and C₁-C₃ perfluoro alkyl;
R⁴ is selected from the group consisting of H, C₁-C₃ alkyl, —CN, halo, —OH, —O—(C₁-C₆ alkyl), —O—(C₁-C₆ alkyl)-O—(C₁-C₆ alkyl), —NR³¹R³², C₁-C₃ perfluoro alkyl, —O—(CH₂)ₐNR³¹R2, aryl, C₃-C₇ cycloalkyl, a three to twelve membered heterocyclic ring having up to 3 heteroatoms which is optionally substituted from 1 to 3 substituents independently selected from halo and C₁-C₆ alkyl;
R³¹ and R³² are independently selected from the group consisting of H, C₁-C₈ alkyl, C₂-C₈ alkenyl, C₂-C₈ alkynyl, and —(C₁-C₆ alkyl)-O—(C₁-C₆ alkyl);
or R³¹ and R³² may be taken together to form a three to twelve membered heterocyclic ring having up to 3 heteroatoms which is optionally substituted from 1 to 3 substituents independently selected from halo and C₁-C₆ alkyl;
R⁵ is selected from H and C₁-C₆ alkyl.

9. The method of claim 1, wherein the rho kinase inhibitor is a compound of Formula XXI: 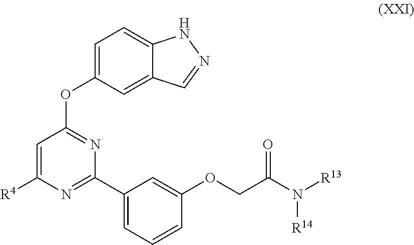
or pharmaceutically acceptable salt thereof, wherein:
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), heteroaryl, C₃-C₇ cycloalkyl, a three to twelve membered heterocyclic or aromatic ring containing up to 3 heteroatoms, each of which may be optionally substituted by from 1 to 3 substituents independently selected from halo, oxo, C₁-C₆ alkyl, C₂-C₆, alkenyl, C₃-C₇ cycloalkyl, C₁-C₆ alkoxy, CN and C₁-C₃ perfluoro alkyl;
or R¹³ and R¹⁴ may be taken together form a three to twelve membered heterocyclic ring having up to 3 heteroatoms which is optionally substituted by from 1 to 3 substituents independently selected from halo, C₁-C₆ alkyl, C₂-C₆, alkenyl, C₁-C₆ alkoxy, C₃-C₇ cycloalkyl, oxo, —OH, —NH₂, CN and C₁-C₃ perfluoro alkyl;
R⁴ is selected from the group consisting of H, C₁-C₈ alkyl, —CN, halo, —OH, —O—(C₁-C₆ alkyl), —O—(C₁-C₆ alkyl)-O—(C₁-C₆ alkyl), —NR³¹R³², C₁-C₃ perfluoro alkyl, —O—(CH₂)ₐNR³¹R³², aryl, C₃-C₇ cycloalkyl, a three to twelve membered heterocyclic ring having up to 3 heteroatoms which is optionally substituted from 1 to 3 substituents independently selected from halo and C₁-C₆ alkyl;
R³¹ and R³² are independently selected from the group consisting of H, C₁-C₈ alkyl, C₂-C₈ alkenyl, C₂-C₈ alkynyl, and —(C₁-C₆ alkyl)-O—(C₁-C₆ alkyl);
or R³¹ and R³² may be taken together to form a three to twelve membered heterocyclic ring having up to 3 heteroatoms which is optionally substituted from 1 to 3 substituents independently selected from halo and C₁-C₆ alkyl;
a is selected from 0 to 6.

10. The method of claim 1, wherein the rho kinase inhibitor is a compound of Formula XXII: 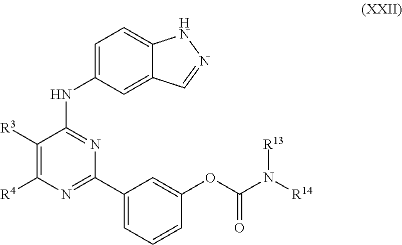
or pharmaceutically acceptable salt thereof, wherein:
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), heteroaryl, C₃-C₇ cycloalkyl, a three to twelve membered heterocyclic or aromatic ring containing up to 3 heteroatoms, each of which may be optionally substituted by from 1 to 3 substituents independently selected from halo, oxo, C₁-C₆ alkyl, C₂-C₆, alkenyl, C₃-C₇ cycloalkyl, C₁-C₆ alkoxy, CN and C₁-C₃ perfluoro alkyl;
or R¹³ and R¹⁴ may be taken together form a three to twelve membered heterocyclic ring having up to 3 heteroatoms which is optionally substituted by from 1 to 3 substituents independently selected from halo, C₁-C₆ alkyl, C₂-C₆, alkenyl, C₁-C₆ alkoxy, C₃-C₇ cycloalkyl, oxo, —OH, —NH₂, CN and C₁-C₃ perfluoro alkyl;
R³ is H;
R⁴ is selected from the group consisting of H, C₁-C₈ alkyl, —CN, halo, —OH, —O—(C₁-C₆ alkyl), —O—(C₁-C₆ alkyl)-O—(C₁-C₆ alkyl), —NR³¹R³², C₁-C₃ perfluoro alkyl, —O—(CH₂)ₐNR³¹R³², aryl, C₃-C₇ cycloalkyl, a three to twelve membered heterocyclic ring having up to 3 heteroatoms which is optionally substituted from 1 to 3 substituents independently selected from halo and C₁-C₆ alkyl;
R³¹ and R³² are independently selected from the group consisting of H, C₁-C₈ alkyl, C₂-C₈ alkenyl, C₂-C₈ alkynyl, and —(C₁-C₆ alkyl)-O—(C₁-C₆ alkyl);
or R³¹ and R³² may be taken together to form a three to twelve membered heterocyclic ring having up to 3 heteroatoms which is optionally substituted from 1 to 3 substituents independently selected from halo and C₁-C₆ alkyl;
a is selected from 0 to 6.

11. The method of claim 1, wherein the rho kinase inhibitor is a compound of Formula XXIII: 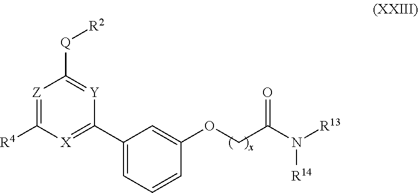
or pharmaceutically acceptable salt thereof, wherein:
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), heteroaryl, C₃-C₇ cycloalkyl, a three to twelve membered heterocyclic or aromatic ring containing up to 3 heteroatoms, each of which may be optionally substituted by from 1 to 3 substituents independently selected from halo, oxo, C₁-C₆ alkyl, C₂-C₆, alkenyl, C₃-C₇ cycloalkyl, C₁-C₆ alkoxy, CN and C₁-C₃ perfluoro alkyl;
or R¹³ and R¹⁴ may be taken together form a three to twelve membered heterocyclic ring having up to 3 heteroatoms which is optionally substituted by from 1 to 3 substituents independently selected from halo, C₁-C₆ alkyl, C₂-C₆, alkenyl, C₁-C₆ alkoxy, C₃-C₇ cycloalkyl, oxo, —OH, —NH₂, CN and C₁-C₃ perfluoro alkyl;
x is selected from 0 to 1;
R² is selected from the group consisting of cyclohexylpyridine, 1H-pyrazole, and pyridine; 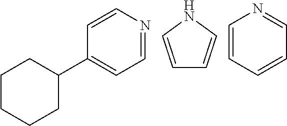
X is selected from N or CR³;
Y is selected from N or CR³;
Z is selected from N or CR⁴;
wherein at least one of X, Y, and Z is N;
R⁴ is selected from the group consisting of H, C₁-C₈ alkyl, —CN, halo, —OH, —O—(C₁-C₆ alkyl), —O—(C₁-C₆ alkyl)-O—(C₁-C₆ alkyl), —NR³¹R³², C₁-C₃ perfluoro alkyl, —O—(CH₂)ₐNR³¹R³², NR³¹—(CH₂)ₐNR³³R³⁴, —NR³¹—(CH₂)ₐOR³³, aryl, C₃-C₇ cycloalkyl, a three to twelve membered heterocyclic ring having up to 3 heteroatoms which is optionally substituted from 1 to 3 substituents independently selected from halo, C₁-C₆ alkyl, and —(C₁-C₆ alkyl)-O—(C₁-C₆ alkyl);
R³¹ and R³² are independently selected from the group consisting of H, C₁-C₈ alkyl, C₂-C₈ alkenyl, C₂-C₈ alkynyl, and —(C₁-C₆ alkyl)-O—(C₁-C₆ alkyl);
or R³¹ and R³² may be taken together to form a three to twelve membered heterocyclic ring having up to 3 heteroatoms which is optionally substituted from 1 to 3 substituents independently selected from halo and C₁-C₆ alkyl;
a is selected from 0 to 6;
Q is selected from the group NR⁵ and 0;
R⁵ is selected from H and C₁-C₆ alkyl;

12. The method of claim 1, wherein the rho kinase inhibitor is a compound of Formula XXIV: 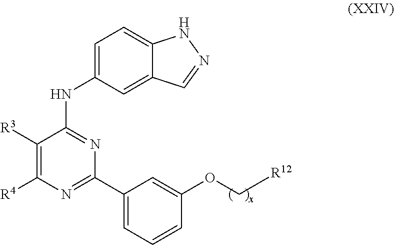
or pharmaceutically acceptable salt thereof, wherein:
R¹² is selected from the group consisting of H, C₁-C₈ alkyl, C₂-C₈ alkenyl, C₂-C₈ alkynyl, —(C₁-C₆ alkyl)-O—(C₁-C₆ alkyl), amino, NR³¹R³², heteroaryl, C₃-C₇ cycloalkyl, a three to twelve membered heterocyclic or aromatic ring containing up to 3 heteroatoms, each of which may be optionally substituted by from 1 to 3 substituents independently selected from halo, oxo, C₁-C₆ alkyl, C₂-C₆, alkenyl, C₃-C₇ cycloalkyl, C₁-C₆ alkoxy, CN and C₁-C₃ perfluoro alkyl;
x is selected from 0 to 2;
each R³ and R⁴ is independently selected from the group consisting of H, C₁-C₈ alkyl, —CN, halo, —OH, —O—(C₁-C₆ alkyl), —O—(C₁-C₆ alkyl)-O—(C₁-C₆ alkyl), —NR³¹R³², C₁-C₃ perfluoro alkyl, —O—(CH₂)ₐNR³¹R³², aryl, C₃-C₇ cycloalkyl, a three to twelve membered heterocyclic ring having up to 3 heteroatoms which is optionally substituted from 1 to 3 substituents independently selected from halo and C₁-C₆ alkyl;
R³¹ and R³² are independently selected from the group consisting of H, C₁-C₈ alkyl, C₂-C₈ alkenyl, C₂-C₈ alkynyl, C₃-C₇ cycloalkyl and C₃-C₇ cycloalkyl —(C₁-C₆ alkyl)-O—(C₁-C₆ alkyl);
or R³¹ and R³² may be taken together to form a three to twelve membered heterocyclic ring having up to 3 heteroatoms which is optionally substituted from 1 to 3 substituents independently selected from halo and C₁-C₆ alkyl;
a is selected from 1 to 6.

13. The method of claim 1, wherein the rho kinase inhibitor is a compound of Formula XXV: 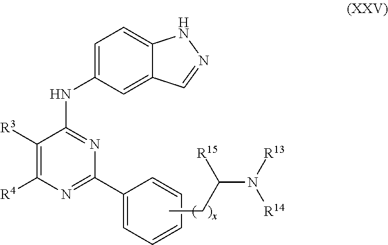
or pharmaceutically acceptable salt thereof, wherein:
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), heteroaryl, C₃-C₇ cycloalkyl, a three to twelve membered heterocyclic or aromatic ring containing up to 3 heteroatoms, each of which may be optionally substituted by from 1 to 3 substituents independently selected from halo, oxo, C₁-C₆ alkyl, C₂-C₆, alkenyl, C₃-C₇ cycloalkyl, C₁-C₆ alkoxy, CN and C₁-C₃ perfluoro alkyl;
or R¹³ and R¹⁴ may be taken together form a three to twelve membered heterocyclic ring having up to 3 heteroatoms which is optionally substituted by from 1 to 3 substituents independently selected from halo, C₁-C₆ alkyl, C₂-C₆, alkenyl, C₁-C₆ alkoxy, C₃-C₇ cycloalkyl, oxo, —OH, —NH₂, CN and C₁-C₃ perfluoro alkyl;
x is selected from 0 to 3;
R¹⁵ is selected from the group consisting of H, C₁-C₈ alkyl, C₂-C₈ alkenyl, C₂-C₈ alkynyl, aryl, heteroaryl, heterocyclic ring, and C₃-C₇ cycloalkyl;
each R³ and R⁴ is independently selected from the group consisting of H, C₁-C₈ alkyl, —CN, halo, —OH, —O—(C₁-C₆ alkyl), —O—(C₁-C₆ alkyl)-O—(C₁-C₆ alkyl), —NR³¹R³², C₁-C₃ perfluoro alkyl, —O—(CH₂)ₐNR³¹R³², aryl, C₃-C₇ cycloalkyl, a three to twelve membered heterocyclic ring having up to 3 heteroatoms which is optionally substituted from 1 to 3 substituents independently selected from halo and C₁-C₆ alkyl;
R³¹ and R³² are independently selected from the group consisting of H, C₁-C₈ alkyl, C₂-C₈ alkenyl, C₂-C₈ alkynyl, and —(C₁-C₆ alkyl)-O—(C₁-C₆ alkyl);
or R³¹ and R³² may be taken together to form a three to twelve membered heterocyclic ring having up to 3 heteroatoms which is optionally substituted from 1 to 3 substituents independently selected from halo and C₁-C₆ alkyl;
a is selected from 1 to 6.

14. The method of claim 1, wherein the angiogenesis inhibitor is a VEGR antagonist.

15. The method of claim 1, wherein the angiogenesis inhibitor is an antibody or antigen binding fragment thereof that binds to VEGFR2.

16. The method of claim 15, wherein the VEGFR2 antibody blocks ligand binding.

17. The method of claim 15, wherein the VEGR2 antibody inhibits VEGFR2 activation.

18. The method of claim 15, wherein the antibody comprises a CDR-1H, CDR-2H, and CDR-3H sequence, wherein:
(i) the CDR-1H sequence is GFTFSWYX₁MX₂ (SEQ ID NO:185), wherein X₁ is V or I, X₂ is G or L,
(ii) the CDR-2H sequence is SIX₁X₂SGGX₃TX₄YADSVKG (SEQ ID NO:186), wherein X₁ is Y or G, X₂ is P or S, X₃ is A or F, X₄ is N or D, and
(iii) the CDR-3H sequence is GNYFDY (SEQ ID NO:3) or GLAAPRS (SEQ ID NO:11).

19. The method of claim 15, wherein the antibody comprises a CDR-1L, CDR-2L, and CDR-3L, wherein
(i) the CDR-1L sequence is X₁GX₂X₃LX₄X₅X₆X₇X₈S (SEQ ID NO:187), wherein X₁ is S, Q, or T, X₂ is D, E, or Q, X₃ is K, S, N, I, or A, X₄ is G or R, X₅ is D, S, H, E, or N, X₆ is E, Y, Q, R, or N, X₇ is Y, F, or S, and X₈ is A or S, or SGSX₁SNX₂X₃X₄X₅X₆X₇X₈ (SEQ ID NO: 188), wherein X₁ is S, or T, X₂ is I or L, X₃ is E or G, X₄ is T, S, or N, X₅ is N or Y, X₆ is T. P, A, or Y, X₇ is V or L, and X₈ is N, I, or Y, or X₁GX₂SX₃DX₄GX₅YDYVS (SEQ ID NO:189), wherein X₁ is A or T, X₂ is S or T, X₃ is H, S, or N, X₄ is I or V, and X₅ is S or A,
(ii) the CDR-2L sequence is X₁X₂X₃X₄X₅PS (SEQ ID NO:190), wherein X₁ is Q, D, T, Y, S, or A, X₂ is D, N, S, T, V, or V, X₃ is D, N, S, T, or Y, X₄ is Q, K, N, or L, and X₅ is R or L, and
(iii) the CDR-3L sequence is QX₁WX₂X₃X₄X₅X₆X₇X₈ (SEQ ID NO:191), wherein X₁ is A or T, X₂ is D or G, X₃ is R or no amino acid, X₄ is S, F, on N, X₅ is S, T, on N, X₆ is S, T, or P, X₇ is A, V, L, I, or Y, and X₈ is V or L, or AX₁WDDX₂LX₃X₄X₅X₆ (SEQ ID NO:192), wherein X₁ is A, S, or T, X₂ is N or S, X₃ is N, I, or G, X₄ is G or S, X₅ is P, W, or V, and X₆ is V or L, or MYSTITX₁LL (SEQ ID NO:193), wherein X₁ is A or T.

20. The method of claim 15, wherein the antibody comprises a CDR-1L, CDR-2L, and CDR-3L, wherein
(i) the CDR-1L sequence is RASX₁X₂X₃X₄X₅X₆X₇YX₈X₉ (SEQ ID NO:194), wherein X₁ is Q, E, or H, X₂ is S, R, or N, X₃ is V, I, or L, X₄ is S, R, G or N, X₅ is S or N, X₆ is S, N, W, or D, X₇ is G or no amino acid, X₈ is L or F, and X₉ is A, G, M, or S,
(ii) the CDR-2L sequence is GASX₁RAT (SEQ ID NO:195), wherein X₁ is S, T, I, or N, and
(iii) the CDR-3L sequence is QQX₁X₂X₃X₄X₅X₆X₇X₈ (SEQ ID NO:196), wherein X₁ is F or Y, X₂ is D, G, or Y, X₃ is S, T, or N, X₄ is S, L, or W, X₅ is P or no amino acid, X₆ is P or T, X₇ is L, I, V, P, W, or Y, and X₈ is T or S.

21. The method of claim 15, wherein the antibody comprises a CDR-1H having SEQ ID NO:1, a CDR-2L having SEQ ID NO:2, and a CDR-3L having SEQ ID NO:3.

22. The method of claim 15, wherein the antibody comprises a CDR-1L having SEQ ID NO:5, a CDR-2L having SEQ ID NO:6, and a CDR-3L having SEQ ID NO:7.

23. The method of claim 15, wherein the antibody comprises a heavy chain variable domain having SEQ ID NO:4.

24. The method of claim 15, wherein the antibody comprises a light chain variable domain having SEQ ID NO:8.

25. The method of claim 15, wherein the antibody comprises a CDR-1H having SEQ ID NO:9, a CDR-2L having SEQ ID NO:10, and a CDR-3L having SEQ ID NO:11.

26. The method of claim 15, wherein the antibody comprises a CDR-1L having SEQ ID NO:13, a CDR-2L having SEQ ID NO:14, and a CDR-3L having SEQ ID NO:15.

27. The method of claim 15, wherein the antibody comprises a heavy chain variable domain having SEQ ID NO: 12.

28. The method of claim 15, wherein the antibody comprises a light chain variable domain having SEQ ID NO:16.

29. The method of claim 15, wherein the antibody comprises a CDR-1L having SEQ ID NO:25, a CDR-2L having SEQ ID NO:26, and a CDR-3L having SEQ ID NO:27.

30. The method of claim 15, wherein the antibody comprises a light chain variable domain having SEQ ID NO:28.

31. The method of claim 15, wherein the antibody comprises a CDR-1L having SEQ ID NO:29, a CDR-2L having SEQ ID NO:30, and a CDR-3L having SEQ ID NO:31.

32. The method of claim 15, wherein the antibody comprises a light chain variable domain having SEQ ID NO:32.

33. The method of claim 1, which further comprises administration of a TGF-β antagonist.

34. A method of treating a disorder having an angiogenic component in a subject, which comprises administering to the subject an effective amount of a rho kinase inhibitor and an angiogenesis inhibitor.

35. The method of claim 34, wherein the disorder is atherosclerosis, rheumatoid arthritis (RA), hemangiomas, angiofibromas, psoriasis, corneal graft rejection, insulin-dependent diabetes mellitus, multiple sclerosis, myasthenia gravis, Chron's disease, autoimmune nephritis, primary biliary cirrhosis, acute pancreatitis, allograph rejection, allergic inflammation, contact dermatitis, delayed type hypersensitivity, inflammatory bowel disease, septic shock, osteoporosis, osteoarthritis, neuronal inflammation, Osler-Weber syndrome, restenosis, or fungal, parasitic or viral infection.

36. The method of claim 34, wherein the rho kinase inhibitor is ROCK2 selective.


SEQUENCE LISTING (196 sequences)

SEQ ID NO: 1 - protein, 7 aa, Artificial Sequence
  Human antibody library
GFTFSWY

SEQ ID NO: 2 - protein, 7 aa, Artificial Sequence
  Human antibody library
IYPSGGA

SEQ ID NO: 3 - protein, 6 aa, Artificial Sequence
  Human antibody library
GNYFDY

SEQ ID NO: 4 - protein, 115 aa, Artificial Sequence
  Human antibody library
EVQLLESGGGLVQPGGSLRLSCAASGFTFSWYVMGWVRQAPGKGLEWVSSIYPSGGATNYADSVKGRFTISRDNSKNTLYLQMNSLRAEDTAVYYCARGNYFDYWGQGTLVTVSS

SEQ ID NO: 5 - protein, 11 aa, Artificial Sequence
  Human antibody library
QGDSLRSYYAS

SEQ ID NO: 6 - protein, 7 aa, Artificial Sequence
  Human antibody library
QDTNRPS

SEQ ID NO: 7 - protein, 9 aa, Artificial Sequence
  Human antibody library
QAWDSNTAV

SEQ ID NO: 8 - protein, 109 aa, Artificial Sequence
  Human antibody library
QSVLTQDPAVSVALGQTVRITCQGDSLRSYYASWYQQKPGQSPLVVIYQDTNRPSGIPERFSGSNSGNTATLTISETQAMDEADYYCQAWDSNTAVFGGGTKLTVLGQP

SEQ ID NO: 9 - protein, 7 aa, Artificial Sequence
  Human antibody library
GFTFSWY

SEQ ID NO: 10 - protein, 6 aa, Artificial Sequence
  Human antibody library
GSSGGF

SEQ ID NO: 11 - protein, 7 aa, Artificial Sequence
  Human antibody library
GLAAPRS

SEQ ID NO: 12 - protein, 116 aa, Artificial Sequence
  Human antibody library
EVQLLESGGGLVQPGGSLRLSCAASGFTFSWYIMLWVRQAPGKGLEWVSSIGSSGGFTDYADSVKGRFTISRDNSKNTLYLQMNSLRAEDTAVYYCARGLAAPRSWGRGTLVTVSS

SEQ ID NO: 13 - protein, 13 aa, Artificial Sequence
  Human antibody library
SGSTSNIGNNAVI

SEQ ID NO: 14 - protein, 7 aa, Artificial Sequence
  Human antibody library
YDDLLPS

SEQ ID NO: 15 - protein, 11 aa, Artificial Sequence
  Human antibody library
ASWDDNLNGPL

SEQ ID NO: 16 - protein, 113 aa, Artificial Sequence
  Human antibody library
QSALTQPPSVSEAPGQRVTISCSGSTSNIGNNAVIWYQQLPGKAPKLLIYYDDLLPSGVSDRFSGSKSGTSGSLAISGLQSEDEADYYCASWDDNLNGPLFGGGTKLTVLRQP

SEQ ID NO: 17 - protein, 13 aa, Artificial Sequence
  Human antibody library
SGSSSNIGTYPVN

SEQ ID NO: 18 - protein, 7 aa, Artificial Sequence
  Human antibody library
STDQRPS

SEQ ID NO: 19 - protein, 9 aa, Artificial Sequence
  Human antibody library
QAWDSSTVV

SEQ ID NO: 20 - protein, 111 aa, Artificial Sequence
  Human antibody library
QSALTQPPSASGTPGQRVTISCSGSSSNIGTYPVNWYQQLPGAAPKLLIYSTDQRPSGVPDRFSGSNSGNTATLTISGTQAMDEADYYCQAWDSSTVVFGGGTKLTVLGQP

SEQ ID NO: 21 - protein, 11 aa, Artificial Sequence
  Human antibody library
SGDKLGDEYAS

SEQ ID NO: 22 - protein, 7 aa, Artificial Sequence
  Human antibody library
QDNKRPS

SEQ ID NO: 23 - protein, 9 aa, Artificial Sequence
  Human antibody library
QAWDSSTVV

SEQ ID NO: 24 - protein, 109 aa, Artificial Sequence
  Human antibody library
QSALTQPPSVSVSPGQTASITCSGDKLGDEYASWYQQKPGQSPVLVIYQDNKRPSGIPERFSGSNSGNTATLTISGTQAMDEADYYCQAWDSSTVVFGGGTKLTVLGQP

SEQ ID NO: 25 - protein, 11 aa, Artificial Sequence
  Human antibody library
SGDNLRHEYSS

SEQ ID NO: 26 - protein, 7 aa, Artificial Sequence
  Human antibody library
QDSKRPS

SEQ ID NO: 27 - protein, 9 aa, Artificial Sequence
  Human antibody library
QAWGSSTVV

SEQ ID NO: 28 - protein, 109 aa, Artificial Sequence
  Human antibody library
QYELTQPPSVSVSPGQTASITCSGDNLRHEYSSWYQQRPGQSPVLVIYQDSKRPSGIPERFSGSNSGNTATLTISGTQALDEADYYCQAWGSSTVVFGGGTKLTVLRQP

SEQ ID NO: 29 - protein, 11 aa, Artificial Sequence
  Human antibody library
SGEKLGDEYAS

SEQ ID NO: 30 - protein, 7 aa, Artificial Sequence
  Human antibody library
QDNKRPS

SEQ ID NO: 31 - protein, 9 aa, Artificial Sequence
  Human antibody library
QAWDSSTLL

SEQ ID NO: 32 - protein, 109 aa, Artificial Sequence
  Human antibody library
QSVLTQPPSVSVSPGQTASITCSGEKLGDEYASWYQQKPGQSPVLVIYQDNKRPSGIPERFSGSNSGNTATLTISGTQAMDEADYYCQAWDSSTLLFGGGTKLTVLGQP

SEQ ID NO: 33 - protein, 11 aa, Artificial Sequence
  from human Fab library
SGEKLGDEYAS

SEQ ID NO: 34 - protein, 7 aa, Artificial Sequence
  from human Fab library
QDNKRPS

SEQ ID NO: 35 - protein, 9 aa, Artificial Sequence
  from human Fab library
QAWDSSTLL

SEQ ID NO: 36 - protein, 109 aa, Artificial Sequence
  from human Fab library
QSELTQPPSVSVSPGQTASITCSGEKLGDEYASWYQQKPGQSPVLVIYQDNKRPSGIPERFSGSNSGNTATLTISGTQAMDEADYYCQAWDSSTLLFGGGTKLTVLGQP

SEQ ID NO: 37 - protein, 11 aa, Artificial Sequence
  Human antibody library
TGDKLGDQFAS

SEQ ID NO: 38 - protein, 7 aa, Artificial Sequence
  Human antibody library
QNDKRPS

SEQ ID NO: 39 - protein, 9 aa, Artificial Sequence
  Human antibody library
QAWDFSSAL

SEQ ID NO: 40 - protein, 109 aa, Artificial Sequence
  Human antibody library
QYELTQPPSVSVSPGQTATITCTGDKLGDQFASWYQHKPGQSPILLIYQNDKRPSGIPDRFSGSDSGNTATLTISGTQAMDEAHYYCQAWDFSSALFGGGTKLTVLGQP

SEQ ID NO: 41 - protein, 11 aa, Artificial Sequence
  Human antibody library
SGQILGERSAS

SEQ ID NO: 42 - protein, 7 aa, Artificial Sequence
  Human antibody library
QSSQRPS

SEQ ID NO: 43 - protein, 8 aa, Artificial Sequence
  Human antibody library
QTWDTSIL

SEQ ID NO: 44 - protein, 108 aa, Artificial Sequence
  Human antibody library
QSALTQPPSVSVSPGHTATITCSGQILGERSASWYQQRPGQAPVLVLYQSSQRPSGIPERFSGSISGNTATLTISGAQSIDEADYYCQTWDTSILFGGGTKLTVLSQP

SEQ ID NO: 45 - protein, 11 aa, Artificial Sequence
  Human antibody library
SGDALGNNYAS

SEQ ID NO: 46 - protein, 7 aa, Artificial Sequence
  Human antibody library
QDTKRPS

SEQ ID NO: 47 - protein, 10 aa, Artificial Sequence
  Human antibody library
QTWDRNTPYV

SEQ ID NO: 48 - protein, 110 aa, Artificial Sequence
  Human antibody library
QSALTQPPSVSVSPGQTAIITCSGDALGNNYASWYQQKPGQSPVLVIYQDTKRPSGIPERFSGSSSGNTATLTISETQTMDEADYYCQTWDRNTPYVFGAGTKVTVLGQP

SEQ ID NO: 49 - protein, 13 aa, Artificial Sequence
  Human antibody library
SGSSSNIGTNTLN

SEQ ID NO: 50 - protein, 7 aa, Artificial Sequence
  Human antibody library
ANNQRPS

SEQ ID NO: 51 - protein, 11 aa, Artificial Sequence
  Human antibody library
ATWDDSLIGPV

SEQ ID NO: 52 - protein, 113 aa, Artificial Sequence
  Human antibody library
QSALTQPPSVSGTPGQRVTISCSGSSSNIGTNTLNWYQQLPGTAPKLLIYANNQRPSGVPDRFSGSRSGTSASLAISGLQSDDEADYYCATWDDSLIGPVFGGGTKLTVLGQP

SEQ ID NO: 53 - protein, 13 aa, Artificial Sequence
  from human Fab library
SGSTSNIGNNAVI

SEQ ID NO: 54 - protein, 7 aa, Artificial Sequence
  from human Fab library
YDDLLPS

SEQ ID NO: 55 - protein, 11 aa, Artificial Sequence
  from human Fab library
ASWDDNLNGPL

SEQ ID NO: 56 - protein, 113 aa, Artificial Sequence
  from human Fab library
QYELTQPPSVSEAPGQRVTISCSGSTSNIGNNAVIWYQQLPGKAPKLLIYYDDLLPSGVSDRFSGSKSGTSGSLAISGLQSEDEADYYCASWDDNLNGPLFGGGTKLTVLRQP

SEQ ID NO: 57 - protein, 13 aa, Artificial Sequence
  Human antibody library
SGSSSNLGSNTVN

SEQ ID NO: 58 - protein, 7 aa, Artificial Sequence
  Human antibody library
TNSQRPS

SEQ ID NO: 59 - protein, 11 aa, Artificial Sequence
  Human antibody library
AAWDDSLNGWV

SEQ ID NO: 60 - protein, 113 aa, Artificial Sequence
  Human antibody library
QSELTQPPSASGTPGQRVTISCSGSSSNLGSNTVNWYQQLPGTAPKLLIYTNSQRPSGVPDRFSGLQSGTSASLAISGLQSEDEADYYCAAWDDSLNGWVFGGGTKLTVLSQP

SEQ ID NO: 61 - protein, 13 aa, Artificial Sequence
  Human antibody library
SGSSSNIESNYVY

SEQ ID NO: 62 - protein, 7 aa, Artificial Sequence
  Human antibody library
TNNQRPS

SEQ ID NO: 63 - protein, 11 aa, Artificial Sequence
  Human antibody library
ASWDDSLSGVV

SEQ ID NO: 64 - protein, 113 aa, Artificial Sequence
  Human antibody library
QSVLTQPPSASGTPGQRVTISCSGSSSNIESNYVYWYQQLPGTAPKLLIYTNNQRPSGVPDRFSGSKSGTSASLAISGLRSEDEADYYCASWDDSLSGVVFGGGTKLTVLRQP

SEQ ID NO: 65 - protein, 14 aa, Artificial Sequence
  Human antibody library
TGSSNDIGSYDYVS

SEQ ID NO: 66 - protein, 7 aa, Artificial Sequence
  Human antibody library
DVNNRPS

SEQ ID NO: 67 - protein, 9 aa, Artificial Sequence
  Human antibody library
MSYTITALL

SEQ ID NO: 68 - protein, 112 aa, Artificial Sequence
  Human antibody library
QSELTQPDSVSGSPGQSITISCTGSSNDIGSYDYVSWYQQHPGRAPKFILYDVNNRPSGVADRFSGFKSGNTASLTISGLQPDDEADYFCMSYTITALLFGGGTRVTVLGQP

SEQ ID NO: 69 - protein, 14 aa, Artificial Sequence
  Human antibody library
TGSSHDIGSYDYVS

SEQ ID NO: 70 - protein, 7 aa, Artificial Sequence
  Human antibody library
DVNNRPS

SEQ ID NO: 71 - protein, 9 aa, Artificial Sequence
  Human antibody library
MSYTITTLL

SEQ ID NO: 72 - protein, 112 aa, Artificial Sequence
  Human antibody library
QSALTQPDSVSGSPGQSITISCTGSSHDIGSYDYVSWYQYHPGKAPKFILYDVNNRPSGVSDRFSGSKSGNTASLTISGLQPDDEADYFCMSYTITTLLFGTGTRVTVLSQP

SEQ ID NO: 73 - protein, 14 aa, Artificial Sequence
  Human antibody library
AGTSSDVGAYDYVS

SEQ ID NO: 74 - protein, 7 aa, Artificial Sequence
  Human antibody library
DVYNRPS

SEQ ID NO: 75 - protein, 9 aa, Artificial Sequence
  Human antibody library
MSYTITTLL

SEQ ID NO: 76 - protein, 112 aa, Artificial Sequence
  Human antibody library
QSALTQPASMSGSRGQSITISCAGTSSDVGAYDYVSWYKHLPGNAPKFILYDVYNRPSGVSDRFSGSKSGNTASLTISGLQPDDEADYFCMSYTITTLLFGTGTRVTVLSQP

SEQ ID NO: 77 - protein, 14 aa, Artificial Sequence
  Human antibody library
TGSSHDIGAYDYVS

SEQ ID NO: 78 - protein, 7 aa, Artificial Sequence
  Human antibody library
DVYNRPS

SEQ ID NO: 79 - protein, 9 aa, Artificial Sequence
  Human antibody library
MSYTITTLL

SEQ ID NO: 80 - protein, 112 aa, Artificial Sequence
  Human antibody library
QSVLTQPDSVSGSPGQSITISCTGSSHDIGAYDYVSWYKHLPGNAPKFILYDVYNRPSGVSDRFSGSKSGNTASLTISGLQPDDEADYFCMSYTITTLLFGTGTRVTVLSQP

SEQ ID NO: 81 - protein, 14 aa, Artificial Sequence
  from human Fab library
TGSSHDIGAYDYVS

SEQ ID NO: 82 - protein, 7 aa, Artificial Sequence
  from human Fab library
DVYNRPS

SEQ ID NO: 83 - protein, 9 aa, Artificial Sequence
  from human Fab library
MSYTITTLL

SEQ ID NO: 84 - protein, 112 aa, Artificial Sequence
  from human Fab library
QSVLTQPASVSGSPGQSITISCTGSSHDIGAYDYVSWYKHLPGNAPKFILYDVYNRPSGVSDRFSGSKSGNTASLTISGLQPDDEADYFCMSYTITTLLFGTGTRVTVLSQP

SEQ ID NO: 85 - protein, 14 aa, Artificial Sequence
  from human Fab library
TGSSHDIGAYDYVS

SEQ ID NO: 86 - protein, 7 aa, Artificial Sequence
  from human Fab library
DVYNRPS

SEQ ID NO: 87 - protein, 9 aa, Artificial Sequence
  from human Fab library
MSYTITTLL

SEQ ID NO: 88 - protein, 112 aa, Artificial Sequence
  from human Fab library
QSVLTQPYSVSGSPGQSITISCTGSSHDIGAYDYVSWYKHLPGNAPKFILYDVYNRPSGVSDRFSGSKSGNTASLTISGLQPDDEADYFCMSYTITTLLFGTGTRVTVLSQP

SEQ ID NO: 89 - protein, 14 aa, Artificial Sequence
  Human antibody library
TGSSHDIGAYDYVS

SEQ ID NO: 90 - protein, 7 aa, Artificial Sequence
  Human antibody library
DVYNRPS

SEQ ID NO: 91 - protein, 9 aa, Artificial Sequence
  Human antibody library
MSYTITTLL

SEQ ID NO: 92 - protein, 112 aa, Artificial Sequence
  Human antibody library
QSALTQPDSVSGSPGQSITISCTGSSHDIGAYDYVSWYKHLPGNAPKFILYDVYNRPSGVSDRFSGSKSGNTASLTISGLQPDDEADYFCMSYTITTLLFGTGTRVTVLSQP

SEQ ID NO: 93 - protein, 14 aa, Artificial Sequence
  from human Fab library
TGSSHDIGAYDYVS

SEQ ID NO: 94 - protein, 7 aa, Artificial Sequence
  from human Fab library
DVYNRPS

SEQ ID NO: 95 - protein, 9 aa, Artificial Sequence
  from human Fab library
MSYTITTLL

SEQ ID NO: 96 - protein, 112 aa, Artificial Sequence
  from human Fab library
QSELTQPDSVSGSPGQSITISCTGSSHDIGAYDYVSWYKHLPGNAPKFILYDVYNRPSGVSDRFSGSKSGNTASLTISGLQPDDEADYFCMSYTITTLLFGTGTRVTVLSQP

SEQ ID NO: 97 - protein, 12 aa, Artificial Sequence
  Human antibody library
RASQSVSSSYLA

SEQ ID NO: 98 - protein, 7 aa, Artificial Sequence
  Human antibody library
GASSRAT

SEQ ID NO: 99 - protein, 9 aa, Artificial Sequence
  Human antibody library
QQFDSSPPT

SEQ ID NO: 100 - protein, 109 aa, Artificial Sequence
  Human antibody library
DIQMTQSPGTLSLSPGERATLSCRASQSVSSSYLAWYQQKPGQAPRLLMYGASSRATGFPDRFSGSGSGTDFSLTISRLEPEDFAVYYCQQFDSSPPTFGGGTKVEIKR

SEQ ID NO: 101 - protein, 12 aa, Artificial Sequence
  Human antibody library
RASERISSNYLM

SEQ ID NO: 102 - protein, 7 aa, Artificial Sequence
  Human antibody library
GASIRAT

SEQ ID NO: 103 - protein, 9 aa, Artificial Sequence
  Human antibody library
QQYYSSPLT

SEQ ID NO: 104 - protein, 109 aa, Artificial Sequence
  Human antibody library
DIQMTQSPGTLSVLPGERATLSCRASERISSNYLMWYQQKPGQAPRLLMYGASIRATGIPDRFSGSESGTDFTLTISRVEPEDFAVYYCQQYYSSPLTFGGGTKVEMKR

SEQ ID NO: 105 - protein, 12 aa, Artificial Sequence
  Human antibody library
RASQSISSNYLA

SEQ ID NO: 106 - protein, 7 aa, Artificial Sequence
  Human antibody library
GASSRST

SEQ ID NO: 107 - protein, 9 aa, Artificial Sequence
  Human antibody library
QQFDTLPIT

SEQ ID NO: 108 - protein, 109 aa, Artificial Sequence
  Human antibody library
DIQMTQSPGTLSLSPGERATLSCRASQSISSNYLAWYQQRPGQAPRLLIYGASSRSTGTPDRFSGSGSGTDFTLTISRLEPEDFAIYYCQQFDTLPITFGQGTRLDIKR

SEQ ID NO: 109 - protein, 13 aa, Artificial Sequence
  Human antibody library
RASQSIRSSGYLS

SEQ ID NO: 110 - protein, 7 aa, Artificial Sequence
  Human antibody library
GASTRAT

SEQ ID NO: 111 - protein, 9 aa, Artificial Sequence
  Human antibody library
QQYGSSTIT

SEQ ID NO: 112 - protein, 110 aa, Artificial Sequence
  Human antibody library
DIQMTQSPATLSLSPGERATLSCRASQSIRSSGYLSWFQQKPGQAPRLLIYGASTRATGTPARFSGSGSGTDFTLTIDRLESEDFAVYFCQQYGSSTITFGQGTRLEIKR

SEQ ID NO: 113 - protein, 12 aa, Artificial Sequence
  Human antibody library
RASQSVSSNYLG

SEQ ID NO: 114 - protein, 7 aa, Artificial Sequence
  Human antibody library
GASSRAT

SEQ ID NO: 115 - protein, 9 aa, Artificial Sequence
  Human antibody library
QQFDNLPVT

SEQ ID NO: 116 - protein, 109 aa, Artificial Sequence
  Human antibody library
DIQMTQSPGTLSLSPGERATLSCRASQSVSSNYLGWYQQKPGQAPRLLIYGASSRATGIPDRFSGSGSGTDFTLTISRLEPEDFAVYYCQQFDNLPVTFGGGTKVEMKR

SEQ ID NO: 117 - protein, 12 aa, Artificial Sequence
  Human antibody library
RASQSVSSNYLA

SEQ ID NO: 118 - protein, 7 aa, Artificial Sequence
  Human antibody library
GASSRAT

SEQ ID NO: 119 - protein, 9 aa, Artificial Sequence
  Human antibody library
QQFDTSPLT

SEQ ID NO: 120 - protein, 109 aa, Artificial Sequence
  Human antibody library
DIQMTQSPGTLSLSPGERATLSCRASQSVSSNYLAWYQQKPGQAPRLLIYGASSRATGIPDRFSGSGSGTDFTLTISRLEPEDFAVYYCQQFDTSPLTIGGGTRVDIKR

SEQ ID NO: 121 - protein, 12 aa, Artificial Sequence
  Human antibody library
RASQSVSSNYLA

SEQ ID NO: 122 - protein, 7 aa, Artificial Sequence
  Human antibody library
GASSRAT

SEQ ID NO: 123 - protein, 9 aa, Artificial Sequence
  Human antibody library
QQFDSSPLS

SEQ ID NO: 124 - protein, 109 aa, Artificial Sequence
  Human antibody library
DIQMTQSPGTLSLSPGERATLSCRASQSVSSNYLAWYQQKPGQAPRLLIYGASSRATGIPDRFSGSGSGTDFSLTISRLEPEDSAVYYCQQFDSSPLSFGGGTKVEIKR

SEQ ID NO: 125 - protein, 12 aa, Artificial Sequence
  Human antibody library
RASQSVSSWYLA

SEQ ID NO: 126 - protein, 7 aa, Artificial Sequence
  Human antibody library
GASNRAT

SEQ ID NO: 127 - protein, 9 aa, Artificial Sequence
  Human antibody library
QQFDSSPLT

SEQ ID NO: 128 - protein, 109 aa, Artificial Sequence
  Human antibody library
DIQMTQSPGTLSLSPGERATLSCRASQSVSSWYLAWYQQKPGQAPRLLMYGASNRATGIPDRFSGSGSGTDFTLTISRLEPEDSAVYYCQQFDSSPLTIGGGTKVEIKR

SEQ ID NO: 129 - protein, 12 aa, Artificial Sequence
  Human antibody library
RASQNVGSSYLA

SEQ ID NO: 130 - protein, 7 aa, Artificial Sequence
  Human antibody library
GASSRAT

SEQ ID NO: 131 - protein, 9 aa, Artificial Sequence
  Human antibody library
QQFDSSPPT

SEQ ID NO: 132 - protein, 109 aa, Artificial Sequence
  Human antibody library
DIQMTQSPGTLSLSPGERATLSCRASQNVGSSYLAWYQQKPGQAPRLLMYGASSRATGFPDRFSGSGSGTDFSLTISRLEPEDFAVYYCQQFDSSPPTFGGGTKVEIKR

SEQ ID NO: 133 - protein, 12 aa, Artificial Sequence
  Human antibody library
RASQSVSSSYLA

SEQ ID NO: 134 - protein, 7 aa, Artificial Sequence
  Human antibody library
GASSRAT

SEQ ID NO: 135 - protein, 9 aa, Artificial Sequence
  Human antibody library
QQFDSSPPT

SEQ ID NO: 136 - protein, 109 aa, Artificial Sequence; identical to SEQ ID NO: 100
  Human antibody library

SEQ ID NO: 137 - protein, 12 aa, Artificial Sequence
  from human antibody library
RASQSVSSSYLA

SEQ ID NO: 138 - protein, 7 aa, Artificial Sequence
  from human antibody library
GASSRAT

SEQ ID NO: 139 - protein, 9 aa, Artificial Sequence
  from human antibody library
QQFDSSPPT

SEQ ID NO: 140 - protein, 109 aa, Artificial Sequence
  from human antibody library
DIQMTQSPGTLSLSPGERATLSCRASQSVSSSYLAWYQQKPGRAPRLLMYGASSRATGFPDRFSGSGSGTDFSLTISRLEPEDFAVYYCQQFDSSPPTFGGGTKVEIKR

SEQ ID NO: 141 - protein, 12 aa, Artificial Sequence
  Human antibody library
RASQSVSSSYLA

SEQ ID NO: 142 - protein, 7 aa, Artificial Sequence
  Human antibody library
GASSRAT

SEQ ID NO: 143 - protein, 10 aa, Artificial Sequence
  Human antibody library
QQFGSSPPYT

SEQ ID NO: 144 - protein, 110 aa, Artificial Sequence
  Human antibody library
DIQMTQSPGTLSLSPGERATLSCRASQSVSSSYLAWYQQKPGQAPRLLIYGASSRATGIPDRFSGSGSGTDYTLTINRLEPEDFAVYYCQQFGSSPPYTFGQGTKLEIKR

SEQ ID NO: 145 - protein, 12 aa, Artificial Sequence
  Human antibody library
RASQSVSSSYLA

SEQ ID NO: 146 - protein, 7 aa, Artificial Sequence
  Human antibody library
GASTRAT

SEQ ID NO: 147 - protein, 10 aa, Artificial Sequence
  Human antibody library
QQFDNWPPWT

SEQ ID NO: 148 - protein, 110 aa, Artificial Sequence
  Human antibody library
DIQMTQSPGTLSLSPGERATLSCRASQSVSSSYLAWYQQKPGQAPRLLIYGASTRATGIPPRFSGSGSGTEFTLTISSVQSEDFAIYYCQQFDNWPPWTFGQGTKVEIKR

SEQ ID NO: 149 - protein, 12 aa, Artificial Sequence
  Human antibody library
RASQSVSSNYFG

SEQ ID NO: 150 - protein, 7 aa, Artificial Sequence
  Human antibody library
GASSRAT

SEQ ID NO: 151 - protein, 9 aa, Artificial Sequence
  Human antibody library
QQFDSSPLT

SEQ ID NO: 152 - protein, 109 aa, Artificial Sequence
  Human antibody library
DIQMTQSPGTLSLSPGEGATLSCRASQSVSSNYFGWYQQKPGQAPRLLIYGASSRATGIPDRFSGSGSGTDFTLTISRLEPEDSAVYYCQQFDSSPLTFGGGTKVEIKR

SEQ ID NO: 153 - protein, 12 aa, Artificial Sequence
  from human antibody library
RASQSVSSNYLA

SEQ ID NO: 154 - protein, 7 aa, Artificial Sequence
  Human antibody library
GASSRAT

SEQ ID NO: 155 - protein, 9 aa, Artificial Sequence
  Human antibody library
QQFDSSPLS

SEQ ID NO: 156 - protein, 109 aa, Artificial Sequence; identical to SEQ ID NO: 124
  Human antibody library

SEQ ID NO: 157 - protein, 12 aa, Artificial Sequence
  Human antibody library
RASQSVSSNYLA

SEQ ID NO: 158 - protein, 7 aa, Artificial Sequence
  Human antibody library
GASTRAT

SEQ ID NO: 159 - protein, 9 aa, Artificial Sequence
  Human antibody library
QQFDSSPLS

SEQ ID NO: 160 - protein, 109 aa, Artificial Sequence
  Human antibody library
DIQMTQSPGTLSLSPGDRATLSCRASQSVSSNYLAWYQQKPGQAPRVLIYGASTRATGIPDRFTGSGSGTDFTLTISRLEPEDSAVYYCQQFDSSPLSFGGGTKVEIKR

SEQ ID NO: 161 - protein, 12 aa, Artificial Sequence
  Human antibody library
RASQSLNNNYLA

SEQ ID NO: 162 - protein, 7 aa, Artificial Sequence
  Human antibody library
GASTRAT

SEQ ID NO: 163 - protein, 9 aa, Artificial Sequence
  Human antibody library
QQFDSSPPT

SEQ ID NO: 164 - protein, 109 aa, Artificial Sequence
  Human antibody library
DIQMTQSPGTLSLSPGERATLSCRASQSLNNNYLAWYQQKPGQAPRLLMYGASTRATGIPARFSGSGSGTEFTLTISSLQSEDFAVYYCQQFDSSPPTFGGGTKVEIKR

SEQ ID NO: 165 - protein, 12 aa, Artificial Sequence
  Human antibody library
RASHSVSSDYLA

SEQ ID NO: 166 - protein, 7 aa, Artificial Sequence
  Human antibody library
GASSRAT

SEQ ID NO: 167 - protein, 9 aa, Artificial Sequence
  Human antibody library
QQFDSSPPT

SEQ ID NO: 168 - protein, 109 aa, Artificial Sequence
  Human antibody library
DIQMTQSPATLSLSPGERATLSCRASHSVSSDYLAWYQQKPGRAPRLLMYGASSRATGFPDRFSGSGSGTDFSLTISRLEPEDFAMYYCQQFDSSPPTFGGGTKVEIKR

SEQ ID NO: 169 - protein, 12 aa, Artificial Sequence
  from human antibody library
RASHSVSSDYLA

SEQ ID NO: 170 - protein, 7 aa, Artificial Sequence
  from human antibody library
GASSRAT

SEQ ID NO: 171 - protein, 9 aa, Artificial Sequence
  from human antibody library
QQFDSSPPT

SEQ ID NO: 172 - protein, 109 aa, Artificial Sequence
  from human antibody library
DIQMTQSPATLSVSPGERATLSCRASHSVSSDYLAWYQQKPGRAPRLLMYGASSRATGFPDRFSGSGSGTDFSLTISRLEPEDFAMYYCQQFDSSPPTFGGGTKVEIKR

SEQ ID NO: 173 - protein, 12 aa, Artificial Sequence
  Human antibody library
RASHSVSSDYLA

SEQ ID NO: 174 - protein, 7 aa, Artificial Sequence
  Human antibody library
GASSRAT

SEQ ID NO: 175 - protein, 9 aa, Artificial Sequence
  Human antibody library
QQFDSSPPT

SEQ ID NO: 176 - protein, 109 aa, Artificial Sequence
  Human antibody library
DIQMTQSPGTLSLSPGERATLSCRASHSVSSDYLAWYQQKPGRAPRLLMYGASSRATGFPDRFSGSGSGTDFSLTISRLEPEDFAVYYCQQFDSSPPTFGGGTKVEIKR

SEQ ID NO: 177 - protein, 12 aa, Artificial Sequence
  Human antibody library
RASHSVSSDYLA

SEQ ID NO: 178 - protein, 7 aa, Artificial Sequence
  Human antibody library
GASSRAT

SEQ ID NO: 179 - protein, 9 aa, Artificial Sequence
  Human antibody library
QQFDSSPPT

SEQ ID NO: 180 - protein, 111 aa, Artificial Sequence
  from human Fab library
IADIQMTQSPDTLSLSPGERATLSCRASHSVSSDYLAWYQQKPGRAPRLLMYGASSRATGFPDRFSGSGSGTDFSLTISRLEPEDFAVYYCQQFDSSPPTFGGGTKVEIKR

SEQ ID NO: 181 - protein, 12 aa, Artificial Sequence
  from human antibody library
RASHSVSSDYLA

SEQ ID NO: 182 - protein, 7 aa, Artificial Sequence
  from human antibody library
GASSRAT

SEQ ID NO: 183 - protein, 9 aa, Artificial Sequence
  from human antibody library
QQFDSSPPT

SEQ ID NO: 184 - protein, 109 aa, Artificial Sequence
  from human antibody library
DIQMTQSPDTLSLSPGERATLSCRASHSVSSDYLAWYQQKPGRAPRLLMYGASSRATGFPDRFSGSGSGTDFSLTISRLEPEDFAVYYCQQFDSSPPTFGGGTRIDIKR

SEQ ID NO: 185 - protein, 10 aa, Artificial Sequence
  consensus
GFTFSWYXMX

SEQ ID NO: 186 - protein, 17 aa, Artificial Sequence
  consensus
SIXXSGGXTXYADSVKG

SEQ ID NO: 187 - protein, 11 aa, Artificial Sequence
  consensus
XGXXLXXXXXS

SEQ ID NO: 188 - protein, 13 aa, Artificial Sequence
  consensus
SGSXSNXXXXXXX

SEQ ID NO: 189 - protein, 14 aa, Artificial Sequence
  consensus
XGXSXDXGXYDYVS

SEQ ID NO: 190 - protein, 7 aa, Artificial Sequence
  consensus
XXXXXPS

SEQ ID NO: 191 - protein, 10 aa, Artificial Sequence
  consensus
QXWXXXXXXX

SEQ ID NO: 192 - protein, 11 aa, Artificial Sequence
  consensus
AXWDDXLXXXX

SEQ ID NO: 193 - protein, 9 aa, Artificial Sequence
  consensus
MYSTITXLL

SEQ ID NO: 194 - protein, 13 aa, Artificial Sequence
  consensus
RASXXXXXXXYXX

SEQ ID NO: 195 - protein, 7 aa, Artificial Sequence
  consensus
GASXRAT

SEQ ID NO: 196 - protein, 10 aa, Artificial Sequence
  consensus
QQXXXXXXXX